(19) United States
(12) Patent Application Publication
(10) Pub. No.: US 2003/0064369 A1
(43) Pub. Date: Apr. 3, 2003

(54) Novel proteins and nucleic acids encoding same

(76) Inventors: Raymond J. Taupier JR., East Haven, CT (US); Muralidhara Padigaru, Branford, CT (US); Luca Rastelli, Guilford, CT (US); Steven Kurt Spaderna, Berlin, CT (US); Richard A. Shimkets, West Haven, CT (US); Bryan D. Zerhusen, Branford, CT (US); Kimberly Ann Spytek, New Haven, CT (US); Suresh G. Shenoy, Branford, CT (US); Li Li, Cheshire, CT (US); Vladimir Y. Gusev, Madison, CT (US); William M. Grosse, Branford, CT (US); John P. Alsobrook II, Madison, CT (US); Denise M. Lepley, Branford, CT (US); Catherine E. Burgess, Wethersfield, CT (US); Valerie L. Gerlach, Branford, CT (US); Karen Ellerman, Branford, CT (US); John R. MacDougall, Hamden, CT (US); David J. Stone, Guilford, CT (US); Glennda Smithson, Guilford, CT (US)

Correspondence Address: MINTZ, LEVIN, COHN, FERRIS,, GLOVSKY AND POPEO, P.C., One Financial Center, Boston, MA, 02111, US

(21) Appl. No.: 09/918,779

(22) Filed: Jul. 30, 2001

Related U.S. Application Data
(60) Provisional application No. 60/221,409, filed on Jul. 28, 2000.
(60) Provisional application No. 60/222,840, filed on Aug. 4, 2000.
(60) Provisional application No. 60/223,752, filed on Aug. 8, 2000.
(60) Provisional application No. 60/223,762, filed on Aug. 8, 2000.
(60) Provisional application No. 60/223,770, filed on Aug. 8, 2000.
(60) Provisional application No. 60/223,769, filed on Aug. 8, 2000.
(60) Provisional application No. 60/225,146, filed on Aug. 14, 2000.
(60) Provisional application No. 60/225,392, filed on Aug. 15, 2000.
(60) Provisional application No. 60/225,470, filed on Aug. 15, 2000.
(60) Provisional application No. 60/225,697, filed on Aug. 16, 2000.
(60) Provisional application No. 60/263,662, filed on Feb. 1, 2001.
(60) Provisional application No. 60/281,645, filed on Apr. 5, 2001.

(51) Int. Cl. C12Q 1/68; C07H 21/04; C12N 9/00; C07K 14/435; C12P 21/02; C12N 5/06

(52) U.S. Cl. 435/6; 435/69.1; 435/325; 435/320.1; 435/183; 530/350; 536/23.2

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from Applications U.S. Ser. No. 60/221,409, filed Jul. 28, 2000 (21402-074); U.S. Ser. No. 60/222,840, filed Aug. 4, 2000 (21402-076); U.S. Ser. No. 60/223,752, filed Aug. 8, 2000 (21402-077); U.S. Ser. No. 60/223,762, filed Aug. 8, 2000 (21402-081); U.S. Ser. No. 60/223,770, filed Aug. 8, 2000 (21402-082); U.S. Ser. No. 60/223,769, filed Aug. 8, 2000 (21402-083); U.S. Ser. No. 60/225,146, filed Aug. 14, 2000 (21402-087); U.S. Ser. No. 60/225,392, filed Aug. 15, 2000 (21402-088); U.S. Ser. No. 60/225,470, filed Aug. 15, 2000 (21402-089); U.S. Ser. No. 60/225,697, filed Aug. 16, 2000 (21402-090); U.S. Ser. No. 60/263,662, filed Feb. 1, 2001 (21402-088A); and 60/281,645, filed Apr. 5, 2001, each of which is incorporated by reference in its entirety.

### FIELD OF THE INVENTION

The invention generally relates to nucleic acids and polypeptides encoded therefrom.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9, NOV10, NOV11, NOV12, NOV13 and NOV14 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:1, 3, 5, 7, 9, 11, 13, 15, 17, 19, 21, 23, 25, and 27. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 20, 22, 24, 26 and 28. The nucleic acid can be, for example, a genomic DNA fragment or a cDNA molecule that includes the nucleic acid sequence of any of SEQ ID NOS:1, 3, 5, 7, 9, 11, 13, 15, 17, 19, 21, 23, 25, and 27.

Also included in the invention is an oligonucleotide, e.g., an oligonucleotide which includes at least 6 contiguous nucleotides of a NOVX nucleic acid (e.g., SEQ ID NOS:1, 3, 5, 7, 9, 11, 13, 15, 17, 19, 21, 23, 25, and 27) or a complement of said oligonucleotide.

Also included in the invention are substantially purified NOVX polypeptides (SEQ ID NOS:2, 4, 6, 8, 10, 12, 14, 16, 18, 20, 22, 24, 26 and 28). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, or other disorders related to cell signal processing and metabolic pathway modulation. The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from: diabetes, infections, neurological disorders, cancer, renal disease, hypertension and/or other pathologies and disorders of the like.

The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders.

The invention further includes a method for screening for a modulator of disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders or other disorders related to cell signal processing and metabolic pathway modulation. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders or other disorders related to cell signal processing and metabolic pathway modulation by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby.

Included in the invention are the novel nucleic acid sequences and their polypeptides. The sequences are collectively referred to as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ | SEQ | |
|---|---|---|---|---|
| | | ID NO | ID NO | |
| NOVX | Internal | (nucleic | (poly- | |
| Assignment | Identification | acid) | peptide) | Homology |
| 1 | SC_105828681_A | 1 | 2 | Transmembrane |
| | | | | receptor UNC5H1 |
| 2 | GM_ba113d19_A | 3 | 4 | Interferon Beta |
| 3 | ac009238_gene_{—} | 5 | 6 | Prominin |
| | 5_EXT | | | |
| 4 | ac009238_gene_5 | 7 | 8 | Prominin |
| 5 | SC_87081869_A | 9 | 10 | Glucose |
| | | | | Transporter |
| 6 | SC71046974_EXT | 11 | 12 | Na + H + |
| | | | | Exchanger |
| 7 | GMAC040907.3_A | 13 | 14 | Thymosin-Beta 4 |
| 8 | 20760813_EXT/ | 15 | 16 | Leucine-rich |
| | CG5151 4-01, | | | Repeat Protein |
| | CG51514-02 | | | |
| 9 | CG51514-03 | 17 | 18 | Leucine-rich |
| | | | | Repeat Protein |
| 10 | SC128855163_A | 19 | 20 | WNT-5A-like |
| 11 | CG56768-01 | 21 | 22 | WNT-5A-like |
| 12 | SC128855163_B | 23 | 24 | WNT-5A-like |
| 13 | SC55003337_A | 25 | 26 | Procollagen 1 |
| | | | | N-Protease |
| 14 | GMAC073150_A | 27 | 28 | 26S Protease |
| | | | | Regulatory |
| | | | | Subunit |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also used to identify proteins that are members of the family to which the NOVX polypeptides belong.

For example, NOV1 is homologous to the UNC5H1 transmembrane receptor family of protein which are crucial for axon guidance by interacting with netrin-1. Thus, the NOV1 nucleic acids, polypeptides, antibodies and related compounds according to the invention will be useful in therapeutic and diagnostic applications implicated in, for example; neurogenesis, nerve regenertion, retinal lesions and/or other pathologies/disorders.

Also, NOV2 is homologous to the interferon beta family of proteins. Thus NOV2 nucleic acids, polypeptides, antibodies and related compounds according to the invention will be useful in therapeutic and diagnostic applications implicated in, for example; viral infections, autoimmune disease, multiple sclerosis, optic neuritis, focal epithelial hyperplasia and subacute sclerosing panencephalitis and/or other pathologies/disorders.

Further, NOV3 and NOV4 are homologous to a family of prominin-like membrane proteins which are important in cholesterol transport and the biogenesis of rod photoreceptor cells. Thus, the NOV3 NOV4 nucleic acids and polypeptides, antibodies and related compounds according to the invention will be useful in therapeutic and diagnostic applications implicated in, for example; retinal degeneration and/or other pathologies/disorders.

Also, NOV5 is homologous to a glucose transport family of proteins which are important in glucose storage and transport. Thus, NOV5 nucleic acids, polypeptides, antibodies and related compounds according to the invention will be useful in therapeutic and diagnostic applications implicated in, for example; diabetes, renal disease, vascular disease and/or other pathologies/disorders.

Additionally, NOV6 is homologous to a Na+H+ Exchanger family of proteins. These proteins are important in establishing and maintaining intracellular sodium and hydrogen levels. Thus NOV6 nucleic acids, polypeptides, antibodies and related compounds according to the invention will be useful in treating a variety of conditions, including renal disease, hypertension, myocardial ischemia, congenital sodium diarrhea, diffuse corporal gastritis and/or other pathologies/disorders.

Also, NOV7 is homologous to the beta thymosin family of proteins. Thus NOV7 nucleic acids, polypeptides, antibodies and related compounds according to the invention will be useful in therapeutic and diagnostic applications implicated in, for example; prostate cancer, apoptosis, angiogenesis and wound healing, neurodegenerative and neuropsychiatric disease, immune and autoimmune disorders, age-related disorders and/or other pathologies/disorders.

Still further, NOV8 and NOV9 are homologous to a family of Leucine-rich repeat proteins that mediate protein-protein interactions and are important in a variety of functions such as cell adhesion, motility, cell signalling and proliferation. Thus, NOV8 and NOV9 nucleic acids and polypeptides, antibodies and related compounds according to the invention will be useful in therapeutic and diagnostic applications in disorders including, for example, Crohn's disease, embryogenesis, pathogen infection, and/or other pathologies/disorders.

Also, NOV10-12 are homologous to the WNT-5A family of nuclear signaling proteins. Thus NOV10-12 nucleic acids, polypeptides, antibodies and related compounds according to the invention will be useful in therapeutic and diagnostic applications implicated in, for example; autism, Alzheimer's disease, Abeta neurotoxicity, familial adenomatous polyposis coli, cancer and/or other pathologies/disorders.

Additionally, NOV13 is homologous to the procollagen I-N-proteinase family of proteins, which are zinc-binding metalloproteinases. Thus NOV13 nucleic acids, polypeptides, antibodies and related compounds according to the invention will be useful in therapeutic and diagnostic applications implicated in, for example; Ehlers-Danlos syndrome type VII C and/or other pathologies/disorders.

Finally, NOV14 is homologous to the 26S protease regulatory subunit family of proteins. Thus, NOV14 nucleic acids and polypeptides, antibodies and related compounds according to the invention will be useful in therapeutic and diagnostic applications in various disorders including, for example, Alzheimer's disease, Parkinson's disease, cutaneous malignant melanoma and/or other pathologies/disorders.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

### NOV1

A NOV1 sequence (also referred to as SC_{—}105828681_A) according to the invention includes a nucleic acid sequence encoding a polypeptide related to the UNC5H1 family of proteins. Tables 1A and 1B show a NOV1 nucleic acid and its encoded polypeptide sequence, respectively. A disclosed NOV1 nucleic acid of 2752 nucleotides is shown in Table 1A. The disclosed NOV1 open reading frame (“ORF”) was identified beginning with an ATG initiation codon at nucleotides 46-48 and ending with a TGA codon at nucleotides 2740-2742. As shown in Table 1A, the start and stop codons are in bold letters.

TABLE 1A
NOV1 nucleotide sequence (SEQ ID NO:1).

CCGCGGGGCCCCGCGCCCGGCCCGCCCGCCTGCCCGCCCGCGGCCATGGCCGTCCGGCCCGGCCTGTGGC
CAGCGCTCCTGGGCATAGTCCTCGCCGCTTGGCTCCGCGGCTCGGGTGCCCAGCAGAGTGCCACCGTGGC
CAACCCAGTGCCTGGTGCCAACCCGGACCTGCTTCCCCACTTCCTGGTGGAGCCCGAGGATGTGTACATC
GTCAAGAACAAGCCAGTGCTGCTTGTGTGCAAGGCCGTGCCCGCCACGCAGATCTTCTTCAAGTGCAACG
GGGAGTGGGTGCGCCAGGTGGACCACGTGATCGAGCGCAGCACAGACGGGAGCAGTGGGCTGCCCACCAT
GGAGGTCCGCATTAATGTCTCAAGGCAGCAGGTCGAGAAGGTGTTCGGGCTGGAGGAATACTGGTGCCAG
TGCGTGGCATGGAGCTCCTCGGGCACCACCAAGAGTCAGAAGGCCTACATCCGCATAGCCAGATTGCGCA
AGAACTTCGAGCAGGAGCCGCTGGCCAAGGAGGTGTCCCTGGAGCAGGGCATCGTGCTGCCCTGCCGTCC
ACCGGAGGGCATCCCTCCAGCCGAGGTGGAGTGGCTCCGGAACGAGGACCTGGTGGACCCGTCCCTGGAC
CCCAATGTATACATCACGCGGGAGCACAGCCTGGTGGTGCGACAGGCCCGCCTTGCTGACACGGCCAACT
ACACCTGCGTGGCCAAGAACATCGTGGCACGTCGCCGCAGCGCCTCCGCTGCTGTCATCGTCTACGTGAA
CGGTGGGTGGTCGACGTGGACCGAGTGGTCCGTCTGCAGCGCCAGCTGTGGGCGCGGCTGGCAGAAACGG
AGCCGGAGCTGCACCAACCCGGCGCCTCTCAACGGGGGCGTTTCTGTGAGGGGCAGAATGTCCAGAAAA
CAGCCTGCGCCACCCTGTGCCCAGTAGACGGCAGCTGGAGCCCGTGGAGCAAGTGGTCGGCCTGTGGGCT
GGACTGCACCCACTGGCGGAGCCGTGAGTGCTCTGACCCAGCACCCCGCAACGGAGGGGAGGAGTGCCAG
GGCACTGACCTGGACACCCGCAACTGTACCAGTGACCTCTGTGTACACAGYGCTTCTGGCCCTGAGGACG
TGGCCCTCTATGTGGGCCTCATCGCCGTGGCCGTCTGCCTGGTCCTGCTGCTGCTTGTCCTCATCCTCGT
TTATTGCCGGAAGAAGGAGGGGCTGGACTCAGATGTGGCTGACTCGTCCATTCTCACCTCAGGCTTCCAG
CCCGTCAGCATCAAGCCCAGCAAAGCAGACAACCCCCATCTGCTCACCATCCAGCCGGACCTCAGCACCA
CCACCACCACCTACCAGGGCAGTCTCTGTCCCCGGCAGGATGGGCCCAGCCCCAAGTTCCAGCTCACCAA
TGGGCACCTGCTCAGCCCCCTGGGTGGCGGCCGCCACACACTGCACCACAGCTCTCCCACCTCTGAGGCC
GAGGAGTTCGTCTCCCGCCTCTCCACCCAGAACTACTTCCGCTCCCTGCCCCGAGGCACCAGCAACATGA
CCTATGGGACCTTCAACTTCCTCGGGGGCCGGCTGATGATCCCTAATACAGGTATCAGCCTCCTCATCCC
CCCAGATGCCATACCCCGAGGGAAGATCTATGAGATCTACCTCACGCTGCACAAGCCGGAAGACGTGAGG
TTGCCCCTAGCTGGCTGTCAGACCCTGCTGAGTCCCATCGTTAGCTGTGGACCCCCTGGCGTCCTGCTCA
CCCGGCCAGTCATCCTGGCTATGGACCACTGTGGGGAGCCCAGCCCTGACAGCTGGAGCCTGCGCCTCAA
AAAGCAGTCGTGCGAGGGCAGCTGGGAGGATGTGCTGCACCTGGGCGAGGAGGCGCCCTCCCACCTCTAC
TACTGCCAGCTGGAGGCCAGTGCCTGCTACGTCTTCACCGAGCAGCTGGGCCGCTTTGCCCTGGTGGGAG
AGGCCCTCAGCGTGGCTGCCGCCAAGCGCCTCAAGCTGCTTCTGTTTGCGCCGGTGGCCTGCACCTCCCT
CGAGTACAACATCCGGGTCTACTGCCTGCATGACACCCACGATGCACTCAAGGAGGTGGTGCAGCTGGAG
AAGCAGCTGGGGGGACAGCTGATCCAGGAGCCACGGGTCCTGCACTTCAAGGACAGTTACCACAACCTGC
GCCTATCCATCCACGATGTGCCCAGCTCCCTGTGGAAGAGTAAGCTCCTTCGTCAGCTACCAGGAGATCCC
CTTTTATCACATCTGGAATGGCACGCAGCGGTACTTGCACTGCACCTTCACCCTGGAGCGTGTCAGCCCC
AGCACTAGTGACCTGGCCTGCAAGCTGTGGGTGTGGCAGGTGGAGGGCGACGGGCAGAGCTTCAGCATCA
ACTTCAACATCACCAAGGACACAAGGTTTGCTGAGCTGCTGGCTCTGGAGAGTGAAGCGGGGGTCCCAGC
CCTGGTGGGCCCCAGTGCCTTCAAGATCCCCTTCCTCATTCGGCAGAAGATAATTTCCAGCCTGGACCCA
CCCTGTAGGCGGGGTGCCGACTGGCGGACTCTGGCCCAGAAACTCCACCTGGACAGCCATCTCAGCTTCT
TTGCCTCCAAGCCCAGCCCCACAGCCATGATCCTCAACCTGTGGGAGGCGCGGCACTTCCCCAACGGCAA
CCTCAGCCAGCTGGCTGCAGCAGTGGCTGGACTGGGCCAGCCAGACGCTGGCCTCTTCACAGTGTCGGAG
GCTGAGTGCTGAGGCCGGCCAG

In a search of public sequence databases, it was found, for example, that the NOV1 nucleic acid sequence disclosed in this invention has 2419 of 2697 bases (89 %) identical to one region of a Rattus novegicus Unc5H1 mRNA, 2697 bp, with an E-value of 0.0 (GENBANK-ID: RNU87305). Public nucleotide databases include all GenBank databases and the GeneSeq patent database.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 BLAST analysis, e.g., Rattus novegicus Unc5H1 mRNA, matched the Query NOV1 sequence purely by chance is 1.0×10⁻⁹⁹. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. Wootton and Federhen, Methods Enzymol 266:554-571, 1996.

A disclosed encoded NOV1 protein has 898 amino acid residues and a predicted molecular weight of 98,841.9 Daltons, referred to as the NOV1 protein. The NOV1 protein was analyzed for signal peptide prediction and cellular localization. PSORT analysis predicts the protein of the invention to be localized in the plasma membrane with a certainty of 0.4600. Using the SIGNALP analysis, it is predicted that the protein of the invention has a signal peptide with most likely cleavage site between pos. 25 and 26 of SEQ ID NO.:2. The disclosed NOV1 polypeptide sequence is presented in Table 1B using the one-letter amino acid code.

TABLE 1B
Encoded NOV1 protein sequence (SEQ ID NO:2).

MAVRPGLWPALLGIVLAAWIRGSGAQQSATVANPVPGANPDLLPHFLVEPEDVYIVKNKPVLLV
CKAVPATQIFFKCNGEWVRQVDHVIERSTDGSSGLPTMEVRINVSRQQVEKVFGLEEYWCQCVA
WSSSGTTKSQKAYIRIARLRKNFEQEPLAKEVSLEQGIVLPCRPPEGIPPAEVEWLRNEDLVDP
SLDPNVYITREHSLVVRQARLADTANYTCVAKNIVARRRSASAAVIVYVNGGWSTWTEWSVCSA
SCGRGWQKRSRSCTNPAPLNGGAFCEGQNVQKTACATLCPVDGSWSPWSKWSACGLDCTHWRSR
ECSDPAPRNGGEECQGTDLDTRNCTSDLCVHSASGPEDVALYVGLIAVAVCLVLLLLVLILVYC
RKKEGLDSDVADSSILTSGFQPVSIKPSKADNPHLLTIQPDLSTTTTTYQGSLCPRQDGPSPKF
QLTNGHLLSPLGGGRHTLHHSSPTSEAEEFVSRLSTQNYFRSLPRGTSNMTYGTFNFLGGRLMI
PNTGISLLIPPDAIPRGKIYEIYLTLHKPEDVRLPLAGCQTLLSPIVSCGPPGVLLTRPVILAM
DHCGEPSPDSWSLRLKKQSCEGSWEDVLHLGEEAPSHLYYCQLEASACYVFTEQLGRFALVGEA
LSVAAAKRLKLLLFAPVACTSLEYNIRVYCLHDTHDALKEVVQLEKQLGGQLIQEPRVLHFKDS
YHNLRLSIHDVPSSLWKSKLLVSYQEIPPYHIWNGTQRYLHCTFTLERVSPSTSDLACKLWVWQ
VEGDGQSFSINFNITKDTRFAELLALESEAGVPALVGPSAFKIPFLIRQKIISSLDPPCRRGAD
WRTLAQKLHLDSHLSFFASKPSPTAMILNLWEARHFPNGNLSQLAAAVAGLGQPDAGLFTVSEA
EC

NOV1 sequences were initially identified by searching a proprietary sequence file database for DNA sequences which translate into proteins with similarity to a protein family of interest. NOV1 was identified as having suitable similarity. NOV1 was analyzed further to identify any open reading frames encoding novel full length proteins, as well as, novel splice forms of NOV1. This was done by extending the identified NOV1 using suitable sequences from additional proprietary assemblies, publicly available EST sequences and public genomic sequences. In a search of CuraGen's proprietary human expressed sequence assembly database, assembly s3aq:105828681 (342 nucleotides) was identified as having >95% homology to this predicted TRANSMEMBRANE RECEPTOR UNC5H1 sequence. This database is composed of the expressed sequences (as derived from isolated mRNA) from more than 96 different tissues.

The genomic clones were analysed by Genscan and Grail to identify exons and putative coding sequences/open reading frames. This clone was also analyzed by TblastN, BlastX and other homology programs to identify regions translating to proteins with similarity to the original protein/protein family of interest. Expressed sequences from both public and proprietary databases were also added when available to further define and complete the gene sequence. The DNA sequence was then manually corrected for apparent inconsistencies thereby obtaining the sequences encoding the full-length protein.

A NOV1 nucleic acid has homology (310/310 bases) with a region of human chromosome 5 (GenBank Accession Number NT_{—}006725.4), which specifically maps NOV1 to 5q35. This locus is associated with a number of pathologies, including neurogenic arthrogryposis multiplex congenita, type 2 craniosynostosis, NPM/RARA type acute promyelocytic leukemia, and leukotriene C4 synthase deficiency.

A BLASTX search was performed against public protein databases. The disclosed NOV1 protein (SEQ ID NO:2) has good identity with Unc5H1-like proteins. For example, the full amino acid sequence of the protein of the invention was found to have 862 of 898 amino acid residues (95%) identical to, and 879 of 898 residues (97%) similar to, the 898 amino acid residue Unc4H1 protein from Rattus norvegicus (SPTREMBL-ACC:008721; E=0.0). Public amino acid databases include the GenBank databases, SwissProt, PDB and PIR.

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/ interpro). A NOV1 protein contains the following protein domains (as defined by Interpro) at the indicated nucleotide positions: ZU5 domain (IPR000906) at amino acid positions 495 to 598, Thrombospondin type I domain (IPR000884) at amino acid positions 246 to 295, Death domain (IPR000488) at amino acid positions 817 to 897, Immunoglobulin domain (IPR003006) at amino acid positions 163 to 223, Somatotropin hormone family (IPR001400) at amino acid positions 372 to 389, Keratin, high sulfur B2 protein (IPR002494) at amino acid positions 232 to 348.

ZU5 domain is a domain of unknown function, present in ZO-1 and Unc5-like netrin receptors. It is also found in different variants of ankyrin, which are responsible for attaching integral membrane proteins to cytoskeletal elements.

The Thrombospondin type 1 domain is found in the thrombospondin protein where it is repeated 3 times. Now a number of proteins involved in the complement pathway (properdin, C6, C7, C8A, C8B, C9) as well as extracellular matrix protein like mindin, F-spondin, SCO-spondin and even the circumsporozoite surface protein 2 and TRAP proteins of Plasmodium, contain one or more instance of this repeat. It has been involved in cell-cell interraction, inhibition of angiogenesis and apoptosis.

The death domain (FAS/TNF cytosolic interaction domain) has first been described as a region in the cytoplasmic tail of the 75 Kd TNF receptor (TNFR-1) which is involved in TNF-mediated cell death signaling. A corresponding region is found in the cytoplasmic tail of FAS/APO1 another surface receptor inducing apoptotic cell death. This region mediates self-association of these receptors, thus giving the signal to downstream events leading to apoptosis. Subsequently, a number of other proteins have been found to interact with the cytoplasmic part of either FAS or the TNF receptor in the region of the death domain. Overexpression of these proteins usually leads to cell death. By profile analysis, it has been shown that a number of other proteins contain regions with significant similarity to the death domain. Interestingly, several of these proteins also work in the context of cell death signaling. In most of these proteins, the death domain is located at the extreme C- terminus. Exceptions are ankyrin, MyD88 and pelle, all protein probably not directly involved in cell death signaling. In the case of ankyrin, the isoform 2.1 is a splice variant which has the death domain located at the C-terminus.

Members of the immunoglobulin superfamily are found in hundreds of proteins of different functions. Examples include antibodies, the giant muscle kinase titin and receptor tyrosine kinases. Immunoglobulin-like domains may be involved in protein-protein and protein-ligand interactions. The Pfam alignments do not include the first and last strand of the immunoglobulin-like domain.

Somatotropin is a hormone that plays an important role in growth control. It belongs to a family that includes choriomammotropin (lactogen), its placental analogue; prolactin, which promotes lactation in the mammary gland, and placental prolactin-related proteins; proliferin and proliferin related protein; and somatolactin from various fish. The 3D structure of bovine somatotropin has been predicted using a combination of heuristics and energy minimization.

High sulfur proteins are cysteine-rich proteins synthesized during the differentiation of hair matrix cells, and form hair fibers in association with hair keratin intermediate filaments. This family has been divided up into four regions, with the second region containing 8 copies of a short repeat . This family is also known as B2 or KAP1.

BLAST results include sequences from the Patp database, which is a proprietary database that contains sequences published in patents and patent publications. Patp results include those listed in Table 1C.

TABLE 1C

Patp alignments of NOV1

| | | | |
|---|---|---|---|
| | | | Smallest |
| | | | Sum |
| | Reading | High | Prob. |
| Sequences producing High-scoring Segment Pairs: | Frame | Score | P(N) |

| | | | |
|---|---|---|---|
| Patp:AAW78898 Rat UNC-5 homologue UNC5H1-Rattus sp. 898aa | +1 | 4638 | 0.0 |

Quantitative gene expression analysis (TaqMan) was performed on a NOV1 nucleic acid. The general method is described in Example 1. Results of TaqMan analysis of a NOV1 nucleic acid are described in Example 2.

Unc5H1-like proteins are important in neurogenesis due to their interactions with the netrin family of proteins. The netrins comprise a small phylogenetically conserved family of guidance cues important for guiding particular axonal growth cones to their targets. Two netrin genes, netrin-1 and netrin-2, have been described in chicken. In mouse an ortholog of chick netrin-1 and a second mouse netrin gene, netrin-3, have been reported. Netrin-3 does not appear to be the ortholog of chick netrin-2 but is the ortholog of a recently identified human netrin gene termed NTN2L (“netrin-2-like”), as evidenced by a high degree of sequence conservation and by chromosomal localization. Netrin-3 is expressed in sensory ganglia, mesenchymal cells, and muscles during the time of peripheral nerve development but is largely excluded from the CNS at early stages of its development. The murine netrin-3 protein binds to netrin receptors of the DCC (deleted in colorectal cancer) family [DCC and neogenin] and the UNC5 family (UNC5H1, UNC5H2 and UNC5H3). Unlike chick netrin-1, however, murine netrin-3 binds to DCC with lower affinity than to the other four receptors. Consistent with this finding, although murine netrin-3 can mimic the outgrowth-promoting activity of netrin-1 on commissural axons, it has lower specific activity than netrin-1. Thus, like netrin-1, netrin-3 may also function in axon guidance during development but may function predominantly in the development of the peripheral nervous system and may act primarily through netrin receptors other than DCC.

Migration of neurons from proliferative zones to their functional sites is fundamental to the normal development of the central nervous system. Mice homozygous for the rostral cerebellar malformation (rcm) mutation exhibit cerebellar and midbrain defects, apparently as a result of abnormal neuronal migration. It has been reported that in rcm-mutant mice, the cerebellum is smaller and has fewer folia than in wildtype, ectopic cerebellar cells are present in midbrain regions by 3 days after birth, and there are abnormalities in postnatal cerebellar-neuronal migration. The authors isolated cDNAs encoding the rcm protein (Rcm). Sequence analysis revealed that the predicted 931-amino acid mouse protein is a transmembrane protein that contains 2 immunoglobulin (Ig)-like domains and 2 type I thrombospondin (THBS1) motifs in the extracellular region. Ig and THBS 1 domains are also found in the extracellular region of the C. elegans UNC5 transmembrane protein, and the C-terminal 865-amino acid region of Rcm is 30% identical to UNC5. UNC5 protein is essential for dorsal guidance of pioneer axons and for the movement of cells away from the netrin ligand. In the developing brain of vertebrates, netrin-1 plays a role in both cell migration and axonal guidance. Rcm binds netrin-1 in vitro. Rcm and its ligand are important in critical migratory and/or cell-proliferation events during cerebellar development. Disruption of the mouse rcm gene, also called the Unc5h3 gene, resulted in a failure of tangentially migrating granule cells to recognize the rostral boundary of the cerebellum.

Netrins are bifunctional: they attract some axons and repel others. Netrin receptors of the Deleted in Colorectal Cancer (DCC) family are implicated in attraction and those of the UNC5 family in repulsion, but genetic evidence also suggests involvement of the DCC protein UNC-40 in some cases of repulsion. Attraction is converted to repulsion by expression of UNC5 proteins in these cells, that this repulsion requires DCC function, that the UNC5 cytoplasmic domain is sufficient to effect the conversion, and that repulsion can be initiated by netrin-1 binding to either UNC5 or DCC. The isolated cytoplasmic domains of DCC and UNC5 proteins interact directly, but this interaction is repressed in the context of the full-length proteins. Netrin- 1 triggers the formation of a receptor complex of DCC and UNC5 proteins and simultaneously derepresses the interaction between their cytoplasmic domains, thereby converting DCC-mediated attraction to UNC5/DCC-mediated repulsion.

The above defined information for this invention suggests that this novel UNC5H1-like protein (NOV1) may function as a member of a UNC5H 1 family. Therefore, the expression nucleic acids and proteins of NOV1 re useful in potential therapeutic applications implicated in various UNC5H1-related pathologies and/or disorders. For example, a cDNA encoding the UNC5H1-like protein may be useful in gene therapy, and the UNC5H1-like protein may be useful when administered to a subject in need thereof. The novel nucleic acid encoding NOV1 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The NOVX nucleic acids and proteins of the invention are useful in potential therapeutic applications implicated in various diseases and disorders described below and/or other pathologies and disorders. For example, but not limited to, a cDNA encoding the Unc5H1-like protein may be useful in gene therapy, and the Unc5H 1-like protein may be useful when administered to a subject in need thereof. By way of nonlimiting example, the compositions of the present invention will have efficacy for treatment of patients suffering from neurological and/or other pathologies/disorders. The novel nucleic acid encoding the Unc5H1-like protein, and the Unc5H1-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

Further, the protein similarity information, expression pattern, and map location for NOV1 suggests that NOV1 may have important structural and/or physiological functions characteristic of the Unc5H1 family. Therefore, the nucleic acids and proteins of the invention are useful in potential diagnostic and therapeutic applications and as a research tool. Potential therapeutic uses for the compositions of the invention included, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

These materials are further useful in the generation of antibodies that bind immuno-specifically to the novel NOV1 substances for use in therapeutic or diagnostic methods. These antibodies may be generated according to methods known in the art, using prediction from hydrophobicity charts, as described in the “Anti-NOVX Antibodies” section below. The disclosed NOV1 protein has multiple hydrophilic regions, each of which can be used as an immunogen. In one embodiment, a contemplated NOV1 epitope is from about amino acids 495 to 598. In another embodiment, a NOV1 epitope is from about amino acids 246 to 295. In additional embodiments, NOV1 epitopes are from amino acids 817 to 897, 163-223, 372-389, and from amino acids 232 to 348. These novel proteins can be used in assay systems for functional analysis of various human disorders, e.g. neurological disorders, which will help in understanding of pathology of the disease and development of new drug targets for various disorders.

### NOV2

A novel nucleic acid was identified on chromosome 9 by TblastN using CuraGen Corporation's sequence file for interferon-beta protein or homolog as run against the Genomic Daily Files made available by GenBank or from files downloaded from the individual sequencing centers. The nucleic acid sequence was predicted from the genomic file GB ACCNO:ba 113d19 by homology to a known interferon beta or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added, when available, to further define and complete the gene sequence.

The novel nucleic acid of 604 nucleotides (also referred to as GM_ba113d19_A) encoding a novel interferon beta-like protein is shown in Table 2A. An open reading frame was identified beginning with an ATG initiation codon at nucleotides 12-14 and ending with a TAA codon at nucleotides 591-593. A putative untranslated region upstream from the initiation codon and downstream from the termination codon is underlined in Table 2A, and the start and stop codons are in bold letters.

TABLE 2A
NOV2 Nucleotide Sequence (SEQ ID NO:3)

AGGAACTCAACATGACTAGTCAATGCTTGCTGGATTGGGCCTTGGTGCTACTTCTCACCACTA
CTGCATTCTCTCTGGACTGTCACTTTCAAAGGTGCAAGGGCAACTGGGAGATTTTAGAACATT
TAAAAAACCTAGGAGAAAAATTTCCTCTGCAATGTCTAAAGGACAGGAGCAACTTCAGATTCT
TCCAGGTTTCTAAAAGTAACCTGTTTTCAAAGGAAAATGCCCTCATTGCCAAAAAAGAAATGT
TACAGCAGATATTCAACACTTTCAGCCTTAATGTCTCCCAATCTTTTTGGAATGAAAGCAGCTT
GGAGAGATTCCTAAGTAGACTTTATCAGCAAATAGAGAAGACAGAGGTGTGTTTGGAGCAGG
AAACCAGGAAAGAGGGCCGTTCACTCTTGCAAAGGGGGAATACCATATTTAGACTAAAAAATT
ATTTCCAAGGGATTCACAACTACTTACACCACCAAAATTATAGCAACTGTGCCTGGGAGGTCA
TCCATGTTGAAATCCGAAGGGGTCTACTATTTATTGAACAGTGCACAAGAAGACTCCAATACC
AAGAAACAGGTTATTTACATAAATAA+E,uns1 CTAATTTGGAG

In a search of public sequence databases, it was found, for example, that the nucleic acid sequence (NOV2) has 361 of 583 bases (61%) identical to a human interferon beta mRNA (GENBANK-ID: HSIFNA6; acc:X02958) (E=3.4 e−16). Public nucleotide databases include all GenBank databases and the GeneSeq patent database.

A NOV2 nucleic acid maps to the p21-22 region of human chromosome 9. Loci in this region are associated with diaphyseal medullary stenosis with malignant fibrous histiocytoma, cartilage-hair hypoplasia, immotile cilia syndrome-1, Kartagener syndrome and/or other dieases/pathologies.

The disclosed NOV2 polypeptide (SEQ ID NO:4) encoded by SEQ ID NO:3 is 193 amino acid residues with a predicted molecular weight of 23,123.3 Da, and is presented using the one-letter code in Table 2B. The NOV2 protein was analyzed for signal peptide prediction and cellular localization. SignalP, Psort and Hydropathy results predict that NOV2 containS a predicted signal peptide with the most likely cleavage site between positions 21 and 22 of SEQ ID NO. 4 and that NOV2 is likely to be extracellular with a certainty of 0.5135.

TABLE 2B
Encoded NOV2 protein sequence (SEQ ID NO:4).

MTSQCLLDWALVLLLTTTAFSLDCHFQRCKGNWEILEHLKNLGEKFPLQCLKDRSNFRFFQVSKSN
LFSKENALIAKKEMLQQIFNTFSLNVSQSFWNESSLERFLSRLYQQIEKTEVCLEQETRKEGRSLLQR
GNTIFRLKNYFQGIHNYLHHQNYSNCAWEVIHVEIRRGLLFIEQCTRRLQYQETGYLHK

The full amino acid sequence of the NOV2 protein of the invention was found to have 68 of 186 amino acid residues (36%) identical to, and 102 of 186 residues (54%) positive with, the 187 amino acid residue human interferon beta protein (ptnr: PIR: SWISSPROT-ACC: P01574; E=1.2 e−22). The global sequence homology is 40% amino acid similarity and 46% amino acid identity. In addition, this protein contains the interferon alpha/beta protein domain (as defined by Interpro# IPR00471) at amino acid positions 1 to 44. Public amino acid databases include the GenBank databases, SwissProt, PDB and PIR.

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

SNPs are confirmed employing a validated method know as Pyrosequencing (See Alderbom et al., Genome Research 10(8):1249-65, 2000). In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfuylase, which result in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels.

Possible SNPs found for NOV2 are listed in Table 2C.

TABLE 2C
NOV2 SNPs

| Base | Base | Alteration |
|---|---|---|
| Position | Before/After | effect |
| 93 | A/G | Arg to Gly |
| 213 | T/C | Phe to Leu |
| 316 | T/C | Leu to Ser |
| 391 | A/G | Glu to Gly |
| 466 | T/C | Leu to Ser |

Quantitative gene expression analysis (TaqMan) was performed on a NOV2 nucleic acid. The general method is described in Example 1. Results of TaqMan analysis of a NOV2 nucleic acid are described in Example 3.

By study of human-mouse cell hybrids, Meager et al. (1979) concluded that chromosome 5 is not involved in production of interferon. Instead they found correlation between interferon production and chromosome 9. The interferon produced by the hybrids was predominantly of the fibroblast type. From the nucleotide sequence of the gene for fibroblast interferon, cloned by recombinant DNA technology, Derynck et al. (1980) deduced the complete amino acid sequence of the protein. It is 166 amino acids long. Cavalieri et al. (1977) showed that leukocyte and fibroblast interferon are encoded by different species of mRNA. That these arise from separate genes (rather than being derived from the same gene through a common precursor which is processed or spliced in different modes)was demonstrated by Taniguchi et al. (1980). Between leukocyte and fibroblast interferon, they also found 45% homology at the nucleotide level and 29% at the amino acid level. Chany et al. (1980) likewise concluded that chromosome 9 carries a locus for an interferon, which they referred to as beta. Chromosome 13 also appeared to be involved. Chany et al. (1980) suggested that the locus on chromosome 13 might have something to do with alpha-interferon (147660) synthesis. Tavernier et al. (1981) presented evidence for a single fibroblast interferon gene. As in the case of IFN-alpha, no intervening sequences were discovered. Houghton et al. (1981) independently arrived at the same findings. Leukocyte interferon is produced predominantly by B lymphocytes. Immune interferon (IFN-gamma; 147570) is produced by mitogen- or antigen-stimulated T lymphocytes. Using radioactive probes from purified cDNA clones of interferons, Owerbach et al. (1981) located at least 8 leukocyte interferon genes and a fibroblast interferon gene on chromosome 9.

Ohno and Taniguchi (1981) also showed that the beta-interferon gene(s), like the alpha-interferon genes, lack intervening sequences. As noted above, comparison of the cDNA sequence of alpha and beta interferons shows apparent homology in amino acid sequence and in nucleotide sequence. They were presumably derived from a common ancestor. The fact that they are syntenic supports that conclusion. By in situ hybridization, Trent et al. (1982) confirmed the location of IFF and IFL on 9p and concluded that IFF is distal to IFL. They mapped IFB to 9p21-pter. Studying 2 patients with unbalanced rearrangements of 9p, Henry et al. (1984) used a genomic clone for IFB1 and concluded that the gene is located on 9p21. The presence of functional interferon-beta genes on chromosomes 2, 5 and 9 had been suggested. Sagar et al. (1984) concluded that IFN-beta-related DNA is dispersed in the human genome. The data from study of human-rodent somatic cell hybrids induced with poly(I)poly(C) or with viral inducers are consistent with assignment of IFB mRNA species of different lengths to chromosome 9 (IFB1), chromosome 5 (IFB2) and chromosome 2 (IFB3) (reviewed by Sagar et al., 1984). Another (IFB4) had been assigned to chromosome 4 (Sehgal et al., 1983). Ohlsson et al. (1985) identified 5 RFLPs associated with the alpha- and beta-interferon gene cluster. Heterozygosities made them excellent markers for the short arm of chromosome 9. In a study of 25 Caucasian families, no recombination was found between the alpha and beta markers. Furthermore, 12 of 32 possible haplotypes were found, indicating linkage disequilibrium which was of similar magnitude between various alpha markers as it was between alpha and beta markers. Thus, the alpha and beta genes must be clustered within a few hundred kilobases. Duplication of the beta gene, apparently of recent origin, was found in some persons and segregated regularly.

The above defined information for this invention suggests that this interferon beta-like protein may function as a member of an “interferon beta family”. Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of the invention are useful in potential therapeutic applications implicated in cancer including but not limited to prostate cancer, immunological and autoimmune disorders (i.e. hyperthyroidism), angiogenesis and wound healing, modulation of apoptosis, neurodegenerative and neuropsychiatric disorders, age-related disorders, and other pathological disorders involving spleen, thymus, lung, and peritoneal macrophages and/or other pathologies and disorders. For example, a cDNA encoding the interferon beta-like protein may be useful in gene therapy, and the interferon beta-like protein may be useful when administered to a subject in need thereof. By way of nonlimiting example, the compositions of the present invention will have efficacy for treatment of patients suffering from cancer including but not limited to prostate cancer, immunological and autoimmune disorders (ie hyperthyroidism), angiogenesis and wound healing, modulation of apoptosis, neurodegenerative and neuropsychiatric disorders, age-related disorders, and other pathological disorders involving spleen, thymus, lung, and peritoneal macrophages. The novel nucleic acid encoding interferon beta-like protein, and the interferon beta-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The novel nucleic acid encoding the interferon beta-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV2 proteins have multiple hydrophilic regions, each of which can be used as an immunogen. These novel proteins can be used in assay systems for functional analysis of various human disorders, which will help in understanding of pathology of the disease and development of new drug targets for various disorders.

### NOV3 and NOV4

A NOV3 nucleic acid was identified on chromosome 2 by TblastN using CuraGen Corporation's sequence file for prominin or homologs as run against the Genomic Daily Files made available by GenBank or from files downloaded from the individual sequencing centers. The nucleic acid sequence was predicted from the genomic file Sequencing Center accession number: AC009238 by homology to a known prominin or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The NOV3 nucleic acid of 3465 nucleotides (also referred to as ac009238_gene_{—}5_EXT) encoding a novel prominin-like protein is shown in FIG. 3A. An open reading frame was identified beginning with an ATG initiation codon at nucleotides 67-69 and ending with a TGA codon at nucleotides 2589-2591. A putative untranslated region upstream from the initiation codon and downstream from the termination codon is underlined in FIG. 3A, and the start and stop codons are in bold letters.

TABLE 3A
NOV3 Nucleotide Sequence (SEQ ID NO:5)

GATGTATGGTCTGCCCTGGGCTTGTCTGTTCCCTCCTGAGCCTGAGCCCCTTACCTTCCTGACCCCATGA
AGCACACACTGCCTCTGCTGGCTCCCCTGCTGGGCCTGGGCCTCGGGCTGGCCCTGAGTCAGCTGGCTGC
AGGGGCCACAGACTGCAAGTTCCTTGGCCCCGCAGAGCACCTGACATTCACCCCAGCAGCCAGCGCCCGG
TCGCTGGCCCCTCGAGTTCGTGCCCCAGGACTCCTGGACTCCCTCTATGGCACCGTGCGCCGCTTCCTCT
CGGTGGTGCAGCTCAATCCTTTCCCTTCAGAGTTGGTAAAGGCCCTACTGAATGAGCTGGCCTCCGTGAA
GGTGAATGAGGTCCTGCGGTACGAGGCGGCCTACGTGCTATCCGCTGTCATCGCGCCCCTCTACCTGCTG
CTGGTGCCCACTGCCGGGCTTTGCTTCTGCTGCTGCCGCTGCCACCGGCGCTGCGCGGGACGAGTGAAGA
CAGAGCACAAGGCGCTGGCCTGTGACCGCCCGGCCCTCATGGTCTTCCTGCTGCTGACCACCCTCTTCCT
GCTGATTGGTGTGGTCTGTGCCTTTGTCACCAACCAGCGCACCCATGAACAGATGGGCCCCAGCATCGAG
GCCATGCCTGAGACCCTGCTCAGCCTCTCGGGCCTGGTCTCTCATGTCCCCCAAGAGCTGCAGGCCGTGG
CACAGCAATTCTCCCTGCCCCAGGAGCAAGTCTCAGAGGAGCTGGATGGTGTTGGTGTGAGCATTGGGAG
CGCGATCCACACTCAGCTCAGGAGCTCCGTGTACCCCTTGCTGGCCGCCGTCGGCAGTTTGGGCCACGTC
CTGCAGGTCTCCGTGCACCACCTGCAAACCTTGAATGCTACAGTGGTAGAGCTGCAGGCCGGGCAGCAGG
ACCTGGAGCCAGCCATCCGGGAACACCGGGACCGCCTCCTTGAGCTGCTGCAGGAGGCCAGGTGCCAGGG
AGATTGTGCAGGGGCCCTGAGCTGGGCCCGCACCCTGGAGCTGGGTGCTGACTTCAGCCAGCTGCCCTCT
GTGGACCATGTCCTGCACCAGCTAAAAGGTGTCCCCGAGGCCAACTTCTCCAGCATGGTCCAGGAGGAGA
ACAGCACCTTCAACGCCCTTCCAGCCCTGGCTGCCATGCAGACATCCAGCGTGGTGCAAGAGCTGAAGAA
GGCAGTGGCCCAGCAGCCCGAAGGGGTGAGGACACTGGCTGAAGGGTTCCCGCGCTTGGAGGCAGCTTCC
CGCTGGGCCCAGGCACTCCAGGAGGTGGAGCAGAGCAGCCGCCCCTACCTGCAGGACGTGCACAGATACG
AGACCTACAGGTGGATCGTGGGCTGCGTGCTGTGCTCCGTGGTCCTATTCGTGGTGCTCTGCAACCTGCT
GGGCCTCAATCTGGGCATCTGGGGCCTGTCTGCCAGGGACGACCCCAGCCACCCAGAAGCCAAGGGCGAG
GCTGGAGCCCGCTTCCTCATGGCAGGTGTGGGCCTCAGCTTCCTCTTTGCTGCACCCCTCATCCTCCTGG
TGTTCGCCACCTTCCTGGTGGGTGGCAACGTGCAGACGCTGGTGTGCCAGAGCTGGGAGAACGGCGAGCT
CTTTGAGTTTGCAGACACCCCAGGGAACCTGCCCCCGTCCATGAACCTGTCGCAACTTCTTCGCCTGAGG
AAGAACATCAGCATCCACCAAGCCTATCAGCAGTGCAAGGAAGGGGCAGCGCTCTGGACAGTCCTGCAGC
TCAACGACTCCTACGACCTGGAGGAGCACCTGGATATCAACCAGTATACCAACAAGCTACGGCAGGAGTT
GCAGAGCCTGAAAGTAGACACACAGAGCCTGGACCTGCTGAGCTCAGCCGCCCGCCGGGACCTGGAGGCC
CTGCAGAGCAGTGGGCTTCAGCGCATCCACTACCCCGACTTCCTCGTTCAGATCCAGAGGCCCGTGGTGA
AGACCAGCATGGAGCAGCTGGCCCAGGAGCTGCAAGGACTGGCCCAGGCCCAAGACAATTCTGTGCTGGG
GCAGCGGCTGCAGGAGGAGGCCCAAGGACTCAGAAACCTTCACCAGGAGAAGGTCGTCCCCCAGCAGAGC
CTTGTGGCAAAGCTCAACCTCAGCGTCAGGGCCCTGGAGTCCTCTGCCCCGAATCTCCAGGTGGCTGCTG
TTGGTGGGGACCTGGAGACCTCAGATGTCCTAGCCAATGTCACCTACCTGAAAGGAGAGCTGCCTGCCTG
GGCAGCCAGGATCCTGAGGAATGTGAGTGAGTGTTTCCTGGCCCGGGAGATGGCCTACTTCTCCCAGTAC
GTGGCCTCGGTGAGACAGGAGGTGACTCAGCGCATTCCCACCTGCCAGCCCCTCTCCGGAGCCCTCGACA
ACAGCCGTGTGATCCTGTGTGACATGATGGCTGACCCCTGGAATGCCTTCTGGTTCTGCCTGGCATCCTG
CACCTTCTTCCTGATCCCCAGCATCATCTTTGCCGTCAAGACCTCCAAATACTTCCGTCCTATCCGGAAA
CGCCTCAGCTCCACCAGCTCTGAGGAGACTCAGCTCTTCCACATCCCCCGGGTTACCTCCCTGAAGCTGT
AGGGCCTTGTGGGTGTGNTCTNTTGCCCTGNTGCNAATTTTCCANGCCCCGATTTAACCCTGCCNNGTGG
AAACGCGCAGGGAGTTGGGGTCTCGGGAGCCTANCTCCACAATATCCCTGGGTCCCATGCATGACCACCG
GCAGTTGCTGCGGCCCACCCCGCTCCTCCGCCTGTGCCTCCCTTGCCTCCTCCAGATGGCCGCCTGCCTC
ATCGAACCCCCAATCTGATCTGCACATTCCACCAGGCCACCCTTCTGAGGCAGCTGCGAGTCCAGCTGGA
CTTGAGTGGCAGAGAGCAGCTGGGGCGGCTGTGCCCTGCCAGGAGGATGCTGCCCCAAGCCTGCCGGCTG
GCAGGTCTGAGAACCATCCGGATCAGTCCTGTCCAATAGAGACATGATGCAAGCCACAGATGTCACTTAA
AATGAGCCAGTAGGCACAGTAAAGAAAGAAAAAAAGGTTAAATAATTTCAACGATATGTTTTATTAACCC
CATTGTAAATGATTAGCACTCAACCCTTAGATTGAAATAGGGTATTAAGAGTGAGAGGCCGAGGCTCAGC
CGCCAGGCTTTGATGAGATGCTGCCTGGTCAAGTGGATCCTGTCCAGCACAGCCCCACAGGGCTCAGGCA
GAGGTGGCTCAGGACGGGTGGGGCTGGTGTGCATCCTTTGCCGAAGCTTTCTGCACACCCGTGACAGCAG
CAGCTATGCTGAGTGGGGTGGACGGGGAGAAAGGTGAAGGGGCTTTAAGAATGAGTGTCCCACGGGCCTG
GTGTACGAAGACACCTTCAAACTCATTTACGCGCAGTTCTTCTCTCAGGGAGATGGCACCACCTATGCAC
ACTTCCTCTTCAACGCCTGTGATGCGTACGGGAAC

In a search of public sequence databases, it was found, for example, that the disclosed NOV3 nucleic acid sequence has 354 of 414 bases (85%) identical to a Mus musculus prominin-like mRNA (GENBANK-ID: AF128113)(E=6.1e−110). Public nucleotide databases include all GenBank databases and the GeneSeq patent database.

The disclosed NOV3 polypeptide (SEQ ID NO:6) encoded by SEQ ID NO:5 is 841 amino acid residues with a predicted molecular weight of 92451.6 and is presented using the one-letter code in Table 3B. The NOV3 protein was analyzed for signal peptide prediction and cellular localization. SignalP, Psort and Hydropathy results predict that NOV3 has a signal peptide, with a cleavage site most likely between positions 21 and 22 of SEQ ID NO:6 and is most likely a Type IIIa membrane protein.

TABLE 3B
Encoded NOV3 protein sequence (SEQ ID NO:6).

MKHTLALLAPLLGLGLGLALSQLAAGATDCKFLGPAEHLTFTPAARARWLAPRVRAPGLLDSLYGTVRRF
LSVVQLNPFPSELVKALLNELASVKVNEVVRYEAGYVVCAVIAGLYLLLVPTAGLCFCCCRCHRRCGGRV
KTEHKALACERAALMVFLLLTTLLLLIGVVCAFVTNQRTHEQMGPSIEAMPETLLSLWGLVSDVPQELQA
VAQQFSLPQEQVSEELDGVGVSIGSAIHTQLRSSVYPLLAAVGSLGQVLQVSVHHLQTLNATVVELQAGQ
QDLEPAIREHRDRLLELLQEARCQGDCAGALSWARTLELGADFSQVPSVDHVLHQLKGVPEANFSSMVQE
ENSTFNALPALAAMQTSSVVQELKKAVAQQPEGVRTLAEGFPGLEAASRWAQALQEVEESSRPYLQEVQR
YETYRWIVGCVLCSVVLFVVLCNLLGLNLGIWGLSARDDPSHPEAKGEAGARFLMAGVGLSFLFAAPLIL
LVFATFLVGGNVQTLVCQSWENGELFEFADTPGNLPPSMNLSQLLGLRKNISIHQAYQQCKEGAALWTVL
QLNDSYDLEEHLDINQYTNKLRQELQSLKVDTQSLDLLSSAARRDLEALQSSGLQRIHYPDFLVQIQRPV
VKTSMEQLAQELQGLAQAQDNSVLGQRLQEEAQGLRNLHQEKVVPQQSLVAKLNLSVRALESSAPNLQVA
AVGGDLETSDVLANVTYLKGELPAWAARILRNVSECFLAREMGYFSQYVAWVREEVTQRIATCQPLSGAL
DNSRVILCDMMADPWNAFWFCLAWCTFFLIPSIIFAVKTSKYFRPIRKRLSSTSSEETQLFHIPRVTSLK
L

A BLASTX search was performed against public protein databases (Table 3C). The fill amino acid sequence of the protein of the invention was found to have 201 of 266 amino acid residues (75%) identical to, and 224 of 266 residues (84%) positive with, the 259 amino acid residue prominin protein from Mus musculus (ptnr: SPTREMBL-ACC: Q9WUC7; E=5.2e−101).

TABLE 3C

BLASTX results for NOV3

| | | | | |
|---|---|---|---|---|
| | | | Smallest | |
| | | | Sum | |
| | Reading | High | Prob | |
| Sequences producing High-scoring Segment Pairs: | Frame | Score | P(N) | N |

| | | | |
|---|---|---|---|
| ptnr:SPTREMBL-ACC:Q9WTUC7 PROMININ-LIKE PROTEIN—Mus musc. . . | 1003 | 5.2e-101 | 1 |
| ptnr:PIR-ID:T08881 prominin—mouse | 959 | 2.4e-96 | 1 |
| ptnr:SPTREMBL-ACC:O54990 PROMININ PRECURSOR (AC133 ANTIGE. . . | 886 | 1.3e-88 | 1 |
| ptnr:SPTREMBL-ACC:O43490 PROMININ-LIKE PROTEIN 1 PRECURSO. . . | 879 | 7.1e-88 | 1 |
| ptnr:SPTREMBL-ACC:Q9W735 PROMININ-LIKE PROTEIN—Brachyda. . . | 772 | 1.6e-76 | 1 |
| ptnr:TREMBLNEW-ACC:AAF73049 FUDENINE—Rattus norvegicus. . . | 700 | 6.6e-69 | 1 |
| ptnr:SPTREMBL-ACC:Q19188 F08B12.1 PROTEIN—Caenorhabditi. . . | 281 | 1.1e-20 | 1 |
| ptnr:SPTREMBL-ACC:P82295 PROMININ-LIKE PROTEIN—Orosophi. . . | 229 | 6.0e-15 | 1 |
| ptnr:SPTREMBL-ACC:Q9W175 EYC PROTEIN—Drosophila melanog. . . | 182 | 7.4e-14 | 2 |

BLAST results include sequences from the Patp database, which is a proprietary database that contains sequences published in patents and patent publications. Patp results include those listed in Table 3D.

TABLE 3D
Patp alignments of NOV3

| | | | Smallest |
|---|---|---|---|
| | | | Sum |
| Sequences producing | Reading | High | Prob. |
| High-scoring Segment Pairs: | Frame | Score | P(N) |
| Patp:AAB64995 Human secreted | +1 | 4226 | 0.0 |
| protein #3, 834 a.a. | | | |

The NOV3 gene is expressed in the following tissues by EST analysis: Adult, squamous cell carcinoma Genbank EST AI285647 Adult, colon adenocarcinoma. Genbank EST AI792608. Brain Genbank EST AI523747 Kidney Tumor Genbank EST AI523747. Using Genbank EST AI699155, a sequence corresponding to the 3′ of the gene, it is possible to perform SAGE analysis using the NCI-CGAP tool. This analysis indicates that this novel sequence is expressed in brain tumors (SAGE Duke H1020, SAGE Duke H392, and SAGE Duke 1273) and ovarian tumors (SAGE OVT-7 and SAGE OVT-8).

Quantitative gene expression analysis (TaqMan) was performed on a NOV3 nucleic acid. The general method is described in Example 1. Results of TaqMan analysis of a NOV3 nucleic acid are described in Example 4.

### NOV4

In the present invention, Genescan analysis of a NOV3 nucleic acid sequence generates an alternatively spliced form, designated NOV4. A NOV4 (also referred to as ac009238_gene_{—}5) nucleic acid of 3258 nucleotides is shown in Table 4A. An open reading frame was identified beginning with an ATG initiation codon at nucleotides 1-3 and ending with a TGA codon at nucleotides 3256-3258.

TABLE 4A
NOV4 Nucleotide Sequence (SEQ ID NO:7)

ATGAAGCACACACTGGCTCTGCTGGCTCCCCTGCTGGGCCTGGGCCTGGGGCTGGCCCTGAGTCAGCTGG
CTGCAGGGGCCACAGACTGCAAGTTCCTTGGCCCGGCAGAGCACCTGACATTCACCCCAGCAGCCAGGGC
CCGGTGGCTGGCCCCTCGAGTTCGTGCGCCAGGACTCCTGGACTCCCTCTATGGCACCGTGCGCCGCTTC
CTCTCGGTGGTGCAGCTCAATCCTTTCCCTTCAGAGTTGGTAAAGGCCCTACTGAATGAGCTGGCCTCCG
TGAAGGTGAATCAGGTGGTGCGGTACGAGCCGCGCTACGTCGTATGCGCTCTGATCGCGGGCCTCTACCT
GCTCCTGGTGCCCACTGCCGGGCTTTGCTTCTGCTCCTGCCGCTGCCACCCGCCCTGCGGGGGACGAGTG
AAGACACACCACAAGGCGCTGGCCTGTGAGCCCGCGCCCCTCATGGTCTTCCTGCTGCTCACCACCCTCT
TGCTGCTGATTGGTGTGGTCTGTGCCTTTGTCACCAACCAGCGCACGCATGAACAGATGGGCCCCAGCAT
CGAGGCCATGCCTGAGACCCTGCTCAGCCTCTGGGGCCTGGTCTCTGATGTCCCCCAAGAGCTGCAGGCC
GTGGCACAGCAATTCTCCCTGCCCCAGGAGCAAGTCTCAGAGGAGCTGGATGGTGTTGGTGTGAGCATTG
GGAGCGCGATCCACACTCACCTCAGGAGCTCCGTGTACCCCTTGCTGGCGGCCGTCGGCAGTTTGGGCCA
GGTCCTGCAGGTCTCCGTGCACCACCTGCAAACCTTGAATGCTACAGTGGTAGAGCTGCAGGCCGGGCAG
CAGGACCTGGAGCCAGCCATCCGGGAACACCGGGACCGCCTCCTTGAGCTGCTGCAGGAGGCCAGGTGCC
AGGGAGATTGTGCAGGGGCCCTGAGCTGGGCCCGCACCCTGGAGCTGGGTGCTGACTTCAGCCAGGTGCC
CTCTGTGGACCATGTCCTGCACCAGCTAAAAGGTGTCCCCGAGGCCAACTTCTCCAGCATGGTCCAGGAG
GAGAACAGCACCTTCAACGCCCTTCCAGCCCTGGCTGCCATGCAGACATCCAGCGTGGTGCAAGAGCTGA
AGAAGGCAGTGGCCCAGCAGCCGGAAGGGGTGAGGACACTGGCTGAAGGGTTCCCGGGCTTGGAGGCAGC
TTCCCGCTGGCCCCAGGCACTGCAGGAGGTGGAGGAGACCAGCCGCCCCTACCTGCAGGAGGTGCAGAGA
TACGAGACCTACAGGTGGATCGTCGGCTGCGTGCTGTGCTCCGTGGTCCTATTCGTGGTGCTCTGCAACC
TGCTGGGCCTCAATCTGGGCATCTGGGGCCTGTCTGCCAGGGACGACCCCAGCCACCCAGAAGCCAAGGG
CGAGGCTGGAGCCCGCTTCCTCATGGCACCTGTGCGCCTCAGCTTCCTCTTTGCTGCACCCCTCATCCTC
CTGGTGTTCGCCACCTTCCTGGTGGGTGGCAACGTGCAGACGCTGGTGTGCCAGAGCTGGGAGAACGGCG
AGCTCTTTGAGTTTGCAGACACCCCAGGGAACCTGCCCCCGTCCATGAACCTGTCGCAACTTCTTGGCCT
GAGGAAGAACATCAGCATCCACCAAGCCTATCAGCAGTGCAAGGAAGGGGCAGCGCTCTGGACAGTCCTG
CAGCTCAACGACTCCTACGACCTGGAGGAGCACCTGGATATCAACCAGTATACCAACAAGCTACGGCAGG
AGTTGCAGAGCCTGAAAGTAGACACACAGAGCCTGGACCTGCTGAGCTCAGCCGCCCGCCGGGACCTGGA
GGCCCTGCAGAGCAGTGGGCTTCAGCGCATCCACTACCCCGACTTCCTCGTTCAGATCCAGAGGCCCGTG
GTGAAGACCAGCATGGAGCAGCTGGCCCAGGAGCTGCAAGGACTGGCCCAGGCCCAAGACAATTCTGTGC
TGGGGCAGCGGCTGCAGGAGGAGGCCCAAGGACTCAGAAACCTTCACCAGGAGAAGGTCGTCCCCCAGCA
GAGCCTTGTGGCAAAGCTCAACCTCAGCGTCAGGGCCCTGGAGTCCTCTGCCCCGAATCTCCAGGTGGCT
GCTGTTGGTGGGGACCTGGAGACCTCAGATGTCCTAGCCAATGTCACCTACCTGAAAGGAGAGCTGCCTG
CCTGGGCAGCCAGGATCCTGAGGAATGTGAGTGAGTGTTTCCTGGCCCGGGAGATGGGCTACTTCTCCCA
GTACGTGGCCTGCGTGAGAGAGGAGGTGACTCAGCCCATTGCCACCTGCCAGCCCCTCTCCGGAGCCCTG
GACAACAGCCGTGTGATCCTCTGTGACATGATCGCTGACCCCTGGAATGCCTTCTGGTTCTCCCTGGCAT
GGTGCACCTTCTTCCTGATCCCCAGCATCATCTTTGCCGTCAAGACCTCCAAATACTTCCGTCCTATCCG
GAAACGCCTCAGGGCTTCCGAGGAGAAACCCTCAGGGCTCTGGGTTTCGTCCTGTGTCAGGGCTGAGGGT
CTGGGGGGAAGGTCCCCTCTTCACCATATCTCCACTGCTACCTTGCTGGCCCCAGAGACCACCCTGCCCA
ACCAAACCACTCAGGCCCTTGGGCCCTCTGCAGATCTCATCCAGGATTTATTGGTGTCCAGTGGGAGAAT
ACTGGGAATTGCCAAGGCCGTCTCTGGGAAGTCTGCAGATGCCCGTGTGCCCACAACAGATGGCCGCCTG
CCTCATCGAACCCCCAATCTGATCTGCACATTCCACCAGGCCACCCTTCTGAGGCAGCTGCGAGTCCAGC
TGGACTTGAGTGGCAGAGAGCACCTGGCCCGCCTGTGCCCTGCCAGGAGGATGCTGCCCCAAGCCTGCCG
GCTGGCAGGACTCGCTCTCTGTGCTTCCCTGCAGGCTGTGACTGGCCCGGGCTCCCTGCTGCCAATTCTT
CCAGTTCAAATCAACCTGGGGGTCCCATCTTACACCATAGCCAGGGAAGTGACAAAGGCGTCGGACGGCA
GCCTCCTGGGGGACCTCGGGCACACACCACTTAGCAAGAAGGAGGGTATCAAGTGGCAGAGGCCGAGGCT
CAGCCGCCAGGCTTTGATGAGATGCTGCCTGGTCAAGTGGATCCTGTCCAGCACAGCCCCACAGGGCTCA
GGCAGAGGTGGCTCAGGACGGGTGGGGCTGGTGTGCATCCTTTGCCGAAGCTTTCTGCACACCCGTGACA
GCAGCAGCTATGCTGAGTGGGGTGGACGGGGAGAAAGTGA

The disclosed NOV4 polypeptide (SEQ ID NO:8) encoded by SEQ ID NO:7 is 1086 amino acid residues with a predicted molecular weight of 118,462.4 Da and is presented using the one-letter code in Table 4B. The NOV4 protein was analyzed for signal peptide prediction and cellular localization. SignalP, Psort and Hydropathy results predict that NOV4 has a signal peptide with the most likely cleavage point between positions 21 and 22 in SEQ ID NO:8 and is most likely a Type IIIa membrane protein.

TABLE 4B
Encoded NOV4 protein sequence (SEQ ID NO:8).

MKHTLALLAPLLGLGLGLALSQLAAGATDCKFLGPAEHLTFTPAARARWLAPRVRAPGLLDSLYGTVRRF
LSVVQLNPFPSELVKALTMELASVKVNEVVRYEAGYVVCAVIAGLYLLLVPTACLCFCCCRCHRRCGGRV
KTEHKALACERAALMVFLLLTTLLLLIGVVCAFVTNQRTHEQMGPSIEAMPETLLSLWGLVSDVPQELQA
VAQQFSLPQEQVSEELDGVGVSIGSAIHTQLRSSVYPLLAAVGSLGQVLQVSVHHLQTLNATVVELQAGQ
QDLEPAIREHRDRLLELLQEARCQGDCAGALSWARTLELGADFSQVPSVDHVLHQLKGVPEANFSSMVQE
ENSTFNALPALAAMQTSSVVQELKKAVAQQPEGVRTLAEGFPGLESRWAQALQEVEESSRPYLQEVQR
YETYRWIVGCVLCSVVLFVVLCNLLGLNLGIWGLSARDDPSHPEAKGEAGARFLMAGVGLSFLFAAPLIL
LVFATFLVGGNVQTLVCQSWENGELFEFADTPGNLPPSMNLSQLLGLRKNISIHQAYQQCKEGAALWTVL
QLNDSYDLEEHLDINQYTNKLRQELQSLKVDTQSLDLLSSAARRDLEALQSSGLQRIHYPDFLVQIQRPV
VKTSMEQLAQELQGLAQAQDNSVLGQRLQEEAQGLRNLHQEKVVPQQSLVAKLNLSVRALESSAPNLQVA
AVGGDLETSDVLANVTYLKGELPAWAARILRNVSECFLAREMGYFSQYVAWVREEVTQRIATCQPLSGAL
DNSRVILCDMMADPWNAFWFCLAWCTFFLIPSIIFAVKTSKYFRPIRKRLSSTSSEETQLFHIPRVTSLK
L

BLAST results include sequences from the Patp database, which is a proprietary database that contains sequences published in patents and patent publications. Patp results include those listed in Table 4C.

TABLE 4C
Patp alignments of NOV4

| | | | Smallest |
|---|---|---|---|
| | | | Sum |
| Sequences producing | Reading | High | Prob. |
| High-scoring Segment Pairs: | Frame | Score | P(N) |
| Patp:AAB64995 Human secreted | +1 | 4134 | 0.0 |
| protein #3, 834 a.a. | | | |
| PatP:AAB65031 Gene #3 | +1 | 1448 | 1.9e-147 |
| associated protein, 287 a.a. | | | |

Prominin is a novel plasma membrane protein with an N-terminal extracellular domain, five transmembrane segments flanking two short cytoplasmic loops and two large glycosylated extracellular domains, and a cytoplasmic C-terminal domain. Prominin is found not only in the neuroepithelium but also in various other epithelia of the mouse embryo. In the adult mouse, prominin has been detected in the brain ependymal layer, and in kidney tubules. In these epithelia, prominin is specific to the apical surface, where it is selectively associated with microvilli and microvilli-related structures. Remarkably, upon expression in CHO cells, prominin is preferentially localized to plasma membrane protrusions such as filopodia, lamellipodia, and microspikes. These observations imply that prominin contains information to be targeted to, and/or retained in, plasma membrane protrusions rather than the planar cell surface.

The human AC133 antigen and mouse prominin are structurally related plasma membrane proteins. The human AC133 antigen shows the features characteristic of mouse prominin in epithelial and transfected non-epithelial cells, i.e. a selective association with apical microvilli and plasma membrane protrusions, respectively. Conversely, flow cytometry of murine CD34(+) bone marrow progenitors revealed the cell surface expression of prominin. Taken together, the data strongly suggest that the AC133 antigen is the human orthologue of prominin.

The above defined information for this invention suggests that these prominin-like proteins (NOV3 and NOV4) may function as members of the “prominin family”. Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of the invention are useful in potential therapeutic applications implicated in and disorders. For example, a cDNA encoding the prominin-like protein may be useful in gene therapy, and the prominin-like protein may be useful when administered to a subject in need thereof. By way of nonlimiting example, the compositions of the present invention will have efficacy for treatment of patients suffering from Clouston syndrome and deafness, mutilating palmoplantar keratoderma (PPK), X-linked Charcot-Marie-Tooth neuropathy, hereditary peripheral neuropathy. The novel nucleic acid encoding prominin-like protein, and the prominin-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The novel nucleic acids encoding the prominin-like proteins of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. These novel proteins can be used in assay systems for functional analysis of various human disorders, which will help in understanding of pathology of the disease and development of new drug targets for various disorders.

### NOV5

A novel nucleic acid was identified on chromosome 1 by TblastN using CuraGen Corporation's sequence file for Glucose Transport Protein or homolog as run against the Genomic Daily Files made available by GenBank or from files downloaded from the individual sequencing centers. The nucleic acid sequence was predicted from the genomic file Sequencing Center accession number: ba252a4 by homology to a known Glucose Transport Protein or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The disclosed NOV5 nucleic acid of 2007 nucleotides (also referred to as CuraGen Acc. No. SC87081869_A) encoding a novel glucose transport protein-like protein is shown in Table 5A. An ORF begins with an ATG initiation codon at nucleotides 5-7 and ends with a TGA codon at nucleotides 1997-1999. The start and stop codons are in bold letters in Table 5A.

TABLE 5A
NOV5a Nucleotide Sequence (SEQ ID NO:9)

AGCAATGGGGCCTGGAGCTTCAGGGGACGGGGTCAGGACTGAGACAGCTCCACACATAGCACTGGACTCC
AGAGTTGGTCTGCACGCCTACGACATCAGCGTGGTGGTCATCTACTTTGTCTTCGTCATTGCTGTGGGGA
TCTGGTCGTCCATCCGTGCAAGTCGAGGGACCATTGGCGGCTATTTCCTGGCCGGOAGGTCCATGAGCTG
GTCGCCAGTGATTGGAGCATCTCTGATGTCCAGCAATGTGGGCAGTGGCTTGTTCATCGGCCTGGCTGGG
ACAGGGGCTGCCGGAGGCCTTGCCGTAGGTGGCTTCGAGTGGAACGCAACCTGGCTGCTCCTGGCCCTTG
GCTGGGTCTTCGTCCCTGTGTACATCGCAGCAGGTGTGGTCACAATGCCGCAGTATCTGAAGAAGCGATT
TGGGGGCCAGAGGATCCAGATGTACATGTCTGTCCTGTCTCTCATCCTCTACATCTTCACCAAGATCTCG
GTAGACATCTTCTCTGGAGCCCTCTTCATCCACATGGCATTCGGCTGGAACCTGTACCTCTCCACAGGGA
TCCTGCTGGTGGTGACTGCCGTCTACACCATTGCAGCTGCTGGCCTCATCGCCGTGATCTACACAGATGC
TCTGCAGACGGTGATCATGGTAGGGCGAGCCCTGGTCCTCATGTTTCTCTGGGTTAAGGAAGAGACCGGCTGG
TACCCACGCCTGGAGCAGCCGTACACGCACGCCATCCCTAATGTCACAGTCCCCAACACCACCTGTCACC
TCCCACGGCCCGATGCTTTCCACATGCTTCGGGACCCTGTGAGCGGGGACATCCCTTGGCCAGGTCTCAT
TTTCGGGCTCACAGTGCTCGCCACCTGGTGTTGGTGCACAGACCACGTAATCGTGCAGCCGTCTCTCTCC
GCCAAGAGTCTGTCTCATGCCAAGGCAGGCTCCGTGCTGGCGGGCTACCTGAAGATCCTCCCCATGTTCT
TCATCGTCATGCCTGGCATGATCAGCCGGGCCCTCTTCCCAGAGATTGCGTGTATGTGTGTGCCTGTGTC
TACACATGCATGTGCAGCAAGGAAGAGGAACGAAGGAGTCCTGCAGGGGTTGGTGGTGGCAGTTCGTCTC
TCCCCAGGTCTGCGGGGGCTGATGATTGCCGTGATCATGGCCGCTCTCATGACCTCACTCACCTCCATCT
TCAACAGCAGCAGCACCCTGTTCACCATTGATGTGTGGCAGCGCTTCCGCAGGAAGTCAACAGAGCAGGA
GCTGATGGTGGTGGGCAGGGTGTTTGTGGTGTTCCTGGTTGTCATCAGCATCCTCTGGATCCCCATCATC
CAAAGCTCCAACAGTGGGCAGCTCTTCGACTACATCCAGGCTGTCACCAGTTACCTGGCCCCACCCATCA
CCGCTCTCTTCCTGCTGGCCATCTTCTGCAAGAGGGTCACAGAGCAGGGAGCTTTCTGGGGCCTCGTGTT
TGGCCTGGGAGTGGGGCTTCTGCGTATGATCCTGGAGTTCTCATACCCAGCGCCAGCCTGTGGGGAGGTG
GACCGGAGGCCAGCAGTGCTGAAGGACTTCCACTACCTGTACTTTGCAATCCTCCTCTGCGGGCTCACTG
CCATCGTCATTGTCATTGTCAGCCTCTGTACAACTCCCATCCCTGAGCTCCACACATACATTTATTGTGG
CACTATTCACAATAGCAAAGACTTTGAACCAATCCAAATATCCAACAATGAGCAGGCCCTGAGCCCAGCA
GAGAAGGCTGCGCTAGAACAGAAGCTGACAAGCATTGAGGAGGAGTCTTCTGGCTTTGTCCCTCCAGCCT
GGAGCTGGTTCTGTGGGCTCTCTGGAACACCGGAGCAGGCCCTGAGCCCAGCAGAGAAGGCTGCGCTAGA
ACAGAAGCTGACAAGCATTGAGGAGGAGCCACTCTGGAGACATGTCTGCAACATCAATGCTGTCCTTTTG
CTGGCCATCAACATCTTCCTCTGGGGCTATTTTGCGTGATTCCACAG

In a search of sequence databases, it was found, for example, that the nucleic acid sequence has 1118 of 1625 bases (68%) identical to a Oryctolagus cuniculus Glucose Transport Protein mRNA (GENBANK-ID: U08813).

The NOV5 protein encoded by SEQ ID NO:9 has 664 amino acid residues with a predicted molecular weight of 72,303.4 Daltons and is presented using the one-letter code in Table 5B. PSORT analysis suggests that the NOV5 protein is a plasma membrane protein (certainty 0.8000). SIGNALP analysis suggests that the NOV5 protein has a signal peptide, with the most likely cleavage site occuring between positions 52 and 53 of SEQ ID NO. 10.

A NOV5 nucleic acid has 100% identity to a human chromosome 1 genomic DNA segment (Genbank Accession No. NT004525.4), and a NOV5 nucleic acid maps to the p13 locus of chromosome 1. This locus is associated with retinitis pigmentosa, Stargardt disease, vesicoureteral reflux, cone-rod dystrophy and/or other disorders.

TABLE 5B
Encoded NOV5 protein sequence (SEQ ID NO:10)

MGPGASGDGVRTETAPHIALDSRVGLHAYDISVVVIYFVFVIAVGIWSSIRASRGTIGGYFLAGRSMSWW
PVIGASLMSSNVGSGLFIGLAGTGAAGGLAVGGFEWNATWLLLALGWVFVPVYIAAGVVTMPQYLKKRFG
GQRIQMYMSVLSLILYIFTKISVDIFSGALFIQMALGWNLYLSTGILLVVTAVYTIAGGGLMAVIYTDAL
QTVIMVGGALVLMFLGKEETGWYPGLEQRYRQAIPNVTVPNTTCHLPRPDAFHMLRDPVSGDIPWPGLIF
GLTVLATWCWCTDQVIVQRSLSAKSLSHAKGGSVLGGYLKILPMFFIVMPGMISRALFPEIACMCVPVCT
HACAARKRKEGVLQGLVVAVRLSPGLRGLMIAVIMAALMSSLTSIFNSSSTLFTIDVWQRFRRKSTEQEL
MVVGRVFVVFLVVISILWIPIIQSSNSGQLFDYIQAVTSYLAPPITALFLLAIFCKRVTEQGAFWGLVFG
LGVGLLRMILEFSYPAPACGEVDRRPAVLKDFHYLYFAILLCGLTAIVIVIVSLCTTPIPELHTYIYCGT
IHNSKDFEPIQISNNEQALSPAEKAALEQKLTSIEEESSGFVPPAWSWFCGLSGTPEQALSPAEKAALEQ
KLTSIEEEPLWRHVCNINAVLLLAINIFLWGYFA

The full amino acid sequence of the NOV5 protein was found to have 366 of 654 amino acid residues (55%) identical to, and 477 of 654 residues (72%) positive with, the 654 amino acid residue Glucose Transport Protein protein from Ovis aries (Sheep) (ptnr:SPTREMBL-ACC: S59638). The global sequence homology (as defined by FASTA alignment with the full length sequence of this protein) is 66% amino acid homology and 56% amino acid identity.

BLAST results include sequences from the Patp database, which is a proprietary database that contains sequences published in patents and patent publications. Patp results include those listed in Table 5C.

TABLE 5C
Patp alignments of NOV5

| | | | Smallest |
|---|---|---|---|
| | | | Sum |
| Sequences producing | Reading | High | Prob. |
| High-scoring Segment Pairs: | Frame | Score | P(N) |
| >patp:AAR73595 Cotransporter | +2 | 1856 | 1.1e-190 |
| protein SGLT1 - 662 aa. | | | |

Quantitative gene expression analysis (TaqMan) was performed on a NOV5 nucleic acid. The general method is described in Example 1. Results of TaqMan analysis of a NOV5 nucleic acid are described in Example 5.

The Na(+)-dependent D-glucose symporter has been shown to be located on the basolateral domain of the plasma membrane of ovine parotid acinar cells. This is in contrast to the apical location of this transporter in the ovine enterocyte. The amino acid sequences of these two proteins are identical. The results indicated that the signals responsible for the differential targeting of these two proteins to the apical and the basal domains of the plasma membrane are not contained within the primary amino acid sequence.

Intestinal sodium/glucose cotransporter is responsible for ‘active’ glucose absorption across the brush-border membrane. The transepithelial absorption is then completed at the basal lateral membrane through the facilitated glucose transporter, which is similar if not identical to the 55-kD glucose carrier in erythrocytes. Southern blot analysis of DNA from a panel of mouse-human hybrids demonstrates that only those hybrids containing chromosome 22 showed the characteristic bands identified by Southern analysis of human DNA. The SGLT 1 gene maps to 22q11.2-qter by study of DNA from somatic cell hybrids. A RFLP was identified with EcoRI. Unexpectedly, the sodium-glucose transporter showed no homology with the facilitated glucose carrier or with any other known protein. By fluorescence in situ hybridization the SGLT1 gene was localized to 22q13.1. The SGLT1 gene comprises 15 exons spanning 72 kb. Transcription initiation occurs from a site 27 bp 3-prime of a TATAA sequence. Sequence considerations and comparison of exons against protein secondary structure suggested a possible evolutionary origin of the SGLT1 gene from a 6-membrane-span ancestral precursor via a gene duplication event.

The expression pattern, map location and protein similarity information for the invention suggest that this Glucose transport protein-like protein may function as a member of the Glucose transport protein-like protein family. Therefore, the nucleic acids and proteins of the invention are useful in potential therapeutic applications implicated, for example but not limited to, in various pathologies/disorders as described below and/or other pathologies/disorders. Potential therapeutic uses for the invention(s) are, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention are useful in potential therapeutic applications implicated in various diseases and disorders described below and/or other pathologies and disorders. For example, but not limited to, a cDNA encoding the Glucose transport protein-like protein may be useful in gene therapy, and the Glucose transport protein-like protein may be useful when administered to a subject in need thereof. By way of nonlimiting example, the compositions of the present invention will have efficacy for treatment of patients suffering from Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, memory/perception/attention disorders, and/or neuroprotection. The novel nucleic acid encoding the a Glucose transport protein-like protein, and the a Glucose transport protein-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The novel nucleic acid encoding glucose transport protein-like protein, and the glucose transport protein-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV5 protein has multiple hydrophilic regions, each of which can be used as an immunogen. In one embodiment, a contemplated NOV5 epitope is from about amino acids 12 to 28. In another embodiment, a NOV5 epitope is from about amino acids 58 to 77. This novel protein also has value in development of powerful assay system for functional analysis of various human disorders, which will help in understanding of pathology of the disease and development of new drug targets for various disorders.

### NOV6

A NOV6 nucleic acid sequence (also called SC71046974_EXT) encodes Na+H+ Exchanger-like proteins. These sequences were initially identified by searching CuraGen's Human SeqCalling database for DNA sequences which translate into proteins with similarity to Na+H+ Exchanger-like proteins. SeqCalling assembly 71046974 was identified as having suitable similarity. SeqCalling assembly 71046974 was analyzed further to identify any open reading frames encoding novel full length proteins as well as novel splice forms of these genes. The SeqCalling assembly was extend using one or more sequences taken from additional SeqCalling assemblies, publicly available EST sequences and public genomic sequences. Public ESTs and additional CuraGen SeqCalling assemblies were identified by the CuraTools™ program SeqExtend. Such fragments were included in the DNA sequence extension for SeqCalling assembly 71046974 only when the extent of identity in the putative overlap region was high. The extent of identity may be, for example, about 90% or higher, preferably about 95% or higher, and even more preferably close to or equal to 100%. These inclusions, if used, are described below.

The following genomic clones were identified as having regions with 100% identity to the SeqCalling assembly 71046974 and it was selected for analysis because this identity indicates that this clone represents the genomic locus for SeqCalling assembly 71046974. Genomic clones gb:GENBANK-ID:AC007278|acc:AC007278 Homo sapiens clone NH0308G20, complete sequence—Homo sapiens, 162508 bp.

The results of these analyses were integrated and manually corrected for apparent inconsistencies that may have arisen, for example, from miscalled bases in the original fragments used. The sequences obtained encode the full-length proteins disclosed herein. When necessary, the process to identify and analyze cDNAs, ESTs and genomic clones was reiterated to derive the full length sequence.

AC007278 was analyzed by Genscan and Grail to identify exons and putative coding sequences. This clone was also analyzed by TblastN, BlastX and other programs to identify genomic regions translating to proteins with similarity to the original protein or protein family of interest. The following regions of genomic clone AC007278 were assembled together manually using the methods described above and are predicted to represent a full length transcript for SC71046974_EXT: bp117325-117068, 112244-111780, 87634-87377, 85830-85612, 83004-82804, 82238-82152, 78882-78811, 76994 76833, 71225-71130, 66061-65930, 64826-64716, 58488-58397.

The NOV6 nucleic acid of 2153 nucleotides has an open reading frame was identified beginning with an ATG initiation codon at nucleotides 1-3 and ending with a TAG codon at nucleotides 2151-2153. A putative untranslated region upstream from the initiation codon and downstream from the termination codon is underlined in Table 6A, and the start and stop codons are in bold letters.

TABLE 6A
NOV6 Nucleotide Sequence (SEQ ID NO:11)

ATGGCTCTGCAGATGTTCGTGACTTACAGTCCTTGGAATTGTTTGCTACTGCTAGTGGCTCTTGAGTGTT
CTGAAGCATCTTCTGATTTGAATGAATCTGCAAATTCCACTGCTCAGTATGCATCTAACGCTTGGTTTGC
TGCTGCCAGCTCAGAGCCAGAGGAAGGGATATCTGTTTTTGAACTGGATTATGACTATGTGCAAATTCCT
TATGAGGTCACTCTCTGGATACTTCTAGCATCCCTTGCAAAAATAGGTTTCCACCTCTACCACAGGCTGC
CAGGCCTCATGCCAGAAAGCTGCCTCCTCATCCTGGTGGGGGCGCTGGTGGGCGGCATCATCTTCGGCAC
CGACCACAAATCGCCTCCGGTCATGGACTCCAGCATCTACTTCCTGTATCTCCTGCCACCCATCGTTCTG
GAGGGCGGCTACTTCATGCCCACCCGGCCCTTCTTTGAGAACATCGGCTCCATCCTGTGGTGGGCAGTAT
TGGGGGCCCTGATCAACGCCTTGGGCATTGGCCTCTCCCTCTACCTCATCTGCCAGGTGAAGGCCTTTGG
CCTGGGCGACGTCAACCTGCTGCAGAACCTGCTGTTCGGCAGCCTGATCTCCGCCGTGGACCCAGTGGCC
GTGCTAGCCGTGTTTGAGGAAGCGCGCGTGAACGAGCAGCTCTACATGATGATCTTTGGGGAGGCCCTGC
TCAATGATGGCATTACTGTGGTGTTATACAATATGTTAATTGCCTTTACAAAGATGCATAAATTTGAAGA
CATAGAAACTGTCGACATTTTGGCTGGATGTGCCCGATTCATCGTTGTGGGGCTTGGAGGGGTATTGTTT
GGCATCGTTTTTGGATTTATTTCTGCATTTATCACACGTTTCACTCAGAATATCTCTGCAATTGAGCCAC
TCATCGTCTTCATGTTCAGCTATTTGTCTTACTTAGCTGCTGAAACCCTCTATCTCTCCGGCATCCTGGC
GATCACAGCCTGCGCAGTAACAATGAAAAAGTACGTGGAAGAAAACGTGTCCCAGACATCATACACGACC
ATCAAGTACTTCATGAAGATGCTGAGCAGCGTCAGCGAGACCTTGATCTTCATCTTCATGGGTGTGTCCA
CTGTGGGCAAGAATCACGAGTGGAACTGGGCCTTCATCTGCTTCACCCTGGCCTTCTGCCAAATCTGGAG
AGCCATCAGTGTATTTGCTCTCTTCTATATCAGTAACCAGTTTCGGACTTTCCCCTTCTCCATCAAGGAC
CAGTGCATCATTTTCTACAGTGGTGTTCGAGGAGCTGGAAGTTTTTCACTTGCATTTTTGCTTCCTCTGT
CTCTTTTTCCTAGGAAGAAAATGTTTGTCACTGCTACTCTAGTAGTTATATACTTTACTGTATTTATTCA
GGGAATCACAGTTGGCCCTCTGGTCAGGTACCTGGATGTTAAAAAAACCAATAAAAAAGAATCCATCAAT
GAAGAGCTTCATATTCGTCTGATGGATCACTTAAAGGCTGGAATCGAAGATGTGTGTGGGCACTGGAGTC
ACTACCAAGTGAGAGACAAGTTTAAGAAGTTTGATCATAGATACTTACGGAAAATCCTCATCAGAAAGAA
CCTACCCAAATCAAGCATTGTTTCTTTGTACAAGAAGCTGGAAATGAAGCAAGCCATCGAGATGGTGGAG
ACTGGGATACTGAGCTCTACAGCTTTCTCCATACCCCATCAGGCCCAGAGGATACAAGGAATCAAAAGAC
TTTCCCCTGAAGATGTGGAGTCCATAAGGGACATTCTGACATCCAACATGTACCAAGTTCGGCAAAGGAC
CCTGTCCTACAACAAATACAACCTCAAACCCCAAACAAGTGAGAAGCAGGCTAAAGAGATTCTGATCCGC
CGCCAGAACACCTTAAGGGAGAGCATGAGGAAAGGTCACAGCCTGCCCTGGGGAAAGCCGGCTGGCACCA
AGAATATCCGCTACCTCTCCTACCCCTACGGGAATCCTCAGTCTGCAGGAAGAGACACAAGGGCTGCTGG
GTTCTCAGGTAAGCTGCCCACCTGGCTGCTCCTTTGGTTGAGGTTCGGTCGAGGTGGACAGCTGACCATG
GACACGGCAGGGACCATCACAGGTCCCATAGTCCTTTGCTCCAAAAAAAATAG

In a search of sequence databases, it was found, for example, that the NOV6 nucleic acid sequence has 1772 of 2092 bases (84%) identical to a Rattus norvegicus Na+H+ Exchanger-like protein mRNA (GENBANK-ID:RATNHEXIV|acc:M85301). Also, the NOV6 nucleic acid maps to the q11 locus of human chromosome 2. This locus has been associated with autosomal dominant hypohidrotic ectodennal dysplasia, achromatopsia and/or other dieases/disorders.

Based on CuraGen Seqcalling data the Na+H+ Exchanger-like protein disclosed in this invention is expressed in at least lymphatic tissues. Based on expression data of the rat homolog to SC71046974_EXT, other tissues in which this gene is likely to be expressed include; stomach, small intestine, colon, kidney, brain, uterus, and skeletal muscle.

The NOV6 protein encoded by SEQ ID NO:11 has 717 amino acid residues and a predicted molecular weight of 80,733.6 Da, and is presented using the one-letter code in Table 6B (SEQ ID NO:12). The SignalP, Psort and/or Hydropathy profile for NOV6 predict that NOV6 has a signal peptide is likely to be localized at the plasma membrane (certainty=0.8200). A likely signal peptide cleavage site is between amino acids 26 and 27 of SEQ ID NO. 12.

TABLE 6B
Encoded NOV6 protein sequence (SEQ ID NO:12).

MALQMFVTYSPWNCLLLLVALECSEASSDLNESANSTAQYASNAWFAAASSEPEEGISVFELDYDYVQIP
YEVTLWILLASLAKIGFHLYHRLPGLMPESCLLILVGALVGGIIFGTDHKSPPVMDSSIYELYLLPPIVL
EGGYFMPTRPFFENIGSILWWAVLGALINALGIGLSLYLICQVKAFGLGDVNLLQNLLFGSLISAVDPVA
VLAVFEEARVNEQLYMMIFGEALLNDGITVVLYNMLIAFTKMHKFEDIETVDILAGCARFIVVGLGGVLF
GIVFGFISAFITRFTQNISAIEPLIVFMFSYLSYLAAETLYLSGILAITACAVTMKKYVEENVSQTSYTT
IKYFMKMLSSVSETLIFIFMGVSTVGKNHEWNWAFICFTLAFCQIWRAISVFALFYISNQFRTFPFSIKD
QCIIFYSGVRGAGSFSLAFLLPLSLFPRKKMFVTATLVVIYFTVFIQGITVGPLVRYLDVKKTNKKESIN
EELHIRLMDHLKAGIEDVCGHWSHYQVRDKFKKFDHRYLRKILIRKNLPKSSIVSLYKKLEMKQAIEMVE
TGILSSTAFSIPHQAQRIQGIKRLSPEDVESIRDILTSNMYQVRQRTLSYNKYNLKPQTSEKQAKEILIR
RQNTLRESMRKGHSLPWGKPAGTKNIRYLSYPYGNPQSAGRFTRAAGFSGKLPTWLLLWLRFGRGGQLTM
DTAGTITGPIVLCSKKN

The full amino acid sequence of the NOV6 protein was found to have 606 of 717 amino acid residues (84%) identical to, and 641 of 717 residues (89%) similar to, the 717 amino acid residue Na+H+ Exchanger-like protein from Rattus norvegicus (SWISSPROT-ACC:P26434).

Quantitative gene expression analysis (TaqMan) was performed on a NOV6 nucleic acid. The general method is described in Example 1. Results of TaqMan analysis of a NOV6 nucleic acid are described in Example 6.

Biochemical and pharmacological data support the existence of multiple forms of the Na/H exchanger (NHE). Two isoforms, termed NHE-1 and NHE-2, have recently been isolated from rabbit ileal villus epithelial cells. To identify additional molecular forms of the exchanger, rat brain, heart, kidney, stomach, and spleen cDNA libraries were screened for their presence using an NHE-1 cDNA probe under low stringency hybridization conditions. cDNAs encoding rat NHE-1 and two structurally related proteins, designated NHE-3 and NHE-4, have been isolated. Based on the deduced amino acid sequences, NHE-1, -3, and -4 are similar in size, having relative molecular masses of 91,506, 92,997, and 81,427, respectively. Overall, the proteins exhibit approximately 40% amino acid identity to each other and have similar hydropathy profiles, suggesting that they have the same transmembrane organization. The predicted N-terminal transmembrane regions of the three proteins, which span between 453 and 503 amino acids, exhibit the highest degree of identity (45-49%). In contrast, the C-terminal cytoplasmic regions, which span between 247 and 378 amino acids, exhibit very low amino acid identity (24-31%). Tissue distribution studies reveal that the NHE-1 mRNA is present at varying levels in all tissues examined, whereas NHE-3 and NHE-4 mRNAs exhibit a more limited distribution. NHE-3 mRNA is expressed at high levels in colon and small intestine, with significant levels also present in kidney and stomach. NHE-4 mRNA is most abundant in stomach, followed by intermediate levels in small intestine and colon and lesser amounts in kidney, brain, uterus, and skeletal muscle. The molecular basis for the functional diversity of the Na/H exchanger in mammals is based, at least in part, on expression of multiple members of a gene family.

The disclosed NOV6 protein of the invention has homology to the rat Na+H+ Exchanger-like protein. The rat Na+H+ Exchanger-like protein has characteristic properties, thus the NOV6 protein of the invention therefore is predicted to have characteristic properties homologous to the rat Na+H+ Exchanger-like protein. The expression pattern, map location, and protein similarity information for the invention(s) suggest that NOV6 may function as an RAT NA+H+ Exchanger-like protein family member.

The nucleic acids and proteins of the invention are useful in potential therapeutic applications implicated in various diseases and disorders and/or other pathologies and disorders. For example, a cDNA encoding the Rat NA+H+Exchanger-like protein -like protein may be useful in gene therapy, and the Rat NA+H+ Exchanger-like protein -like protein may be useful when administered to a subject in need thereof. By way of nonlimiting example, the compositions of the present invention will have efficacy for treatment of patients suffering from from Von Hippel-Lindau (VHL) syndrome, Cirrhosis, Transplantation, Hemophilia, Hypercoagulation, Idiopathic thrombocytopenic purpura, autoimmume disease, allergies, immunodeficiencies, transplantation, Graft vesus host, Diabetes, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome, Alzheimer's disease, Stroke, Tuberous sclerosis, Parkinson's disease, Huntington's disease, Cerebral palsy, Epilepsy, Multiple sclerosis, Ataxia-telangiectasia, Leukodystrophies, Behavioral disorders, Addiction, Anxiety, Pain, Xerostomia, Neuroprotection, Diabetes, Autoimmune disease, Renal artery stenosis, Interstitial ephritis, Glomerulonephritis, Polycystic kidney disease, Systemic lupus erythematosus, Renal tubular acidosis, Adrenoleukodystrophy, Congenital Adrenal Hyperplasia, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect VSD), valve diseases, Scleroderma, Obesity, Transplantation, Hyperthyroidism , Hypothyroidism, Fertility, Pancreatitis and/or other diseases/pathologies. The novel nucleic acid encoding the rat NA+H+ Exchanger-like protein-like protein, and the rat NA+H+ Exchanger-like protein-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

Potential therapeutic uses for the invention(s) are, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues). The nucleic acids and proteins of the invention are useful in potential therapeutic applications implicated in various diseases and disorders described below and/or other pathologies and disorders.

The nucleic acids and proteins of the invention are useful in potential diagnostic and therapeutic applications implicated in various diseases and disorders described above and/or other pathologies. Moreover, the polypeptides can be used as immunogens to produce antibodies specific for the invention, and as vaccines. They can also be used to screen for potential agonist and antagonist compounds. The novel nucleic acid encoding a sialoadhesin-like protein, and the sialoadhesin-like protein of the invention, or fragments thereof, may further be useful in diagnostic applications, wherein the presence or amount of the nucleic acid or the protein are to be assessed.

These materials are further useful in the generation of antibodies that bind immuno-specifically to the novel NOV6 substances for use in therapeutic or diagnostic methods. These antibodies may be generated according to methods known in the art, using prediction from hydrophobicity charts, as described in the “Anti-NOVX Antibodies” section below.

### NOV7

The NOV7 nucleic acid of 251 nucleotides (designated CuraGen Acc. No. GMAC040907.3_A) encoding a novel THYMOSIN BETA-4-like protein is shown in Table 7A. An open reading frame was identified beginning with an ATG initiation codon at nucleotides 49-51 and ending with a TGA codon at nucleotides 187-189. A putative untranslated region upstream from the initiation codon and downstream from the termination codon is underlined in Table 7A, and the start and stop codons are in bold letters.

TABLE 7A
NOV7 Nucleotide Sequence (SEQ ID NO:13)

GACAGGATTCCACACCTTTQCACTCCTGGCTCTGCTTTCTCTGCAACCATGTCTGACAAACCCAGCATGG
CTGAGATTGAGACACTCAATAAGCAGAGATTGAAGAAGGCAGAAACACAAGAGATAAATCCACCGCCTTC
AAGAGAAACAAACGAAGAACCACAGGTGAATTATAATGAGCTGTGAGCTGCGAATAGGTACTGCACA
TTCCATGGGCATTGCCTTCTTATTTTACTTCTTTTAGCTGT

In a search of sequence databases, it was found, for example, that the NOV7 nucleic acid sequence has 193 of 240 bases (80%) identical to a Rattus norvegicus THYMOSIN BETA-4 mRNA (GENBANK-ID: M34043). The NOV7 nucleic acid sequence maps to

The encoded NOV7 protein having 46 amino acid residues and a predicted molecular weight of 5,374.0 Da is presented using the one-letter code in Table 7B.

TABLE 7B
Encoded NOV7 protein sequence (SEQ ID NO:14).

MSDKPSMAEIETLNKQRLKKAETQEINPPPSRETNERSKQVNYNEL

The full amino acid sequence of a NOV7 protein was found to have 29 of 42 amino acid residues (69%) identical to, and 33 of 42 residues (78%) positive with, the 50 amino acid residue THYMOSIN BETA-4 protein from Mus musculus (ptnr:SPTREMBL-ACC:P20065). The global sequence homology (as defined by FASTA alignment with the full length sequence of the NOV7 protein) is 65.909% amino acid homology and 61.364% amino acid identity. Futher, the NOV7 protein is predicted to be a cytoplasmic protein (certainty of 0.6500) by PSORT analysis. In addition, the NOV7 protein contains the following protein domains (as defined by Interpro) at the indicated nucleotide positions: Thymosin domain (IPR001152) at amino acid positions 2 to 42 of SEQ ID NO. 14.

Other BLAST results include sequences from the Patp database, which is a proprietary database that contains sequences published in patents and patent publications. Patp results include those listed in Table 7C.

TABLE 7C
Patp alignments of NOV7

| | | | Smallest |
|---|---|---|---|
| | | | Sum |
| Sequences producing | Reading | High | Prob. |
| High-scoring Segment Pairs: | Frame | Score | P(N) |
| Patp:AYY76578 Huamn | −1 | 133 | 1.4e-10 |
| ovarian tumor EST. 86 aa | | | |
| patp:AAP81169 Protein produced | +1 | 147 | 1.4e-09 |
| in myeloma cell diff. 68 aa. | | | |

For example, a BLAST against patp: Aay76578, a 86 amino acid ovarian tumor EST fragment encoding protein 74 (DE19817557-A1), produced good identity (E=1.4c−10).

Quantitative gene expression analysis (TaqMan) was performed on a NOV7 nucleic acid. The general method is described in Example 1. Results of TaqMan analysis of a NOV7 nucleic acid are described in Example 7.

Thymosin beta-4 is a small polypeptide whose exact physiological role is not yet fully known It was first isolated as a thymic hormone that induces terminal deoxynucleotidyltransferase. It is found in high quantity in thymus and spleen but is widely distributed in many tissues. It has also been shown to bind to actin monomers and thus to inhibit actin polymerization. Thymosin-beta(4) (Theta(4)) binds actin monomers stoichiometrically and maintains the bulk of the actin monomer pool in metazoan cells. Theta(4) binding quenches the fluorescence of N-iodoacetyl-N′-(5-sulfo-1-naphthyl)ethylenediamine (AEDANS) conjugated to Cys(374) of actin monomers. The K(d) of the actin-Theta(4) complex depends on the cation and nucleotide bound to actin but is not affected by the AEDANS probe. The different stabilities are determined primarily by the rates of dissociation. At 25 degrees C., the free energy of Tbeta(4) binding MgATP-actin is primarily enthalpic in origin but entropic for CaATP-actin. Binding is coupled to the dissociation of bound water molecules, which is greater for CaATP-actin than MgATP-actin monomers. Proteolysis of MgATP-actin, but not CaATP-actin, at Gly(46) on subdomain 2 is >12 times faster when Theta(4) is bound. The C terminus of Tbeta(4) contacts actin near this cleavage site, at His(40). By tritium exchange, Theta(4) slows the exchange rate of approximately eight rapidly exchanging amide protons on actin. Tbeta(4) changes the conformation and structural dynamics (“breathing”) of actin monomers. The conformational change may reflect the unique ability of Tbeta(4) to sequester actin monomers and inhibit nucleotide exchange.

Thymosin beta-4 may also be important in angiogensesis. Angiogenesis is an essential step in the repair process that occurs after injury. In a rat full thickness wound model, addition of Tbeta4 topically or intraperitoneally increased reepithelialization by 42% over saline controls at 4 d and by as much as 61% at 7 d post-wounding. Treated wounds also contracted at least 11% more than controls by day 7. Increased collagen deposition and angiogenesis were observed in the treated wounds. Tbeta4 stimulated keratinocyte migration in the Boyden chamber assay. After 4-5 h, migration was stimulated 2-3-fold over migration with medium alone when as little as 10 pg of Tbeta4 was added to the assay.

The similarity information for the NOV7 protein and nucleic acid disclosed herein suggest that NOV7 may have important structural and/or physiological functions characteristic of Thymosin beta-4-like proteins. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon. The novel nucleic acid encoding NOV7, and the disclosed NOV7 protein, or fragments thereof, may further be useful in diagnostic applications, wherein the presence or amount of the nucleic acid or the protein are to be assessed.

The disclosed NOV7 polypeptides can be used as immunogens to produce vaccines. The novel nucleic acid encoding NOV-like protein, and the NOV-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. For example the disclosed NOV7 protein has multiple hydrophilic regions, each of which can be used as an immunogen. These novel proteins can also be used to develop assay system for functional analysis. These antibodies may be generated according to methods known in the art, using prediction from hydrophobicity charts, as described in the “Anti-NOVX Antibodies” section below.

### NOV8

The NOV8 nucleic acid of 2144 nucleotides (designated CuraGen Acc. No. 20760813_EXT) encoding a novel Leucine rich repeats protein is shown in Table 8A. An open reading frame was identified beginning with an ATG initiation codon at nucleotides 1 and ending with a TGA codon at nucleotides 1819.

TABLE 8A
NOV8 Nucleotide Sequence (SEQ ID NO:15)

ATGCTTCACACGGCCATATCATGCTCGCAGCCATTCCTGGGTCTGGCTGTGGTGTTAATCTTCATGGGAT
CCACCATTGGCTGCCCCGCTCGCTGTGAGTGCTCTCCCCAGAACAAATCTGTTAGCTGTCACAGAAGGCG
ATTGATCGCCATCCCAGAGGGCATTCCCATCGAAACCAAAATCTTGGACCTCAGTAAAAACAGGCTAAAA
AGCGTCAACCCTGAAGAATTCATATCATATCCTCTGCTGCAAGACATAGACTTCAGTGACAACATCATTG
CCAATGTCGAACCAGGAGCATTCAACAATCTCTTTAACCTGCGTTCCCTCCGCCTAAAAGGCAATCGTCT
AAAGCTGGTCCCTTTGGGAGTATTCACGGGGCTGTCCAATCTCACTAAGCTTCACATTAGTGAGAATAAC
ATTGTCATTTTACTAGACTACATGTTCCAAGATCTACATAACCTGAAGTCTCTAGAAGTGGGGGACAATG
ATTTGGTTTATATATCACACAGGGCATTCAGTGGGCTTCTTAGCTTGGAGCAGCTCACCCTGGAGAAATG
CAACTTAACACCAGTACCAACAGAAGCCCTCTCCCACCTCCGCACCCTCATCAGCCTGCATCTGAAGCAT
CTCAATATCAACAATATGCCTGTGTATGCCTTTAAAAGATTGTTCCACCTGAAACACCTAGAGATTGACT
ATTGGCCTTTACTGGATATGATGCCTCCCAATAGCCTCTACCGTCTCAACCTCACATCCCTTTCAGTCAC
CAACACCAATCTGTCTACTGTACCCTTCCTTGCCTTTAAACACCTGGTATACCTGACTCACCTTAACCTC
TCCTACAATCCCATCAGCACTATTGAAGCAGGCATGTTCTCTGACCTGATCCGCCTTCAGGAGCTTCATA
TAGTGGGGCCCCAGCTTCGCACCATTCAGCCTCACTCCTTCCAAGGGCTCCGCTTCCTACGCCTGCTCAA
TGTGTCTCAGAACCTCCTGGAAACTTTGGAAGAGAATGTCTTCTCCTCCCCTAGGGCTCTGGAGGTCTTG
AGCATTAACAACAACCCTCTGGCCTGTGACTGCCGCCTTCTCTGGATCTTGCAGCCACAGCCCACCCTGC
AGTTTGGTGGCCAGCAACCTATGTGTGCTGGCCCAGACACCATCCGTGAGACGTCTTTCAAGGATTTCCA
TAGCACTGCCCTTTCTTTTTACTTTACCTGCAAAAAACCCAAAATCCGTGAAAAGAAGTTGCAGCATCTG
CTAGTAGATGAAGGGCAGACAGTCCACCTAGAATGCAGTGCAGATGGAGACCCCCACCCTGTGATTTCCT
GGGTGACACCCCCAAGGCGTTTCATCACCACCAAGTCCAATGGAAGAGCCACCGTGTTGGGTGATGGCAC
CTTGGAAATCCGCTTTGCCCAGGATCAAGACAGCGCGATGTATGTTTGCATCGCTAGCAATGCTGCTGGG
AATGATACCTTCACAGCCTCCTTAACTGTGAAACGATTCGCTTCAGATCGTTTTCTTTATCCGAACAGGA
CCCCTATGTACATGACCGACTCCAATGACACCATTTCCAATGGCAGCAATGCCAATACTTTTTCCCTGGA
CCTTAAAACAATACTGGTGTCTACAGCTATGCGCTGCTTCACATTCCTGGGAGTGGTTTTATTTTGTTTT
CTTCTCCTTTTTGTGTGGAGCCGAGGGAAAGCCAAGCACAAAAACACCATTGACCTTGAGTATGTGCCCA
AAAAAAACCATGCTGCTGTTGTGGAAGGGGAGGTAGCTGGACCCAGGACGTTCAACATGAAAATGATTTG
AAGGCCCACCCCTCACATTACTGTCTCTTTGTCAATGTGGGTAATCAGTAAGACAGTATGGCACACTAAA
TTACTAGATTAAGAGGCAGCCATGTGCAGCTGCCCCTGTATCAAAAGAGGGTCTATGGAAGCACGAGGA
CTTCCAATGGAGACTCTCCATCGAAAGGCAGGCAGGCAGGCATGTGTCACAGCCCTTCACACAGTGGCAT
ACTAAGTGTTTGCGTTGCAAATATTGGCGTTCTGGGGATCTCAGTAATGAACCTGAATATTTGACTCACA
CTCACGGACAATTATTCAGCATTTTCTACCACTCCAAAAAAAAA

The NOV8 encoded protein having 606 amino acid residues and a predicted molecular weight of 68,046.0 is presented using the one-letter code in Table 8B.

TABLE 8B
Encoded NOV8 protein sequence (SEQ ID NO:14).+HZ,1/44

MLHTAISCWQPFLGLAVVLIFMGSTIGCPARCECSAQNKSVSCHRRRLIAIPEGIPIETKILDLSKNRLK
SVNPEEFISYPLLEEIDLSDNIIANVEPGAFNNLFNLRSLRLKGNRLKLVPLGVFTGLSNLTKLDISENK
IVTLLDYMFQDLHNLKSLEVGDNDLVYISHRAFSGLLSLEQLTLEKCNLTAVPTEALSHLRSLISLHLKH
LNINNMPVYAFKRLFHLKHLEIDYWPLLDMMPANSLYGLNLTSLSVTNTNLSTVPFLAFKHLVYLTHLNL
SYNPISTIEAGMFSDLIRLQELHIVGAQLRTIEPHSFQGLRFLRVLNVSQNLLETLEENVFSSPRALEVL
SINNNPLACDCRLLWILQRQPTLQFGGQQPMCAGPDTIRERSFKDFHSTALSFYFTCKKPKIREKKLQHL
LVDEGQTVQLECSADGDPQPVISWVTPRRRFITTKSNGRATVLGDGTLEIRFAQDQDSGMYVCIASNAAG
NDTFTASLTVKGFASDRFLYANRTPMYMTDSNDTISNGSNANTFSLDLKTILVSTAMGCFTFLGVVLFCF
LLLFVWSRGKGKHKNSIDLEYVPKKNHGAVVEGEVAGPRRFNMKMI

Quantitative gene expression analysis (TaqMan) was performed on a NOV8 nucleic acid. The general method is described in Example 1. Results of TaqMan analysis of a NOV8 nucleic acid are described in Example 8.

The present invention also includes variants of the NOV8 sequence, including SNPs. Possible SNPs found for NOV8 are listed in Table 8C.

TABLE 8C
NOV8 SNPs

| Base | Base | Alteration |
|---|---|---|
| Position | Before/After | effect |
| 389 | A/G | Asn to Ser |

The full amino acid sequence of the NOV8 protein was found to have 363 of 601 amino acid residues ( 60%) identical to, and 466 of 601 residues (77%) positive with, the 614 amino acid residue BAB03557 HYPOTHETICAL 69.2 KDA PROTEIN protein from macaca (ptnr:SPTREMBL-ACC:BAB03557; E=2.6e−¹⁹⁹). In addition, this protein contains the following protein domains (as defined by Interpro): 12 Leucine Rich Repeat (LRR) 1 Immunoglobulin domain, 1 Leucine rich repeat (LRR) N-terminal domain (LRRNT) and 1 Leucine rich repeat C-terminal domain (LRRCT). Further, PSORT analysis suggests that the NOV8 protein is a plasma membrane protein (certainty of 0.4600) and SIGNALP analysis suggests that NOV8 has a signal peptide, with the most likely cleavage site between positions 27 and 28 of SEQ ID NO:16.

Other BLAST results include sequences from the Patp database, which is a proprietary database that contains sequences published in patents and patent publications. Patp results include those listed in Table 8C.

TABLE 8C
Patp alignments of NOV8

| | | | Smallest |
|---|---|---|---|
| | | | Sum |
| Sequences producing | Reading | High | Prob. |
| High-scoring Segment Pairs: | Frame | Score | P(N) |
| >patp:AAB31161 Amino acid sequence | +1 | 2137 | 1.9e-220 |
| of a human TOLL protein 548 aa | | | |

Leucine rich repeats (LRR) mediate reversible protein-protein interactions. LRR proteins have diverse cellular functions, like cell adhesion and cellular signaling. Several of these proteins, such as connectin, slit, chaoptin, and Toll have pivotal roles in neuronal development in Drosophila and may play significant but distinct roles in neural development and in the adult nervous system of humans. The NOV8 nucleic acids and proteins are useful in potential diagnostic and therapeutic applications implicated in various diseases and disorders described below and/or other pathologies. For example, the compositions of the present invention will have efficacy for treatment of patients suffering from: Inflamation, Autoimmune disorders, Aging, cancer, Cardiomyopathy, Atherosclerosis,Hypertension, Congenital heart defects, Aortic stenosis ,Atrial septal defect (ASD),Atrioventricular (A-V) canal defect, Ductus arteriosus , Pulmonary stenosis, Subaortic stenosis, Ventricular septal defect (VSD), valve diseases,Tuberous sclerosis, Scleroderma, Obesity,Transplantation, Diabetes,Von Hippel-Lindau (VHL) syndrome, Pancreatitis,Obesity, Endometriosis,Fertility, Hemophilia, Hypercoagulation,Idiopathic thrombocytopenic purpura, Immunodeficiencies,Graft vesus host, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan as well as other diseases, disorders and conditions.

The novel nucleic acid encoding the NOV8 proteins, and the NOV8 proteins of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The novel nucleic acid encoding NOV8 proteins, and the NOV8 proteins of the invention, or fragments thereof, may further be useful in diagnostic applications, wherein the presence or amount of the nucleic acid or the protein are to be assessed. These materials are further useful in the generation of antibodies that bind immuno-specifically to the novel NOV8 substances for use in therapeutic or diagnostic methods. These antibodies may be generated according to methods known in the art, using prediction from hydrophobicity charts, as described in the “Anti-NOVX Antibodies” section below.

### NOV9

In the present invention a NOV8 nucleic acid was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain-amygdala, brain-cerebellum, brain-hippocampus, brain-substantia nigra, brain-thalamus, brain -whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated NOV9 (Accession Number CG51514-03). This differs from the previously identified sequence (NOV8; Accession Number 20760813_EXT) at aminoacid positions 24 S->P, 63 D->N, 220 A->T, 253 S->P, 529 S->T, 584 K->R amd 587 H->S.

The NOV9 nucleic acid of 2187 nucleotides encoding a novel Leucine rich repeats protein is shown in Table 9A. An open reading frame was identified beginning with an ATG initiation codon at nucleotides 83-85 and ending with a TGA codon at nucleotides 1901.

TABLE 9A
NOV9 Nucleotide Sequence (SEQ ID NO:17)

AATCATGAGGAACCTATAACCCTTTTGGCCACATGCAAAAAAGCAAGACCCGTGACCAAG
GTGTAGACTAAGAAGTGGAGTCATGCTTCACACGGCCATATCATGCTGGCAGCCATTCCT
GGGTCTGGCTGTGGTGTTAATCTTCATGGGACCCACCATTGGCTGCCCCGCTCGCTGTGA
GTGCTCTGCCCAGAACAAATCTGTTAGCTGTCACAGAAGGCGATTGATCGCCATCCCAGA
GGGCATTCCCATCGAAACCAAAATCTTGAACCTCAGTAAAAACAGGCTAAAAAGCGTCAA
CCCTGAAGAATTCATATCATATCCTCTGCTGGAAGAGATAGACTTGAGTGACAACATCAT
TGCCAATGTGGAACCAGGAGCATTCAACAATCTCTTTAACCTGCGTTCCCTCCGCCTAAA
AGGCAATCGTCTAAAGCTGGTCCCTTTGGGAGTATTCACGGGGCTGTCCAATCTCACTAA
GCTTGACATTAGTGAGAATAAGATTGTCATTTTACTAGACTACATGTTCCAAGATCTACA
TAACCTGAAGTCTCTAGAAGTGGGGGACAATGATTTGGTTTATATATCACACAGGGCATT
CAGTGGGCTTCTTAGCTTGGAGCAGCTCACCCTGGAGAAATGCAACTTAACAGCAGTACC
AACAGAAGCCCTCTCCCACCTCCGCAGCCTCATCAGCCTGCATCTGAAGCATCTCAATAT
CAACAATATGCCTGTGTATACCTTTAAAAGATTGTTCCACCTGAAACACCTAGAGATTGA
CTATTGGCCTTTACTGGATATGATGCCTGCCAATAGCCTCTACGGTCTCAACCTCACACC
CCTTTCAGTCACCAACACCAATCTGTCTACTGTACCCTTCCTTGCCTTTAAACACCTGGT
ATACCTGACTCACCTTAACCTCTCCTACAATCCCATCAGCACTATTGAAGCAGGCATGTT
CTCTCACCTGATCCGCCTTCAGGAGCTTCATATAGTGGGGGCCCAGCTTCCCACCATTGA
GCCTCACTCCTTCCAAGGGCTCCGCTTCCTACGCGTGCTCAATGTGTCTCAGAACCTGCT
GGAAACTTTGGAAGAGAATGTCTTCTCCTCCCCTAGGGCTCTGGAGGTCTTGAGCATTAA
CAACAACCCTCTGGCCTGTGACTGCCGCCTTCTCTGGATCTTGCAGCGACAGCCCACCCT
GCAGTTTGGTGGCCAGCAACCTATGTGTGCTGGCCCAGACACCATCCGTGAGAGGTCTTT
CAAGGATTTCCATAGCACTGCCCTTTCTTTTTACTTTACCTGCAAAAAACCCAAAATCCG
TGAAAAGAAGTTGCAGCATCTGCTAGTAGATGAAGGGCAGACAGTCCAGCTAGAATGCAG
TGCAGATGGAGACCCGCAGCCTGTGATTTCCTGGGTGACACCCCGAACGCCTTTCATCAC
CACCAAGTCCAATGGAAGAGCCACCGTGTTGGGTGATGGCACCTTGGAAATCCGCTTTGC
CCAGGATCAAGACAGCGGGATGTATGTTTGCATCGCTAGCAATGCTGCTGGGAATGATAC
CTTCACACCCTCCTTAACTGTGAAAGGATTCGCTTCAGATCGTTTTCTTTATGCGAACAG
GACCCCTATGTACATGACCGACTCCAATGACACCATTTCCAATGGCACCAATGCCAATAC
TTTTTCCCTGGACCTTAAAACAATACTGGTGTCTACAGCTATGGGCTGCTTCACATTCCT
CGGACTGGTTTTATTTTGTTTTCTTCTCCTTTTTCTGTGCAGCCGAGGGAAAGGCAAGCA
CAAAAACAGCATTGACCTTGAGTATGTGCCCAGAAAAAACAGTGGTCCTGTTGTGGAAGG
GGAGGTAGCTGGACCCAGGAGGTTCAACATGAAAATGATTTGAAGGCCCACCCCTCACAT
TACTGTCTCTTTGTCAATGTGGGTAATCAGTAAGACAGTATGGCACAGTAAATTACTAGA
TTAAGAGGCAGCCATGTGCAGCTGCCCCTGTATCAAAAGCAGGGTCTATGGAAGCAGGAG
GACTTCCAATGGAGACTCTCCATCGAAAGGCAGGCAGGCAGGCATGTGTCAGAGCCCTTC
ACACAGTGGGATACTAAGTGTTTGCGTTGCAAATATTGGCGTTCTGGGGATCTCAGTAAT
GAACCTGAATATTTGGCTCACACTCAC

The NOV9 encoded protein having 606 amino acid residues is presented using the one-letter code in Table 9B.

TABLE 9B
Encoded NOV9 protein sequence (SEQ ID NO:18).

MLHTAISCWQPFLGLAVVLIFMGPTIGCPARCECSAQNKSVSCHRRRLTATPEGIPIETK
ILNLSKNRLKSVNPEEFISYPLLEEIDLSDNIIANVEPGAFNNLFNLRSLRLKGNRLKLV
PLGVFTGLSNLTKLDISENKIVILLDYMFQDLHNLKSLEVGDNDLVYISHRAFSGLLSLE
QLTLEKCNLTAVPTEALSHLRSLISLHLKHLNINNMPVYTFKRLFHLKHLEIDYWPLLDM
MPANSLYGLNLTPLSVTNTNLSTVPFLAFKHLVYLTHLNLSYNPISTIEAGMFSDLIRLQ
ELHIVGAQLRTIEPHSFQGLRFLRVLNVSQNLLETLEENVFSSPRALEVLSINNNPLACD
CRLLWILQRQPTLQFGGQQPMCAGPDTIRERSFKDFHSTALSFYFTCKKPKIREKKLQHL
LVDEGQTVQLECSADGDPQPVISWVTPRRRFITTKSNGRATVLGDGTLEIRFAQDQDSGM
YVCIASNAAGNDTFTASLTVKGFASDRFLYANRTPMYMTDSNDTISNGTNANTFSLDLKT
ILVSTAMGCFTFLGVVLFCFLLLFVWSRGKGKHKNSIDLEYVPRKNSGAVVEGEVAGPRR
FNMKMI

The present invention also includes variants of the NOV9 sequence, including SNPs. Possible SNPs found for NOV9 are listed in Table 9C.

TABLE 9C
NOV9 SNPs

| Base | Base | Base |
|---|---|---|
| Position | Before | After |
| 346 | T | C |
| 1449 | C | T |
| 1718 | T | C |
| 1920 | C | T |
| 2037 | A | G |
| 2160 | G | A |

The full amino acid sequence of the protein of the invention was found to have 601 of 606 amino acid residues (99%) identical to, and 603 of 606 amino acid residues (99%) similar to, the 606 amino acid residue ptnr:TREBLNEW-ACC:CAC22713 protein from Homo sapiens (BA438B23.1; NEURONAL LEUCINE-RICH REPEAT PROTEIN).

The pattern of expression of this gene and its family members, and its similarity to the TRG family of genes suggests that if may function as a TRG family protein Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of the invention are useful in potential therapeutic applications implicated in hypo- and hyperthyroidism, disorders of the thyroid, cancer including but not limited to thyroid-related cancers, and/or other pathologies and disorders. For example, a cDNA encoding the TRG-like protein may be useful in gene therapy, and the TRG-like protein may be useful when administered to a subject in need thereof. By way of nonlimiting example, the compositions of the present invention will have efficacy for treatment of patients suffering from hypo- and hyperthyroidism, disorders of the thyroid, cancer including but not limited to thyroid-related cancers. The novel nucleic acid encoding TRG-like protein, and the TRG-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

These materials are further useful in the generation of antibodies that bind immuno-specifically to the novel NOV9 substances for use in therapeutic or diagnostic methods. These antibodies may be generated according to methods known in the art, using prediction from hydrophobicity charts, as described in the “Anti-NOVX Antibodies” section below.

### NOV10-12

Three nucleic acids and proteins encoded thereby of the present invention belong to the WNT-5A family of proteins, and are designated NOV10, NOV11 and NOV 12.

### NOV10

A NOV10 nucleic acid of 1215 nucleotides (also designated CuraGen Acc. No. SC128855163_A) encoding a novel WNT-5A-like protein is shown in Table 1A. An open reading frame was identified beginning with an CTC initiation codon at nucleotides 16-18 and ending with a TAG codon at nucleotides 1156-1158. A putative untranslated region upstream from the initiation codon and downstream from the termination codon is underlined in Table 10A, and the start and stop codons are in bold letters.

TABLE 10A
NOV10 Nucleotide Sequence (SEQ ID NO:19)

GCTCCTTTCTTCCCTCTCCAGAAGTCCATTGGAATATTAAGCCCAGGAGTTGCTTTGGGGATGGCTGGAA
GTGCAATGTCTTCCAAGTTCTTCCTAGTGGCTTTGGCCATATTTTTCTCCTTCGCCCAGGTTGTAATTGA
AGCCAATTCTTGGTGGTCGCTAGGTATGAATAACCCTGTTCAGATGTCAGAAGTATATATTATAGGAGCA
CAGCCTCTCTGCAGCCAACTGGCAGGACTTTCTCAAGGACAGAAGAAACTGTGCCACTTGTATCAGGACC
ACATGCAGTACATCGGAGAAGGCGCGAAGACAGGCATCAAAGAATGCCAGTATCAATTCCGACATCGAAG
GTGGAACTGCAGCACTGTGGATAACACCTCTGTTTTTGGCAGGGTGATGCAGATAGGTAGCCGCGAGACG
GCCTTCACATACGCGGTGAGCGCAGCAGGGGTGGTGAACGCCATGAGCCGGGCGTGCCGCGAGGGCGAGC
TGTCCACCTGCGGCTGCAGCCGCGCCGCGCGCCCCAAGGACCTGCCGCGGGACTGGCTCTGGGGCGGCTC
CGGCGCCACCAACAAAAAAGGCTACCGCTCCGCCAAGGAGATCGTGCACGCCCGCGAACGAGGACGCATC
CACGCCAAGGGCTCCTACGAGAGTGCTCGCATCCTCATGAACCTGCACAACAACGAGGCCGGCCGCAGGA
CGGTGTACAACCTGGCTGATGTGGCCTGCAAGTGCCATGGGGTGTCCGGCTCATGTAGCCTGAAGACATG
CTGGCTGCAGCTGGCAGACTTCCGCAAGGTGGGTGATGCCCTGAAGGAGAAGTACGACAGCGCGGCGGCC
ATGCGGCTCAACAGCCGGGGCAAGTTGGTACAGGTCAACAGCCGCTTCAACTCGCCCACCACACAAGACC
TGGTCTACATCGACCCCAGCCCTGACTACTGCGTGCGCAATGAGAGCACCGGCTCGCTGGGCACGCAGGG
CCGCCTGTGCAACAAGACGTCGGAGGGCATGGATGGCTGCGAGCTCATGTGCTGCGGCCGTGGCTACGAC
CAGTTCAAGACCGTGCAGACGGAGCGCTGCCACTGCAAGTTCCACTGGTGCTGCTACGTCAAGTGCAAGA
AGTGCACGGAGATCGTGGACCAGTTTGTGTGCAAGTAGTGGGTGCCACCCAGCACTCAGCCCCGCTCCCA
GGACCCGCTTATTTATAGAAAGTAC

In a search of sequence databases, it was found, for example, that the NOV10 nucleic acid sequence has 1194 of 1213 bases (98%) identical to a Homo sapiens WNT-5A mRNA (GENBANK-ID: acc:L20861). Although there is high homology to the human WNT-5A protein, there is significant variation in exon 3 from nucleotide position 528 to nucleotide position 624, and therefore, this sequence appears to be a splice variant.

In a search of CuraGen's proprietary human expressed sequence assembly database, assemblies s3aq:126056009 (472 nucleotides) and s3aq:128855163 (4113 nucleotides) were identified as having high homology to a NOV10 nucleic acid. This database is composed of the expressed sequences (as derived from isolated mRNA) from more than 96 different tissues. The mRNA is converted to cDNA and then sequenced. These expressed DNA sequences are then pooled in a database and those exhibiting a defined level of homology are combined into a single assembly with a common consensus sequence. The consensus sequence is representative of all member components. Since the nucleic acid of the described invention has >95% sequence identity with the CuraGen assembly, the nucleic acid of the invention represents an expressed gene sequence. The DNA assembly s3aq:126056009 has 2 and the DNA assembly s3aq:128855163 has 18 components and were found by CuraGen to be expressed in at least kidney, brain, and hematopoietic tissues.

The encoded NOV10 protein having 380 amino acid residues and a predicted molecular weight of 42,082.8 Da is presented using the one-letter code in Table 10B.

TABLE 10B
Encoded NOV10 protein sequence (SEQ ID NO:20).

LQKSIGILSPGVALGMAGSAMSSKFFLVALAIFFSFAQVVIEANSWWSLGMNNPVQMSEVYIIGAQPLCSQLAGLSQGQK
KLCHLYQDHMQYIGEGAKTGIKECQYQFRHRRWNCSTVDNTSVFGRVMQIGSRETAFTYAVSAAGVVNAMSRACREGELS
TCGCSRAARPKDLPRDWLWGGSGATNKKGYRSAKEIVHARERGRIHAKGSYESARILMNLHNNEAGRRTVYNLADVACKC
HGVSGSCSLKTCWLQLADFRKVGDALKEKYDSAAAMRLNSRGLVQVNSRFNSPTTQDLVYIDPSPDYCVRNESTGSLGT
QGRLCNKTSEGMDGCELMCCGRGYDQFKTVQTERCHCKFHWCCYVKCKKCTEIVDQFVCK

The full amino acid sequence of the NOV10 protein was found to have 360 of 380 amino acid residues (94% %) identical to, and 364 of 380 residues (95%) positive with, the 379 amino acid residue WNT-5A protein from Rattus norvegicus (ptnr:SPTREMBL-ACC: Q9QXQ7). The global sequence homology (as defined by FASTA alignment with the fill length sequence of this protein) is 95.251% amino acid homology and 94.987% amino acid identity. PSORT analysis predicts the NOV10 protein to be localized outside of the cell with a certainty of 0.8200. Using the SIGNALP analysis, it is predicted that the NOV10 protein has a signal peptide with most likely cleavage site between pos. 42 and 43. In addition, the NOV10 protein contains a Wnt (IPR000970) at amino acid positions 68 to 380 (as defined by Interpro).

Other BLAST results include sequences from the Patp database, which is a proprietary database that contains sequences published in patents and patent publications. Patp results include those listed in Table 10C.

TABLE 10C
Patp alignments of NOV10

| | | | Smallest |
|---|---|---|---|
| | | | Sum |
| Sequences producing | Reading | High | Prob. |
| High-scoring Segment Pairs: | Frame | Score | P(N) |
| >patp:AAY70739 Human Wnt-5a | +1 | 1914 | 7.9e-197 |
| protein - Homo sapiens, 365 aa | | | |
| >patp:AAY57600 Human Wnt-5a | +1 | 1914 | 7.9e-197 |
| protein - Homo sapiens, 365 aa. | | | |

Quantitative gene expression analysis (TaqMan) was performed on a NOV10 nucleic acid. The general method is described in Example 1. Results of TaqMan analysis of a NOV10 nucleic acid are described in Example 9.

### NOV11

The NOV11 nucleic acid of 4113 nucleotides (also designated CuraGen Acc. No. SC128855163_B) encoding a novel WNT-5A-like protein is shown in Table 11A. An open reading frame was identified beginning with an CTC initiation codon at nucleotides 439-441 and ending with a TAG codon at nucleotides 1579-1581. A putative untranslated region upstream from the initiation codon and downstream from the termination codon is underlined in Table 11A, and the start and stop codons are in bold letters.

TABLE 11A
Encoded NOV11 nucleic acid sequence (SEQ ID NO:21).

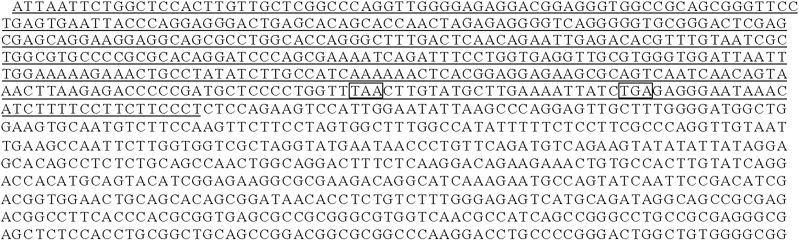

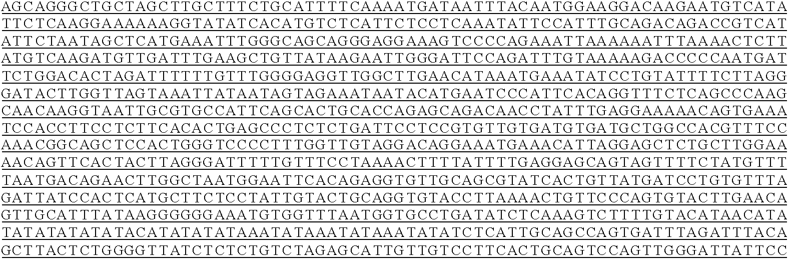

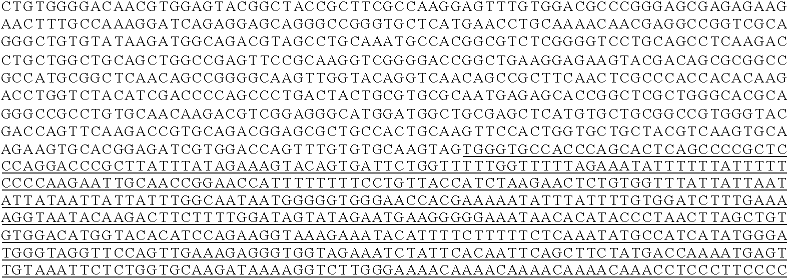

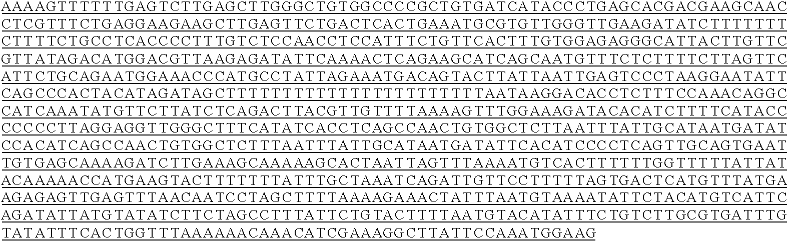

In a search of sequence databases, it was found, for example, that the nucleic acid sequence has 4035 of 4114 bases (98%) identical to a Homo sapiens WNT-5A mRNA (GENBANK-ID:

acc:L20861).

The encoded protein having 380 amino acid residues and a predicted molecular weight of 42,333.0 Da is presented using the one-letter code in Table 11B.

TABLE 11B
Encoded NOV11 protein sequence (SEQ ID NO:22).

LQKSIGILSPGVALGMAGSAMSSKFFLVALAIFFSFAQVVIEANSWWSLGMNNPVQMSEVYIIGAQPLCSQLAGLSQGQK
KLCHLYQDHMQYIGEGAKTGIKECQYQFRHRRWNCSTADNTSVFGRVMQIGSRETAFTHAVSAAGVVNAISRACREGELS
TCGCSRTARPKDLPRDWLWGGCGDNVEYGYRFAKEFVDAREREKNFAKGSEEQGRVLMNLQNNEAGRRAVYKMADVACKC
HGVSGSCSLKTCWLQLAEFRKVGDRLKEKYDSAAAMRLNSRGKLVQVNSRFNSPTTQDLVYIDPSPDYCVRNESTGSLGT
QGRLCNKTSEGMDGCELMCCGRGYDQFKTVQTERCHCKFHWCCYVKCKKCTEIVDQFVCK

The full amino acid sequence of the NOV11 protein was found to have 352 of 380 amino acid residues (92%) identical to, and 364 of 380 residues (95%) positive with, the 379 amino acid residue WNT-5A protein from Rattus norvegicus (ptnr:SPTREMBL-ACC: Q9QXQ7). The global sequence homology (as defined by FASTA alignment with the fill length sequence of the NOV11 protein) is 94.987% amino acid homology and 92.876% amino acid identity. In addition, the NOV11 protein contains the following protein domains (as defined by Interpro) at the indicated nucleotide positions: Wnt domain (IPR000970) at amino acid positions 68 to 380 of SEQ ID NO. 22.

PSORT analysis predicts the NOV11 protein to be localized outside of the cell with a certainty of 0.8200. Using the SIGNALP analysis, it is predicted that the NOV11 protein has a signal peptide with most likely cleavage site between pos. 42 and 43 of SEQ ID NO. 22.

The present invention also includes variants of the NOV11 sequence, including SNPs. Possible SNPs found for NOV11 are listed in Table 11C.

TABLE 11C
NOV11 SNPs

| Base | | |
|---|---|---|
| Position | Base Before | Base After |
| 1185 | C | T |

Other BLAST results include sequences from the Patp database, which is a proprietary database that contains sequences published in patents and patent publications. Patp results include those listed in Table 11D.

TABLE 11D
Patp alignments of NOV11

Sequences producing High-scoring
Segment Pairs:

| | | | Smallest |
|---|---|---|---|
| | | | Sum |
| | Reading | High | Prob. |
| | Frame | Score | P(N) |
| >patp:AAY70739 Human Wnt-5a protein - | +1 | 1900 | 7.9e−197 |
| Homo sapiens, 365 aa. | | | |
| >patp:AAY57600 Human Wnt-5a protein - | +1 | 1900 | 7.9e−197 |
| Homo sapiens, 365 aa. | | | |

### NOV12

The NOV12 nucleic acid of 1214 nucleotides (also designated CuraGen Acc. No. CG56768-01) encoding a novel Wnt-5A-like protein is shown in Table 12A. An open reading frame was identified beginning with an ATG initiation codon at nucleotides 60-62 and ending with a TAG codon at nucleotides 1155-1157. Putative untranslated regions are found upstream from the initiation codon and downstream from the termination codon.

TABLE 12A
Encoded NOV12 nucleic acid sequence (SEQ ID NO:23).

CTCCTTTCTTCCCTCTCCAGAAGTCCATTGGAATATTAAGCCCAGGAGTTGCTTTGGGGA
TGGCTGGAAGTGCAATGTCTTCCAAGTTCTTCCTAGTGGCTTTGGCCATATTTTTCTCCT
TCGCCCAGGTTGTAATTGAAGCCAATTCTTGGTGGTCGCTAGGTATGAATAACCCTGTTC
AGATGTCAGAAGTATATATTATAGGAGCACAGCCTCTCTGCAGCCAACTGGCAGGACTTT
CTCAAGGACAGAAGAAACTGTGCCACTTGTATCAGGACCACATGCAGTACATCGGAGAAG
GCGCGAAGACAGGCATCAAAGAATGCCAGTATCAATTCCGACATCGAAGGTGGAACTGCA
GCACTGTGGATAACACCTCTGTTTTTGGCAGGGTGATGCAGATAGGCAGCCGCGAGACGG
CCTTCACATACGCGGTGAGCGCAGCAGGGGTGGTGAACGCCATGAGCCGGGCGTGCCGCG
AGGGCGAGCTGTCCACCTGCGGCTGCAGCCGCGCCGCGCGCCCCAAGGACCTGCCGCGGG
ACTGGCTCTGGGGCGGCTGCGGCGACAACATCGACTATGGCTACCGCTTTGCCAAGGAGT
TCGTGGACGCCCGCGAGCGGGAGCGCATCCACGCCAAGGGCTCCTACGAGAGTGCTCGCA
TCCTCATGAACCTGCACAACAACGAGGCCGGCCGCAGGACGGTGTACAACCTGGCTGATG
TGGCCTGCAAGTGCCATGGGGTGTCCGGCTCATGTAGCCTGAAGACATGCTGGCTGCAGC
TGGCAGACTTCCGCAAGGTGGGTGATGCCCTGAAGGAGAAGTACGACAGCGCGGCGGCCA
TGCCGCTCAACAGCCGGGCCAAGTTGGTACAGGTCAACAGCCGCTTCAACTCGCCCACCA
CACAAGACCTGGTCTACATCGACCCCAGCCCTGACTACTGCGTGCGCAATGAGAGCACCG
GCTCGCTGGGCACGCAGGGCCGCCTGTGCAACAAGACGTCGGACCGCATGGATGGCTGCG
AGCTCATGTGCTGCGGCCGTGGCTACGACCAGTTCAAGACCGTGCAGACGGAGCGCTGCC
ACTGCAAGTTCCACTGGTGCTGCTACGTCAAGTGCAAGAAGTGCACGGAGATCGTGGACC
AGTTTGTGTGCAAGTAGTGGGTGCCACCCAGCACTCAGCCCCGCCCCCAGGACCCGCTTA
TTTATAGAAAGTAC

In a search of sequence databases, it was found, for example, that the NOV12 nucleic acid sequence has 1208 of 1213 bases (99%) identical to a omo sapiens Wnt-5a mRNA (GENBANK-ID:HUMWNT5A|acc:L20861.1). The NOV12 nucleic acid is expressed in at least the following tissues: aorta, brain, heart, kidney, and lymph node. This information was derived by determining the tissue sources of the sequences that were included in the invention including but not limited to SeqCalling sources, Public EST sources, Literature sources, and/or RACE sources.

The encoded protein having 365 amino acid residues is presented using the one-letter code in Table 12B.

TABLE 12B
Encoded NOV12 protein sequence (SEQ ID NO:24).

MAGSAMSSKFFLVALAIFFSFAQVVIEANSWWSLGMNNPVQMSEVYIIGAQPLCSQLAGL
SQGQKKLCHLYQDHMQYIGEGAKTGIKECQYQFRHRRWNCSTVDNTSVFGRVMQIGSRET
AFTYAVSAAGVVNAMSRACREGELSTCGCSRAARPKDLPRDWLWGGCGDNIDYGYRFAKE
FVDARERERIHAKGSYESARILMNLHNNEAGRRTVYNLADVACKCHGVSGSCSLKTCWLQ
LADFRKVGDALKEKYDSAAAMRLNSRGKLVQVNSRFNSPTTQDLVYIDPSPDYCVRNEST
GSLGTQGRLCNKTSEGMDGCELMCCGRGYDQFKTVQTERCHCKFHWCCYVKCKKCTEIVD
QFVCK

The full amino acid sequence of the NOV12 protein was found to have 365 of 365 amino acid residues (100%) identical to, and 365 of 365 amino acid residues (100%) similar to, the 365 amino acid residue protein from the human WNT-5A protein precursor (ptnr:SWISSPROT-ACC:P41221).

The presence of identifiable domains in the NOV12 protein was determined by searches using algorithms such as PROSITE, Blocks, Pfam, ProDomain, Prints and then determining the Interpro number by crossing the domain match (or numbers) using the Interpro website. The results indicate that the NOV12 protein contains the following protein domains (as defined by Interpro) at the indicated positions: WNT domain (IPR000970) at amino acid positions 53 to 365 of SEQ ID NO:24. The SignalP, Psort and/or Hydropathy profile for the NOV12 protein predicts that NOV12 has a signal peptide and is likely to be localized extracellularly with a certainty of 0.8200.

The NOV12 nucleic acid maps to human chromosome 3p21.1-p14. This information was assigned using OMIM, the electronic northern bioinformatic tool implemented by CuraGen Corporation, public ESTs, public literature references and/or genomic clone homologies. This was executed to derive the chromosomal mapping of the SeqCalling assemblies, Genomic clones, literature references and/or EST sequences that were included in the invention. This locus is associated with Aicardi-Goutieres syndrome, Brugada syndrome, congenital nonbullous ichthyosiform erythroderma, Long QT syndrome, congenital stationary night blindness and/or other diseases/disorders.

Possible SNPs found for NOV12 are listed in Table 12C.

TABLE 12 C
NOV12 SNPs

| Base | | |
|---|---|---|
| Position | Base Before | Base After |
| 1190 | T | C |

The Wnt-5a family of proteins are important in diverse cellular processes including proliferation, migration and differentiation. For example, it is well known that inhibition of alanyl-aminopeptidase gene expression or enzymatic activity compromises T cell proliferation and function. Molecular mechanisms mediating these effects are not known as yet. Wnt-5a is strongly affected by APN-inhibition. Wnt-5a is moderately expressed in resting T cells, but strongly down-regulated in response to activation by OKT3/IL-4/IL-9. Actinonin increases Wnt-5a-mRNA content. In addition, expression of GSK-3 beta, an inherent component of the Wnt-pathway, was found to be increased in response to activation, but suppressed by actinonin at both the mRNA and protein level.

Also, the beta-catenin signal transduction pathway, which can be activated by secreted Wnt proteins, plays a key role in normal embryonic development and in malignant transformation of the mammary gland and colon. Wnt and beta-catenin signaling also function in cells of the vasculature. RT-PCR analysis showed that primary endothelial and smooth muscle cell cultures, of both mouse and human origin, express members of the Wnt and Wnt receptor (Frizzled) gene families. Transfection of an expression vector for Wnt-1 into primary endothelial cells increased both the free pool of beta-catenin and the transcription from a Lef/tcf-dependent reporter gene construct. Expression of Wnt-1, but not Wnt-5a, also stimulates proliferation of primary endothelial cell cultures. Thus, Wnt and Frizzled proteins can regulate signal transduction, via beta-catenin, in endothelial cells.

The above defined information for this invention suggests that NOV 10-12 proteins may function as member of a “WNT-5A family”. Therefore, the NOV 10-12 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of the invention are useful in potential therapeutic applications implicated in in psychotic and neurological disorders including but not limited to Parkinson's disease and Alzheimers disease, neurodegenerative disorders, epilepsy, cancers including but not limited to brain tumor, colon cancer and breast cancer, developmental disorders, neural tube defects, trauma, regeneration (in vitro and in vivo), cardiac defects including cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation and viral/bacterial/parasitic infections. and/or other pathologies and disorders. For example, a cDNA encoding the WNT-5A-like protein may be useful in gene therapy, and the WNT-5A-like protein may be useful when administered to a subject in need thereof. By way of nonlimiting example, the compositions of the present invention will have efficacy for treatment of patients suffering from in psychotic and neurological disorders including but not limited to Parkinson's disease and Alzheimers disease, neurodegenerative disorders, epilepsy, cancers including but not limited to brain tumor, colon cancer and breast cancer, developmental disorders, neural tube defects, trauma, regeneration (in vitro and in vivo), cardiac defects including cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation and viral/bacterial/parasitic infections. The novel nucleic acid encoding WNT-5A-like protein, and the WNT-5A-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

### NOV13

The NOV13 nucleic acid of 4213 nucleotides (also designated CuraGen Acc. No. SC55003337_A) encoding a novel PROCOLLAGEN IN-PROTEINASE-like protein is shown in Table 13A. An open reading frame was identified beginning with an ATG initiation codon at nucleotides 1-3 and ending with a TAG codon at nucleotides 3631-3633. A putative untranslated region upstream from the initiation codon and downstream from the termination codon is underlined in FIG. 1, and the start and stop codons are in bold letters.

TABLE 13A
Encoded NOV13 nucleic acid sequence (SEQ ID NO:25).

ATGGCTCCACTCCGCGCGCTGCTGTCCTACCTGCTGCCTTTGCACTGTGCGCTCTGCGCCGCCGCGGGCAGCCGGACCCC
AGAGCTGCACCTCTCTGGAAAGCTCAGTGACTATGGTGTGACAGTGCCCTGCAGCACAGACTTTCGGGGACGCTTCCTCT
CCCACGTGGTGTCTGGCCCAGCAGCAGCCTCTGCAGGGAGCATGGTAGTGGACACGCCACCCACACTACCACGACACTCC
AGTCACCTCCGGGTGGCTCGCAGCCCTCTGCACCCAGGAGGGACCCTGTGGCCTGGCAGGGTGGGGCGCCACTCCCTCTA
CTTCAATGTCACTGTTTTCGGGAAGGAACTGCACTTGCGCCTGCGGCCCAATCGGAGGTTGGTAGTGCCAGGATCCTCAG
TGGAGTGGCAGGAGGATTTTCGGGAGCTGTTCCGGCAGCCCTTACGGCAGGAGTGTGTGTACACTGGAGGTGTCACTGGA
ATGCCTGGGGCAGCTGTTGCCATCAGCAACTGTGACGGATTGTGTGCAGGCCCTGCGGGCCTCATCCGCACAGACAGCAC
CGACTTCTTCATTGAGCCTCTGGAGCGGGGCCAGCAGGAGAAGGAGGCCAGCGGGAGGACACATGTGGTGTACCGCCGGG
AGGCCGTCCAGCAGGACTTTGGCCTGGGAGACCTTCCCAACCTGCTGGGCCTGGTGGGGGACCAGCTGGGCGACACAGAG
CGGAAGCGGCGGCATGCCAAGCCAGGCAGCTACAGCATCGAGGTGCTGCTGGTGGTGCACGACTCGGTGGTTCGCTTCCA
TGGCAAGGAGCATGTGCAGAACTATGTCCTCACCCTCATGAATATCGTGACTGTAGATGAGATTTACCACGATGAGTCCC
TGGGGGTTCATATAAATATTGCCCTCGTCCGCTTGATCATGGTTGGCTACCGACAGTCCCTGAGCCTGATCGAGCGCGGG
AACCCCTCACGCAGCCTGGAGCAGGTGTGTCGCTGGGCACACTCCCAGCAGCGCCAGGACCCCAGCCACGCTGAGCACCA
TGACCACGTTCTGTTCCTCACCCGGCAGGACTTTGGGCCCTCAGGGTATGCACCCGTCACTGGCATGTGTCACCCCCTGA
GGAGCTGTGCCCTCAACCATGAGGATGGCTTCTCCTCAGCCTTCGTGATAGCTCATGAGACCGGCCACGTGCTCGGCATG
GAGCATGACGGTCAGGGGAATGGCTGTGCAGATGAGACCAGCCTGGGCAGCGTCATGGCGCCCCTGGTGCAGGCTGCCTT
CCACCGCTTCCATTGGTCCCGCTGCAGCAAGCTGGAGCTCAGCCGCTACCTCCCCTCCTACGACTGCCTCCTCGATGACC
CCTTTGATCCTGCCTGGCCCCAGCCCCCAGAGCTGCCTGGGATCAACTACTCAATGGATGAGCAGTGCCGCTTTGACTTT
GGCAGTGGCTACCAGACCTGCTTGGCATTCAGGACCTTTGAGCCCTGCAAGCAGCTGTGGTGCAGCCATCCTGACAACCC
GTACTTCTGCAAGACCAAGAAGGGGCCCCCGCTGGATGGGACTGAGTGTGCACCCGGCAAGTGGTGCTTCAAAGGTCACT
GCATCTGGAAGTCGCCGGAGCAGACATATGGCCAGGATGGAGGCTGGAGCTCCTGGACCAAGTTTGGGTCATGTTCGCGG
TCATGTGGGGGCGGGGTGCGATCCCGCAGCCGGAGCTGCAACAACCCCTCCCCAGCCTATGGAGGCCGCCTCTGCTTAGG
GCCCATCTTCGAGTACCAGGTCTGCAACAGCGAGGAGTGCCCTGGGACCTACGAGGACTTCCGGGCCCAGCAGTGTGCCA
AGCGCAACTCCTACTATGTGCACCAGAATGCCAAGCACAGCTGGGTGCCCTACGAGCCTGACGATGACGCCCAGAAGTGT
GAGCTGATCTGCCAGTCGGCGGACACGGGGGACGTGGTGTTCATGAACCAGGTGGTTCACCATCGGACACGCTGCAGCTA
CCGGGACCCATACAGCGTCTGTGCGCGTGGCGAGTGTGTGCCTGTCGGCTGTGACAAGGAGGTGGGGTCCATCAAGGCGG
ATGACAAGTGTGGAGTCTGCGGGGGTGACAACTCCCACTGCAGGACTGTGAAGGGGACGCTGGGCAAGGCCTCCAAGCAG
GCACGAGCTCTCAAGCTCCTGCAGATCCCAGCAGGTGCCAGGCACATCCAGATTGAGGCACTGGAGAAGTCCCCCCACCG
CATTGTGGTGAAGAACCAGGTCACCGGCAGCTTCATCCTCAACCCCAAGGGCAAGGAAGCCACAAGCCGGACCTTCACCG
CCATGGGCCTGGAGTGGGAGGATGCGGTGGAGGATGCCAAGGAAAGCCTCAAGACCAGCGGGCCCCTGCCTGAAGCCATT
GCCATCCTGGCTCTCCCCCCAACTGAGGGTGGCCCCCGCAGCAGCCTGGCCPACAAGTACGTCATCCATGAGGACCTGCT
GCCCCTTATCGGCAGCAACAATGTGCTCCTGGAGGAGATCGACACCTATGAGTGGGCGCTCAAGAGCTGGGCCCCCTGCA
GCAACGCCTGTCGACGAGCGATCCACTTCACCAAATACGGCTGCCGGCGCAGACGAGACCACCACATCCTCCACCGACAC
CTGTCTGACCACAAGAAGAGGCCCAAGCCCATCCGCCGGCGCTCCAACCACCACCCGTGCTCTCAGCCTGTGTCGGTGAC
GGAGGAGTGGGGTGCCTGCAGCCGGAGCTGTGGGAAGCTGGGGGTGCAGACACGGGGGATACAGTGCCTGCTCCCCCTCT
CCAATGGACCCACAAGGTCATGCCGGCCAAAGCCTGCGCCGGGGACCGGCCTGAGGCCCGACGGCCCTGTCTCCGAGTG
CCCTGCCCAGCCCAGTGGAGGCTGGGAGCCTGGTCCCAGTGCTCTGCCACCTGTGGAGAGGGCATCCAGCAGCGGCAGGT
GGTGTGCACCACCAACGCCAACAGCCTCGGGCATTGCGAGGCGGATAGGCCAGACACTGTCCAGCTCTGCAGCCTGCCCG
CCTGTAACAAGATATCATCAACGGAGCCCTGCACGGGAGACAGGTCTGTCTTCTGCCAGATGGAAGTGCTCCATCGCTAC
TGCTCCATTCCCGGCTACCACCGGCTCTGCTGTGTGTCCTCCATCAAGAAGGCCTCGGGCCCCAACCCTGGCCCAGACCC
TCGCCCAACCTCACTGCCCCCCTTCTCCACTCCTGGAAGCCCCTTACCAGGACCCCAGGACCCTGCAGATGCTGCAGAGC
CTCCTGGAAAGCCAACGCGATCAGAGGACCATCAGCATGGCCGAGCCACACAGCTCCCAGGAGCTCTGGATACAAGCTCC
CCAGGGACCCAGCATCCCTTTGCCCCTGAGACACCAATCCCTGGAGCATCCTGGAGCATCTCCCCTACCACCCCCGGGGG
GCTGCCTTGGGGCTGGACTCAGACACCTACGCCAGTCCCTGAGGACAAAGGGCAACCTGGAGAAGACCTGAGACATCCCG
GCACCAGCCTCCCTGCTGCCTCCCCGGTGACATGAGCTGTGCCCTCCCATCCCACTGGCACGTTTACACTCTCTGTACTG
CCCCGTGACTCCCAGCTCAGAGGACACACATAGCAGGGCAGGCGCAAGCACAGACTTCATTTTAAATCATTCCCCTTCTT
CTCGTTTGGGGCTCTGATGCTCTTTACCCCACAAAGCGGGGTGGGACGAACACAAAGATCAGGGAAAGCCCTAATCGGAG
ATACCTCAGCAAGCTGCCCCCGGCGCGACTGACCCTCTCACGGCCCCTGTTGGTCTCCCCTGCCAAGACCAGCGTCAACT
ATTCCTCCCTCCTCACAGACCCTGGGCCTGGGCAGGTCTGAATCCCGCCTGGTCTGTAGCTAGAAGCTGTCACGGCTGCC
TGCCTTCCCCCAACTGTGAGCACCCCTGTGGAGGCCCTGCATATTTGCCCCCTCTCCCCAGAAAGCCAAAGCAGGGCCAG
GGTAGGTGGGGCACTGTTCACACCCACGCCGAGAGGAGGGGGGCCTGGCAATGTGGCATGAGGCTTCCCAGCTCCACGCC
TCGAGGGGGTCGAACACAAGGTGATCGCAGGCCCAACTCCTGGAAGCCAAGAGCTCCATCCAGTTCCACCAGCTGAGGCC
AGGCAGCAGAGGCCAGTTTGTCTTTGCTGGCCAGAAGATGGTGCTCATGGCCA

In a search of sequence databases, it was found, for example, that the NOV13 nucleic acid sequence has 1817 of 2801 bases (64%) identical to a Homo sapiens Procollagen I N-Proteinase mRNA (GENBANK-ID: HSAJ3125). The NOV13 nucleic acid is predicted to be expressed in at least small intestine, heart, pancreas, lung, and hippocampus.

The NOV13 encoded protein having 1210 amino acid residues and a predicted molecular weight of 132,122.2 Da is presented using the one-letter code in Table 13B.

TABLE 13B
Encoded NOV13 protein sequence (SEQ ID NO:26).

MAPLRALLSYLLPLHCALCAAAGSRTPELHLSGKLSDYGVTVPCSTDFRGRFLSHVVSGPAAASAGSMVVDTPPTLPRHS
SHLRVARSPLHPGGTLWPGRVGRHSLYFNVTVFGKELHLRLRPNRRLVVPGSSVEWQEDFRELFRQPLRQECVYTGGVTG
MPGAAVAISNCDGLCAGPAGLIRTDSTDFFIEPLERGQQEKEASGRTHVVYRREAVQQDFGLGDLPNLLGLVGDQLGDTE
RKRRHAKPGSYSIEVLLVVDDSVVRFHGKEHVQNYVLTLMNIVSVDEIYHDESLGVHINIALVRLIMVGYRQSLSLIERG
NPSRSLEQVCRWAHSQQRQDPSHAEHHDHVVFLTRQDFGPSGYAPVTGMCHPLRSCALNHEDGFSSAFVIAHETGHVLGM
EHDGQGNGCADETSLGSVMAPLVQAAFHRFHWSRCSKLELSRYLPSYDCLLDDPFDPAWPQPPELPGINYSMDEQCRFDF
GSGYQTCLAFRTFEPCKQLWCSHPDNPYFCKTKKGPPLDGTECAPGKWCFKGHCIWKSPEQTYGQDGGWSSWTKFGSCSR
SCGGGVRSRSRSCNNPSPAYGGRLCLGPMFEYQVCNSEECPGTYEDFRAQQCAKRNSYYVHQNAKHSWVPYEPDDDAQKC
ELICQSADTGDVVFMNQVVHDGTRCSYRDPYSVCARGECVPVGCDKEVGSMKADDKCGVCGGDNSHCRTVKGTLGKASKQ
AGALKLVQIPAGARHIQIEALEKSPHRIVVKNQVTGSFILNPKGKEATSRTFTAMGLEWEDAVEDAKESLKTSGPLPEAI
AILALPPTEGGPRSSLAYKYVIHEDLLPLIGSNNVLLEEMDTYEWALKSWAPCSKACGGGIQFTKYGCRRRRDHHMVQRH
LCDHKKRPKPIRRRCNQHPCSQPVWVTEEWGACSRSCGKLGVQTRGIQCLLPLSNGTHKVMPAKACAGDRPEARRPCLRV
PCPAQWRLGAWSQCSATCGEGIQQRQVVCRTNANSLGHCEGDRPDTVQVCSLPACNKISSTEPCTGDRSVFCQMEVLDRY
CSIPGYHRLCCVSCIKKASGPNPGPDPGPTSLPPFSTPGSPLPGPQDPADAAEPPGKPTGSEDHQHGRATQLPGALDTSS
PGTQHPFAPETPIPGASWSISPTTPGGLPWGWTQTPTPVPEDKGQPGEDLRHPGTSLPAASPVTXAVPCHPTGTFTLCVL
PRDSQLRGHT

The full amino acid sequence of the NOV13 protein was found to have 578 of 1003 amino acid residues (57%) identical to, and 716 of 1003 residues (71%) positive with, the 1211 amino acid residue human procollagen IN-proteinase protein (ptnr:SPTREMBL-ACC: 095450). The global sequence homology (as defined by FASTA alignment with the full length sequence of this protein) is 65.849% amino acid homology and 58.391% amino acid identity.

Other BLAST results include sequences from the Patp database, which is a proprietary database that contains sequences published in patents and patent publications. Patp results include those listed in Table 13C.

TABLE 13C
Patp alignments of NOV13

Sequences producing High-scoring Segment Pairs:

| | | | Smallest |
|---|---|---|---|
| | | | Sum |
| | Reading | High | Prob. |
| | Frame | Score | P(N) |
| >patp:AAB21254 Human metalloproteinase KIAAO366, 1201 aa. | +1 | 3422 | 0.0 |

The NOV13 protein contains the following protein domains (as defined by Interpro) at the indicated nucleotide positions of SEQ ID NO.:26: Thrombospondin type 1 domain (IPR000884) at amino acid positions 550 to 600, 843 to 900, 905 to 962, and 964 to 1015; Reprolysin family propeptide domain (IPR002870) at amino acid positions 253 to 454; Reprolysin (M12B) family zinc metallo domain (IPR001590) at amino acid positions 120 to 243; and heratin, high sulfur B2 protein domain (IPR002494) at amino acid positions 910 to 1058.

PSORT analysis predicts that the NOV13 protein is localized in the nucleus with a certainty of 0.9640. SIGNALP analysis predicts that the NOV13 protein has a signal peptide with the most likely cleavage site between positions 22 and 23 of SEQ ID NO:26.

Procollagen I N-proteinase (EC 3.4.24.14), the enzyme that specifically processes type I and type II procollagens to collagen, the enzyme is extensively characterized. It has a molecular mass of 107 kDa as determined by polyacrylamide gel electrophoresis in presence of SDS and of about 130 kDa when estimated by gel filtration on a Sephacryl-S300. Also, in standard assay (pH 7.5, 0.2 M NaCl, 35 degrees C.), the activation energy for reaction with amino procollagen type I was 17,000 calories per mole. In the same conditions, Km and Vmax values were, respectively, 435 and 39 nM per hour but varied strongly with pH and salt concentration. Further, the enzyme cleaved the NH2-terminal propeptide of type I procollagen at the specific site, the Pro-Gln bond in the alpha 1 type I procollagen chain. Still further, the enzyme contained a high proportion of Gly, Asx, and Glx residues but no Hyp or Hyl,. Finally, partial amino acid sequences obtained from internal peptides of the enzyme displayed no significant homology with known sequences. The association of procollagen I N-proteinase with a FACIT (fibril-associated collagens with interrupted triple helices) collagen as found here might be of physiological significance.

Procollagen N-proteinase cleaves the amino-propeptides in the processing of type I and type II procollagens to collagens. Deficiencies of the enzyme cause dermatosparaxis in cattle and sheep, and they cause type VIIC Ehlers-Danlos syndrome in humans, heritable disorders characterized by accumulation of pNcollagen and severe skin fragility. Amino acid sequences for the N-proteinase were used to obtain cDNAs from bovine skin. Three overlapping cDNAs had an ORF coding for a protein of 1205 residues. Mammalian cells stably transfected with a complete cDNA secreted an active recombinant enzyme that specifically cleaved type I procollagen. The protein contained zinc-binding sequences of the clan MB of metallopeptidases that includes procollagen C-proteinase/BMP-1. The protein also contained four repeats that are homologous to domains found in thrombospondins and in properdin and that can participate in complex intermolecular interactions such as activation of latent forms of transforming growth factor beta or the binding to sulfatides. Therefore, the enzyme may play a role in development that is independent of its role in collagen biosynthesis. In some tissues the levels of mRNA for the enzyme are disproportionately high relative to the apparent rate of collagen biosynthesis.

Ehlers-Danlos syndrome (EDS) type VIIC is a recessively inherited connective-tissue disorder, characterized by extreme skin fragility, characteristic facies, joint laxity, droopy skin, umbilical hernia, and blue sclera. Like the animal model dermatosparaxis, EDS type VIIC results from the absence of activity of procollagen I N-proteinase (pNPI), the enzyme that excises the N-propeptide of type I and type II procollagens. The pNPI enzyme is a metalloproteinase containing properdin repeats and a cysteine-rich domain with similarities to the disintegrin domain of reprolysins. Mutations that cause EDS type VIIC in the six known affected human individuals and also in one strain of dermatosparactic calf. Five of the individuals with EDS type VIIC were homozygous for a C-->T transition that results in a premature termination codon, Q225X. Four of these five patients were homozygous at three downstream polymorphic sites. The sixth patient was homozygous for a different transition that results in a premature termination codon, W795X. In the dermatosparactic calf, the mutation is a 17-bp deletion that changes the reading frame of the message. This is direct evidence that EDS type VIIC and dermatosparaxis result from mutations in the pNPI gene.

The above defined information for this invention suggests that this procollagen I N-proteinase-like protein may function as a member of a “procollagen I N-proteinase family”. Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (procollagen I N-proteinase gene delivery/ procollagen I N-proteinase gene ablation), research tools, tissue regeneration in vivo and in vitro of all tissues and cell types composing (but not limited to) those defined here.

The nucleic acids and proteins of the invention are useful in potential therapeutic applications implicated in cancer, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation trauma, regeneration (in vitro and in vivo), viral/bacterial/parasitic infections, Adrenoleukodystrophy, Congenital Adrenal Hyperplasia, Inflammatory bowel disease, Diverticular disease, Von Hippel-Lindau (VHL) syndrome, Stroke, Tuberous sclerosis, hypercalceimia, Parkinson's disease, Huntington's disease, Cerebral palsy, Epilepsy,Lesch-Nyhan syndrome, Multiple sclerosis, Ataxia-telangiectasia, Leukodystrophies, Behavioral disorders, Addiction, Anxiety, Pain, Neuroprotection, tumorgenesis, Alzheimer's disease, Ehlers-Danlos syndrome (EDS) type VIIC, and dermatosparaxis and/or other pathologies and disorders. For example, a cDNA encoding the procollagen I N-proteinase-like protein may be useful in gene therapy, and the procollagen I N-proteinase-like protein may be useful when administered to a subject in need thereof. By way of nonlimiting example, the compositions of the present invention will have efficacy for treatment of patients suffering from cancer, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation trauma, regeneration (in vitro and in vivo), viral/bacterial/parasitic infections, Adrenoleukodystrophy, Congenital Adrenal Hyperplasia, Inflammatory bowel disease, Diverticular disease, Von Hippel-Lindau (VHL) syndrome, Stroke, Tuberous sclerosis, hypercalceimia, Parkinson's disease, Huntington's disease, Cerebral palsy, Epilepsy,Lesch-Nyhan syndrome, Multiple sclerosis, Ataxia-telangiectasia, Leukodystrophies, Behavioral disorders, Addiction, Anxiety, Pain, Neuroprotection, tumorgenesis, Alzheimer's disease, Ehlers-Danlos syndrome (EDS) type VIIC, and dermatosparaxis. The novel nucleic acid encoding procollagen I N-proteinase-like protein, and the procollagen I N-proteinase-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

### NOV14

The NOV14 nucleic acid of 1390 nucleotides (also designated CuraGen Acc. No. GMAC073150_A) encoding a novel 26S protease regulatory subunit-like protein is shown in Table 14A. An open reading frame was identified beginning with an ATG initiation codon at nucleotides 20-23 and ending with a TAA codon at nucleotides 1376-1379. A putative untranslated region upstream from the initiation codon and downstream from the termination codon is underlined in Table 14A, and the start and stop codons are in bold letters.

TABLE 14A
Encoded NOV14 nucleic acid sequence (SEQ ID NO:27).

CTTGAGTGGCCAAGGCAAGATGGGTCAAAGTCAAAGTGGTCCTCATGGTCTTGGAGCTGGAAAGAAGGATGATAGGGACA
AGAAAAAGAAATATGAACCTCCTATACCAGCTAGAGTGAGGAAGAAGAAGAAAACAAAGGGACCAGATGCTCCCAGCAAA
CTGCCACTGATGACACCTCACACTCTGTGCCAGTTAAAATTATTCAAATTAGAGATAATTAAATACTGTCTTCTCATGAA
GGAAGAATTCATTAGAAATCAGGAACAAATGAAACTATTAGAAGGAAAGCAAGAGGAGGAAAGATCAAAACTGGATGATC
TGAGGGGGACCCCCATGTCAGTAGTAACCTTGGAAGAGATTATTGATGACAATCATGCCATCATGTCTACATCTGTCGGC
TCAGAGCATCTGTGGGCTCAGAGCATTCTTGTAGACAAGGATCTGCTGGAACCTGGCTGCTCGGTCCTGCTCAACCACAA
GGTTCGTGCTGTGATATGGGTGCTGATGGATGACACGGATACCCTAGTCACAATGATGAAGGTGGAAAAGACCCCCCAGG
AGACCTGTGTTGATACTGGGGGGTTGGACAGCCAAATTCAGGAAATTAAGGAATTTGTGGACCTTCCTCTCACACATTCT
GAATATTATGAAGAGATCGGTATAAAGCCCCCTAAGGGAGTCATTCACTATGGTCCACCTCCCACAGGTAPAACCTTGTT
AGCCAAAGCAGTAGCAAACCACATCTTAGCCACTTTCTTGCAAGTGATCAGCTCTGAATTTATTCAGAAATACCTACATG
ATGGGCCCAAACTCATATCGGAATTGTTTCTAGTTGCTGAAGAACATGCACCTTCCATCATGTTTATTCATCAAATTGAT
GCTATTAGGACAAAAAGATGTGACTCAAATTCTGATAGTGAGAGAGAAATTCACCAAATAATCCTCGAAATCTTCAACCA
GTTGGATGGATTTGATTCAAGGGGAGATGTGAAAGTTATCATATCCACAAGCCGAATAGAAACTTTGGATCTAGCACTTA
TCAGACCAGGCTACACTGACAGGAAGCTCAAGTTCCCCCTGCCTGATGAAAAGACTAAGAACCACATCTTTCAGATGCAC
ACAAGCAGGATTACGCTGGCCAATGATACAATCCTGGACAACTCCATCATGGCTAAAGATGACCTCTCTTCTACAGACCT
CAAGGCAATCTGCACAGAAGCTAGTCTGATGGCCTTAAAAGAACATGGAATGAAAGTAACAAATGAAAACTTCAAAAAAT
CTCAAGAAAATGTTCTTTATAAAGAACAGGAAGACACCCCCAAGGGGCTCTGTCTCCGAACCAAGACAAAGAAGGGGAAG
GCGCCACACTCATTTTAACAACCAGATATT

In a search of sequence databases, it was found, for example, that the NOV14 nucleic acid sequence has 1209 of 1342 bases (90%) identical to a human 26S protease (S4) regulatory subunit mRNA (GENBANK-ID: HUM26SPSIV).

The encoded protein having 452 amino acid residues and a predicted molecular weight of 50,985.4 Da is presented using the one-letter code in Table 14B.

TABLE 14B
Encoded NOV14 protein sequence (SEQ ID NO:28).

MGQSQSGGHGLGAGKKDDRDKKKKYEPPIPARVRKKKKTKGPDAASKLPLMTPHTLCQLKLLKLEIIKYCLLMKEEFIRN
QEQMKLLEGKQEEERSKVDDLRGTPMSVVTLEEIIDDNHAIMSTSVGSEHLWAQSILVDKDLLEPGCSVLLNHKVRAVIW
VLMDDTDTLVTMMKVEKTPQETCVDTGGLDSQIQEIKEFVELPLTHSEYYEEMGIKPPKGVIHYGPPGTGKTLLAKAVAN
HILATFLQVISSEFIQKYLHDGPKLIWELFLVAEEHAPSIMFIDEIDAIRTKRCDSNSDSEREIQQIMLEMLNQLDGFDS
RGDVKVIISTSRIETLDLALIRPGYTDRKLKFPLPDEKTKKHIFQMHTSRITLANDTILDNSIMAKDDLSCTDLKAICTE
ASLMALKEHGMKVTNENFKKSQENVLYKEQEDTPKGLCLGSKRKKGKGPDSF

The full amino acid sequence of the NOV14 protein was found to have 355 of 440 amino acid residues (80%) identical to, and 386 of 440 residues (87%) positive with, the 440 amino acid residue human 26S PROTEASE REGULATORY SUBUNIT 4 (P26S4) protein (ptnr:SPTREMBL-ACC:P49014). The global sequence homology (as defined by FASTA alignment with the full length sequence of the NOV14 protein) is 82.916% amino acid homology and 80.866% amino acid identity.

Other BLAST results include sequences from the Patp database, which is a proprietary database that contains sequences published in patents and patent publications. Patp results include those listed in Table 14C.

TABLE 14C
Patp alignments of NOV14

Sequences producing High-scoring
Segment Pairs:

| | | |
|---|---|---|
| | | Smallest |
| | | Sum |
| Reading | High | Prob. |
| Frame | Score | P(N) |

>patp:AAR94600 S4 protein - Homo

| | | | |
|---|---|---|---|
| sapiens, 440 aa. | +2 | 1770 | 1.4e−181 |

The NOV14 protein contains the following protein domains (as defined by Interpro) at the indicated amino acid positions of SEQ ID NO:28: NB-ARC domain (IPR002182) at amino acid positions 224 to 242, AAA domain (IPR001939) at amino acid positions 220 to 407, RNA_helicase domain (IPR000605) at amino acid positions 112 to 434.

PSORT analysis predicts the NOV14 protein to be localized in the nucleus with a certainty of 0.9800.

The 26S proteasome is the major non-lysosomal protease in eukaryotic cells. This multimeric enzyme is the integral component of the ubiquitin-mediated substrate degradation pathway. It consists of two subcomplexes, the 20S proteasome, which forms the proteolytic core, and the 19S regulator (or PA700), which confers ATP dependency and ubiquitinated substrate specificity on the enzyme. Recent biochemical and genetic studies have revealed many of the interactions between the 17 regulatory subunits, yielding an approximation of the 19S complex topology. Inspection of interactions of regulatory subunits with non-subunit proteins reveals patterns that suggest these interactions play a role in 26S proteasome regulation and localization.

The ATP/ubiquitin-dependent 26S proteasome is a central regulator of cell cycle progression and stress responses. While investigating the application of peptide aldehyde proteasome inhibitors to block signal-induced IkappaBalpha degradation in human LNCaP prostate carcinoma cells, it was observed that persistent inhibition of proteasomal activity signals a potent cell death program. Biochemically, this program included substantial upregulation of PAR-4 (prostate apoptosis response−4), a putative pro-apoptotic effector protein and stabilization of cjun protein, a potent pro-death effector in certain cells. Also observed was modest downregulation of bcl-XL, a pro-survival effector protein. However, in contrast to some recent reports stable, high level, expression of functional bcl-2 protein in prostate carcinoma cells failed to signal protection against cell death induction by proteasome inhibitors. Also in disagreement to a recent report, no evidence was found for activation of the JNK stress kinase pathway. A role for p53, a protein regulated by the proteasome pathway, was ruled out, since comparable cell death induction by proteasome inhibitors occurred in PC-3 cells that do not express functional p53 protein. Thus, the ubiquitin/proteasome pathway represents a potential therapeutic target for prostate cancers irrespective of bcl-2 expression or p53 mutations.

The above defined information for this invention suggests that this 26S protease regulatory subunit-like protein may function as a member of a “26S protease regulatory subunit-like family”. Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV14 nucleic acids and proteins are useful in potential therapeutic applications implicated in eye/lens disorders including but not limited to cataract and Aphakia, Alzheimer's disease, neurodegenerative disorders, inflammation and modulation of the immune response, viral pathogenesis, aging-related disorders, neurologic disorders, cancer and/or other pathologies and disorders. For example, a cDNA encoding the 26S protease regulatory subunit-like protein may be useful in gene therapy, and the 26S protease regulatory subunit-like protein may be useful when administered to a subject in need thereof. By way of nonlimiting example, the compositions of the present invention will have efficacy for treatment of patients suffering from eye/lens disorders including but not limited to cataract and Aphakia, Alzheimer's disease, neurodegenerative disorders, inflammation and modulation of the immune response, viral pathogenesis, aging-related disorders, neurologic disorders, cancer. The novel nucleic acid encoding 26S protease regulatory subunit-like protein, and the 26S protease regulatory subunit-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

### NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:1, 3, 5, 7, 9, 11, 13, 15, 17, 19, 21, 23, 25, or 27, or a complement of this aforementioned nucleotide sequence, can be isolated using standard molecular biology techniques and the sequence information provided herein. Using all or a portion of the nucleic acid sequence of SEQ ID NOS 1, 3, 5, 7, 9, 11, 13, 15, 17, 19, 21, 23, 25, or 27 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, eg., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS:1, 3, 5, 7, 9, 11, 13, 15, 17, 19, 21, 23, 25, and 27, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17, 19, 21, 23, 25, or 27, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS:1, 3, 5, 7, 9, 11, 13, 15, 17, 19, 21, 23, 25, or 27 is one that is sufficiently complementary to the nucleotide sequence shown SEQ ID NOS:1, 3, 5, 7, 9, 11, 13, 15, 17, 19, 21, 23, 25, or 27 that it can hydrogen bond with little or no mismatches to the nucleotide sequence shown SEQ ID NOS:1, 3, 5, 7, 9, 11, 13, 15, 17, 19, 21, 23, 25, or 27, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS:1, 3, 5, 7, 9, 11, 13, 15, 17, 19, 21, 23, 25, or 27, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS:1, 3, 5, 7, 9, 11, 13, 15, 17, 19, 21, 23, 25, or 27; or an anti-sense strand nucleotide sequence of SEQ ID NOS:1, 3, 5, 7, 9, 11, 13, 15, 17, 19, 21, 23, 25, or 27; or of a naturally occurring mutant of SEQ ID NOS:1, 3, 5, 7, 9, 11, 13, 15, 17, 19, 21, 23, 25, or 27.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:1, 3, 5, 7, 9, 11, 13, 15, 17, 19, 21, 23, 25, or 27, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

### NOVX Nucleic Acid and Polypeptide Variants

The invention further encompasses nucleic acid molecules that differ from the nucleotide sequences shown in SEQ ID NOS:1, 3, 5, 7, 9, 11, 13, 15, 17, 19, 21, 23, 25, or 27 due to degeneracy of the genetic code and thus encode the same NOVX proteins as that encoded by the nucleotide sequences shown in SEQ ID NOS:1, 3, 5, 7, 9, 11, 13, 15, 17, 19, 21, 23, 25, or 27. In another embodiment, an isolated nucleic acid molecule of the invention has a nucleotide sequence encoding a protein having an amino acid sequence shown in SEQ ID NOS:2, 4, 6, 8, 10, 12, 14, 16, 18,20,22,24,26 or 28.

In addition to the human NOVX nucleotide sequences shown in SEQ ID NOS:1, 3, 5, 7, 9, 11, 13, 15, 17, 19, 21, 23, 25, or 27, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS:1, 3, 5, 7, 9, 11, 13, 15, 17, 19, 21, 23, 25, or 27 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17, 19, 21, 23, 25, or 27. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS:1, 3, 5, 7, 9, 11, 13, 15, 17, 19, 21, 23, 25, or 27, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17, 19, 21, 23, 25, or 27, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS:1, 3, 5, 7, 9, 11, 13, 15, 17, 19, 21, 23, 25, or 27, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

### Conservative Mutations

In addition to naturally-occurring allelic variants of NOVX sequences that may exist in the population, the skilled artisan will further appreciate that changes can be introduced by mutation into the nucleotide sequences SEQ ID NOS:1, 3, 5, 7, 9, 11, 13, 15, 17, 19, 21, 23, 25, or 27, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 16, 18, 20, 22, 24, 26 or 28. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:1, 3, 5, 7, 9, 11, 13, 15, 17, 19, 21, 23, 25, or 27 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 16, 18, 20, 22, 24, 26 and 28. Preferably, the protein encoded by the nucleic acid molecule is at least about 60% homologous to SEQ ID NOS:2, 4, 6, 8, 10, 12, 14, 16, 18, 20, 22, 24, 26 and 28; more preferably at least about 70% homologous SEQ ID NOS:2, 4, 6, 8, 10, 12, 14, 16, 18, 20, 22, 24, 26 or 28; still more preferably at least about 80% homologous to SEQ ID NOS:2, 4, 6, 8, 10, 12, 14, 16, 18, 20, 22, 24, 26 or 28; even more preferably at least about 90% homologous to SEQ ID NOS:2, 4, 6, 8, 10, 12, 14, 16, 18, 20, 22, 24, 26 or 28; and most preferably at least about 95% homologous to SEQ ID NOS:2, 4, 6, 8, 10, 12, 14, 16, 18, 20, 22, 24, 26 or 28.

An isolated nucleic acid molecule encoding an NOVX protein homologous to the protein of SEQ ID NOS:2, 4, 6, 8, 10, 12, 14, 16, 18, 20, 22, 24, 26 or 28 can be created by introducing one or more nucleotide substitutions, additions or deletions into the nucleotide sequence of SEQ ID NOS: 1, 3, 5, 7, 9, 11, 13, 15, 17, 19, 21, 23, 25, or 27, such that one or more amino acid substitutions, additions or deletions are introduced into the encoded protein.

Mutations can be introduced into SEQ ID NOS:1, 3, 5, 7, 9, 11, 13, 15, 17, 19, 21, 23, 25, or 27 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS:1, 3, 5, 7, 9, 11, 13, 15, 17, 19, 21, 23, 25, or 27, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof, (e.g. avidin proteins).

In yet another embodiment, a mutant NOVX protein can be assayed for the ability to regulate a specific biological function (e.g., regulation of insulin release).

### Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17, 19, 21, 23, 25, or 27, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18, 20, 22, 24, 26 or 28, or antisense nucleic acids complementary to an NOVX nucleic acid sequence of SEQ ID NOS:1, 3, 5, 7, 9, 11, 13, 15, 17, 19, 21, 23, 25, or 27, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyturacil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

### Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS:1, 3, 5, 7, 9, 11, 13, 15, 17, 19, 21, 23, 25, or 27). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. W088/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

### NOVX Polypeptides

A polypeptide according to the invention includes a polypeptide including the amino acid sequence of NOVX polypeptides whose sequences are provided in SEQ ID NOS:2, 4, 6, 8, 10, 12, 14, 16, 18, 20, 22, 24, 26 or 28. The invention also includes a mutant or variant protein any of whose residues may be changed from the corresponding residues shown in SEQ ID NOS:2, 4, 6, 8, 10, 12, 14, 16, 18, 20, 22, 24, 26 or 28 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 16, 18, 20, 22, 24, 26 or 28) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 4, 6, 8, 10, 12, 14, 16, 18, 20, 22, 24, 26 or 28. In other embodiments, the NOVX protein is substantially homologous to SEQ ID NOS:2, 4,6,8, 10, 12, 14, 16, 18,20,22,24,26 or 28, and retains the functional activity of the protein of SEQ ID NOS:2, 4, 6, 8, 10, 12, 14, 16, 18, 20, 22, 24, 26 or 28, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 4, 6, 8, 10, 12, 14, 16, 18, 20, 22, 24, 26 or 28, and retains the functional activity of the NOVX proteins of SEQ ID NOS:2, 4, 6, 8, 10, 12, 14, 16, 18, 20, 22, 24, 26 or 28.

### Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encodin of the DNA sequence shown in SEQ ID NOS:1, 3, 5, 7, 9, 11, 13, 15, 17, 19, 21, 23, 25, or 27.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

### Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 4, 6, 8, 10, 12, 14, 16, 18, 20, 22, 24, 26 or 28), whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

### NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

### Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

### Anti-NOVX Antibodies

The invention encompasses antibodies and antibody fragments, such as F_{ab} or (F_{ab})₂, that bind immunospecifically to any of the NOVX polypeptides of said invention.

An isolated NOVX protein, or a portion or fragment thereof, can be used as an immunogen to generate antibodies that bind to NOVX polypeptides using standard techniques for polyclonal and monoclonal antibody preparation. The full-length NOVX proteins can be used or, alternatively, the invention provides antigenic peptide fragments of NOVX proteins for use as immunogens. The antigenic NOVX peptides comprises at least 4 amino acid residues of the amino acid sequence shown SEQ ID NOS:2, 4, 6, 8, 10, 12, 14, 16, 18, 20, 22, 24, 26 or 28 and encompasses an epitope of NOVX such that an antibody raised against the peptide forms a specific immune complex with NOVX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of NOVX that is located on the surface of the protein (e.g., a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

As disclosed herein, NOVX protein sequences of SEQ ID NOS:2, 4, 6, 8, 10, 12, 14, 16, 18, 20, 22, 24, 26 and 28, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as NOVX. Such antibodies include, but are not limited to, polyclonal, monoclonal, chimeric, single chain, F_{ab} and F_{(ab′)2} fragments, and an F_{ab} expression library. In a specific embodiment, antibodies to human NOVX proteins are disclosed. Various procedures known within the art may be used for the production of polyclonal or monoclonal antibodies to an NOVX protein sequence of SEQ ID NOS: 2, 4, 6, 8, 10, 12, 14, 16, 18, 20, 22, 24, 26 and 28, or a derivative, fragment, analog or homolog thereof Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed NOVX protein or a chemically-synthesized NOVX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against NOVX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of NOVX. A monoclonal antibody composition thus typically displays a single binding affinity for a particular NOVX protein with which it immunoreacts. For preparation of monoclonal antibodies directed towards a particular NOVX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to an NOVX protein (see, e.g., U.S. Pat. No. 4,946,778). In addition, methods can be adapted for the construction of Fab expression libraries (see, e.g., Huse, et al., 1989. Science 246: 1275-1281) to allow rapid and effective identification of monoclonal Fab fragments with the desired specificity for an NOVX protein or derivatives, fragments, analogs or homologs thereof. Non-human antibodies can be “humanized” by techniques well known in the art. See, e.g., U.S. Pat. No. 5,225,539. Antibody fragments that contain the idiotypes to an NOVX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) Fᵥ fragments.

Additionally, recombinant anti-NOVX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314 :446-449; Shaw, et al., 1988. J Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I ³⁵S or ³H.

### NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec 1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

### Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:1, 3, 5, 7, 9, 11, 13, 15, 17, 19, 21, 23, 25, or 27 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:1,3,5,7,9, 11, 13, 15, 17, 19, 21, 23, 25, or 27), but more preferably, is a non-human homologue of a human NOVX gene. For example, a mouse homologue of human NOVX gene of SEQ ID NOS:1, 3, 5, 7, 9, 11, 13, 15, 17, 19, 21, 23, 25, or 27 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

### Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

### Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

### Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12:145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i. e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

### Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

### Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:1, 3, 5, 7, 9, 11, 13, 15, 17, 19, 21, 23, 25, or 27, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17, 19, 21, 23, 25, or 27 are used, a more appropriate number of primers for positive individual identification would be 500-2,000.

### Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

### Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:1, 3, 5, 7, 9, 11, 13, 15, 17, 19, 21, 23, 25, or 27, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

### Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al, 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7:5.

In yet another embodiment, the movement of mutant or wild-type fragments in polyacrylamide gels containing a gradient of denaturant is assayed using denaturing gradient gel electrophoresis (DGGE). See, e.g., Myers, et al, 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11:238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

### Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

### Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

### Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

### Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i. e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

### Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

### Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

### Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

### Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLE 1

### Quantitative Expression Analysis (TaqMan) of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources), Panel 4 (containing cells and cell lines from normal cells and cells related to inflammatory conditions) and Panel CNSD.01 (containing samples from normal and diseased brains).

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1×TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

In the results for Panel 1, the following abbreviations are used:

ca.=carcinoma,

*=established from metastasis,

met=metastasis,

s cell var=small cell variant,

non-s=non-sm=non-small,

squam=squamous,

pl. eff pl effusion=pleural effusion,

glio=glioma,

astro=astrocytoma, and

neuro=neuroblastoma.

### Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

### PANEL 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1 .3D are of the most common cell lines used in the scientific literature.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

### Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 IL-13 at approximately 5-10 ng/ml. Endothelial cells were sometimes starved for various times by culture in the basal media from Clonetics with 0. 1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, UT), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10 cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M(Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Trl. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Trl after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 107 cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

### Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

### EXAMPLE 2

### Quantitative Expression Analysis (TaqMan) of NOV1

Expression of NOV1 (SC_{—}105828681_A) was assessed using the primer-probe set Ag1395, described in Table AA. Results of the RTQ-PCR runs are shown in Tables AB, AC, AD, AE, and AF.

TABLE AA
Probe Name Ag 1395

| Primers | Sequences | | TM | Length | Start Position |
|---|---|---|---|---|---|
| Forward | 5′-CTGCACTTCAAGGACAGTTACC-3′ | (SEQ ID NO. 29) | 58.9 | 22 | 2184 |
| Probe | FAM-5′-CTATCCATCCACGATGTGCCCAGCT-3′-TAMRA | (SEQ ID NO. 30) | 71.1 | 25 | 2217 |
| Reverse | 5′-TGACAAGGAGCTTACTCTTCCA-3′ | (SEQ ID NO. 31) | 59.1 | 22 | 2247 |

TABLE AB
Panel 1.2

| | Relative Expression(%) | |
|---|---|---|
| | 1.2tm1636f_{—} | 1.2tm1675f_{—} |
| Tissue Name | ag1395 | ag1395* |
| Endothelial cells | 0.0 | 0.0 |
| Heart (fetal) | 0.2 | 0.1 |
| Pancreas | 0.0 | 0.0 |
| Pancreatic ca. CAPAN 2 | 0.4 | 0.6 |
| Adrenal Gland (new lot*) | 1.1 | 3.6 |
| Thyroid | 0.0 | 0.0 |
| Salavary gland | 0.2 | 0.3 |
| Pituitary gland | 0.0 | 0.0 |
| Brain (fetal) | 1.8 | 1.9 |
| Brain (whole) | 11.3 | 3.3 |
| Brain (amygdala) | 9.8 | 18.2 |
| Brain (cerebellum) | 3.1 | 3.6 |
| Brain (hippocampus) | 31.4 | 42.6 |
| Brain (thalamus) | 2.1 | 2.9 |
| Cerebral Cortex | 100.0 | 100.0 |
| Spinal cord | 0.1 | 0.0 |
| CNS ca. (glio/astro) U87-MG | 0.0 | 0.0 |
| CNS ca. (glio/astro) U-118-MG | 0.0 | 0.0 |
| CNS ca. (astro) SW1783 | 0.0 | 0.0 |
| CNS ca.* (neuro; met) SK-N-AS | 0.1 | 0.3 |
| CNS ca. (astro) SF-539 | 0.0 | 0.0 |
| CNS ca. (astro) SNB-75 | 0.0 | 0.0 |
| CNS ca. (glio) SNB-19 | 0.0 | 0.0 |
| CNS ca. (glio) U251 | 0.0 | 0.0 |
| CNS ca. (glio) SF-295 | 0.1 | 0.1 |
| Heart | 0.0 | 0.3 |
| Skeletal Muscle (new lot*) | 0.0 | 0.0 |
| Bone marrow | 0.9 | 0.8 |
| Thymus | 0.0 | 0.0 |
| Spleen | 0.0 | 0.1 |
| Lymph node | 0.0 | 0.0 |
| Colorectal | 0.0 | 0.0 |
| Stomach | 0.3 | 0.1 |
| Small intestine | 0.2 | 0.2 |
| Colon Ca. SW480 | 0.5 | 0.1 |
| Colon ca.* (SW480 met) SW620 | 0.2 | 0.1 |
| Colon Ca. HT29 | 0.0 | 0.0 |
| Colon Ca. HCT-116 | 1.3 | 1.8 |
| Colon Ca. CaCo-2 | 0.0 | 0.0 |
| 83219 CC Well to Mod Diff | 0.0 | 0.0 |
| (ODO3866) | | |
| Colon Ca. HCC-2998 | 3.2 | 3.4 |
| Gastric ca.* (liver met) NCI-N87 | 0.0 | 0.0 |
| Bladder | 0.8 | 0.8 |
| Trachea | 0.0 | 0.0 |
| Kidney | 0.0 | 0.0 |
| Kidney (fetal) | 0.0 | 0.0 |
| Renal ca. 786-0 | 0.1 | 0.1 |
| Renal ca. A498 | 6.0 | 4.7 |
| Renal ca. RXF 393 | 0.0 | 0.0 |
| Renal ca. ACHN | 0.8 | 1.0 |
| Renal ca. UO-31 | 0.3 | 0.2 |
| Renal ca. TK-10 | 6.0 | 3.0 |
| Liver | 0.3 | 0.3 |
| Liver (fetal) | 0.0 | 0.1 |
| Liver ca. (hepatoblast) HepG2 | 0.0 | 0.0 |
| Lung | 0.0 | 0.0 |
| Lung (fetal) | 0.0 | 0.0 |
| Lung ca. (small cell) LX-1 | 0.0 | 0.0 |
| Lung ca. (small cell) NCI-H69 | 16.3 | 9.3 |
| Lung ca. (s.cell var.) SHP-77 | 0.4 | 0.4 |
| Lung ca. (large cell) NCI-H460 | 0.0 | 0.0 |
| Lung ca. (non-sm. cell) A549 | 0.0 | 0.0 |
| Lung ca. (non-s.cell) NCI-H23 | 0.4 | 0.4 |
| Lung ca (non-s.cell) HOP-62 | 0.0 | 0.0 |
| Lung ca. (non-s.cl) NCI-H522 | 9.0 | 11.5 |
| Lung ca. (squam.) SW 900 | 1.5 | 0.9 |
| Lung ca. (squam.) NCI-H596 | 18.8 | 16.6 |
| Mammary gland | 0.1 | 0.1 |
| Breast ca.* (pl. effusion) MCF-7 | 0.0 | 0.2 |
| Breast ca.* (pl.ef) MDA-MB-231 | 0.0 | 0.0 |
| Breast ca.* (pl. effusion) T47D | 0.5 | 1.3 |
| Breast Ca. BT-549 | 0.0 | 0.0 |
| Breast Ca. MDA-N | 0.0 | 0.0 |
| Ovary | 0.4 | 0.3 |
| Ovarian ca. OVCAR-3 | 0.0 | 0.0 |
| Ovarian ca. OVCAR-4 | 0.2 | 0.3 |
| Ovarian ca. OVCAR-5 | 18.4 | 11.7 |
| Ovarian ca. OVCAR-8 | 1.0 | 1.4 |
| Ovarian ca. IGROV-1 | 20.2 | 11.7 |
| Ovarian ca.* (ascites) SK-OV-3 | 0.4 | 0.6 |
| Uterus | 0.0 | 0.0 |
| Placenta | 0.0 | 0.0 |
| Prostate | 0.2 | 0.2 |
| Prostate ca.* (bone met) PC-3 | 0.0 | 0.0 |
| Testis | 0.2 | 0.0 |
| Melanoma Hs688 (A).T | 0.0 | 0.0 |
| Melanoma* (met) Hs688 (B).T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.0 |
| Adipose | 6.5 | 7.0 |

TABLE AC
Panel 2D

| | Relative Expression(%) | |
|---|---|---|
| | 2dtm2448f_{—} | 2dx4tm4720f_{—} |
| Tissue Name | ag1395 | ag1395_a2 |
| Normal Colon GENPAK 061003 | 4.2 | 1.9 |
| 83219 CC Well to Mod Diff (ODO3866) | 0.7 | 1.8 |
| 83220 CC NAT (ODO3866) | 0.0 | 1.0 |
| 83221 CC Gr.2 rectosigmoid (ODO3868) | 0.0 | 1.1 |
| 83222 CC NAT (ODO3868) | 0.0 | 0.0 |
| 83235 CC Mod Diff (ODO3920) | 0.0 | 1.2 |
| 83236 CC NAT (ODO3920) | 0.0 | 0.8 |
| 83237 CC Gr.2 ascend colon (ODO3921) | 0.9 | 2.3 |
| 83238 CC NAT (ODO3921) | 0.0 | 0.4 |
| 83241 CC from Partial Hepatectomy (ODO4309) | 0.7 | 0.2 |
| 83242 Liver NAT (ODO4309) | 0.0 | 0.9 |
| 87472 Colon mets to lung (OD04451-01) | 0.0 | 2.3 |
| 87473 Lung NAT (OD04451-02) | 0.8 | 0.0 |
| Normal Prostate Clontech A+ 6546-1 | 9.0 | 8.2 |
| 84140 Prostate Cancer (OD04410) | 0.0 | 3.5 |
| 84141 Prostate NAT (OD04410) | 2.0 | 1.7 |
| 87073 Prostate Cancer (OD04720-01) | 0.8 | 1.7 |
| 87074 Prostate NAT (OD04720-02) | 0.0 | 1.5 |
| Normal Lung GENPAK 061010 | 3.1 | 10.9 |
| 83239 Lung Met to Muscle (ODO4286) | 4.4 | 4.1 |
| 83240 Muscle NAT (ODO4286) | 0.0 | 0.5 |
| 84136 Lung Malignant Cancer (OD03126) | 2.2 | 2.6 |
| 84137 Lung NAT (OD03126) | 3.2 | 4.6 |
| 84871 Lung Cancer (OD04404) | 2.4 | 1.1 |
| 84872 Lung NAT (OD04404) | 3.3 | 4.2 |
| 84875 Lung Cancer (OD04565) | 0.0 | 1.6 |
| 84876 Lung NAT (OD04565) | 1.7 | 1.7 |
| 85950 Lung Cancer (OD04237-01) | 0.8 | 4.5 |
| 85970 Lung NAT (OD04237-02) | 3.8 | 7.1 |
| 83255 Ocular Mel Met to Liver (ODO4310) | 0.0 | 0.0 |
| 83256 Liver NAT (ODO4310) | 6.2 | 2.1 |
| 84139 Melanoma Mets to Lung (OD04321) | 0.8 | 0.0 |
| 84138 Lung NAT (OD04321) | 3.8 | 5.3 |
| Normal Kidney GENPAK 061008 | 0.8 | 1.6 |
| 83786 Kidney Ca, Nuclear grade 2 (OD04338) | 1.2 | 2.8 |
| 83787 Kidney NAT (OD04338) | 0.0 | 1.8 |
| 83788 Kidney Ca Nuclear grade 1/2 (OD04339) | 5.9 | 5.4 |
| 83789 Kidney NAT (OD04339) | 0.0 | 0.0 |
| 83790 Kidney Ca, Clear cell type (OD04340) | 1.3 | 7.5 |
| 83791 Kidney NAT (OD04340) | 0.0 | 0.3 |
| 83792 Kidney Ca, Nuclear grade 3 (OD04348) | 0.0 | 2.1 |
| 83793 Kidney NAT (OD04348) | 0.8 | 0.8 |
| 87474 Kidney Cancer (OD04622-01) | 2.2 | 4.1 |
| 87475 Kidney NAT (OD04622-03) | 0.7 | 0.4 |
| 85973 Kidney Cancer (OD04450-01) | 0.0 | 0.4 |
| 85974 Kidney NAT (OD04450-03) | 0.0 | 0.0 |
| Kidney Cancer Clontech 8120607 | 27.9 | 60.6 |
| Kidney NAT Clontech 8120608 | 0.8 | 2.1 |
| Kidney Cancer Clontech 8120613 | 0.8 | 1.7 |
| Kidney NAT Clontech 8120614 | 0.7 | 0.7 |
| Kidney Cancer Clontech 9010320 | 4.7 | 6.4 |
| Kidney NAT Clontech 9010321 | 0.0 | 2.7 |
| Normal Uterus GENPAK 061018 | 0.0 | 2.2 |
| Uterus Cancer GENPAK 064011 | 0.0 | 8.9 |
| Normal Thyroid Clontech A + 6570-1 | 8.7 | 1.2 |
| Thyroid Cancer GENPAK 064010 | 0.0 | 0.0 |
| Thyroid Cancer INVITROGEN A302152 | 0.0 | 2.5 |
| Thyroid NAT INVITROGEN A302153 | 1.1 | 0.8 |
| Normal Breast GENPAK 061019 | 2.8 | 4.1 |
| 84877 Breast Cancer (OD04566) | 0.0 | 1.8 |
| 85975 Breast Cancer (OD04590-01) | 28.3 | 27.5 |
| 85976 Breast Cancer Mets (OD04590-03) | 13.3 | 14.2 |
| 87070 Breast Cancer Metastasis (OD04655-05) | 37.9 | 100.0 |
| GENPAK Breast Cancer 064006 | 12.0 | 19.3 |
| Breast Cancer Res. Gen. 1024 | 33.9 | 25.2 |
| Breast Cancer Clontech 9100266 | 6.7 | 7.7 |
| Breast NAT Clontech 9100265 | 0.5 | 9.1 |
| Breast Cancer INVITROGEN A209073 | 3.7 | 6.9 |
| Breast NAT INVITROGEN A2090734 | 0.7 | 0.0 |
| Normal Liver GENPAK 061009 | 0.0 | 2.6 |
| Liver Cancer GENPAK 064003 | 0.0 | 1.3 |
| Liver Cancer Research Genetics RNA 1025 | 0.4 | 2.0 |
| Liver Cancer Research Genetics RNA 1026 | 0.0 | 1.6 |
| Paired Liver Cancer Tissue Research Genetics RNA 6004-T | 1.6 | 3.4 |
| Paired Liver Tissue Research Genetics RNA 6004-N | 1.4 | 0.7 |
| Paired Liver Cancer Tissue Research Genetics RNA 6005-T | 0.8 | 0.8 |
| Paired Liver Tissue Research Genetics RNA 6005-N | 0.0 | 0.0 |
| Normal Bladder GENPAK 061001 | 3.5 | 3.8 |
| Bladder Cancer Research Genetics RNA 1023 | 0.8 | 0.5 |
| Bladder Cancer INVITROGEN A302173 | 3.2 | 1.1 |
| 87071 Bladder Cancer (OD04718-01) | 3.8 | 2.3 |
| 87072 Bladder Normal Adjacent (OD04718-03) | 5.2 | 7.4 |
| Normal Ovary Res. Gen. | 3.0 | 2.9 |
| Ovarian Cancer GENPAK 064008 | 3.2 | 2.9 |
| 87492 Ovary Cancer (OD04768-07) | 3.5 | 4.6 |
| 87493 Ovary NAT (OD04768-08) | 0.9 | 2.2 |
| Normal Stomach GENPAK 061017 | 2.7 | 3.7 |
| Gastric Cancer Clontech 9060358 | 0.4 | 0.2 |
| NAT Stomach Clontech 9060359 | 4.3 | 1.3 |
| Gastric Cancer Clontech 9060395 | 3.0 | 1.2 |
| NAT Stomach Clontech 9060394 | 2.5 | 1.0 |
| Gastric Cancer Clontech 9060397 | 100.0 | 48.0 |
| NAT Stomach Clontech 9060396 | 1.0 | 2.2 |
| Gastric Cancer GENPAK 064005 | 4.9 | 6.7 |

TABLE AD
Panel 3D

| | Relative | | Relative |
|---|---|---|---|
| | Expression(%) | | Expression(%) |
| | 3dtm4781f_{—} | | 3dtm4781f_{—} |
| Tissue Name | ag1395 | Tissue Name | ag1395 |
| 94905_Daoy_Medulloblastoma/ | 0.0 | 94954_Ca Ski_Cervical | 0.0 |
| Cerebellum_sscDNA | | epidermoid carcinoma | |
| | | (metastasis)_sscDNA | |
| 94906_TE671_Medulloblastoma/ | 0.6 | 94955_ES-2_Ovarian clear cell | 0.2 |
| Cerebellum_sscDNA | | carcinoma_sscDNA | |
| 94907_D283 | 0.5 | 94957_Ramos/6h stim_″; | 0.0 |
| Med_Medulloblastoma/Cerebell | | Stimulated with | |
| um_sscDNA | | PMA/ionomycin 6h_sscDNA | |
| 94908_PFSK-1_Primitive | 0.0 | 94958_Ramos/14h stim_″; | 0.0 |
| Neuroectodermal/Cerebellum_s | | Stimulated with | |
| scDNA | | PMA/ionomycin 14h_sscDNA | |
| 94909_XF-498_CNS_sscDNA | 0.6 | 94962_MEG-01_Chronic | 0.0 |
| | | myelogenous leukemia | |
| | | (megokaryoblast)_sscDNA | |
| 94910_SNB- | 0.0 | 94963_Raji_Burkitt's | 0.0 |
| 78_CNS/glioma_sscDNA | | lymphoma_sscDNA | |
| 94911_SF- | 0.0 | 94964_Daudi_Burkitt's | 0.0 |
| 268_CNS/glioblastoma_sscDNA | | lymphoma_sscDNA | |
| 94912_T98G_Glioblastoma_ssc | 6.5 | 94965_U266_B-cell | 0.0 |
| DNA | | plasmacytoma/myeloma_sscDNA | |
| 96776_SK-N- | 10.3 | 94968_CA46_Burkitt's | 0.0 |
| SH_Neuroblastoma | | lymphoma_sscDNA | |
| (metastasis)_sscDNA | | | |
| 94913_SF- | 0.8 | 94970_RL_non-Hodgkin's B- | 0.0 |
| 295_CNS/glioblastoma_sscDNA | | cell lymphoma_sscDNA | |
| 94914_Cerebellum_sscDNA | 46.0 | 94972_JM1_pre-B-cell | 0.0 |
| | | lymphoma/leukemia_sscDNA | |
| 96777_Cerebellum_sscDNA | 41.5 | 94973_Jurkat_T cell | 0.0 |
| | | leukemia_sscDNA | |
| 94916_NCI- | 0.4 | 94974_TF- | 0.0 |
| H292_Mucoepidermoid lung | | 1_Erythroleukemia_sscDNA | |
| carcinoma_sscDNA | | | |
| 94917_DMS-114_Small cell | 9.0 | 94975_HUT 78_T-cell | 0.0 |
| lung cancer_sscDNA | | lymphoma_sscDNA | |
| 94918_DMS-79_Small cell | 100.0 | 94977_U937_Histiocytic | 5.3 |
| lung | | lymphoma_sscDNA | |
| cancer/neuroendocrine_sscDNA | | | |
| 94919_NCI-H146_Small cell | 4.2 | 94980_KU-812_Myelogenous | 0.0 |
| lung | | leukemia_sscDNA | |
| cancer/neuroendocrine_sscDNA | | | |
| 94920_NCI-H526_Small cell | 16.5 | 94981_769-P_Clear cell renal | 6.3 |
| lung | | carcinoma_sscDNA | |
| cancer/neuroendocrine_sscDNA | | | |
| 94921_NCI-N417_Small cell | 22.8 | 94983_Caki-2_Clear cell renal | 15.8 |
| lung | | carcinoma_sscDNA | |
| cancer/neuroendocrine_sscDNA | | | |
| 94923_NCI-H82_Small cell | 9.5 | 94984_SW 839_Clear cell renal | 5.9 |
| lung | | carcinoma_sscDNA | |
| cancer/neuroendocrine_sscDNA | | | |
| 94924_NCI-H157_Squamous | 0.0 | 94986_G401_Wilms' | 1.2 |
| cell lung cancer | | tumor_sscDNA | |
| (metastasis)_sscDNA | | | |
| 94925_NCI-H1155_Large cell | 27.7 | 94987_Hs766T_Pancreatic | 0.0 |
| lung | | carcinoma (LN | |
| cancer/neuroendocrine_sscDNA | | metastasis)_sscDNA | |
| 94926_NCI-H1299_Large cell | 0.0 | 94988_CAPAN-1_Pancreatic | 27.7 |
| lung | | adenocarcinoma (liver | |
| cancer/neuroendocrine_sscDNA | | metastasis)_sscDNA | |
| 94927_NCI-H727_Lung | 14.5 | 94989_SU86.86_Pancreatic | 11.0 |
| carcinoid_sscDNA | | carcinoma (liver | |
| | | metastasis)_sscDNA | |
| 94928_NCI-UMC-11_Lung | 22.1 | 94990_BxPC-3_Pancreatic | 0.0 |
| carcinoid_sscDNA | | adenocarcinoma_sscDNA | |
| 94929_LX-1_Small cell lung | 0.2 | 94991_HPAC_Pancreatic | 0.0 |
| cancer_sscDNA | | adenocarcinoma_sscDNA | |
| 94930_Colo-205_Colon | 0.2 | 94992_MIA PaCa-2_Pancreatic | 0.0 |
| cancer_sscDNA | | carcinoma_sscDNA | |
| 94931_KM12_Colon | 0.0 | 94993_CFPAC-1_Pancreatic | 2.8 |
| cancer_sscDNA | | ductal | |
| | | adenocarcinoma_sscDNA | |
| 94932_KM2OL2_Colon | 0.2 | 94994_PANC-1_Pancreatic | 0.2 |
| cancer_sscDNA | | epitheliold ductal | |
| | | carcinoma_sscDNA | |
| 94933_NCI-H716_Colon | 0.4 | 94996_T24_Bladder carcinma | 0.0 |
| cancer_sscDNA | | (transitional cell)_sscDNA | |
| 94935_SW-48_Colon | 0.0 | 94997_5637_Bladder | 0.2 |
| adenocarcinoma_sscDNA | | carcinoma_sscDNA | |
| 94936_SW1116_Colon | 7.2 | 94998_HT-1197_Bladder | 0.0 |
| adenocarcinoma_sscDNA | | carcinoma_sscDNA | |
| 94937_LS 174T_Colon | 0.9 | 94999_UM-UC-3_Bladder | 0.3 |
| adenocarcinoma_sscDNA | | carcinoma (transitional | |
| | | cell)_sscDNA | |
| 94938_SW-948_Colon | 0.0 | 95000_A204_Rhabdomyosarco | 1.0 |
| adenocarcinoma_sscDNA | | ma_sscDNA | |
| 94939_SW-480_Colon | 0.0 | 95001_HT- | 0.0 |
| adenocarcinoma_sscDNA | | 1080_Fibrosarcoma_sscDNA | |
| 94940_NCI-SNU-5_Gastric | 3.5 | 95002_MG-63_Osteosarcoma | 0.2 |
| carcinoma_sscDNA | | (bone)_sscDNA | |
| 94941_KATO III_Gastric | 0.3 | 95003_SK-LMS- | 0.0 |
| carcinoma_sscDNA | | 1_Leiomyosarcoma | |
| | | (vulva)_sscDNA | |
| 94943_NCI-SNU- 16_Gastric | 2.1 | 95004_SJRH30_Rhabdomyosar | 0.1 |
| carcinoma_sscDNA | | coma (met to bone | |
| | | marrow)_sscDNA | |
| 94944_NCI-SNU-1 Gastric | 0.0 | 95005_A431_Epidermoid | 0.0 |
| carcinoma_sscDNA | | carcinoma_sscDNA | |
| 94946_RF-1_Gastric | 0.0 | 95007_WM266- | 0.0 |
| adenocarcinoma_sscDNA | | 4_Melanoma_sscDNA | |
| 94947_RF-48_Gastric | 0.0 | 95010_DU 145_Prostate | 0.0 |
| adenocarcinoma_sscDNA | | carcinoma (brain | |
| | | metastasis)_sscDNA | |
| 96778_MKN-45_Gastric | 2.9 | 95012_MDA-MB-468_Breast | 9.2 |
| carcinoma_sscDNA | | adenocarcinoma_sscDNA | |
| 94949_NCI-N87_Gastric | 0.0 | 95013_SCC-4_Squamous cell | 0.0 |
| carcinoma_sscDNA | | carcinoma of tongue_sscDNA | |
| 94951_OVCAR-5_Ovarian | 11.2 | 95014_SCC-9_Squamous cell | 0.0 |
| carcinoma_sscDNA | | carcinoma of tongue_sscDNA | |
| 94952_RL95-2_Uterine | 0.0 | 95015_SCC-15_Squamous cell | 0.0 |
| carcinoma_sscDNA | | carcinoma of tongue_sscDNA | |
| 94953_HelaS3_Cervical | 0.0 | 95017_CAL 27_Squamous cell | 0.0 |
| adenocarcinoma sscDNA | | carcinoma of tongue_sscDNA | |

TABLE AE
Panel 4D

| | Relative Expression(%) | |
|---|---|---|
| | 4dtm2005f_{—} | 4dtm2198f_{—} |
| Tissue Name | ag1395 | ag1395 |
| 93768_Secondary Th1_anti-CD28/anti-CD3 | 0.0 | 1.5 |
| 93769_Secondary Th2_anti-CD28/anti-CD3 | 0.0 | 0.0 |
| 93770_Secondary Tr1_anti-CD28/anti-CD3 | 0.0 | 0.0 |
| 93573_Secondary Th1_resting day 4-6 in IL-2 | 0.0 | 0.0 |
| 93572_Secondary Th2_resting day 4-6 in IL-2 | 0.0 | 0.0 |
| 93571_Secondary Tr1_resting day 4-6 in IL-2 | 0.0 | 0.0 |
| 93568_primary Th1_anti-CD28/anti-CD3 | 0.0 | 0.0 |
| 93569_primary Th2_anti-CD28/anti-CD3 | 0.0 | 0.0 |
| 93570_primary Tr1_anti-CD28/anti-CD3 | 1.6 | 0.0 |
| 93565_primary Th1_resting dy 4-6 in IL-2 | 1.7 | 0.0 |
| 93566_primary Th2_resting dy 4-6 in IL-2 | 0.0 | 0.0 |
| 93567_primary Tr1_resting dy 4-6 in IL-2 | 1.3 | 0.0 |
| 93351_CD45RA CD4 lymphocyte_anti-CD28/anti-CD3 | 0.0 | 0.7 |
| 93352_CD45RO CD4 lymphocyte_anti-CD28/anti-CD3 | 1.2 | 0.0 |
| 93251_CD8 Lymphocytes_anti-CD28/anti-CD3 | 0.0 | 0.0 |
| 93353_chronic CD8 Lymphocytes 2ry_resting dy 4-6 in IL-2 | 1.2 | 0.0 |
| 93574_chronic CD8 Lymphocytes 2ry_activated CD3/CD28 | 0.0 | 0.0 |
| 93354_CD4_none | 0.0 | 0.0 |
| 93252_Secondary Th1/Th2/Tr1_anti-CD95 CH11 | 1.6 | 0.0 |
| 93103_LAK cells_resting | 41.8 | 49.7 |
| 93788_LAK cells_IL-2 | 0.0 | 0.0 |
| 93787_LAK cells_IL-2 + IL-12 | 0.0 | 0.0 |
| 93789_LAK cells_IL-2 + IFN gamma | 4.2 | 2.9 |
| 93790_LAK cells_IL-2 + IL-18 | 2.7 | 1.5 |
| 93104_LAK cells_PMA/ionomycin and IL-18 | 63.3 | 100.0 |
| 93578_NK Cells IL-2_resting | 0.0 | 0.0 |
| 93109_Mixed Lymphocyte Reaction_Two Way MLR | 21.8 | 16.7 |
| 93110_Mixed Lymphocyte Reaction_Two Way MLR | 4.6 | 2.2 |
| 93111_Mixed Lymphocyte Reaction_Two Way MLR | 5.0 | 2.5 |
| 93112_Mononuclear Cells (PBMCs)_resting | 3.6 | 9.5 |
| 93113_Mononuclear Cells (PBMCs)_PWM | 0.0 | 97.9 |
| 93114_Mononuclear Cells (PBMCs)_PHA-L | 2.9 | 2.7 |
| 93249_Ramos (B cell)_none | 0.0 | 0.0 |
| 93250_Ramos (B cell)_ionomycin | 0.0 | 0.0 |
| 93349_B lymphocytes_PWM | 13.8 | 0.0 |
| 93350_B lymphocytes_CD40L and IL-4 | 0.0 | 0.0 |
| 92665_EOL-1 (Eosinophil)_dbcAMP differentiated | 0.0 | 4.4 |
| 93248_EOL-1 (Eosinophil)_dbcAMP/PMAionomycin | 0.0 | 1.3 |
| 93356_Dendritic Cells_none | 31.9 | 34.4 |
| 93355_Dendritic Cells_LPS 100 ng/ml | 36.3 | 23.3 |
| 93775_Dendritic Cells_anti-CD40 | 39.8 | 40.3 |
| 93774_Monocytes_resting | 55.9 | 58.6 |
| 93776_Monocytes_LPS 50 ng/ml | 13.3 | 11.3 |
| 93581_Macrophages_resting | 93.3 | 96.6 |
| 93582_Macrophages_LPS 100 ng/ml | 38.7 | 28.3 |
| 93098_HUVEC (Endothelial)_none | 0.0 | 4.0 |
| 93099_HUVEC (Endothelial)_starved | 2.6 | 0.0 |
| 93100_HUVEC (Endothelial)_IL-1b | 0.0 | 1.4 |
| 93779_HUVEC (Endothelial)_IFN gamma | 1.5 | 6.3 |
| 93102_HUVEC (Endothelial)_TNF alpha + IFN gamma | 0.0 | 0.0 |
| 93101_HUVEC (Endothelial)_TNF alpha + IL4 | 0.0 | 0.0 |
| 93781_HUVEC (Endothelial)_IL-11 | 1.4 | 0.0 |
| 93583_Lung Microvascular Endothelial Cells_none | 0.0 | 1.4 |
| 93584_Lung Microvascular Endothelial Cells_TNFa (4 ng/ml) | 0.0 | 0.7 |
| and IL1b (1 ng/ml) | | |
| 92662_Microvascular Dermal endothelium_none | 11.0 | 5.5 |
| 92663_Microsvasular Dermal endothelium_TNFa (4 ng/ml) | 7.6 | 17.4 |
| and IL1b (1 ng/ml) | | |
| 93773_Bronchial epithelium_TNFa (4 ng/ml) and IL1b (1 | 0.0 | 0.0 |
| ng/ml)** | | |
| 93347_Small Airway Epithelium_none | 0.0 | 0.0 |
| 93348_Small Airway Epithelium_TNFa (4 ng/ml) and IL1b (1 | 1.1 | 1.9 |
| ng/ml) | | |
| 92668_Coronery Artery SMC_resting | 0.0 | 0.0 |
| 92669_Coronery Artery SMC_TNFa (4 ng/ml) and IL1b (1 | 0.0 | 0.0 |
| ng/ml) | | |
| 93107_astrocytes_resting | 1.5 | 0.0 |
| 93108_astrocytes_TNFa (4 ng/ml) and IL1b (1 ng/ml) | 0.0 | 1.2 |
| 92666_KU-812 (Basophil)_resting | 0.0 | 0.0 |
| 92667_KU-812 (Basophil)_PMA/ionoycin | 0.0 | 2.6 |
| 93579_CCD1106 (Keratinocytes)_none | 0.0 | 1.1 |
| 93580_CCD1106 (Keratinocytes)_TNFa and IFNg ** | 12.5 | 12.3 |
| 93791_Liver Cirrhosis | 23.8 | 21.0 |
| 93792_Lupus Kidney | 3.0 | 2.0 |
| 93577_NCI-H292 | 1.1 | 5.8 |
| 93358_NCI-H292_IL-4 | 2.1 | 6.4 |
| 93360_NCI-H292_IL-9 | 3.8 | 0.0 |
| 93359_NCI-H292_IL-13 | 0.0 | 4.2 |
| 93357_NCI-H292_IFN gamma | 7.1 | 7.1 |
| 93777_HPAEC_- | 10.4 | 13.4 |
| 93778_HPAEC_IL-1 beta/TNA alpha | 0.0 | 0.0 |
| 93254_Normal Human Lung Fibroblast_none | 0.0 | 0.0 |
| 93253_Normal Human Lung Fibroblast_TNFa (4 ng/ml) and | 0.0 | 0.0 |
| IL-1b (1 ng/ml) | | |
| 93257_Normal Human Lung Fibroblast_IL-4 | 0.0 | 0.0 |
| 93256_Normal Human Lung Fibroblast_IL-9 | 0.0 | 5.5 |
| 93255_Normal Human Lung Fibroblast_IL-13 | 1.3 | 0.0 |
| 93258_Normal Human Lung Fibroblast_IFN gamma | 0.0 | 0.0 |
| 93106_Dermal Fibroblasts CCD1070_resting | 0.0 | 0.0 |
| 93361_Dermal Fibroblasts CCD1070_TNF alpha 4 ng/ml | 0.0 | 0.0 |
| 93105_Dermal Fibroblasts CCD1070 IL-1 beta 1 ng/ml | 0.0 | 5.6 |
| 93772_dermal fibroblast_IFN gamma | 0.0 | 0.0 |
| 93771_dermal fibroblast_IL-4 | 0.0 | 0.0 |
| 93259_IBD Colitis 1** | 6.2 | 6.8 |
| 93260_IBD Colitis 2 | 0.0 | 1.5 |
| 93261_IBD Crohns | 1.4 | 0.0 |
| 735010_Colon_normal | 21.3 | 19.2 |
| 735019_Lung_none | 100.0 | 95.3 |
| 64028-1_Thymus_none | 6.9 | 1.2 |
| 64030-1_Kidney_none | 8.8 | 3.8 |

TABLE AF
Panel CNSD.01

| | Relative | | Relative |
|---|---|---|---|
| | Expression(%) | | Expression(%) |
| | cns1x4tm6178f | | cns1x4tm6l78f |
| Tissue Name | _ag1395_b2 | Tissue Name | _ag1395_b2 |
| 102633_BA4 Control | 25.8 | 102605_BA17 PSP | 35.2 |
| 102641_BA4 Control2 | 42.9 | 102612_BA17 PSP2 | 19.0 |
| 102625_BA4 Alzheimer's2 | 7.9 | 102637_Sub Nigra Control | 6.2 |
| 102649_BA4 Parkinson's | 53.0 | 102645_Sub Nigra Control2 | 8.6 |
| 102656_BA4 Parkinson's2 | 100.0 | 102629_Sub Nigra | 1.8 |
| | | Alzheimer's2 | |
| 102664_BA4 Huntington's | 33.6 | 102660_Sub Nigra Parkinson's2 | 10.6 |
| 102671_BA4 Huntington's2 | 13.3 | 102667_Sub Nigra | 11.0 |
| | | Huntington's | |
| 102603_BA4 PSP | 20.0 | 102674_Sub Nigra | 8.1 |
| | | Huntington's2 | |
| 102610_BA4 PSP2 | 57.5 | 102614_Sub Nigra PSP2 | 1.4 |
| 102588_BA4 Depression | 37.1 | 102592_Sub Nigra Depression | 2.1 |
| 102596_BA4 Depression2 | 12.6 | 102599_Sub Nigra Depression2 | 1.5 |
| 102634_BA7 Control | 63.2 | 102636_Glob Palladus Control | 2.1 |
| 102642_BA7 Control2 | 42.7 | 102644_Glob Palladus Control2 | 3.7 |
| 102626_BA7 Alzheimer's2 | 13.8 | 102620_Glob Palladus | 1.8 |
| | | Alzheimer's | |
| 102650_BA7 Parkinson's | 31.3 | 102628_Glob Palladus | 1.8 |
| | | Alzheimer's2 | |
| 102657_BA7 Parkinson's2 | 60.7 | 102652_Glob Palladus | 53.7 |
| | | Parkinson's | |
| 102665_BA7 Huntington's | 56.2 | 102659_Glob Palladus | 4.8 |
| | | Parkinson's2 | |
| 102672_BA7 Huntington's2 | 69.0 | 102606_Glob Palladus PSP | 4.6 |
| 102604_BA7 PSP | 73.2 | 102613_Glob Palladus PSP2 | 2.4 |
| 102611_BA7 PSP2 | 22.4 | 102591_Glob Palladus | 2.3 |
| | | Depression | |
| 102589_BA7 Depression | 16.6 | 102638_Temp Pole Control | 10.3 |
| 102632_BA9 Control | 29.3 | 102646_Temp Pole Control2 | 38.8 |
| 102640_BA9 Control2 | 76.7 | 102622_Temp Pole Alzheimer's | 5.6 |
| 102617_BA9 Alzheimer's | 9.4 | 102630_Temp Pole | 8.6 |
| | | Alzheimer's2 | |
| 102624_BA9 Alzheimer's2 | 22.7 | 102653_Temp Pole Parkinson's | 40.8 |
| 102648_BA9 Parkinson's | 44.1 | 102661_Temp Pole | 42.5 |
| | | Parkinson's2 | |
| 102655_BA9 Parkinson's2 | 67.4 | 102668_Temp Pole | 35.0 |
| | | Huntington's | |
| 102663_BA9 Huntington's | 68.8 | 102607_Temp Pole PSP | 5.5 |
| 102670_BA9 Huntington's2 | 30.3 | 102615_Temp Pole PSP2 | 6.9 |
| 102602_BA9 PSP | 27.5 | 102600_Temp Pole | 16.6 |
| | | Depression2 | |
| 102609_BA9 PSP2 | 11.6 | 102639_Cing Gyr Control | 67.6 |
| 102587_BA9 Depression | 10.4 | 102647_Cing Gyr Control2 | 35.5 |
| 102595_BA9 Depression2 | 26.2 | 102623_Cing Gyr Alzheimers's | 14.9 |
| 102635_BA17 Control | 59.4 | 102631_Cing Gyr Alzheimer's2 | 12.5 |
| 102643_BA17 Control2 | 38.0 | 102654_Cing Gyr Parkinson's | 21.1 |
| 102627_BA17 Alzheimer's2 | 15.7 | 102662_Cing Gyr Parkinson's2 | 36.7 |
| 102651_BA17 Parkinson's | 70.1 | 102669_Cing Gyr Huntington's | 52.0 |
| 102658_BA17 Parkinson's2 | 96.1 | 102676_Cing Gyr | 18.7 |
| | | Huntington's2 | |
| 102666_BA17 Huntington's | 36.0 | 102608_Cing Gyr PSP | 12.2 |
| 102673_BA17 Huntington's2 | 37.1 | 102616_Cing Gyr PSP2 | 5.4 |
| 102590_BA17 Depression | 17.6 | 102594_Cing Gyr Depression | 17.7 |
| 102597_BA17 Depression2 | 62.5 | 102601_Cing Gyr Depression2 | 16.6 |

Panel 1.2 Summary: Ag1395 Results from two replicate experiments using the same probe/primer set are in excellent agreement. The SC_{—}105828681_A gene is most highly expressed in cerebral cortex (CT value=22-23). Within the central nervous system, high expression is also detected in amygdala, cerebellum, thalamus, hippocampus and the spinal cord. In contrast, the gene is expressed at much lower levels in CNS cancer cell lines. Therefore, this gene may play a role in neurological diseases (see Panel CNSD.01 for potential utility).

Among metabolically relevant tissues, this transmembrane protein has low expression in pancreas and thyroid; moderate expression in pituitary and skeletal muscle; and good expression in liver (CT value=31) and adrenal gland (CT value=28-29). Although the role of the SC_{—}105828681_A gene in the physiology of the liver and adrenal gland are unknown, this gene product may be useful as an antibody or small molecule target for the treatment of diseases of these tissues, including Von Hippel-Lindau (VHL) syndrome, cirrhosis, transplantation, adrenoleukodystrophy, and congenital adrenal hyperplasia.

Significant over-expression of the SC_{—}105828681_A gene is also seen in a number of cancer cell lines compared to the normal controls, including ovarian cancer cell lines, lung cancer cell lines, renal cancer cell lines and colon cancer cell lines. Taken together, these data suggest that this gene may play a role in the above listed cancer types and thus therapeutic inhibition of the SC_{—}105828681_A gene product, through the use of antibodies or small molecule drugs, might be of utility in the treatment of ovarian, lung, renal and colon cancers.

Panel 2D Summary: Ag1395 Results from two replicate experiments using the same probe/primer set are in good agreement. Most strikingly, the SC_{—}105828681_A gene is over-expressed in 6/8 samples derived from breast cancer when compared to their associated normal adjacent tissue. In addition, there appears to be a moderate association with over-expression in samples derived from gastric and kidney cancer, when compared to their associated normal adjacent tissues. These results suggest that the expression of the SC_{—}105828681_A gene could be useful as a marker for breast cancer. In addition, therapeutic inhibition of the activity of the product of the SC_{—}105828681_A gene, through the use of antibodies or small molecule drugs, may be useful in the treatment of breast, gastric or kidney cancer.

Panel 3D Summary: Ag1395 The SC_{—}105828681_A gene is expressed in a number of samples in panel 3D. However, it is most highly expressed in a small cell lung cancer sample (CT=28). In addition, this gene shows moderate expression in clusters of cell lines derived from pancreatic cancer, kidney cancer, lung cancer, ovarian cancer and gastric cancer. Thus, the SC_{—}105828681_A gene might play a role in malignant cancer development. Therefore, inhibition of SC_{—}105828681_A protein activity, through the use of antibodies or small molecule drugs, might be of utility in the treatment of multiple forms of cancer.

Panel 4D Summary: Ag1395 The SC_{—}105828681_A transcript is highly expressed in normal lung as well as in resting macrophages, monocytes, dendritic cells and LAK cells. This transcript encodes a UNC5H1 netrin-like receptor. These types of receptors are involved in axonal guidance, neuronal migration and apoptosis in brain and may be involved in similar processes in the immune system such as allowing cells to respond and migrate in response to chemokine gradiants, mature and respond to potentially apoptotic stimuli (see reference 1). Therefore, agonistic small molecule therapeutics (ligand-like) for the protein encoded for by the SC_{—}105828681_A gene could be used for immunosuppression prior to tissue transplant. Alternatively, blocking the expression of the transcript through antisense or blocking the function of this protein with antibody or small molecule therapeutics could induce immune activation by acting like an adjuvant and could greatly enhance vaccination protocols.

Panel CNSD.01 Summary: Ag1395 The UNC5H receptors act both in axon guidance and neuronal migration during development, as well as inducers of apoptosis (except when stimulated by the ligand netrin-1) [see references]. The protein encoded by the SC_{—}105828681_A gene is similar to the UNCH receptor. Interestingly, the SC_{—}105828681_A gene shows an expression profile that is highly brain-preferential, with highest levels detected in the cerebral cortex (Panel 1.2). Panel CNSD.01 confirms this finding, with levels in the cortex approximately 5-fold higher than those found in the substantia nigra and globus palladus. Expression does not appear to be reduced in any disease state represented on this panel. Based upon these observations, the modulation and/or selective stimulation/antagonism of the putative receptor encoded by the SC_{—}105828681_A gene may be of use in enhancing or directing compensatory synatogenesis and axon/dendritic outgrowth in response to neuronal death (stroke, head trauma) neurodegeneration (Alzheimer's, Parkinson's, Huntington's, spinocerebellar ataxia, progressive supranuclear palsy) or spinal cord injury.

### EXAMPLE 3

### Quantitative Expression Analysis (TaqMan) of NOV2

Expression of NOV2 (GM_ba113d19_A) was assessed using the plimer-probe set Ag1449, described in Table BA. Results of the RTQ-PCR runs are shown in Tables BB and BC.

TABLE BA
Probe Name Ag 1449

| Primers | Sequences | | TM | Length | Start Position |
|---|---|---|---|---|---|
| Forward | 5′-CCGTTCACTCTTGCAAAGG-3′ | (SEQ ID NO. 32) | 59.4 | 19 | 395 |
| Probe | TET-5′-TCCAAGGGATTCACAACTACTTACACCA-3′-TAMRA | (SEQ ID NO. 33) | 66.6 | 28 | 445 |
| Reverse | 5′-GGCACAGTTGCTATAATTTTGG-3′ | (SEQ ID NO. 34) | 58.7 | 22 | 473 |

TABLE BB
Panel 1.3D

| | Relative Expression(%) | |
|---|---|---|
| | 1.3dtm2677t_{—} | 1.3dtm4405t_{—} |
| Tissue Name | ag1449 | ag1449 |
| Liver adenocarcinoma | 8.6 | 0.0 |
| Pancreas | 11.0 | 2.8 |
| Pancreatic ca. CAPAN 2 | 0.0 | 0.0 |
| Adrenal gland | 0.0 | 23.0 |
| Thyroid | 0.0 | 0.0 |
| Salivary gland | 0.0 | 0.0 |
| Pituitary gland | 22.5 | 0.0 |
| Brain (fetal) | 11.4 | 0.0 |
| Brain (whole) | 29.9 | 8.5 |
| Brain (amygdala) | 10.9 | 12.7 |
| Brain (cerebellum) | 9.5 | 15.3 |
| Brain (hippocampus) | 10.3 | 23.7 |
| Brain (substantia nigra) | 0.0 | 8.6 |
| Brain (thalamus) | 15.2 | 18.3 |
| Cerebral Cortex | 100.0 | 100.0 |
| Spinal cord | 0.0 | 0.0 |
| CNS ca. (glio/astro) U87-MG | 0.0 | 0.0 |
| CNS ca. (glio/astro) U-118-MG | 0.0 | 0.0 |
| CNS ca. (astro) SW1783 | 8.1 | 9.3 |
| CNS ca.* (neuro; met) SK-N-AS | 0.0 | 0.0 |
| CNS ca. (astro) SF-539 | 0.0 | 13.5 |
| CNS ca. (astro) SNB-75 | 0.0 | 0.0 |
| CNS ca. (glio) SNB-19 | 0.0 | 8.4 |
| CNS ca. (glio) U251 | 30.1 | 15.2 |
| CNS ca. (glio) SF-295 | 9.9 | 0.0 |
| Heart (fetal) | 0.0 | 0.0 |
| Heart | 0.0 | 0.0 |
| Fetal Skeletal | 0.0 | 16.5 |
| Skeletal muscle | 0.0 | 0.0 |
| Bone marrow | 0.0 | 10.7 |
| Thymus | 12.4 | 0.0 |
| Spleen | 0.0 | 0.0 |
| Lymph node | 0.0 | 10.8 |
| Colorectal | 21.9 | 13.9 |
| Stomach | 0.0 | 4.7 |
| Small intestine | 0.0 | 0.0 |
| Colon ca. SW480 | 0.0 | 0.0 |
| Colon ca.* (SW480 met) SW620 | 17.7 | 0.0 |
| Colon ca. HT29 | 11.4 | 0.0 |
| Colon ca. HCT-116 | 9.2 | 0.0 |
| Colon ca. CaCo-2 | 0.0 | 0.0 |
| 83219 CC Well to | 0.0 | 0.0 |
| Mod Diff (ODO3866) | | |
| Colon ca. HCC-2998 | 28.9 | 85.3 |
| Gastric ca.* (liver met) NCI-N87 | 22.7 | 24.7 |
| Bladder | 0.0 | 0.0 |
| Trachea | 0.0 | 12.1 |
| Kidney | 0.0 | 0.0 |
| Kidney (fetal) | 0.0 | 7.9 |
| Renal ca. 786-0 | 15.5 | 0.0 |
| Renal ca. A498 | 10.4 | 17.4 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 8.8 |
| Liver | 0.0 | 0.0 |
| Liver (fetal) | 0.0 | 0.0 |
| Liver ca. (hepatoblast) HepG2 | 0.0 | 6.7 |
| Lung | 0.0 | 0.0 |
| Lung (fetal) | 0.0 | 0.0 |
| Lung ca. (small cell) LX-1 | 0.0 | 0.0 |
| Lung ca. (small cell) NCI-H69 | 0.0 | 6.3 |
| Lung ca. (s.cell var.) SHP-77 | 18.6 | 0.0 |
| Lung ca. (large cell) NCI-H460 | 9.7 | 0.0 |
| Lung ca. (non-sm. cell) A549 | 26.2 | 0.0 |
| Lung ca. (non-s.cell) NCI-H23 | 0.0 | 0.0 |
| Lung ca (non-s.cell) HOP-62 | 22.4 | 0.0 |
| Lung ca. (non-s.cl) NCI-H522 | 0.0 | 0.0 |
| Lung ca. (squam.) SW 900 | 0.0 | 8.5 |
| Lung ca. (squam.) NCI-H596 | 0.0 | 0.0 |
| Mammary gland | 0.0 | 0.0 |
| Breast ca.* (p1. effusion) MCF-7 | 0.0 | 15.8 |
| Breast ca.* (pl. ef) MDA-MB-231 | 0.0 | 0.0 |
| Breast ca.* (p1. effusion) T47D | 0.0 | 0.0 |
| Breast ca. BT-549 | 31.2 | 50.0 |
| Breast ca. MDA-N | 19.2 | 0.0 |
| Ovary | 0.0 | 0.0 |
| Ovarian ca. OVCAR-3 | 17.3 | 5.8 |
| Ovarian ca. OVCAR-4 | 0.0 | 0.0 |
| Ovarian ca. OVCAR-5 | 23.5 | 7.8 |
| Ovarian ca. OVCAR-8 | 0.0 | 0.0 |
| Ovarian ca. IGROV-1 | 0.0 | 6.0 |
| Ovarian ca.* (ascites) SK-OV-3 | 0.0 | 15.3 |
| Uterus | 0.0 | 0.0 |
| Placenta | 0.0 | 0.0 |
| Prostate | 0.0 | 0.0 |
| Prostate ca.* (bone met) PC-3 | 0.0 | 24.1 |
| Testis | 0.0 | 0.0 |
| Melanoma Hs688(A).T | 11.0 | 0.0 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.0 |
| Adipose | 11.7 | 8.8 |

TABLE BC
Panel 4D Summary

| | Relative | | Relative |
|---|---|---|---|
| | Expression | | Expression |
| | (%) | | (%) |
| | 4dtm2679t_{—} | | 4dtm2679t_{—} |
| Tissue Name | ag1449 | Tissue Name | ag1449 |
| 93768_Secondary Th1_anti- | 14.7 | 93100_HUVEC | 6.7 |
| CD28/anti-CD3 | | (Endothelial)_IL-1b | |
| 93769_Secondary Th2_anti- | 11.4 | 93779_HUVEC | 24.1 |
| CD28/anti-CD3 | | (Endothelial)_IFN gamma | |
| 93770_Secondary Tr1_anti- | 6.0 | 93102_HUVEC | 6.5 |
| CD28/anti-CD3 | | (Endothelial)_TNF alpha + IFN | |
| | | gamma | |
| 93573_Secondary Th1_resting | 22.1 | 93101_HUVEC | 5.3 |
| day 4-6 in IL-2 | | (Endothelial)_TNF alpha + IL4 | |
| 93572_Secondary Th2_resting | 8.6 | 93781_HUVEC | 18.7 |
| day 4-6 in IL-2 | | (Endothelial)_IL-11 | |
| 93571_Secondary Tr1_resting | 22.1 | 93583_Lung Microvascular | 6.5 |
| day 4-6 in IL-2 | | Endothelial Cells_none | |
| 93568_primary Th1_anti- | 5.4 | 93584_Lung Microvascular | 7.1 |
| CD28/anti-CD3 | | Endothelial Cells_TNFa (4 | |
| | | ng/ml) and IL 1b (1 ng/ml) | |
| 93569_primary Th2_anti- | 37.6 | 92662_Microvascular Dermal | 5.4 |
| CD28/anti-CD3 | | endothelium_none | |
| 93570_primary Tr1_anti- | 58.6 | 92663_Microsvasular Dermal | 6.3 |
| CD28/anti-CD3 | | endothelium_TNFa (4 ng/ml) | |
| | | and IL 1b (1 ng/ml) | |
| 93565_primary Th1_resting dy | 35.6 | 93773_Bronchial | 0.0 |
| 4-6 in IL-2 | | epithelium_TNFa (4 ng/ml) and | |
| | | IL 1b (1 ng/ml) ** | |
| 93566_primary Th2_resting dy | 51.0 | 93347_Small Airway | 6.5 |
| 4-6 in IL-2 | | Epithelium_none | |
| 93567_primary Tr1_resting dy | 23.3 | 93348_Small Airway | 39.5 |
| 4-6 in IL-2 | | Epithehum_TNFa (4 ng/ml) | |
| | | and IL 1b (1 nglml) | |
| 93351_CD45RA CD4 | 4.8 | 92668_Coronery Artery | 6.1 |
| lymphocyte_anti-CD28/anti- | | SMC_resting | |
| CD3 | | | |
| 93352_CD45RO CD4 | 24.5 | 92669_Coronery Artery | 0.0 |
| lymphocyte_anti-CD2 8/anti- | | SMC_TNFa (4 ng/ml) and IL 1b | |
| CD3 | | (1 ng/ml) | |
| 93251_CD8 Lymphocytes_anti- | 10.6 | 93107_astrocytes_resting | 5.8 |
| CD28/anti-CD3 | | | |
| 93353_chronic CD8 | 100.0 | 93108_astrocytes_TNFa (4 | 13.9 |
| Lymphocytes 2ry_resting dy 4- | | ng/ml and IL 1b (1 ng/ml) | |
| 6 in IL-2 | | | |
| 93574_chronic CD8 | 0.0 | 92666_KU-812 | 2.6 |
| Lymphocytes 2ry_activated | | (Basophil)_resting | |
| CD3/CD28 | | | |
| 93354_CD4_none | 25.0 | 92667_KU_812 | 18.8 |
| | | (Basophil)_PMA/ionoycin | |
| 93252_Secondary | 28.5 | 93579_CCD1106 | 25.3 |
| Th1/Th2/Tr1_anti-CD95 CH11 | | (Keratinocytes)_none | |
| 93103_LAK cells_resting | 6.1 | 93580_CCD1106 | 6.5 |
| | | (Keratinocytes)_TNFa and | |
| | | IFNg** | |
| 93788_LAK cells_IL-2 | 68.8 | 93791_Liver Cirrhosis | 80.7 |
| 93787_LAK cells_IL-2 + IL-12 | 14.1 | 93792_Lupus Kidney | 0.0 |
| 93789_LAK cells_IL-2 + IFN | 12.3 | 93577_NCI-H292 | 19.8 |
| gamma | | | |
| 93790_LAK cells_IL-2 + IL-18 | 81.8 | 93358_NCI-H292_IL-4 | 18.7 |
| 93104_LAK | 12.6 | 93360_NCI-H292_IL-9 | 19.6 |
| cells_PMA/ionomycin and IL-18 | | | |
| 93578_NK Cells IL-2_resting | 57.4 | 93359_NCI-H292_IL-13 | 9.8 |
| 93109_Mixed Lymphocyte | 47.6 | 93357_NCI-H292 IFN gamma | 0.0 |
| Reaction_Two Way MLR | | | |
| 93110_Mixed Lymphocyte | 0.0 | 93777_HPAEC_- | 0.0 |
| Reaction_Two Way MLR | | | |
| 93111_Mixed Lymphocyte | 88.9 | 93778_HPAEC_IL-1 beta/TNA | 10.0 |
| Reaction_Two Way MLR | | alpha | |
| 93112_Mononuclear Cells | 5.8 | 93254_Normal Human Lung | 6.1 |
| (PBMCs)_resting | | Fibroblast_none | |
| 93113_Mononuclear Cells | 40.6 | 93253_Normal Human Lung | 18.2 |
| (PBMCs)_PWM | | Fibroblast_TNFa (4 ng/ml) and | |
| | | IL-1b (1 ng/ml) | |
| 93114_Mononuclear Cells | 32.5 | 93257_Normal Human Lung | 4.7 |
| (PBMCs)_PHA-L | | Fibroblast_IL-4 | |
| 93249_Ramos (B cell)_none | 0.0 | 93256_Normal Human Lung | 0.0 |
| | | Fibroblast_IL-9 | |
| 93250_Ramos (B | 0.0 | 93255_Normal Human Lung | 5.6 |
| cell)_ionomycin | | Fibroblast_IL-13 | |
| 93349_B lymphocytes_PWM | 67.4 | 93258_Normal Human Lung | 10.4 |
| | | Fibroblast_IFN gamma | |
| 93350_B lymphoytes_CD40L | 23.3 | 93106_Dermal Fibroblasts | 10.7 |
| and IL-4 | | CCD1070_resting | |
| 92665_EOL-1 | 11.5 | 93361_Dermal Fibroblasts | 81.2 |
| (Eosinophil)_dbcAMP | | CCD1070_TNF alpha 4 ng/ml | |
| differentiated | | | |
| 93248_EOL-1 | 6.5 | 93105_Dermal Fibroblasts | 0.0 |
| (Eosinophil)_dbcAMP/PMAion | | CCD1070_IL-1 beta 1 ng/ml | |
| omycin | | | |
| 93356_Dendritic Cells_none | 0.0 | 93772_dermal fibroblast_IFN | 6.5 |
| | | gamma | |
| 93355_Dendritic Cells_LPS | 0.0 | 93771_dermal fibroblast_IL-4 | 33.7 |
| 100 ng/ml | | | |
| 93775_Dendritic Cells_anti- | 12.5 | 93259_IBD Colitis 1** | 8.2 |
| CD40 | | | |
| 93774_Monocytes_resting | 0.0 | 93260_IBD Colitis 2 | 17.1 |
| 93776_Monocytes_LPS 50 | 0.0 | 93261_IBD Crohns | 10.4 |
| ng/ml | | | |
| 93581_Macrophages_resting | 21.9 | 735010_Colon_normal | 11.7 |
| 93582_Macrophages_LPS 100 | 0.0 | 735019_Lung_none | 19.3 |
| ng/ml | | | |
| 93098_HUVEC | 23.2 | 64028-1_Thymus none | 22.4 |
| (Endothelial)_none | | | |
| 93099_HUVEC | 12.2 | 64030-1_Kidney_none | 34.6 |
| (Endothelial)_starved | | | |

Panel 1.3D Summary: Ag1449 Results from replicate experiments using the same probe/primer set are in good agreement. Significant expression of the GM_ba113d19_A gene is restricted to cerebral cortex (CT=34.0). The GM_ba113d19_A gene encodes a putative secreted inteferon beta like molecule. Interferon alpha and beta are used as anti-inflammatory agents in the treatment of multiple sclerosis (MS), and these proteins have been shown to decreases relapse rate, numbers of new lesions, and accumulation of disability (Ref. 1). In addition to potential utility in the treatment of MS, the preferential expression of the GM_ba113d19_A gene in the cerebral cortex suggests that this protein may be useful in the reduction of generalized neuroinflammation in this region and thus reduce inflammation-associated neuronal death in response to stroke, head trauma, or Alzheimer's.

Panel 2D Summary: Ag1449 Expression of the GM_ba113d19_A gene was low/undetectable (CT values >35) in all samples on this panel.

Panel 4D Summary: Agl449 The GM_ba113d19_A transcript is expressed at significant levels only in resting secondary CD8 T cells (CT value=34.0) and TNF alpha treated dermal fibroblasts (CT value=34.4). The GM_ba113d19_A gene encodes a putative secreted inteferon beta like molecule. Interferons are stimulated during inflammation particularly during viral infections (Ref. 2) and may be important in immune defense against these organisms. Beta interferons are important in the expansion of T cells. Designing protein therapeutics with the protein encoded for by the GM_ba113d19_A transcript could therefore induce T cell expansion as well as inhibit or block viral infections. Alternatively, antibody therapeutics that block the function of the encoded protein may inhibit inflammation in during psoriasis as well as block tissue damage mediated by CD8 cells during arthritis, delayed hypersensitivity reactions, thyroiditis, diabetes and IBD.

Panel CNSD.01 Summary: Ag1449 The GM_ba113d19_A gene is expressed at low/undetectable levels (CT values>35) in all of the samples on this panel.

### EXAMPLE 4

### Quantitative Expression Analysis (TaqMan) of NOV3

Expression of NOV3 (ac009238_gene_{—}5_EXT) was assessed using the primer-probe sets Ag1317 and Ag1317b, described in Tables CA and CB. Results of the RTQ-PCR runs are shown in Tables CC, CD, and CE.

TABLE CA
Probe Name Ag1317

| | | | | Start |
|---|---|---|---|---|
| Primers | Sequences | TM | Length | Position |
| Forward | 5′-CTCCTGGACTCCCTCTATGG-3′ | 58.7 | 20 | 241 |
| | (SEQ ID NO. 35) | | | |
| Probe | FAM-5′-CTCTCGGTGGTGCAGCTCAATCCTTT- | 71.3 | 26 | 277 |
| | 3-TAMRA (SEQ ID NO. 36) | | | |
| Reverse | 5′-GGGCCTTTACCAACTCTGAA-3′ | 59.2 | 20 | 306 |
| | (SEQ ID NO. 37) | | | |

TABLE CB
Probe Name Ag1317b

| | | | | Start |
|---|---|---|---|---|
| Primers | Sequences | TM | Length | Position |
| Forward | 5′-GACCTCAGATGTCCTAGCCAT-3′ | 59.6 | 22 | 2187 |
| | (SEQ ID NO. 38) | | | |
| Probe | FAM-5′-CACCTACCTGAAAGGAGAGCTGCCTG- | 69.3 | 26 | 2211 |
| | 3′-TAMRA (SEQ ID NO. 39) | | | |
| Reverse | 5′-CCAGGAAACACTCACTCACATT-3′ | 59.1 | 22 | 2260 |
| | (SEG ID NO. 40) | | | |

TABLE CC
Panel 1.2

| | Relative Expression(%) | | |
|---|---|---|---|
| | 1.2tm1446f_{—} | 1.2tm1459f_{—} | 1.2tm1815f_{—} |
| Tissue Name | ag1317 | ag1317 | ag1317b |
| Endothelial cells | 0.0 | 0.1 | 0.0 |
| Heart (fetal) | 0.6 | 0.5 | 0.6 |
| Pancreas | 5.7 | 17.9 | 1.1 |
| Pancreatic ca. CAPAN 2 | 9.7 | 27.2 | 16.6 |
| Adrenal Gland (new lot*) | 0.6 | 0.9 | 0.8 |
| Thyroid | 6.3 | 15.5 | 1.0 |
| Salavary gland | 32.5 | 91.4 | 22.8 |
| Pituitary gland | 1.9 | 2.0 | 0.0 |
| Brain (fetal) | 1.3 | 2.2 | 0.2 |
| Brain (whole) | 1.2 | 3.1 | 0.2 |
| Brain (amygdala) | 1.8 | 1.2 | 0.7 |
| Brain (cerebellum) | 0.5 | 1.4 | 0.4 |
| Brain (hippocampus) | 3.3 | 3.4 | 1.6 |
| Brain (thalamus) | 0.6 | 1.1 | 0.9 |
| Cerebral Cortex | 3.3 | 3.6 | 2.1 |
| Spinal cord | 1.4 | 2.1 | 0.2 |
| CNS ca. (glio/astro) U87-MG | 0.0 | 0.0 | 0.0 |
| CNS ca. (glio/astro) U-1118-MG | 0.0 | 0.0 | 0.0 |
| CNS ca. (astro) SW1783 | 0.0 | 0.0 | 0.2 |
| CNS ca.* (neuro; met) SK-N-AS | 0.4 | 0.4 | 0.3 |
| CNS ca. (astro) SF-539 | 0.0 | 0.0 | 0.0 |
| CNS ca. (astro) SNB-75 | 0.1 | 0.2 | 0.2 |
| CNS ca. (glio) SNB-19 | 0.3 | 0.3 | 0.4 |
| CNS ca. (glio) U25 1 | 0.0 | 0.0 | 0.0 |
| CNS ca. (glio) SF-295 | 0.0 | 0.0 | 0.3 |
| Heart | 1.3 | 3.5 | 2.5 |
| Skeletal Muscle (new lot*) | 0.8 | 1.0 | 0.6 |
| Bone marrow | 0.0 | 0.0 | 0.0 |
| Thymus | 0.9 | 1.2 | 0.2 |
| Spleen | 0.4 | 0.2 | 0.1 |
| Lymph node | 0.2 | 0.4 | 0.0 |
| Colorectal | 13.5 | 4.3 | 1.4 |
| Stomach | 41.2 | 54.0 | 3.2 |
| Small intestine | 5.0 | 8.2 | 5.3 |
| Colon ca. 5W480 | 0.0 | 0.0 | 0.0 |
| Colon ca.* (5W480 met) SW620 | 0.0 | 0.0 | 0.0 |
| Colon ca. HT29 | 5.9 | 10.7 | 11.6 |
| Colon ca. HCT-116 | 1.2 | 1.1 | 0.5 |
| Colon ca. CaCo-2 | 0.6 | 0.9 | 0.5 |
| 83219 CC Well to Mod Diff (ODO3866) | 13.0 | 13.2 | 2.0 |
| Colon ca. HCC-2998 | 2.3 | 4.3 | 3.8 |
| Gastric ca.* (liver met) NCI-N87 | 100.0 | 90.8 | 43.8 |
| Bladder | 13.9 | 19.6 | 8.8 |
| Trachea | 15.8 | 19.5 | 0.9 |
| Kidney | 20.9 | 100.0 | 100.0 |
| Kidney (fetal) | 29.5 | 45.1 | 5.7 |
| Renal ca. 786-0 | 0.0 | 0.0 | 0.0 |
| Renal ca. A498 | 0.0 | 0.1 | 0.2 |
| Renal ca. RXF 393 | 0.3 | 0.5 | 0.9 |
| Renal ca. ACHN | 0.0 | 0.0 | 0.2 |
| Renal ca. UO-31 | 0.2 | 0.0 | 1.2 |
| Renal ca. TK-10 | 0.0 | 0.0 | 0.3 |
| Liver | 1.4 | 2.1 | 0.6 |
| Liver (fetal) | 0.3 | 0.4 | 0.2 |
| Liver ca. (hepatoblast) HepG2 | 0.0 | 0.0 | 0.0 |
| Lung | 1.2 | 1.7 | 0.0 |
| Lung (fetal) | 1.6 | 1.6 | 0.2 |
| Lung ca. (small cell) LX-1 | 0.3 | 1.3 | 3.0 |
| Lung ca. (small cell) NCI-H69 | 0.2 | 0.5 | 0.5 |
| Lung ca. (s.cell var.) SHP-77 | 0.0 | 0.0 | 0.0 |
| Lung ca. (large cell) NCI-H460 | 0.9 | 0.9 | 0.4 |
| Lung ca. (non-sm. cell) A549 | 0.4 | 0.3 | 0.4 |
| Lung ca. (non-s.cell) NCI-H23 | 0.7 | 0.5 | 0.8 |
| Lung ca (non-s.cell) HOP-62 | 0.0 | 0.0 | 0.4 |
| Lung ca. (non-s.cl) NCI-H522 | 0.7 | 1.1 | 2.2 |
| Lung ca. (squam.) SW 900 | 16.2 | 27.9 | 20.6 |
| Lung ca. (squam.) NCI-H596 | 0.0 | 0.2 | 0.2 |
| Mammary gland | 7.6 | 17.4 | 5.3 |
| Breast ca.* (p1. effusion) MCF-7 | 43.8 | 19.3 | 12.6 |
| Breast ca.* (pl. ef) MDA-MB-231 | 0.1 | 0.0 | 0.5 |
| Breast ca.* (p1. effusion) T47D | 12.9 | 25.3 | 30.6 |
| Breast ca. BT-549 | 0.1 | 0.0 | 0.0 |
| Breast ca. MDA-N | 0.0 | 0.0 | 0.0 |
| Ovary | 1.2 | 1.0 | 0.5 |
| Ovarian ca. OVCAR-3 | 4.0 | 6.9 | 3.0 |
| Ovarian ca. OVCAR-4 | 6.2 | 9.9 | 13.5 |
| Ovarian ca. OVCAR-5 | 27.9 | 20.6 | 33.7 |
| Ovarian ca. OVCAR-8 | 2.1 | 1.2 | 1.6 |
| Ovarian ca. IGROV-1 | 0.0 | 0.1 | 0.0 |
| Ovarian ca.* (ascites) SK-OV-3 | 0.7 | 0.5 | 11.9 |
| Uterus | 4.4 | 4.0 | 0.8 |
| Placenta | 17.8 | 31.0 | 0.8 |
| Prostate | 41.8 | 60.7 | 30.6 |
| Prostate ca.* (bone met) PC-3 | 0.0 | 0.1 | 0.4 |
| Testis | 0.7 | 1.7 | 0.3 |
| Melanoma Hs688(A).T | 0.0 | 0.0 | 0.0 |
| Melanoma* (met) Hs688(B).T | 0.0 | 0.0 | 0.0 |
| Melanoma UACC-62 | 0.0 | 0.0 | 0.0 |
| Melanoma M14 | 0.0 | 0.0 | 0.0 |
| Melanoma LOX IMVI | 0.0 | 0.0 | 0.0 |
| Melanoma* (met) SK-MEL-5 | 0.0 | 0.0 | 0.0 |
| Adipose | 0.5 | 0.6 | 0.3 |

TABLE CD
Panel 2D

| | Relative | | Relative |
|---|---|---|---|
| | Expression(%) | | Expression(%) |
| | 2Dtm2512f_{—} | | 2Dtm2512f_{—} |
| Tissue Name | ag1317 | Tissue Name | ag1317 |
| Normal Colon GENPAK | 10.2 | Kidney NAT Clontech 8120608 | 10.2 |
| 061003 | | | |
| 83219 CC Well to Mod Diff | 38.2 | Kidney Cancer Clontech | 4.5 |
| (ODO3866) | | | |
| 83220 CC NAT (ODO3866) | 4.0 | Kidney NAT Clontech 8120614 | 25.3 |
| 83221 CC Gr.2 rectosigmoid | 11.0 | Kidney Cancer Clontech | 0.0 |
| (ODO3868) | | 9010320 | |
| 83222 CC NAT (ODO3868) | 2.0 | Kidney NAT Clontech 9010321 | 27.4 |
| 83235 CC Mod Diff | 3.6 | Normal Uterus GENPAK | 0.4 |
| (ODO3920) | | 061018 | |
| 83236 CC NAT (ODO3920) | 4.6 | Uterus Cancer GENPAK | 4.9 |
| | | 064011 | |
| 83237 CC Gr.2 ascend colon | 10.7 | Normal Thyroid Clontech A + | 56.6 |
| (ODO3921) | | 6570-1 | |
| 83238 CC NAT (ODO3921) | 9.7 | Thyroid Cancer GENPAK | 6.2 |
| | | 064010 | |
| 83241 CC from Partial | 5.0 | Thyroid Cancer INVITROGEN | 7.1 |
| Hepatectomy (ODO4309) | | A302152 | |
| 83242 Liver NAT (ODO4309) | 0.0 | Thyroid NAT INVITROGEN | 2.9 |
| | | A302153 | |
| 87472 Colon mets to lung | 1.8 | Normal Breast GENPAK | 13.5 |
| (OD04451-01) | | 061019 | |
| 87473 Lung NAT (OD04451-02) | 1.3 | 84877 Breast Cancer | 32.8 |
| | | (OD04566) | |
| Normal Prostate Clontech A + 6546-1 | 99.3 | 85975 Breast Cancer | 34.4 |
| | | (OD04590-01) | |
| 84140 Prostate Cancer | 7.3 | 85976 Breast Cancer Mets | 47.3 |
| (OD04410) | | (OD04590-03) | |
| 84141 Prostate NAT | 7.4 | 87070 Breast Cancer Metastasis | 40.6 |
| (OD04410) | | (OD04655-05) | |
| 87073 Prostate Cancer | 18.8 | GENPAK Breast Cancer | 19.2 |
| (OD04720-01) | | 064006 | |
| 87074 Prostate NAT | 15.9 | Breast Cancer Res. Gen. 1024 | 91.4 |
| (OD04720-02) | | | |
| Normal Lung GENPAK 061010 | 1.0 | Breast Cancer Clontech | 16.7 |
| | | 9100266 | |
| 83239 Lung Met to Muscle | 0.0 | Breast NAT Clontech 9100265 | 6.2 |
| (ODO4286) | | | |
| 83240 Muscle NAT | 0.6 | Breast Cancer INVITROGEN | 19.3 |
| (ODO4286) | | A209073 | |
| 84136 Lung Malignant Cancer | 7.1 | Breast NAT INVITROGEN | 11.1 |
| (OD03126) | | A2090734 | |
| 84137 Lung NAT (OD03126) | 2.0 | Normal Liver GENPAK | 0.3 |
| | | 061009 | |
| 84871 Lung Cancer (OD04404) | 51.0 | Liver Cancer GENPAK 064003 | 0.0 |
| 84872 Lung NAT (OD04404) | 10.6 | Liver Cancer Research Genetics | 0.0 |
| | | RNA 1025 | |
| 84875 Lung Cancer (OD04565) | 7.9 | Liver Cancer Research Genetics | 0.0 |
| | | RNA 1026 | |
| 84876 Lung NAT (OD04565) | 0.9 | Paired Liver Cancer Tissue | 0.4 |
| | | Research Genetics RNA 6004-T | |
| 85950 Lung Cancer (OD04237-01) | 7.4 | Paired Liver Tissue Research | 0.0 |
| | | Genetics RNA 6004-N | |
| 85970 Lung NAT (OD04237-02) | 1.7 | Paired Liver Cancer Tissue | 0.0 |
| | | Research Genetics RNA 6005-T | |
| 83255 Ocular Mel Met to Liver | 0.0 | Paired Liver Tissue Research | 0.5 |
| (ODO4310 | | Genetics RNA 6005-N | |
| 83256 Liver NAT (ODO4310) | 0.3 | Normal Bladder GENPAK | 3.9 |
| | | 061001 | |
| 84139 Melanoma Mets to Lung | 0.0 | Bladder Cancer Research | 0.0 |
| (OD04321) | | Genetics RNA 1023 | |
| 84138 Lung NAT (OD04321) | 1.0 | Bladder Cancer INVITROGEN | 7.7 |
| | | A302173 | |
| Normal Kidney GENPAK | 30.4 | 87071 Bladder Cancer | 61.1 |
| 061008 | | (OD04718-01) | |
| 83786 Kidney Ca, Nuclear | 1.6 | 87072 Bladder Normal | 0.0 |
| grade 2 (OD04338) | | Adiacent (OD04718-03) | |
| 83787 Kidney NAT (OD04338) | 29.5 | Normal Ovary Res. Gen. | 0.4 |
| 83788 Kidney Ca Nuclear grade | 0.3 | Ovarian Cancer GENPAK | 7.2 |
| ½ (OD04339) | | 064008 | |
| 83789 Kidney NAT (OD04339) | 21.0 | 87492 Ovary Cancer | 100.0 |
| | | (OD04768-07) | |
| 83790 Kidney Ca, Clear cell | 0.2 | 87493 Ovary NAT (OD04768-08) | 0.5 |
| type (OD04340) | | | |
| 83791 Kidney NAT (OD04340) | 26.2 | Normal Stomach GENPAK | 18.0 |
| | | 061017 | |
| 83792 Kidney Ca, Nuclear | 0.0 | Gastric Cancer Clontech | 1.6 |
| grade 3 (OD04348) | | 9060358 | |
| 83793 KidneyNAT (OD04348) | 21.0 | NAT Stomach Clontech | 23.7 |
| | | 9060359 | |
| 87474 Kidney Cancer | 0.6 | Gastric Cancer Clontech | 17.7 |
| (OD04622-01) | | 9060395 | |
| 87475 Kidney NAT (OD04622-03) | 12.1 | NAT Stomach Clontech | 15.5 |
| | | 9060394 | |
| 85973 Kidney Cancer | 0.0 | Gastric Cancer Clontech | 22.1 |
| (OD04450-01) | | 9060397 | |
| 85974 Kidney NAT (OD04450-03) | 17.3 | NAT Stomach Clontech | 22.4 |
| | | 9060396 | |
| Kidney Cancer Clontech | 44.8 | Gastric Cancer GENPAK | 7.0 |
| 8120607 | | 064005 | |

TABLE CE
Panel 4D

| | Relative | | Relative |
|---|---|---|---|
| | Expression(%) | | Expression(%) |
| | 4dtm2003f_{—} | | 4dtm2003f_{—} |
| Tissue Name | ag1317 | Tissue Name | ag1317 |
| 93768_Secondary Th1_anti- | 0.0 | 93100_HUVEC | 0.0 |
| CD28/anti-CD3 | | (Endothelial)_IL-1b | |
| 93769_Secondary Th2_anti- | 0.0 | 93779_HUVEC | 0.3 |
| CD28/anti-CD3 | | (Endothelial)_IFN gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting | 0.0 | 93101_HUVEC | 0.0 |
| day 4-6 in IL-2 | | (Endothelial)_TNF alpha + 1L4 | |
| 93572_Secondary Th2_resting | 0.0 | 93781_HUVEC | 0.0 |
| day 4-6 in IL-2 | | (Endothelial)_IL-11 | |
| 93571_Secondary Tr1_resting | 0.0 | 93583_Lung Microvascular | 0.0 |
| day 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 | |
| | | ng/ml) and IL1b (1 ng/ml) | |
| 93569_primary Th2_anti- | 0.0 | 92662_Microvascular Dermal | 0.0 |
| CD28/anti-CD3 | | endothelium_none | |
| 93570_primary Tr1_anti- | 0.0 | 92663_Microvascular Dermal | 0.0 |
| CD28/anti-CD3 | | endothelium_TNFa (4 ng/ml) | |
| | | and IL1b (1 ng/ml) | |
| 93565_primary Th1_resting dy | 0.0 | 93773_Bronchial | 3.4 |
| 4-6 in IL-2 | | epithelium_TNFa (4 ng/ml) and | |
| | | IL1b (1 ng/ml)** | |
| 93566_primary Th2_resting dy | 0.0 | 93347_Small Airway | 8.4 |
| 4-6 in IL-2 | | Epithelium_none | |
| 93567_primary Tr1_resting dy | 0.0 | 93348_Small Airway | 21.2 |
| 4-6 in IL-2 | | Epithelium_TNFa (4 ng/ml) and | |
| | | IL1b(1 ng/ml) | |
| 93351_CD45RA CD4 | 0.0 | 92668_Coronery Artery | 0.0 |
| lymphocyte_anti-CD28/anti- | | SMC_resting | |
| CD3 | | | |
| 93352_CD45RO CD4 | 0.0 | 92669_Coronery Artery | 0.0 |
| lymphocyte_anti-CD28/anti- | | SMC_TNFa (4 ng/ml) and IL1b | |
| | | (1 ng/ml) | |
| 93251_CD8 Lymphocytes_anti- | 0.0 | 93107_astrocytes_resting | 0.0 |
| CD28/anti-CD3 | | | |
| 93353_chronic CD8 | 0.0 | 93108_astrocytes_TNFa(4 | 0.0 |
| Lymphocytes 2ry_resting dy 4- | | ng/ml) and IL1b (1 ng/ml) | |
| 6 in IL-2 | | | |
| 93574_chronic CD8 | 0.0 | 92666_KU-812 | 0.0 |
| Lymphocytes 2ry_activated | | (Basophil)_resting | |
| CD3/CD28 | | | |
| 93354_CD4_none | 0.0 | 92667_KU-812 | |
| | | (Basophil)_PMA/ionoycin | |
| 93252_Secondary | 0.0 | 93579_CCD1106 | 11.2 |
| Th1/Th2/Tr1_anti-CD95 CH11 | | (Keratinocytes)_none | |
| 93103_LAK cells resting | 0.0 | 93580_CCD1106 | 27.7 |
| | | (Keratinocytes)_TNFa and | |
| | | IFNg** | |
| 93788_LAK cells_IL-2 | 0.0 | 93791_Liver Cirrhosis | 0.2 |
| 93787_LAK cells_IL-2 + IL-12 | 0.0 | 93792_Lupus Kidney | 1.5 |
| 93789_LAK cells_IL-2 + IFN | 0.0 | 93577_NCI-H292 | 48.6 |
| gamma | | | |
| 93790_LAK cells_IL-2 + IL-18 | 0.0 | 93358_NCI-H292_IL-4 | 29.9 |
| 93104_LAK | 0.0 | 93360_NCI-H292_IL-9 | 30.4 |
| cells_PMA/ionomycin and IL-18 | | | |
| 93578_NK Cells IL-2_resting | 0.3 | 93359_NCI-H292_IL-13 | 100.0 |
| 93109_Mixed Lymphocyte | 0.0 | 93357_NCI-H292_IFN gamma | 50.0 |
| Reaction_Two Way MLR | | | |
| 93110_Mixed Lymphocyte | 0.0 | 93777_HPAEC_- | 0.0 |
| Reaction_Two Way MLR | | | |
| 93111_Mixed Lymphocyte | 0.0 | 93778_HPAEC_IL-1 beta/TNA | 0.0 |
| Reaction_Two Way MLR | | alpha | |
| 93112_Mononuclear Cells | 0.0 | 93254_Normal Human Lung | 0.0 |
| (PBMCs)_resting | | Fibroblast_none | |
| 93113_Mononuclear Cells | 0.0 | 93253_Normal Human Lung | 0.0 |
| (PBMCs)_PWM | | Fibroblast_TNFa (4 ng/ml) and | |
| | | IL-1b (1 ng/ml) | |
| 93114_Mononuclear Cells | 0.0 | 93257_Normal Human Lung | 0.3 |
| (PBMCs)_PHA-L | | Fibroblast_IL-4 | |
| 93249_Ramos (B cell)_none | 0.0 | 93256_Normal Human Lung | 0.0 |
| | | Fibroblast_IL-9 | |
| 93250_Ramos (B | 0.2 | 93255_Normal Human Lung | 0.0 |
| cell)_ionomycin | | Fibroblast_IL-13 | |
| 93349_B lymphocytes_PWM | 0.0 | 93258_Normal Human Lung | 0.0 |
| | | Fibroblast_IFN gamma | |
| 93350_B lymphoytes_CD40L and | 0.0 | 93106_Dermal Fibroblasts | 0.0 |
| IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.0 |
| (Eosinophil)_dbcAMP/PMAion | | CCD1070_IL-1 beta 1 ng/ml | |
| omycin | | | |
| 93356_Dendritic Cells_none | 0.0 | 93772_dermal fibroblast_IFN | 0.0 |
| | | gamma | |
| 93355_Dendritic Cells_LPS | 0.0 | 93771_dermal fibroblast_IL-4 | 0.2 |
| 100 ng/ml | | | |
| 93775_Dendritic Cells_anti- | 0.0 | 93259_IBD Colitis 1** | 0.8 |
| CD40 | | | |
| 93774_Monocytes_resting | 0.0 | 93260_IBD Colitis 2 | 0.0 |
| 93776_Monocytes_LPS 50 | 0.0 | 93261_IBD Crohms | 0.0 |
| ng/ml | | | |
| 93581_Macrophages_resting | 0.0 | 735010_Colon_normal | 0.2 |
| 93582_Macrophages_LPS 100 | 0.0 | 735019_Lung_none | 0.2 |
| ng/ml | | | |
| 93098_HUVEC | 0.0 | 64028-1_Thymus_none | 17.3 |
| (Endothelial)_none | | | |
| 93099_HUVEC | 0.0 | 64030_Kidney_none | 0.3 |
| (Endothelial)_starved | | | |

Panel 1.2 Summary: Ag1317/Ag1317b The ac109238_gene_{—}5_EXT gene encodes a prominin-like protein. Prominin is a plasma membrane protein with an N-terminal extracellular domain, five transmembrane segments flanking two short cytoplasmic loops and two large glycosylated extracellular domains, and a cytoplasmic C-terminal domain (Ref. 1). Prominin is found in the neuroepithelium and in various other epithelia of the mouse embryo. In the adult mouse, prominin has been detected in the brain ependymal layer, and in kidney tubules. In these epithelia, prominin is specific to the apical surface, where it is selectively associated with microvilli and microvilli-related structures.

Replicate experiments using two different probe/primer sets show somewhat consistent results. The ac009238_gene_{—}5_EXT gene encoding a prominin-like protein is moderately to highly expressed in the majority of samples on this panel. This gene is most highly expressed in a sample of adult kidney (CT value=23) in two of three runs and in a sample derived from a gastric cancer cell line in the third run. Overall, the ac009238_gene_{—}5_EXT gene is expressed in selected normal tissues as well as selected clusters of cell lines in panel 1.2. For instance, there is significant expression in prostate, placenta, mammary gland, kidney, bladder, stomach and salivary gland. Interestingly, the ac009238_gene_{—}5_EXT gene is over-expressed in ovarian cancer cell lines and in two breast cancer cell lines that are known to be estrogen receptor positive (T47D and MCF-7), relative to the normal controls. Both the ovary and breast are hormonally active tissues. Thus, the ac009238_gene_{—}5_EXT gene may play a role in ovarian cancer or breast cancer. Therefore, inhibition of the ac009238_gene_{—}5_EXT gene product, through the use of antibodies or small molecule drugs, may be of use in the treatment of ovarian cancer.

Among the samples from the central nervous system, the ac009238_gene_{—}5_EXT gene is moderately expressed in amygdala, cerebellum, hippocampus, thalamus, and spinal cord, with the highest expression in the cerebral cortex (CTs=27-28). Observed expression was dramatically decreased in 3 CNS cancer cell lines. Therefore, this gene may play a role in neurological diseases and expression may be lost during the development of brain cancer.

Among the tissues important for metabolic function, the ac009238_gene_{—}5_EXT gene is highly expressed in pancreas (CT=26-29) and thyroid (CT=26.2-29.8). This gene is also moderately expressed in adrenal gland (CT=30), pituitary (CT=28.6-33.6), skeletal muscle (CT=30) and liver (CT=29.1-30.4). Therefore, the ac009238_gene_{—}5_EXT gene product may be a drug target for treatment of diseases involving any or all of these tissues as well as metabolic diseases such as diabetes and obesity. Interestingly, the rat homolog of prominin was identified in a screen for blood glucose-regulated genes in SD rat skeletal muscle (Ref. 2). Since high blood glucose level induced the expression of the prominin homolog in skeletal muscle, which in turn up-regulated the expression of GAPDH, it has been proposed this gene might be a candidate for diabetes mellitus. Please note that expression in adipose is skewed by the presence of genomic DNA contamination in this sample.

Panel 2D Summary: Ag1317 Expression of the ac009238_gene_{—}5_EXT gene is highest in a sample derived from ovarian cancer and is also very high in normal prostate tissue. This result is consistent with the observations in Panel 1.2. Over-expression of the ac009238_gene_{—}5_EXT gene is seen in breast and ovarian cancers as well as bladder, thyroid, lung and colon cancers in a couple of instances, when compared to their normal adjacent tissues. Therefore, down-regulation of the activity of the ac009238_gene_{—}5_EXT gene product, through the use of antibodies or small molecule drugs, might be of use in the treatment of breast or ovarian cancer, in addition to the others listed. In contrast, there is also a strong association with the under-expression of the ac009238_gene_{—}5_EXT gene in kidney cancers when compared to normal adjacent kidney tissue. Thus, the therapeutic up-regulation of this gene, through the application of the protein itself might be of use in the treatment of kidney cancer.

Panel 4D Summary: Ag1317 The ac009238_gene_{—}5_EXT transcript is detected in NCI-H292 cells and is up-regulated in response to IL-13. NCI-H292 cells can mature into mucus-producing cells in response to IL-13. The ac009238_gene_{—}5_EXT gene encodes a prominin-like molecule that may be involved in establishing cell surface lipid domains associated with the function of these cells that, in turn, is up-regulated in response to IL-13. Antibodies raised against the ac009238_gene_{—}5_EXT gene product may therefore be used to detect goblet cells in the lung. In addition, antagonistic antibodies against the acO09238_gene_{—}5_EXT protein may block activation of goblet cells or subsequent mucus secretion by these cells and be important in the treatment of allergy or asthlna.

### EXAMPLE 5

### Quantitative Expression Analysis (TaqMan) of NOV5

Expression of NOV5 (SC_{—}87081869_A) was assessed using the primer-probe sets Ag1626 and Ag3059, described in Tables DA and DB.

TABLE DA
Probe Name Ag1626

| | | | | | Start |
|---|---|---|---|---|---|
| Primers | Sequences | | TM | Length | Position |
| Forward | 5′-CCAGAGGATCCAGATGTACATG-3′ | (SEQ ID NO. 41) | 59.4 | 22 | 427 |
| Probe | TET-5′-TCCTGTCTCTCATCCTCTACATCTTCACCA-3′-TAMRA | (SEQ ID NO. 45) | 69 | 30 | 453 |
| Reverse | 5′-GGGCTCCAGAGAAGATGTCTAC-3′ | (SEQ ID NO. 43) | 59.3 | 22 | 491 |

TABLE DB
Probe Name Ag3059

| | | | | | Start |
|---|---|---|---|---|---|
| Primers | Sequences | | TM | Length | Position |
| Forward | 5′-CCAGAGGATCCAGATGTACATG-3′ | (SEQ ID NO. 44) | 59.4 | 22 | 427 |
| Probe | TET-5′-TCCTCTACATCTTCACCAAGATCTCGG-3′-TAMRA | (SEQ ID NO. 45) | 66.8 | 27 | 465 |
| Reverse | 5′-AGGGCTCCAGAGAAGATGTCTA-3′ | (SEQ ID NO. 46) | 59.5 | 22 | 492 |

The NOV5 gene is expressed at low/undetectable levels (CT values>35) in all of the samples on Panels 1.3D, 2.2, and 3D.

### EXAMPLE 6

### Quantitative Expression Analysis (TaqMan) of NOV6

Expression of NOV6 (SC71046974_EXT) was assessed using the primer-probe sets Ag1361, described in Tables EA. Results of the RTQ-PCR runs are shown in Tables EB, EC and ED.

TABLE EA
Probe Name Ag1361

| | | | | | Start |
|---|---|---|---|---|---|
| Primers | Sequences | | TM | Length | Position |
| Forward | 5′-CTGGTCAGGTACCTGGATGTTA-3′ | (SEQ ID NO. 47) | 59 | 22 | 1420 |
| Probe | FAM-5′-TCCATCAATGAAGAGCTTCATATTCG-3′-TAMRA | (SEQ ID NO. 48) | 64.6 | 26 | 1462 |
| Reverse | 5′-CAGCCTTTAAGTGATCCATCAG-3′ | (SEQ ID NO. 49) | 58.9 | 22 | 1489 |

TABLE EB
Panel 1.3D

| | Relative | | Relative |
|---|---|---|---|
| | Expression(%) | | Expression(%) |
| | 1.3dtm3411f_{—} | | 1.3dtm3411f_{—} |
| Tissue Name | ag1361 | Tissue Name | ag1361 |
| Liver adenocarcinoma | 0.0 | Kidney (fetal) | 2.1 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 1.6 | Renal ca. UO-31 | 0.0 |
| Pituitary gland | 0.1 | Renal ca. TK-10 | 0.0 |
| Brain (fetal) | 0.0 | Liver | 0.0 |
| Brain (whole) | 0.2 | Liver (fetal) | 0.0 |
| Brain (amygdala) | 0.7 | Liver ca. (hepatoblast) HepG2 | 0.0 |
| Brain (cerebellum) | 0.0 | Lung | 0.0 |
| Brain (hippocampus) | 1.0 | Lung (fetal) | 0.0 |
| Brain (substantia nigra) | 0.0 | Lung ca. (small cell) LX-1 | 0.0 |
| Brain (thalamus) | 0.0 | Lung ca. (small cell) NCI-H69 | 0.0 |
| Cerebral Cortex | 0.0 | Lung ca. (s.cell var.) SHP-77 | 0.0 |
| Spinal cord | 0.2 | Lung ca. (large cell)NCI-H460 | 0.0 |
| CNS ca. (glio/astro) U87-MG | 0.0 | Lung ca. (non-sm. cell) A549 | 0.0 |
| CNS ca. (glio/astro) U-118-MG | 0.0 | Lung ca. (non-s.cell) NCI-H23 | 0.0 |
| CNS ca. (astro) SW1783 | 0.0 | Lung ca (non-s.cell) HOP-62 | 0.0 |
| CNS ca.* (neuro; met) SK-N-AS | 0.0 | Lung ca. (non-s.cl) NCI-H522 | 0.1 |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB-19 | 0.0 | Mammary gland | 0.0 |
| CNS ca. (glio) U251 | 0.0 | Breast ca.* (p1. effusion) MCF-7 | 0.2 |
| CNS ca. (glio) SF-295 | 0.0 | Breast ca.* (p1.ef) MDA-MB-231 | 0.0 |
| Heart (fetal) | 0.0 | Breast ca.* (p1. effusion) T47D | 0.0 |
| Heart | 0.0 | Breast ca. BT-549 | 0.0 |
| Fetal Skeletal | 0.0 | Breast ca. MDA-N | 0.0 |
| Skeletal muscle | 0.0 | Ovary | 0.0 |
| Bone marrow | 0.0 | Ovarian ca. OVCAR-3 | 0.5 |
| Thymus | 0.4 | Ovarian ca. OVCAR-4 | 0.0 |
| Spleen | 0.0 | Ovarian ca. OVCAR-5 | 0.7 |
| Lymph node | 0.0 | Ovarian ca. OVCAR-8 | 0.0 |
| Colorectal | 0.0 | Ovarian ca. IGROV-1 | 0.0 |
| Stomach | 100.0 | Ovarian ca.* (ascites) SK-OV-3 | 0.0 |
| Small intestine | 0.0 | Uterus | 0.0 |
| Colon ca. SW480 | 0.0 | Placenta | 0.0 |
| Colon ca.* (SW480 met)SW620 | 0.0 | Prostate | 0.0 |
| Colon ca. HT29 | 0.0 | Prostate ca.* (bone met)PC-3 | 0.0 |
| Colon ca. HCT-116 | 0.0 | Testis | 2.1 |
| Colon ca. CaCo-2 | 0.0 | Melanoma Hs688(A).T | 0.0 |
| 83219 CC Well to Mod Diff | 0.4 | Melanoma* (met) Hs688(B).T | 0.0 |
| (OD03866) | | | |
| Colon ca. HCC-2998 | 0.0 | Melanoma UACC-62 | 0.0 |
| Gastric ca.* (liver met) NCI-N87 | 0.0 | Melanoma M14 | 0.0 |
| Bladder | 0.0 | Melanoma LOX IMVI | 0.0 |
| Trachea | 0.4 | Melanoma* (met) SK-MEL-5 | 0.0 |
| Kidney | 12.8 | Adipose | 0.0 |

TABLE EC
Panel 2D

| | Relative | | Relative |
|---|---|---|---|
| | Expression(%) | | Expression(%) |
| | 2dtm3412f_{—} | | 2dtm3412f_{—} |
| Tissue Name | ag1361 | Tissue Name | ag1361 |
| Normal Colon GENPAK | 0.2 | Kidney NAT Clontech 8120608 | 2.1 |
| 061003 | | | |
| 83219 CC Well to Mod Diff | 0.0 | Kidney Cancer Clontech | 0.5 |
| (ODO3866) | | 8120613 | |
| 83220 CC NAT (ODO3866) | 0.0 | Kidney NAT Clontech 8120614 | 2.6 |
| 83221 CC Gr.2 rectosigmoid | 1.3 | Kidney Cancer Clontech | 0.0 |
| (ODO3868) | | 9010320 | |
| 83222 CC NAT (ODO3868) | 0.0 | Kidney NAT Clontech 9010321 | 2.9 |
| 83235 CC Mod Diff | 0.6 | Normal Uterus GENPAK | 0.0 |
| (ODO3920) | | 061018 | |
| 83236 CC NAT (ODO3920) | 0.0 | Uterus Cancer GENPAK | 0.2 |
| | | 064011 | |
| 83237 CC Gr.2 ascend colon | 0.2 | Normal Thyroid Clontech A + | 0.0 |
| (ODO3921) | | 6570-1 | |
| 83238 CC NAT (ODO3921) | 0.0 | Thyroid Cancer GENPAK | 0.0 |
| | | 064010 | |
| 83241 CC from Partial | 0.0 | Thyroid Cancer INVITROGEN | 0.0 |
| Hepatectomy (ODO4309) | | A302152 | |
| 83242 Liver NAT (ODO4309) | 0.0 | Thyroid NAT INVITROGEN | 0.0 |
| | | A302153 | |
| 87472 Colon mets to lung | 0.0 | Normal Breast GENPAK | 0.0 |
| (OD04451-01) | | 061019 | |
| 87473 Lung NAT (OD04451-02) | 0.0 | 84877 Breast Cancer | 0.0 |
| | | (OD04566) | |
| Normal Prostate Clontech A + 6546-1 | 0.0 | 85975 Breast Cancer | 0.0 |
| | | (OD04590-01) | |
| 84140 Prostate Cancer | 0.0 | 85976 Breast Cancer Mets | 0.0 |
| (OD04410 | | (OD04590-03) | |
| 84141 Prostate NAT | 0.0 | 87070 Breast Cancer Metastasis | 0.0 |
| (OD04410) | | (OD04655-05) | |
| 87073 Prostate Cancer | 0.0 | GENPAK Breast Cancer | 0.0 |
| (OD04720-01) | | 064006 | |
| 87074 Prostate NAT | 0.1 | Breast Cancer Res. Gen. 1024 | 0.3 |
| (OD04720-02) | | | |
| NormaI Lung GENPAK 061010 | 0.3 | Breast Cancer Clontech | 0.0 |
| | | 9100266 | |
| 83239 Lung Met to Muscle | 0.0 | Breast NAT Clontech 9100265 | 0.0 |
| (ODO4286) | | | |
| 83240 Muscle NAT | 0.0 | Breast Cancer INVITROGEN | 0.0 |
| (ODO4286) | | A209073 | |
| 84136 Lung Malignant Cancer | 0.0 | Breast NAT INVITROGEN | 0.0 |
| (OD03126) | | A2090734 | |
| 84137 Lung NAT (OD03126) | 0.0 | Normal Liver GENPAK | 0.0 |
| | | 061009 | |
| 84871 Lung Cancer (OD04404) | 0.0 | Liver Cancer GENPAK 064003 | 0.0 |
| 84872 Lung NAT (OD04404) | 0.0 | Liver Cancer Research Genetics | 0.0 |
| | | RNA 1025 | |
| 84875 Lung Cancer (OD04565) | 0.0 | Liver Cancer Research Genetics | 0.2 |
| | | RNA 1026 | |
| 84876 Lung NAT (OD04565) | 0.0 | Paired Liver Cancer Tissue | 0.0 |
| | | Research Genetics RNA 6004-T | |
| 85950 Lung Cancer (OD04237-01 | 0.0 | Paired Liver Tissue Research | 0.0 |
| | | Genetics RNA 6004-N | |
| 85970 Lung NAT (OD04237-02 | 0.0 | Paired Liver Cancer Tissue | 0.0 |
| | | Research Genetics RNA 6005-T | |
| 83255 Ocular Mel Met to Liver | 0.0 | Paired Liver Tissue Research | 0.0 |
| (ODO4310) | | Genetics RNA 6005-N | |
| 83256 Liver NAT (ODO4310) | 0.0 | Normal Bladder GENPAK | 0.4 |
| | | 061001 | |
| 84139 Melanoma Mets to Lung | 0.0 | Bladder Cancer Research | 0.0 |
| (OD04321 | | Genetics RNA 1023 | |
| 84138 Lung NAT (ODO4321) | 0.0 | Bladder Cancer INVITROGEN | 0.1 |
| | | A302173 | |
| Normal Kidney GENPAK | 23.3 | 87071 Bladder Cancer | 0.0 |
| 061008 | | (ODO4718-01) | |
| 83786 Kidney Ca, Nuclear | 0.7 | 87072 Bladder Normal | 0.0 |
| grade 2 (OD04338) | | Adjacent (OD04718-03) | |
| 83787 Kidney NAT (OD04338) | 9.2 | Normal Ovary Res. Gen. | 0.0 |
| 83788 Kidney Ca Nuclear grade | 0.0 | Ovarian Cancer GENPAK | 0.2 |
| ½ (OD04339) | | 064008 | |
| 83789 Kidney NAT (OD04339) | 28.3 | 87492 Ovary Cancer | 0.0 |
| | | (OD04768-07) | |
| 83790 Kidney Ca, Clear cell | 0.0 | 87493 Ovary NAT (OD04768-08 | 0.0 |
| type (OD04340) | | | |
| 83791 Kidney NAT (OD04340) | 45.4 | Normal Stomach GENPAK | 100.0 |
| | | 061017 | |
| 83792 Kidney Ca, Nuclear | 0.0 | Gastric Cancer Clontech | 5.3 |
| grade 3 (OD04348) | | 9060358 | |
| 83793 Kidney NAT (OD04348) | 18.6 | NAT Stomach Clontech | 78.5 |
| | | 9060359 | |
| 87474 Kidney Cancer | 0.0 | Gastric Cancer Clontech | 0.3 |
| (OD04622-01) | | 9060395 | |
| 87475 Kidney NAT (OD04622-03) | 3.4 | NAT Stomach Clontech | 31.6 |
| | | 9060394 | |
| 85973 Kidney Cancer | 0.0 | Gastric Cancer Clontech | 0.2 |
| (OD04450-01) | | 9060397 | |
| 85974 Kidney NAT (OD04450-03) | 25.7 | NAT Stomach Clontech | 29.5 |
| | | 9060396 | |
| Kidney Cancer Clontech | 0.0 | Gastric Cancer GENPAK | 4.5 |
| 8120607 | | 064005 | |

TABLE ED
Panel 4D

| | Relative | | Relative |
|---|---|---|---|
| | Expression(%) | | Expression(%) |
| | 4dtm3413f_{—} | | 4dtm3413f_{—} |
| Tissue Name | ag1361 | Tissue Name | ag1361 |
| 93768_Secondary Th1_anti- | 0.0 | 93100_HUVEC | 0.0 |
| CD28/anti-CD3 | | (Endothelial)_IL-1b | |
| 93769_Secondary Th2_anti- | 0.0 | 93779_HUVEC | 0.0 |
| CD28/anti-CD3 | | (Endothelial)_IFN gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting | 0.0 | 93101_HUVEC | 0.0 |
| day 4-6 in IL-2 | | (Endothelial)_TNF alpha + IL4 | |
| 93572_Secondary Th2_resting | 0.0 | 93781_HUVEC | 0.0 |
| day 4-6 in IL-2 | | (Endothelial)_IL-11 | |
| 93571_Secondary Tr1_resting | 0.0 | 93583_Lung Microvascular | 0.0 |
| day 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 | |
| | | ng/ml) and IL1b (1 ng/ml) | |
| 93569_primary Th2_anti- | 0.0 | 92662_Microvascular Dermal | 0.0 |
| CD28/anti-CD3 | | endothelium_none | |
| 93570_primary Tr1_anti- | 0.0 | 92663_Microsvasular Dermal | 0.0 |
| CD28/anti-CD3 | | endothelium_TNFa (4 ng/ml) and | |
| | | IL1b (1 ng/ml) | |
| 93565_primary Th1_resting dy | 0.0 | 93773_Bronchial | 0.0 |
| 4-6 in IL-2 | | epithelium_TNFa (4 ng/ml) and | |
| | | IL1b (1 ng/ml)** | |
| 93566_primary Th2_resting dy | 0.0 | 93347_Small Airway | 0.0 |
| 4-6 in IL-2 | | Epithelium_none | |
| 93567_primary Tr1_resting dy | 0.0 | 93348_Small Airway | 0.0 |
| 4-6 in IL-2 | | Epithelium_TNFa (4 ng/ml) | |
| | | IL1b (1 ng/ml) | |
| 93351_{—CD45RA CD4} | 0.0 | 92668_Coronary Artery | 0.0 |
| lymphocyte_anti-CD28/anti- | | SMC_resting | |
| CD3 | | | |
| 93352_CD45RO CD4 | 0.0 | 92669_Coronery Artery | 0.0 |
| lymphocyte_anti-CD28/anti- | | SMC_TNFa (4 ng/ml) and ILib | |
| CD3 | | (1 ng/ml) | |
| 93251_CD8 Lymphocytes_anti- | 0.0 | 93107_astrocytes_resting | 0.0 |
| CD28/anti-CD3 | | | |
| 93353_chronic CD8 | 0.0 | 93108_astrocytes_TNFa (4 | 0.1 |
| Lymphocytes 2ry_resting dy 4- | ng/ml) and IL1b (1 ng/ml) | | |
| 6 in IL-2 | | | |
| 93574_chronic CD8 | 0.0 | 92666_KU-812 | 0.0 |
| Lymphocytes 2ry_activated | (Basophil)_resting | | |
| CD3/CD28 | | | |
| 93354_CD4_none | 0.0 | 92667_KU-812 | 0.0 |
| | | (Basophil)_PMA/ionoycin | |
| 93252_Secondary | 0.0 | 93579_CCD1106 | 0.0 |
| Th1/Th2/Tr1_anti-CD95 CH11 | | (Keratinocytes)_none | |
| 93103_LAK cells_resting | 0.0 | 93580_CCD11O6 | 0.0 |
| | | (Keratinocytes)_TNFa and | |
| | | IFNg** | |
| 93788_LAK cells_IL-2 | 0.0 | 93791_Liver Cirrhosis | 0.0 |
| 93787_LAK cells_IL-2 + IL-12 | 0.0 | 93792_Lupus Kidney | 1.2 |
| 93789_LAK cells_IL-2 +IFN | 0.0 | 93577_NCI-H292 | 0.2 |
| gamma | | | |
| 93790_LAK cells_IL-2 + IL-18 | 0.0 | 93358_NCI-H292_IL-4 | 0.0 |
| 93104_LAX | 0.0 | 93360_NCI-H292_IK-9 | 0.2 |
| cells_PMA/ionomycin and IL-18 | | | |
| 93578_NK Cells IL-2_resting | 0.0 | 93359_NCI-H292_IL-13 | 0.0 |
| 93109_Mixed Lymphocyte | 0.0 | 93357_NCI-H292_IFN gamma | 0.0 |
| Reaction_Two Way MLR | | | |
| 93110_Mixed Lymphocyte | 0.0 | 93777_HPAEC_IL-1 beta/TNA | 0.0 |
| Reaction_Two Way MLR | | alpha | |
| 93111_Mixed Lymphocyte | 0.0 | 93778_HPAEC_IL-1 beta/TNA | 0.0 |
| Reaction_Two Way MLR | | alpha | |
| 93112_Mononuclear Cells | 0.0 | 93254_Normal Human Lung | 0.0 |
| (PBMCs)_resting | | Fibroblast_none | |
| 93113_Mononuclear Cells | 0.0 | 93253_Normal Human Lung | 0.0 |
| (PBMCs)_PWM | | Fibroblast_TNFa (4 ng/ml) and | |
| | | IL-1b (1 ng/ml) | |
| 93114_Mononuclear Cells | 0.0 | 93257_Normal Human Lung | 0.0 |
| (PBMCs)_PHA-L | | Fibroblast_IL-4 | |
| 93249_Ramos (B cell) none | 0.0 | 93256_Normal Human Lung | 0.0 |
| | | Fibroblast_IL-9 | |
| 93250_Ramos (B | 0.0 | 93255_Normal Human Lung | 0.0 |
| cell)_ionomycin | | Fibroblast_IL-13 | |
| 93349_B lymphocytes_PWM | 0.0 | 93258_Normal Human Lung | 0.0 |
| | | Fibroblast_IFN gamma | |
| 93350_B lymphoytes_CD40L | 0.0 | 93106_Dermal Fibroblasts | 0.0 |
| and IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.0 |
| (Eosinophil)_dbcAMP/PMAion | | CCD1070_IL-1 beta 1 ng/ml | |
| omycin | | | |
| 93356_Dendritic Cells_none | 0.0 | 93772_dermal fibroblast_IFN | 0.0 |
| | | gamma | |
| 93355_Dendritic Cells_LPS | 0.0 | 93771_dermal fibroblast_IL-4 | 0.0 |
| 100 ng/ml | | | |
| 93775_Dendritic Cells anti- | 0.0 | 93259_IBD Colitis 1** | 0.0 |
| CD40 | | 93259_IBD Colitis 1** | |
| 93774_Monocytes_resting | 0.0 | 93260_IBD Colitis 2 | 0.0 |
| 93776_Monocytes_LPS 50 | 0.0 | 93261_IBD Crohms | 0.0 |
| ng/ml | | | |
| 93581_Macrophages_resting | 0.0 | 735010_Colon_normal | 0.0 |
| 93582_Macrophages_LPS 100 | 0.0 | 735019_Lung_none | 0.0 |
| ng/ml | | | |
| 93098_HUVEC | 0.0 | 64028-1_Thymus_none | 100.0 |
| (Endothelial)_none | | | |
| 93099_HUVEC | 0.0 | 64030-1_Kidney_none | 0.0 |
| (Endothelial)_starved | | | |

Panel 1.3D Summary: Ag1361 Expression of the SC71046974_EXT gene is restricted to stomach (CT value=29.9) and kidney (CT value=32.9) tissue. This observation is consistent with the identification of this gene as a sodium/hydrogen ion exchanger because the function of both of these tissues requires sodium/hydrogen ion exchange activity. The inhibition of the SC71046974_EXT protein activity, through the use of antibodies or small molecule drugs, might be of use in the treatment of kidney or gastric diseases related to the function of a sodium/hydrogen ion exchanger. For example, the activity of this gene may be related to over-production of stomach acid leading to acid reflux disease or peptic ulcer.

Panel 2D Summary: Ag1361 Consistent with what was observed in Panel 1.3D, expression of the SC71046974_EXT gene in panel 2D is restricted to both normal kidney and stomach adjacent to tumor tissue. Interestingly, expression of the gene is absent in 4/4 gastric tumors and 10/10 kidney cancers when compared to the normal adjacent tissue controls. Thus, the expression of this gene appears to be a consistent trait of the non-neoplastic kidney and stomach. Therefore the absence of expression of this gene could be used as a diagnostic marker for kidney or gastric cancer. In addition, the replacement of this gene, potentially through the direct application of the protein or using gene replacement therapy, could be of use in the treatment of kidney or gastric cancer. Na+/H+ exchangers have previously been implicated in modulation of cellular adhesion and tumor invasion (Refs. 1 and 2).

Panel 4D Summary: Ag1361 The SC71046974_EXT transcript is expressed in the thymus in Panel 4D (CT=28.6), but not in Panel 1.3D (CT=38). The SC71046974_EXT gene encodes a putative ion exchange molecule and may therefore be important in signal transduction in the thymus. Antibodies against the protein encoded for by the SC71046974_EXT gene may be used to identify thymic tissue. Additionally, small molecule or antibody therapeutics designed against this putative ion exchanger could disrupt T cell development in the thymus and serve an immunosuppresive function that could be important for tissue transplant.

### EXAMPLE 7

### Quantitative Expression Analysis (TaqMan) of NOV7

Expression of NOV7 (GMAC040907.3_A) was assessed using the primer-probe sets Ag1399 and Agl625 (identical sequences), described in Table FA. Results of the RTQ-PCR runs are shown in Tables FB.

TABLE FA
Probe Name Ag1399/Ag1625

| | | | | | Start |
|---|---|---|---|---|---|
| Primers | Sequences | | TM | Length | Position |
| Forward | 5′-TTGAAGAAGGCAGAAACACAA-3′ | (SEQ ID NO. 50) | 58.5 | 21 | 100 |
| Probe | TET-5′-CCGCCTTCAAGAGAAACAAACGAAAG-3′-TAMRA | (SEQ ID NO. 51) | 68.7 | 26 | 133 |
| Reverse | 5′-CGCAGCTCACAGCTCATTAT-3′ | (SEQ ID NO. 52) | 59.2 | 20 | 176 |

TABLE FB
Panel 1.2

| | Relative Expression (%) |
|---|---|
| Tissue Name | 1.2 tm1690t_ ag139 |
| Endothelial cells | 0.0 |
| Heart (fetal) | 0.0 |
| Pancreas | 0.0 |
| Pancreatic ca. CAPAN 2 | 0.0 |
| Adrenal Gland (new lot*) | 0.0 |
| Thyroid | 0.0 |
| Salavary gland | 0.0 |
| Pituitary gland | 0.0 |
| Brain (fetal) | 0.0 |
| Brain (whole) | 0.0 |
| Brain (amygdala) | 0.0 |
| Brain (cerebellum) | 0.0 |
| Brain (hippocampus) | 0.0 |
| Brain (thalamus) | 1.4 |
| Cerebral Cortex | 0.0 |
| Spinal cord | 0.0 |
| CNS ca. (glio/astro) U87-MG | 0.0 |
| CNS ca. (glio/astro) U-118-MG | 0.0 |
| CNS ca. (astro) 5W1783 | 0.0 |
| CNS ca.* (neuro; met) SK-N-AS | 0.0 |
| CNS ca. (astro) SF-539 | 0.0 |
| CNS ca. (astro) SNB-75 | 0.0 |
| CNS ca. (glio) SNB-19 | 0.1 |
| CNS ca. (glio) U251 | 0.0 |
| CNS ca. (glio) SF-295 | 0.0 |
| Heart | 0.0 |
| Skeletal Muscle (new lot*) | 0.0 |
| Bone marrow | 0.0 |
| Thymus | 0.0 |
| Spleen | 0.0 |
| Lymph node | 0.0 |
| Colorectal | 0.3 |
| Stomach | 0.0 |
| Small intestine | 0.0 |
| Colon ca. SW480 | 0.0 |
| Colon ca.* (SW480 met)SW620 | 0.0 |
| Colon ca. HT29 | 0.2 |
| Colon ca. HCT-116 | 0.0 |
| Colon ca. CaCo-2 | 0.0 |
| 83219 CC Well to Mod Diff(ODO3866) | 0.4 |
| Colon ca. HCC-2998 | 0.0 |
| Gastric ca.* (liver met) NCI-N87 | 0.0 |
| Bladder | 0.3 |
| Trachea | 0.0 |
| Kidney | 0.2 |
| Kidney (fetal) | 0.0 |
| Renal Ca. 786-0 | 0.0 |
| Renal Ca. A498 | 0.0 |
| Renal Ca. RXF 393 | 0.0 |
| Renal Ca. ACHN | 0.0 |
| Renal Ca. UO-31 | 0.3 |
| Renal ca. TK-10 | 0.0 |
| Liver | 0.0 |
| Liver (fetal) | 0.0 |
| Liver Ca. (hepatoblast) HepG2 | 0.0 |
| Lung | 0.0 |
| Lung (fetal) | 0.0 |
| Lung ca. (small Cell) LX-1 | 0.0 |
| Lung ca. (small Cell) NCI-H69 | 1.9 |
| Lung ca. (s.Cell var.) SHP-77 | 0.0 |
| Lung ca. (large cell)NCI-H460 | 0.6 |
| Lung ca. (non-sm. Cell) A549 | 0.6 |
| Lung ca. (non-s.cell) NCI-H23 | 0.0 |
| Lung ca (non-s.cell) HOP-62 | 0.2 |
| Lung ca. (non-s.d) NCI-H522 | 0.1 |
| Lung ca. (squam.) SW 900 | 0.1 |
| Lung ca. (squam.) NCI-H596 | 0.2 |
| Mammary gland | 0.0 |
| Breast ca.* (pl. effusion) MCF-7 | 0.0 |
| Breast ca.* (pl.ef) MIDA-MB-23 1 | 0.0 |
| Breast ca.* (pl. effusion) T47D | 0.4 |
| Breast ca. BT-549 | 0.1 |
| Breast ca. MDA-N | 0.4 |
| Ovary | 0.0 |
| Ovarian ca. OVCAR-3 | 0.0 |
| Ovarian ca. OVCAR-4 | 0.0 |
| Ovarian ca. OVCAR-5 | 1.2 |
| Ovarian ca. OVCAR-8 | 0.0 |
| Ovarian ca. IGROV-1 | 0.0 |
| Ovarian ca.* (aseites) SK-OV-3 | 0.0 |
| Uterus | 0.0 |
| Placenta | 0.0 |
| Prostate | 0.0 |
| Prostate ca.* (bone met)PC-3 | 0.0 |
| Testis | 0.0 |
| Melanoma Hs688(A).T | 0.0 |
| Melanoma* (met) Hs688(B).T | 0.4 |
| Melanoma UACC-62 | 0.0 |
| Melanoma M14 | 2.2 |
| Melanoma LOX TMVI | 0.0 |
| Melanoma* (met) SK-MEL-5 | 0.0 |
| Adipose | 100.0 |

Panel 1.2 Summary: Ag1399 Data from a replicate experiment using the same probe/primer set was not included due to artifactual results arising from problems with some of the wells. Expression in this panel is skewed by the presence of genomic DNA contamination in the adipose sample. Disregarding this sample, low expression of the GMAC040907.3_A gene was detected in thalamus (CT=33.9), an ovarian cancer cell line (CT=34.1), a single lung cancer cell line (CT=33.4) and a single melanoma cell line (CT=33.2). Thus, the therapeutic inhibition of GMAC040907.3_A gene activity, through the use of small molecule drugs or antibodies, might be of utility in the treatment of the above listed cancer types. Beta-thymosins have been implicated in metastasis, wound healing, and a variety of other functions (Ref. 1).

Panel 1.3D Summary: Ag1625 Expression of the GMAC040907.3_A gene was low/undetectable (CT values>35) in all samples on this panel.

Panel 2D Summary: Ag1625 Expression of the GMAC040907.3_A gene was low/undetectable (CT values>35) in all samples on this panel.

Panel 4D Summary: Ag1625 Expression of the GMAC040907.3_A gene was low/undetectable (CT values>35) in all samples on this panel.

### EXAMPLE 8

### Quantitative Expression Analysis (TaqMan) of NOV8

Expression of NOV8 (20760813_EXT) was assessed using the primer-probe sets Ag998 and Gpcr10, described in Table GA and GB. Results of the RTQ-PCR runs are shown in Tables GC, GD, GE, GF, GG, GH, and GI.

TABLE GA
Probe Name Ag998

| | | | | |
|---|---|---|---|---|
| | | | | Start |
| Primers | Sequences | TM | Length | Position |

| | | | | | |
|---|---|---|---|---|---|
| Forward | 5′-CAATATGCCTGTGTATGCCTTT-3′ | (SEQ ID NO. 53) | 59 | 22 | 193 |
| Probe | TET-5′-AAAAGATTGTTCCACCTGAAACACCT-3′-TAMRA | (SEQ ID NO. 54) | 64.2 | 26 | 215 |
| Reverse | 5′-TCCAGTAAAGGCCAATAGTCAA-3′ | (SEQ ID NO. 55) | 58.8 | 22 | 246 |

TABLE GB
Probe Name Gpcr10

| | | | | | Start |
|---|---|---|---|---|---|
| Primers | Sequences | | TM | Length | Position |
| Forward | 5′-ACAGCAGTACCAACAGAAGCCC-3′ | (SEQ ID NO. 56) | | 22 | 119 |
| Probe | FAM-5′-TCCCACCTCCGCAGCCTCATCA-3′-TAMRA | (SEQ ID NO. 57) | | 22 | 143 |
| Reverse | 5′-ATATTGACATGCTTCAGATGCAGG-3′ | (SEQ ID NO. 58) | | 24 | 166 |

TABLE GC
Panel 1

| | Relative | | Relative |
|---|---|---|---|
| | Expression(%) | | Expression(%) |
| | tm597f_ | | tm597f_ |
| Tissue Name | gpcr10 | Tissue Name | gpcr10 |
| Endothelial cells | 0.0 | Kidney (fetal) | 0.0 |
| Endothelial cells (treated) | 0.0 | Renal Ca. 786-0 | 0.0 |
| Pancreas | 0.0 | Renal ca. A498 | 0.0 |
| Pancreatic ca. CAPAN 2 | 0.0 | Renal ca. RXF 393 | 0.0 |
| Adipose | 62.8 | Renal ca. ACLIN | 0.0 |
| Adrenal gland | 0.0 | Renal ca. UO-31 | 12.9 |
| Thyroid | 19.5 | Renal ca. TK-10 | 7.1 |
| Salavary gland | 0.0 | Liver | 0.0 |
| Pitnitary gland | 15.5 | Liver (fetal) | 0.0 |
| Brain (fetal) | 27.4 | Liver ca. (hepatoblast) HepG2 | 0.0 |
| Brain (whole) | 11.6 | Lung | 0.0 |
| Brain (amygdala) | 29.9 | Lung (fetal) | 0.0 |
| Brain (cerebellum) | 1.9 | Lung ca. (small cell) LX-1 | 0.0 |
| Brain (hippocampus) | 30.1 | Lung ca. (small cell) NCI-H69 | 100.0 |
| Brain (substantia nigra) | 10.4 | Lung ca. (s.cell var.) SHP-77 | 2.6 |
| Brain (thalamus) | 32.5 | Lung ca. (large cell)NCI-H460 | 2.8 |
| Brain (hypothalamus) | 3.7 | Lung ca. (non-sm. cell) A549 | 12.2 |
| Spinal cord | 2.8 | Lung ca. (non-s.cell) NCI-H23 | 0.0 |
| CNS ca. (glio/astro) U87-MG | 32.5 | Lung ca (non-s.cell) HOP-62 | 1.3 |
| CNS ca. (glio/astro) U-118-MG | 0.0 | Lung ca. (non-s.d) NCI-H522 | 0.0 |
| CNS ca. (astro) SW1783 | 0.0 | Lung ca. (squam.) SW 900 | 25.7 |
| CNS ca.* (neuro; met) SK-N- | 62.8 | Lung Ca. (squam.) NCI-H596 | 86.5 |
| AS | | | |
| CNS ca. (astro) SF-539 | 0.0 | Mammary gland | 0.0 |
| CNS ca. (astro) SNB-752 | 0.9 | Breast ca.* (pl. effusion) MCF- | 0.0 |
| | | 7 | |
| CNS Ca. (glio) SNB-19 | 69.3 | Breast ca.* (pl.ef) MDA-MB- | 0.0 |
| | | 231 | |
| CNS ca. (glio) U251 | 19.3 | Breast ca.* (pl. effusion) T47D | 0.0 |
| CNS ca. (glio) SF-295 | 61.1 | Breast ca. BT-549 | 21.3 |
| Heart | 0.0 | Breast ca. MDA-N | 4.9 |
| Skeletal muscle | 0.0 | Ovary | 4.4 |
| Bone marrow | 0.0 | Ovarian ca. OVCAR-3 | 13.8 |
| Thymus | 0.0 | Ovarian Ca. OVCAR-4 | 0.0 |
| Spleen | 0.0 | Ovarian Ca. OVCAR-5 | 0.0 |
| Lymph node | 0.0 | Ovarian Ca. OVCAR-8 | 42.0 |
| Colon (ascending) | 5.7 | Ovarian ca. IGROV-1 | 0.0 |
| Stomach | 0.1 | Ovarian ca.* (ascites) SK-OV-3 | 0.0 |
| Small intestine | 0.0 | Uterus | 0.6 |
| Colon ca. SW480 | 0.0 | Placenta | 0.0 |
| Colon ca.* (SW480 met)SW620 | 0.0 | Prostate 0.0 | |
| Colon ca. HT29 | 0.0 | Prostate ca.* (bone met)PC-3 | 21.9 |
| Colon ca. IHCT-1 16 | 0.0 | Testis | 20.7 |
| Colon ca. CaCo-2 | 0.0 | Melanoma Hs688(A).T | 0.0 |
| Colon ca. HCT-15 | 0.0 | Melanoma* (met) 11s688(B).T | 0.0 |
| Colon ca. HCC-2998 | 0.0 | Melanoma UACC-62 | 0.0 |
| Gastric ca.* (liver met) NCI- | 0.0 | Melanoma M14 | 6.9 |
| N87 | | | |
| Bladder | 0.2 | Melanoma LOX IMVI | 0.0 |
| Trachea | 0.0 | Melanoma* (met) SK-MEL-5 | 0.0 |
| Kidney | 1.8 | Melanoma SK-MEL-28 | 0.0 |

TABLE GD
Panel 1.1

Relative Expression (%)

| | 1.1tm611f_ | 1.1tm643f_ |
|---|---|---|
| Tissue Name | gpcr10 | gpcr10 |
| Adipose | 12.0 | 7.5 |
| Adrenal gland | 0.0 | 0.8 |
| Bladder | 0.2 | 1.1 |
| Brain (amygdala) | 20.0 | 9.5 |
| Brain (cerebellum) | 19.6 | 8.5 |
| Brain (hippocampus) | 27.0 | 18.8 |
| Brain (substantia nigra) | 13.8 | 7.1 |
| Brain (thalamus) | 27.7 | 10.4 |
| Cerebral Cortex | 95.9 | 51.4 |
| Brain (fetal) | 53.2 | 19.5 |
| Brain (whole) | 54.0 | 24.3 |
| CNS ca. (glio/astro) U-118-MG | 0.0 | 0.0 |
| CNS ca. (astro) SF-539 | 0.0 | 0.0 |
| CNS ca. (astro) SNB-75 | 21.6 | 7.9 |
| CNS ca. (astro) 5W1783 | 0.0 | 0.0 |
| CNS ca. (glio) U251 | 25.2 | 9.5 |
| CNS ca. (giio) SF-295 | 77.4 | 39.2 |
| CNS ca. (giio) SNB-19 | 64.2 | 21.6 |
| CNS ca. (gilo/astro) U87-MG | 32.8 | 12.2 |
| CNS ca.* (neuro; met) SK-N-AS | 79.0 | 35.8 |
| Mammary gland | 0.0 | 0.1 |
| Breast ca. BT-549 | 15.3 | 0.0 |
| Breast cia. MDA-N | 1.8 | 3.6 |
| Breast Ca.* (pl. effusion) T47D | 0.0 | 0.2 |
| Breast ca.* (pl. effusion) MCF-7 | 0.0 | 0.0 |
| Breast ca.* (pl.ef) MIDA-MB-231 | 0.0 | 0.0 |
| Small intestine | 0.0 | 0.7 |
| Colorectal | 0.0 | 0.0 |
| Colon ca. HT29 | 0.0 | 0.0 |
| Colon ca. CaCo-2 | 0.0 | 0.4 |
| Colon ca. ECT-15 | 0.0 | 0.0 |
| Colon ca. HCT-116 | 0.0 | 0.0 |
| Colon ca. HCC-2998 | 0.0 | 0.0 |
| Colon ca. SW480 | 0.0 | 0.0 |
| Colon ca.* (SW480 met)SW620 | 0.0 | 0.0 |
| Stomach | 3.4 | 4.3 |
| Gastric ca.* (liver met) NCI-N87 | 0.0 | 0.3 |
| Heart | 0.0 | 0.0 |
| Fetal Skeletal | 1.0 | 2.7 |
| Skeletal muscle | 0.0 | 0.0 |
| Endothelial cells | 0.0 | 0.0 |
| Heart (fetal) | 0.0 | 0.0 |
| Kidney | 5.7 | 3.6 |
| Kidney (fetal) | 2.6 | 2.9 |
| Renal ca. 786-0 | 0.0 | 0.0 |
| Renal ca. A498 | 0.9 | 3.5 |
| Renal ca. ACHN | 0.0 | 0.8 |
| Renal ca. TK-10 | 6.3 | 5.1 |
| Renal ca. UO-31 | 17.3 | 8.8 |
| Renal ca. RXF 393 | 0.0 | 0.6 |
| Liver | 0.0 | 0.2 |
| Liver (fetal) | 0.0 | 0.0 |
| Liver ca. (hepatoblast) HepG2 | 0.0 | 0.0 |
| Lung | 0.0 | 0.0 |
| Lung (fetal) | 0.0 | 0.3 |
| Lung ca (non-s.cell) HOP-62 | 1.1 | 2.4 |
| Lung ca. (large cell)NCI-H460 | 0.0 | 2.5 |
| Lung ca. (non-s.cell) NCI-H23 | 0.0 | 0.0 |
| Lung ca. (non-s.d) NCI-H522 | 0.0 | 0.0 |
| Lung ca. (non-sm. cell) A549 | 5.0 | 4.8 |
| Lung ca. (s.cell var.) SHP-77 | 6.5 | 5.6 |
| Lung ca. (small cell) LX- 1 | 0.0 | 0.0 |
| Lung ca. (small cell) NCI-H69 | 100.0 | 100.0 |
| Lung ca. (squam.) SW 900 | 9.3 | 7.3 |
| Lung ca. (squam.) NCI-H596 | 77.4 | 41.2 |
| Lymph node | 0.0 | 0.1 |
| Spleen | 0.0 | 1.4 |
| Thymus | 0.0 | 0.9 |
| Ovary | 1.4 | 2.8 |
| Ovarian ca. IGROV-1 | 0.0 | 0.0 |
| Ovarian ca. OVCAR-3 | 14.3 | 9.9 |
| Ovarian ca. OVCAR-4 | 0.0 | 0.0 |
| Ovarian ca. OVCAR-5 | 0.0 | 2.3 |
| Ovarian ca. OVCAR-8 | 10.7 | 5.0 |
| Ovarian ca.* (ascites) SK-OV-3 | 0.0 | 0.8 |
| Pancreas | 1.7 | 4.4 |
| Pancreatic ca. CAPAN 2 | 0.0 | 0.0 |
| Pituitary gland | 4.2 | 4.8 |
| Placenta | 0.4 | 2.4 |
| Prostate | 0.0 | 0.7 |
| Prostate ca.* (bone met)PC-3 | 13.3 | 7.3 |
| Salavary gland | 0.0 | 0.1 |
| Trachea | 0.0 | 1.1 |
| Spinal cord | 1.3 | 8.1 |
| Testis | 16.4 | 9.9 |
| Thyroid | 0.0 | 0.0 |
| Uterus | 40.6 | 24.0 |
| Melanoma M14 | 4.5 | 5.2 |
| Melanoma LOX IMVI | 0.0 | 0.9 |
| Melanoma UACC-62 | 0.0 | 0.0 |
| Melanoma SK-MEL-28 | 34.9 | 12.6 |
| Melanoma* (met) SK-MEL-S | 0.0 | 0.3 |
| Melanoma Hs688(A).T | 0.0 | 0.0 |
| Melanoma* (met) Hs688(B).T | 0.0 | 0.7 |

TABLE GE
Panel 1.3D

| | Relative | Relative |
|---|---|---|
| | Expression | Expression |
| | 1.3Dtm3184f_ | 1.3Dtm3393t_{—} |
| Tissue Name | Gpcr10 | ag998 |
| Liver adenocarcinoma | 0 | 0 |
| Pancreas | 1.7 | 0.8 |
| Pancreatic ca. CAPAN 2 | 0 | 0 |
| Adrenal gland | 1.4 | 0.7 |
| Thyroid | 5.3 | 6.6 |
| Salivary gland | 0 | 0.2 |
| Pituitary gland | 2.5 | 0.9 |
| Brain (fetal) | 11.4 | 10.7 |
| Brain (whole) | 12.6 | 10.4 |
| Brain (amygdala) | 13 | 13.8 |
| Brain (cerebellum) | 1.4 | 0.7 |
| Brain (hippocampus) | 43.2 | 51 |
| Brain (substantia nigra) | 1.2 | 0.9 |
| Brain (thalamus) | 15 | 9.7 |
| Cerebral Cortex | 100 | 100 |
| Spinal cord | 1.4 | 2.5 |
| CNS ca. (glio/astro) U87-MG | 9.3 | 6.1 |
| CNS ca. (glio/astro) U-118-MG | 0.4 | 0.2 |
| CNS ca. (astro) SW1783 | 0 | 0 |
| CNS ca.* (neuro; met) SK-N-AS | 25.5 | 20.4 |
| CNS ca. (astro) SF-539 | 0 | 0 |
| CNS ca. (astro) SNB-75 | 7.4 | 2.7 |
| CNS ca. (glio) SNB-19 | 16.3 | 16.6 |
| CNS ca. (glio) U251 | 8.5 | 6.6 |
| CNS ca. (glio) SF-295 | 39.8 | 27.4 |
| Heart (fetal) | 0.5 | 0.7 |
| Heart | 0.3 | 0 |
| Fetal Skeletal | 10.7 | 9.4 |
| Skeletal muscle | 0 | 0.3 |
| Bone marrow | 0 | 0 |
| Thymus | 1.1 | 0.4 |
| Spleen | 0.5 | 0.5 |
| Lymph node | 0.7 | 0 |
| Colorectal | 1.4 | 1.2 |
| Stomach | 2.7 | 1.4 |
| Small intestine | 0.6 | 0.4 |
| Colon ca. SW480 | 0 | 0 |
| Colon ca.* (SW480 met)SW620 | 0 | 0 |
| Colon ca. HT29 | 0 | 0 |
| Colon ca. HCT-1 16 | 0 | 0 |
| Colon ca. CaCo-2 | 0.7 | 0.2 |
| 83219 CC Well to Mod Diff(ODO3866) | 0.7 | 0.4 |
| Colon ca. HCC-2998 | 0 | 0 |
| Gastric ca.* (liver met) NCI-N87 | 0 | 0 |
| Bladder | 0.4 | 0.6 |
| Trachea | 1.1 | 1.1 |
| Kidney | 0.4 | 0.5 |
| Kidney (fetal) | 2 | 0.9 |
| Renal ca. 786-0 | 0 | 0 |
| Renal ca. A498 | 1.8 | 1.3 |
| Renal ca. RXF 393 | 0.3 | 0.5 |
| Renal ca. ACHN | 0 | 0 |
| Renal ca. UO-31 | 2.7 | 1.2 |
| Renal ca. TK-10 | 1.1 | 1.8 |
| Liver | 0 | 0.2 |
| Liver (fetal) | 0.6 | 0 |
| Liver ca. (hepatoblast) HepG2 | 0.7 | 0 |
| Lung | 0 | 1 |
| Lung (fetal) | 0.4 | 0.5 |
| Lung ca. (small cell) LX-1 | 0 | 0 |
| Lung ca. (small cell) NCI-H69 | 79.6 | 73.7 |
| Lung ca. (scell var.) SHP-77 | 6.3 | 5.3 |
| Lung ca. (large cell)NCI-H460 | 0.4 | 0.2 |
| Lung ca. (non-sm. cell) A549 | 0.7 | 0.6 |
| Lung ca. (non-s.cell) NCI-H23 | 0 | 0.3 |
| Lung ca (non-s.cell) HOP-62 | 0 | 0.2 |
| Lung ca. (non-s.d) NCI-H522 | 1 | 0.2 |
| Lung ca. (squam.) SW 900 | 3.3 | 2.5 |
| Lung ca. (squam.) NCI-H596 | 15.3 | 9.7 |
| Mammary gland | 0.8 | 0 |
| Breast ca.* (pl. effusion) MCF-7 | 0 | 0 |
| Breast ca.* (pl.ef) MDA-MB-23 1 | 0 | 0 |
| Breast ca.* (p1. effusion) T47D | 0 | 0 |
| Breast ca. BT-549 | 9.6 | 8.2 |
| Breast ca. MDA-N | 1.8 | 0.9 |
| Ovary | 4.3 | 2.7 |
| Ovarian ca. OVCAR-3 | 1.8 | 1.6 |
| Ovarian ca. OVCAR-4 | 0 | 0 |
| Ovarian ca. OVCAR-5 | 0 | 0 |
| Ovarian ca. OVCAR-8 | 3.8 | 2.3 |
| Ovarian ca. IGROV-1 | 0 | 0 |
| Ovarian ca.* (ascites) SK-OV-3 | 0 | 0 |
| Uterus | 21.3 | 21 |
| Placenta | 0 | 0 |
| Prostate | 0.7 | 1.5 |
| Prostate ca.* (bone met)PC-3 | 3 | 1.3 |
| Testis | 9.8 | 6.9 |
| Melanoma Hs68 8(A).T | 2.2 | 0.3 |
| Melanoma* (met) Hs688(B).T | 2.2 | 0.8 |
| Melanoma UACC-62 | 0 | 0 |
| Melanoma M14 | 2.5 | 1.8 |
| Melanoma LOX TMVI | 1.1 | 0.9 |
| Melanoma* (met) SK-MEL-5 | 0 | 0 |
| Adipose | 0.3 | 0 |

TABLE GF
Panel 2D

| | Relative | Relative |
|---|---|---|
| | Expression | Expression |
| | 2Dtm3154f_{—} | 2Dtm3394t_{—} |
| Tissue Name | Gpcr10 | ag998 |
| Normal Colon GENPAK 061003 | 8.4 | 1.5 |
| 83219 CC Well to Mod Diff(ODO3866) | 3.1 | 1.5 |
| 83220 CC NAT (ODO3866) | 3.7 | 1.5 |
| 83221 CC Gr.2 rectosigmoid (ODO3868) | 1.3 | 0.5 |
| 83222 CC NAT (ODO3868) | 2.5 | 0.7 |
| 83235 CC Mod Diff (ODO3920) | 0.0 | 0.0 |
| 83236 CC NAT (ODO3920) | 3.9 | 2.7 |
| 83237 CC Gr.2 ascend colon (ODO3921) | 1.0 | 0.0 |
| 83238 CC NAT (ODO3921) | 4.9 | 3.2 |
| 83241 CC from Partial Hepatectomy | 0.7 | 0.0 |
| (ODO4309) | | |
| 83242 Liver NAT (ODO4309) | 0.9 | 0.0 |
| 87472 Colon mets to lung (OD04451-01) | 0.0 | 1.2 |
| 87473 Lung NAT (OD04451-02) | 1.7 | 0.6 |
| Normal Prostate Clontech A+ 6546-1 | 3.1 | 2.0 |
| 84140 Prostate Cancer (OD04410) | 2.3 | 0.7 |
| 84141 Prostate NAT (OD04410) | 21.5 | 12.3 |
| 87073 Prostate Cancer (OD04720-01) | 3.3 | 2.1 |
| 87074 Prostate NAT (OD04720-02) | 6.7 | 6.7 |
| Normal Lung GENPAK 061010 | 2.8 | 1.4 |
| 83239 Lung Met to Muscle (ODO4286) | 11.2 | 11.8 |
| 83240 Muscle NAT (ODO4286) | 2.1 | 1.0 |
| 84136 Lung Malignant Cancer (OD03126) | 2.8 | 0.5 |
| 84137 LungNAT (OD03126) | 2.1 | 2.9 |
| 84871 Lung Cancer (OD04404) | 4.0 | 2.1 |
| 84872 Lung NAT (OD04404) | 1.7 | 0.0 |
| 84875 Lung Cancer (OD04565) | 0.0 | 0.8 |
| 84876 Lung NAT (OD04565) | 3.4 | 2.8 |
| 85950 Lung Cancer (OD04237-01) | 44.4 | 40.6 |
| 85970 Lung NAT (OD04237-02) | 0.6 | 0.5 |
| 83255 Ocular Mel Met to Liver (ODO4310) | 24.3 | 15.8 |
| 83256 Liver NAT (OD04310) | 0.0 | 0.0 |
| 84139 Melanoma Mets to Lung (OD04321) | 100.0 | 100.0 |
| 84138 Lung NAT (OD04321) | 3.1 | 2.6 |
| Normal Kidney GENPAK 061008 | 16.3 | 21.6 |
| 83786 Kidney Ca, Nuclear | 0.0 | 0.8 |
| grade 2 (OD04338) | | |
| 83787 Kidney NAT (OD04338) | 9.9 | 14.0 |
| 83788 Kidney Ca Nuclear | 0.0 | 0.0 |
| grade 1/2 (OD04339) | | |
| 83789 Kidney NAT (OD04339) | 27.5 | 17.8 |
| 83790 Kidney Ca, Clear | 2.3 | 1.6 |
| cell type (OD04340) | | |
| 83791 Kidney NAT (OD04340) | 9.4 | 9.7 |
| 83792 Kidney Ca, Nuclear | 0.7 | 0.0 |
| grade 3 (OD04348) | | |
| 83793 Kidney NAT (OD04348) | 4.9 | 3.7 |
| 87474 Kidney Cancer (OD04622-01) | 0.0 | |
| 87475 Kidney NAT (OD04622-03) | 3.0 | 1.9 |
| 85973 Kidney Cancer (OD04450-01) | 0.0 | 0.0 |
| 85974 Kidney NAT (OD04450-03) | 10.2 | 12.5 |
| Kidney Cancer Clontech 8120607 | 0.8 | 1.6 |
| Kidney NAT Clontech 8120608 | 2.7 | 0.5 |
| Kidney Cancer Clontech 8120613 | 1.3 | 0.0 |
| Kidney NAT Clontech 8120614 | 8.4 | 5.4 |
| Kidney Cancer Clontech 9010320 | 0.3 | 0.3 |
| Kidney NAT Clontech 9010321 | 10.4 | 7.3 |
| Normal Uterus GENPAK 061018 | 58.6 | 45.1 |
| Uterus Cancer GENIPAK 064011 | 41.8 | 43.2 |
| Normal Thyroid Clontech A+ 6570-1 | 32.3 | 27.5 |
| Thyroid Cancer GENPAK 064010 | 0.0 | 0.5 |
| Thyroid Cancer INVITROGEN A302152 | 2.1 | 0.8 |
| Thyroid NAT INVITROGEN A302153 | 18.4 | 13.4 |
| Normal Breast GENPAK 061019 | 2.9 | 0.0 |
| 84877 Breast Cancer (OD04566) | 1.3 | 0.6 |
| 85975 Breast Cancer (OD04590-01) | 3.6 | 0.9 |
| 85976 Breast Cancer Mets (OD04590-03) | 0.8 | 0.0 |
| 87070 Breast Cancer Metastasis | 0.9 | 0.4 |
| (OD04655-05) | | |
| GENPAK Breast Cancer 064006 | 0.9 | 1.1 |
| Breast Cancer Res. Gen. 1024 | 1.7 | 1.2 |
| Breast Cancer Clontech 9100266 | 2.0 | 3.5 |
| Breast NAT Clontech 9100265 | 1.2 | 0.7 |
| Breast Cancer INVITROGEN A209073 | 7.4 | 7.9 |
| Breast NAT INVITROGEN A2090734 | 2.5 | 1.6 |
| Normal Liver GENPAK 061009 | 0.0 | 0.9 |
| Liver Cancer GENPAK 064003 | 1.5 | 0.0 |
| Liver Cancer Research Genetics RNA 1025 | 0.7 | 0.0 |
| Liver Cancer Research Genetics RNA 1026 | 0.0 | 0.6 |
| Paired Liver Cancer Tissue Research | 0.0 | 0.5 |
| Genetics RNA 6004-T | | |
| Paired Liver Tissue Research | 2.6 | 1.5 |
| Genetics RNA 6004-N | | |
| Paired Liver Cancer Tissue | 0.8 | 0.5 |
| Research Genetics RNA 6005-T | | |
| Paired Liver Tissue Research | 0.0 | 0.0 |
| Genetics RNA 6005-N | | |
| Normal Bladder GENPAK 061001 | 4.2 | 4.0 |
| Bladder Cancer Research | 3.7 | 0.7 |
| Genetics RNA 1023 | | |
| Bladder Cancer INVITROGEN A302173 | 20.4 | 21.8 |
| 87071 Bladder Cancer (OD04718-01) | 0.0 | 1.9 |
| 87072 Bladder Normal Adjacent | 1.4 | 0.7 |
| (OD04718-03) | | |
| Normal Ovary Res. Gen. | 1.7 | 4.4 |
| Ovarian Cancer GENPAK 064008 | 11.5 | 12.6 |
| 87492 Ovary Cancer (OD04768-07) | 0.0 | 0.0 |
| 87493 Ovary NAT (OD04768-08) | 1.3 | 0.0 |
| Normal Stomach GENPAK 061017 | 6.9 | 8.0 |
| Gastric Cancer Clontech 9060358 | 0.0 | 1.3 |
| NAT Stomach Clontech 9060359 | 5.3 | 5.4 |
| Gastric Cancer Clontech 9060395 | 1.2 | 0.7 |
| NAT Stomach Clontech 9060394 | 3.1 | 2.6 |
| Gastric Cancer Clontech 9060397 | 0.9 | 2.6 |
| NAT Stomach Clontech 9060396 | 2.2 | 0.7 |
| Gastric Cancer GENPAK 064005 | 2.2 | 4.4 |

TABLE GG
Panel 3D

| | Relative | Relative |
|---|---|---|
| | Expression | Expression |
| | (%) | (%) |
| | 3dx4tm6577f_{—} | 3dx4tm5098t_{—} |
| Tissue Name | Gpcr10_al | ag998_b2 |
| 94905_DaoyMedulloblastoma/Cerebellum_sscDNA | 0.0 | 0.0 |
| 94906_TE671_Medulloblastom/Cerebellum_sscDNA | 0.3 | 0.0 |
| 94907_D283 Med_Medulloblastoma/Cerebellum_sscDNA | 1.6 | 0.1 |
| 94908_PFSK-1_Primitive | 0.2 | 0.0 |
| Neuroectodermal/Cerebellum_sscDNA | | |
| 94909_XE-498_CNS_sscDNA | 0.0 | 0.2 |
| 94910_SNB-78_CNS/glioma_sscDNA | 0.0 | 0.0 |
| 94911_SF-268_CNS/glioblastoma_sscDNA | 0.0 | 0.0 |
| 94912_T98G_Glioblastoma_sscDNA | 0.0 | 0.0 |
| 96776_SK-N-SH_Neuroblastoma (metastasis)_sscDNA | 16.4 | 8.6 |
| 94913_SF-295_CNS/glioblastoma_sscDNA | 13.4 | 6.2 |
| 94914_Cerebellum_sscDNA | 5.5 | 2.8 |
| 96777_Cerebellum_sscDNA | 3.3 | 0.0 |
| 94916 NCI-H292 Mucoepidermoid lung carcinoma sscDNA | 1.2 | 0.0 |
| 94917 DMS-l 14 Small cell lung cancer sscDNA | 0.0 | 0.0 |
| 94918_DMS-79_Small cell lung | 0.3 | 0.0 |
| cancer/neuroendocrine_sscDNA | | |
| 94919_NCI-H_146_Small cell lung | 100.0 | 100.0 |
| cancer/neuroendocrine_sscDNA | | |
| 94920_NCI-H526_Small cell lung | 1.9 | 0.6 |
| cancer/neuroendocrine_sscDNA | | |
| 94921_NCI-N417_Small cell lung | 11.7 | 5.1 |
| cancer/neuroendocrine_sscDNA | | |
| 94923_NCI-H82_Small cell lung | 0.0 | 0.2 |
| cancer/neuroendocrine_sscDNA | | |
| 94924_Nd-H157_Squamous cell lung cancer | 0.0 | 0.0 |
| L(metastasis)_sscDNA | | |
| 94925_Nd-H1155_Large cell lung | 0.2 | 0.3 |
| cancer/neuroendocrine_sscDNA | | |
| 94926_NCI-H1299_Large cell lung | 0.0 | 0.0 |
| cancer/neuroendocrine_sscDNA | | |
| 94927_NCI-H727_Lung carcinoid_sscDNA | 1.0 | 1.1 |
| 94928_NCI-UMC-11_Lung carcinoid_sscDNA | 5.5 | 3.1 |
| 94929_LX-1_Small cell lung cancer_sscDNA | 0.0 | 0.0 |
| 94930_Colo-205_Colon cancer_sscDNA | 0.0 | 0.0 |
| 94931_RM12_Colon cancer sscDNA | 0.0 | 0.0 |
| 94932_KM20L2_Colon cancer_sscDNA | 0.0 | 0.0 |
| 94933_NCI-H716_Colon cancer_sscDNA | 0.9 | 0.2 |
| 94935_SW-48_Colon adenocarcinoma_sscDNA | 0.0 | 0.0 |
| 94936_SW1116_Colon adenocarcinoma_sscDNA | 0.0 | 0.0 |
| 94937_LS174T_Colon adenocarcinoma_sscDNA | 0.0 | 0.0 |
| 94938_SW-948_Colon adenocarcinoma_sscDNA | 0.0 | 0.0 |
| 94939_SW-480_Colon adenocarcinoma_sscDNA | 0.0 | 0.0 |
| 94940_NCI-SNU-5_Gastric carcinoma_sscDNA | 0.0 | 0.0 |
| 94941_KATO III_Gastric carcinoma_sscDNA | 0.0 | 0.0 |
| 94943_NCI-SNU-16_Gastric carcinoma_sscDNA | 0.0 | 0.0 |
| 94944_NCI-SNU-1_Gastric carcinoma_sscDNA | 0.0 | 0.0 |
| 94946_RF-1_Gastric adenocarcinoma_sscDNA | 0.0 | 0.0 |
| 94947_RF-48_Gastric adenocarcinoma_sscDNA 0.0 | 0.0 | |
| 96778_MKN-45_Gastric carcinoma_sscDNA | 0.0 | 0.0 |
| 94949_NCI-N87_Gastric carcinoma_sscDNA | 0.0 | 0.0 |
| 94951_OVCAR-5_Ovarian carcinoma_sscDNA | 0.0 | 0.0 |
| 94952_RL95-2_Uterine carcinoma_sscDNA | 0.0 | 0.0 |
| 94953_HelaS3_Cervical adenocarcinoma_sscDNA | 1.8 | 0.0 |
| 94954_Ca Ski_Cervical epidermoid carcinoma | 0.0 | 0.0 |
| (metastasis)_sscDNA | | |
| 94955_ES-2_Ovarian clear cell carcinoma_sscDNA | 0.0 | 0.2 |
| 94957_Ramos/6h stim_”; Stimulated with PMA/ionomycin | 0.0 | 0.0 |
| 6h_sscDNA | | |
| 94958_Ramos/14h stim_”; Stimulated with PMA/ionomycin | 0.0 | 0.0 |
| 14h_sscDNA | | |
| 94962_MEG-Ol_Chronic myelogenous leukemia | 5.3 | 2.1 |
| (megokaryoblast)_sscDNA | | |
| 94963_Raji_Burkitt's lymphoma_sscDNA | 0.0 | 0.0 |
| 94964_Daudi_Burkitt's lymphoma_sscDNA | 0.0 | 0.0 |
| 94965_U266_B-cell plasmacytoma/myeloma_sscDNA | 0.0 | 0.0 |
| 94968_CA46_Burkitt's_lymphoma_sscDNA | 0.0 | 0.0 |
| 94970_RL_non-Hodgkin's B-cell lymphoma_sscDNA | 0.0 | 0.0 |
| 94972_JM1_pre-B-cell lymphoma/leukemia_sscDNA | 0.0 | 0.0 |
| 94973_Jurkat_T cell leukemia_sscDNA | 0.0 | 0.0 |
| 94974_TF-1_Erythroleukemia_sscDNA | 0.0 | 0.0 |
| 94975_HUT 78_T-cell lymphoma_sscDNA | 1.1 | 0.0 |
| 94977_U937_Histiocytic lymphoma_sscDNA | 0.0 | 0.0 |
| 94980_KU-812_Myelogenous leukemia_sscDNA | 24.2 | 10.2 |
| 94981_769-P_Clear cell renal carcinoma sscDNA | 0.0 | 0.0 |
| 94983_Caki-2_Clear cell renal carcinoma_sscDNA | 0.7 | 0.0 |
| 94984_SW 839_Clear cell renal carcinoma_sscDNA | 0.0 | 0.0 |
| 94986_G401_Wilms' tumor_sscDNA | 0.0 | 0.0 |
| 94987_Hs766T_Pancreatic carcinoma (LN metastasis)_sscDNA | 0.4 | 0.0 |
| 94988_CAPAN-1_Pancreatic adenocarcinoma (liver | 0.0 | 0.0 |
| metastasis)_sscDNA | | |
| 94989_SU86.86_Pancreatic carcinoma (liver | 0.4 | 0.5 |
| metastasis)_sscDNA | | |
| 94990_BxPC-3_Pancreatic adenocarcinoma_sscDNA | 3.4 | 1.4 |
| 94991_HPAC_Pancreatic adenocarcinoma_sscDNA | 0.0 | 0.0 |
| 94992_MIA PaCa-2_Pancreatic carcinoma_sscDNA | 0.3 | 0.0 |
| 94993_CFPAC-1_Pancreatic ductal adenocarcinoma_sscDNA | 4.1 | 1.8 |
| 94994_PANC-1_Pancreatic epithelioid ductal | 0.0 | 0.0 |
| carcinoma_sscDNA | | |
| 94996_T24_Bladder carcinma (transitional cell)_sscDNA | 0.0 | 0.0 |
| 94997_5637_Bladder carcinoma_sscDNA | 4.4 | 1.5 |
| 94998_HT-1197_Bladder carcinoma_sscDNA | 6.4 | 6.0 |
| 94999_UM-UC-3_Bladder carcinma (transitional cell)_sscDNA | 0.8 | 0.0 |
| 95000_A204_Rhabdomyosarcoma_sscDNA | 0.0 | 0.0 |
| 95001_HT-1080_Fibrosarcoma_sscDNA | 0.0 | 0.0 |
| 95002_MG-63_Osteosarcoma_(bone)_sscDNA | 0.0 | 0.0 |
| 95003_SK-LMS-l Leiomyosarcoma (vulva)_sscDNA | 0.0 | 0.0 |
| 95004_SJRH3O_Rhabdomyosarcoma (met to bone | 2.1 | 2.4 |
| marrow)_sscDNA | | |
| 95005_A431_Epidermoid carcinoma_sscDNA | 0.0 | 0.0 |
| 95007_WM266-4_Melanoma_sscDNA | 7.2 | 4.3 |
| 95010_DU 145 Prostate carcinoma (brain metastasis)_sscDNA | 0.0 | 0.0 |
| 95012_MDA-MB-468_Breast adenocarcinoma_sscDNA | 0.0 | 0.3 |
| 95013_SCC-4_Squamous cell carcinoma of tongue_sscDNA | 0.0 | 0.0 |
| 95014_SCC-9_Squamous cell carcinoma of tongue_sscDNA | 0.0 | 0.0 |
| 95015_SCC-15_Squamous cell carcinoma of tongue_sscDNA | 0.0 | 0.0 |
| 95017_CAL 27_Squamous cell carcinoma of tongue_sscDNA | 0.3 | 0.0 |

TABLE GH
Panel 4D

| | Relative | Relative |
|---|---|---|
| | Expression | Expression |
| | (%) | (%) |
| | 4dx4tm5136f_ | 4Dtm3395t_ |
| Tissue Name | gpcr10_b2 | ag998 |
| 93768_Secondary Th1_anti-CD28/anti-CD3 | 0.0 | 0.0 |
| 93769_Secondary Th2_anti-CD28/anti-CD3 | 1.3 | 0.0 |
| 93770_Secondary Tr1_anti-CD28/anti-CD3 | 0.5 | 0.0 |
| 93573_Secondary Th1_resting day 4-6 in IL-2 | 0.0 | 0.0 |
| 93572_Secondary Th2_resting day 4-6 in IL-2 | 0.0 | 0.0 |
| 93571_Secondary Tr1_resting day 4-6 in IL-2 | 0.0 | 0.0 |
| 93S68_primary Th1_anti-CD28/anti-CD3 | 0.0 | 0.0 |
| 93569_primary Th2_anti-CD28/anti-CD3 | 0.0 | 0.0 |
| 93S70_primary Tr1_anti-CD28/anti-CD3 | 0.0 | 0.0 |
| 93565_primary Th1_resting dy 4-6 in IL-2 | 0.0 | 0.0 |
| 93566_primary Th2_resting dy 4-6 in IL-2 | 0.0 | 0.0 |
| 93567_primary Tr1_resting dy 4-6 in IL-2 | 0.0 | 0.0 |
| 93351_CD45RA CD4 lymphocyte_anti-CD28/anti-CD3 | 0.0 | 0.3 |
| 93352_CD45RO CD4 lymphocyte_anti-CD28/anti-CD3 | 0.0 | 1.6 |
| 93251_CD8 Lymphocytes_anti-CD28/anti-CD3 | 1.4 | 0.0 |
| 93353_chronic CD8 Lymphocytes 2ry_resting dy 4-6 in IL-2 | 0.0 | 0.5 |
| 93574_chronic CD8 Lymphocytes 2ry_activated CD3/CD28 | 0.0 | 0.0 |
| 93354_CD4_none | 1.9 | 0.0 |
| 93252_Secondary Th1/Th2/Tr1_anti-CD95 CH11 | 0.0 | 0.0 |
| 93103_LAK cells_resting | 1.6 | 2.3 |
| 93788_LAK cells_IL-2 | 6.7 | 12.2 |
| 93787_LAK cells_IL-2+IL-12 | 1.9 | 0.7 |
| 93789_LAK cells_IL-2+IFN gamma | 2.9 | 4.6 |
| 93790_LAK cells_11-2+IL-18 | 2.6 | 4.4 |
| 93104_LAK cells_PMA/ionomycin and IL-18 | 3.2 | 0.6 |
| 93578_NK Cells IL-2 resting | 6.4 | 4.5 |
| 93109_Mixed Lymphocyte Reaction_Two Way MLR | 10.4 | 9.9 |
| 93110_Mixed Lymphocyte Reaction_Two Way MLR | 2.7 | 3.1 |
| 93111_Mixed Lymphocyte Reaction_Two Way MLR | 0.0 | 0.0 |
| 93112_Mononuclear Cells (PBMCs)_resting | 0.5 | 0.0 |
| 93113_Mononuclear Cells (PBMCs)_PWM | 3.2 | 1.3 |
| 93114_Mononuclear Cells (PBMCs)_PHA-L | 0.0 | 0.0 |
| 93249_Ramos (B cell)_none | 0.0 | 0.0 |
| 93250_Ramos (B cell)_ionomycin | 0.0 | 0.0 |
| 93349_B lymphocytes_PWM | 0.0 | 0.0 |
| 93350_B lymphoytes_CD40L and IL-4 | 0.7 | 0.0 |
| 92665_EOL-1 (Bosinophil)_dbcAMP differentiated | 0.0 | 0.0 |
| 93248_EOL-1 (Eosinophil)_dbcAMP/PMAionomycin | 1.7 | 0.0 |
| 93356_Dendritic Cells_none | 0.81 | 0.9 |
| 93355_Dendritic Cells_LPS 100 ng/ml | 0.0 | 0.6 |
| 93775_Dendritic Cells_anti-CD40 | 0.0 | 0.0 |
| 93774_Monocytes_resting | 0.0 | 0.0 |
| 93776_Monocytes_LPS 50 ng/ml | 0.0 | 0.0 |
| 93581_Macrophages_resting | 0.0 | 0.0 |
| 93582_Macrophages_LPS 100 ng/m1 | 0.0 | 0.0 |
| 93098_HUVEC (Endothelial)_none | 0.0 0.0 | |
| 93099_HUVEC (Endothelial)_starved | 1.0 | 0.0 |
| 93100_HUVEC (Endothelial)_IL-1b | 0.0 | 0.0 |
| 93779_HUVEC (Endothelial)_IFN gamma | 0.6 | 0.0 |
| 93102_HUVEC (Endothelial)_TNF alpha +TEN gamma | 0.0 | 0.0 |
| 93101_HUVEC (Endothelial)_TNF alpha +1L4 | 0.0 | 1.3 |
| 93781_HUVEC (Endothelial)_IL-11 | 1.3 | 0.0 |
| 93583_Lung Microvascular Endothelial Cells_none | 1.0 | 0.0 |
| 93584_Lung Microvascular Endothelial Cells_TNFa (4 ng/ml) | 1.9 | 0.0 |
| and IL 1b (1 ng/ml) | | |
| 92662_Microvascular Dermal endothelium_none | 0.0 | 0.0 |
| 92663_Microsvasular Dermal endothelium_TNFa (4 ng/ml) and | 0.0 | 0.0 |
| IL 1b (1 ng/ml) | | |
| 93773_Bronchial epithelium_TNFa (4 ng/ml) and IL 1b (1 | 2.2 | 1.1 |
| ng/ml) ** | | |
| 93347_Small Airway Epithelium_none | 1.1 | 0.4 |
| 93348_Small Airway Epithelium_TNFa (4 ng/ml) and IL 1b (1 | 0.0 | 0.0 |
| ng/ml) | | |
| 92668_Coronery Artery SMC_resting | 0.0 | 0.0 |
| 92669_Coronery Artery SMC_TNFa (4 ng/ml) and IL 1b (1 | 0.0 | 0.0 |
| ng/ml) | | |
| 93107_astrocytes_resting | 0.0 | 0.3 |
| 93108_astrocytes_TNFa (4 ng/ml) and IL 1b (1 ng/ml) | 0.9 | 0.7 |
| 92666_KU-812 (Basophil)_resting | 42.8 | 43.8 |
| 92667_KU-812 (Basophil)_PMA/ionoycin | 100.0 | 100.0 |
| 93579_CCD1106 (Keratinocytes)_none | 0.0 | 2.6 |
| 93580_CCD1106 (Keratinocytes)_TNFa and IFNg ** | 0.0 | 0.6 |
| 93791_Liver Cirrhosis | 4.6 | 3.4 |
| 93792_Lupus Kidney | 0.0 | 0.0 |
| 93577_NCI-H292 | 1.9 | 0.0 |
| 93358_NCI-H292_IL-4 | 0.0 | 0.0 |
| 93360_NCI-H292 IL-9 | 0.9 | 0.2 |
| 93357_NCI-H292 IFN gamma | 0.0 | 1.0 |
| 93359_NCI-H292 IL-13 | 1.0 | 0.0 |
| 93777_HPAEC_- | 0.0 | 0.0 |
| 93778_HPAEC_IL-1 beta/TNA alpha | 0.0 | 0.0 |
| 93254_Normal Human Lung Fibroblast_none | 0.0 | 0.0 |
| 93253_Normal Human Lung Fibroblast_TNFa (4 ng/ml) and IL- | 0.0 | 0.3 |
| 1b (1 ng/ml) | | |
| 93257_Normal Human Lung Fibroblast_IL-4 | 0.0 | 0.0 |
| 93256_Normal Human Lung Fibroblast_IL-9 | 0.0 | 0.0 |
| 93255_Normal Human Lung Fibroblast_IL-13 | 0.0 | 0.0 |
| 93258_Normal Human Lung Fibroblast_IFN gamma | 0.0 | 0.3 |
| 93106_Dermal Fibroblasts CCD1070_resting | 3.6 | 0.0 |
| 93361_Dermal Fibroblasts CCD1070_TNF alpha 4 ng/ml | 0.0 | 0.0 |
| 93105_Dermal Fibroblasts CCD1070_IL-1 beta 1 ng/ml | 0.0 | 0.7 |
| 93772_dermal fibroblast_IFN gamma | 0.0 | 0.0 |
| 93771_dermal fibroblast_IL-4 | 1.0 | 0.0 |
| 93259_IBD Colitis 1** | 1.3 | 0.0 |
| 93260_IBD Colitis 2 | 1.1 | 1.1 |
| 93261_IBD Crohns | 1.5 | 0.0 |
| 735010_Colon_normal | 0.0 | 0.0 |
| 735019_Lung_none | 0.0 | 0.7 |
| 64028-1_Thymus_none | 12.2 | 17.2 |
| 64030-1_Kidney_none | 4.8 | 5.0 |

TABLE GI
Panel CNSD.01

| | Relative | | Relative |
|---|---|---|---|
| | Expression (%) | | Exression (%) |
| | cns_1x4tm665 | | cns_1x4tm665 |
| Tissue Name | if gpcr10_b1 | Tissue Name | if gpcr10_b1 |
| 102633_BA4 Control | 39.1 | 102605_BA17 PSP | 36.8 |
| 102641_BA4 Control2 | 27.9 | 102612_BA17 PSP2 | 16.2 |
| 102625_BA4 Alzheimer's | 29.8 | 102637_Sub Nigra Control | 18.4 |
| 102649_BA4 Parkinson's | 55.7 | 102645_Sub Nigra Control2 | 12.4 |
| | | 102629_Sub Nigra | |
| 102656_BA4 Parkinson's | 271.6 | Alzheimer's2 | 12.6 |
| 102664_BA4 Huntington's | 40.3 | 102660_Sub Nigra Parkinson's2 | 40.0 |
| | | 102667_Sub Nigra | 34.5 |
| 102671_BA4 Huntington's2 | 10.7 | Huntington's | |
| | | 102674_Sub Nigra | 20.8 |
| 102603_BA4 PSP | 15.3 | Huntington's2 | |
| 102610_BA4PSP2 | 47.2 | 102614_Sub Nigra PSP2 | 2.6 |
| 102588_BA4 Depression | 19.3 | 102592_Sub Nigra Depression | 1.3 |
| 102596_BA4 Depression2 | 10.0 | 102599_Sub Nigra Depression2 | 7.9 |
| 102634_BA7 Control | 49.7 | 102636_Glob Palladus Control | 3.7 |
| 102642_BA7 Control2 | 27.2 | 102644_Glob Palladus Control2 | 9.7 |
| 102626_BA7 Alzheimer's2 | 19.1 | 102620_Glob Palladus | 9.9 |
| | | Alzheimer's | |
| 102650_BA7 Parkinson's | 22.5 | 102628_Glob Palladus | 0.0 |
| | | Alzheimer's2 | |
| 102657_BA7 Parkinson's | 266.8 | 102652_Glob Palladus | 30.3 |
| | | Parkinson's | |
| | | 102659_Glob Palladus | |
| 102665_BA7 Huntington's | 48.2 | Parkinson's2 | 1.4 |
| 102672_BA7 Huntington's | 253.4 | 102606_Glob Pallaclus PSP | 0.0 |
| 102604_BA7PSP | 49.6 | 102613_Glob Palladus PSP2 | 1.5 |
| | | 102591_Glob Palladus | 0.0 |
| 102611_BA7 PSP2 | 39.3 | Depression | |
| 102589_BA7 Depression | 18.1 | 102638_Temp Pole Control | 25.2 |
| 102632_BA9 Control | 37.7 | 102646_Temp Pole Control2 | 81.6 |
| 102640_BA9 Control2 | 69.3 | 102622_Temp Pole Alzheimer's | 12.7 |
| | | 102630_Temp Pole | |
| 102617_BA9 Alzheimer's | 8.9 | Alzheimer's2 | 17.2 |
| 102624_BA9 Alzheimer's | 226.4 | 102653_Temp Pole Parkinson's | 46.6 |
| | | 102661_Temp Pole | 40.7 |
| 102648_BA9 Parkinson's | 29.4 | Parkinson's2 | |
| | | 102668_Temp Pole | 66.3 |
| 102655_BA9 Parkinson's2 | 55.8 | Huntington_s | |
| 102663_BA9 Huntington's | 51.3 | 102607_Temp Pole PSP | 5.7 |
| 102670_BA9 Huntington's2 | 21.1 | 102615_Temp Pole PSP2 | 12.4 |
| | | 102600_Temp Pole | 9.6 |
| 102602_BA9 PSP | 27.6 | Depression2 | |
| 102609_BA9 PSP2 | 13.1 | 102639_Cing Gyr Control | 57.2 |
| 102587_BA9 Depression | 13.8 | 102647_Cing Gyr Control2 | 27.5 |
| 102595_BA9 Depression2 | 7.2 | 102623_Cing Gyr Alzheimer's | 25.1 |
| 102635_BA17 Control | 100.0 | 102631_Cing Gyr Alzheimer's2 | 6.8 |
| 102643_BA17 Control2 | 53.3 | 102654_Cing Gyr Parkinson's | 24.8 |
| 102627_BA17 Alzheimer's2 | 19.6 | 102662_Cing Gyr Parkinson's2 | 36.7 |
| 102651_BAl7 Parkinson's | 67.7 | 102669_Cing Gyr Huntington's | 60.3 |
| | | 102676_Cing Gyr | 16.4 |
| 102658_BA17 Parkinson's2 | 77.0 | Huntington's2 | |
| 102666_BA17 Huntington's | 43.9 | 102608_CingGyrPSP | 19.0 |
| 102673_BA17 Huntington's2 | 23.5 | 102616_Cing Gyr PSP2 | 6.9 |
| 102590_BA17 Depression | 16.9 | 102594_Cing Gyr Depression | 9.3 |
| 102597_BA17 Depression | 233.1 | 102601_Cing Gyr Depression2 | 15.4 |

Panel 1 Summary: Gpcr10 The 20760813_EXT gene is relatively highly expressed in samples from the central nervous system. Among these tissues, moderate expression is detected in thalamus, hippocampus, amygdala and substantia nigra, while lower expression is seen in spinal cord, hypothalamus and cerebellum (please see discussion of Panel 1.3D for potential utility). Among normal tissues, 20760813_EXT gene expression is also detected in colon, kidney, thyroid, testis and uterus

The 20760813_EXT gene is most highly expressed in a sample derived from a lung cancer cell line and shows significant expression in other samples derived from lung cancer cell lines. In addition, there appears to be significant expression of this gene in CNS cancer derived cell lines, ovarian cancer cell lines, and a pancreatic cancer cell line. Thus, based upon this pattern of gene expression, the therapeutic modulation of the activity of the 20760813_EXT gene product might be of use in the treatment of CNS malignancies, lung cancer, pancreatic cancer and/or ovarian cancer.

Panel 1.1 Summary: Gpcr10 Two replicate experiments performed using the same probe/primer set yielded results that are in good agreement. Strong expression of the 20760813_EXT gene is again observed in the CNS, including in amygdala, cerebellum, hippocampus, substantia nigra, thalamus and cerebral cortex (please see discussion of Panel 1.3D for potential utility). Lower expression levels are also seen in the spinal cord.

Among metabolically relevant tissues, 20760813_EXT gene expression is seen in fetal skeletal muscle (CT values=28, 33), pancreas (CT values=32, 27.6), and pituitary gland (CT=30, 27). This observation suggests that therapeutic modulation may aid the treatment of metabolic diseases such as obesity and diabetes as well as neuroendocrine disorders. Glycoprotein hormones influence the development and function of the ovary, testis and thyroid by binding to specific high-affinity receptors. Interestingly, the extracellular domains of these receptors are members of the leucine-rich repeat (LRR) protein superfamily and are responsible for the high-affinity binding (Ref. 1).

Similar to what was observed in Panel 1, the 20760813_EXT gene shows highest expression in a sample derived from a lung cancer cell line and also shows significant over-expression in other samples derived from lung cancer cell lines relative to the normal lung control. Furthermore, it is also highly expressed by brain tumors derived cell lines, indicating a possible role in the development and progression of brain tumors. There appears to be significant expression of the 20760813_EXT gene in a melanoma cell line as well as in uterus and testis tissue. Thus, based upon this pattern of gene expression, the therapeutic modulation of the activity of the 20760813_EXT gene product might be of use in the treatment of CNS malignancies, melanomas and/or lung cancer.

Panel 1.2 Summary: Gpcr10 Expression of the 20760813_EXT gene is low/undetectable (CT values>35) in all samples on this panel.

Panel 1.3D Summary: Gpcr10 /Ag998 Results from two replicate experiments were performed using different probe/primer sets and the results are in excellent agreement. The 20760813_EXT gene is most highly expressed in cerebral cortex (CT=30) and shows moderate expression in other CNS regions as well including, amygdala, hippocampus, and thalamus. The 20760813_EXT gene encodes a leucine-rich repeat protein. Leucine rich repeats (LRR) mediate reversible protein-protein interactions and have diverse cellular functions, including cellular adhesion and signaling. Several of these proteins, such as connectin, slit, chaoptin, and Toll have pivotal roles in neuronal development in Drosophila and may play significant but distinct roles in neural development and in the adult nervous system of humans (Ref. 2). In Drosophilia, the LRR region of axon guidance proteins has been shown to be critical for their function (especially in axon repulsion). Since the leucine-rich-repeat protein encoded by the 20760813_EXT gene shows high expression in the cerebral cortex, it is an excellent candidate neuronal guidance protein for axons, dendrites and/or growth cones in general. Therefore, therapeutic modulation of the levels of this protein, or possible signaling via this protein, may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease).

Among normal tissues, expression of the 20760813_EXT gene is also seen in thyroid (CT=34), fetal skeletal muscle (CT=33), uterus (CT=32) and testis (CT=33). In addition, there is a strong cluster of expression in CNS cancer-derived cell lines and lung cancer cell lines. Thus, based upon this pattern of gene expression, the therapeutic modulation of the activity of the 20760813_EXT gene product might be of use in the treatment of CNS malignancies or lung cancer.

Panel 2D Summary: Gpc10/Ag998 Results from two replicate experiments were performed using different probe/primer sets and the results are in excellent agreement. The 20760813_EXT gene is most highly expressed in a sample derived from a melanoma metastasis (CT=30.9). In addition, this gene appears to be more highly expressed in normal kidney and thyroid tissues when compared to associated cancer tissues. In contrast, the 20760813_EXT gene is more highly expressed in lung cancer tissue when compared to normal adjacent tissue. Thus, therapeutic up-regulation of the activity of this gene, through the application of the protein product itself or by gene replacement therapy, might be of use in the treatment of kidney and thyroid cancer. Alternatively, down-regulation of the activity of the 20760813_EXT gene product, through the use of inhibitory antibodies or small molecule drugs, might be of use in the treatment of melanoma or lung cancer.

Panel 3D Summary: Gpcr10/Ag998 Results from two replicate experiments were performed using different probe/primer sets and the results are in excellent agreement. The highest expression of the 20760813_EXT gene on this panel is detected in a cell line derived from a small cell lung cancer (CT=29.1). In addition, there is expression in a cluster of lung cancer cell lines indicating that the inhibition of this gene activity might be of use in the therapy of lung cancer. This result is consistent with what was observed in Panel 1.3D and Panel 2D.

Panel 4D Summary: Gpcr10/Ag998 Results from two replicate experiments were performed using different probe/primer sets and the results are in excellent agreement. The 20760813_EXT transcript is induced in PMA and ionomycin treated basophil cell line KU-812. Basophils release histamines and other biological modifiers in repose to allergens and play an important role in the pathology of asthma and hypersensitivity reactions. Therefore, antibody therapeutics designed against the putative leucine rich repeat protein encoded for by the 20760813_EXT gene could reduce or inhibit inflammation by blocking basophil function in these diseases.

Panel CNSD.01 Summary: Gpcr10 The 20760813_EXT gene shows highest expression throughout the cortex, with lower levels in the substantia nigra and globus palladus. This result is consistent with what was observed in Panels 1, 1.1, and 1.3D. In addition, there is no apparent association between the 20760813_EXT gene expression pattern and the diseased samples present on this panel.

### EXAMPLE 9

### Quantitative Expression Analysis (TaqMan) of NOV10

Expression of NOV10 (SC128855163_A) was assessed using the primer-probe set Ag1450, described in Table HA. Results of the RTQ-PCR runs are shown in Tables HB, HC, and HD.

TABLE HA
Probe Name Ag1450

| | | | | |
|---|---|---|---|---|
| | | | | Start |
| Primers | Sequences | TM | Length | Position |

| | | | | | | |
|---|---|---|---|---|---|---|
| Forward | 5′-CCAAGTTCTTCCTAGTGGCTTT-3′ | (SEQ ID NO. 59) | 59 | 22 | 83 | |
| Probe | FAM-5′-TTTCTCCTTCGCCCAGGTTGTAATTG-3′-TAMRA | (SEQ ID NO. 60) | 68.8 | 26 | 114 | |
| Reverse | 5′-ATACCTAGCGACCACCAAGAAT-3′ | (SEQ ID NO. 61) | 59 | 22 | 146 | 146 |

TABLE HB
Panel 1.2

| | Relative Expression (%) | |
|---|---|---|
| | 1.2tm2019f_{—} | 1.2tm2079f_ |
| Tissue Name | ag1450 | ag1450 |
| Endothelial cells | 0.5 | 0.5 |
| Heart (fetal) | 1.5 | 2.1 |
| Pancreas | 0.2 | 0.6 |
| Pancreatic ca. CAPAN 2 | 0.0 | 0.0 |
| Adrenal Gland (new lot*) | 0.9 | 1.2 |
| Thyroid | 0.3 | 0.3 |
| Salavary gland | 21.6 | 20.0 |
| Pituitary gland | 2.3 | 0.2 |
| Brain (fetal) | 0.2 | 0.0 |
| Brain (whole) | 0.0 | 0.1 |
| Brain (amygdala) | 0.4 | 0.5 |
| Brain (cerebellum) | 0.6 | 0.4 |
| Brain (hippocampus) | 1.2 | 1.4 |
| Brain (thalamus) | 0.6 | 0.6 |
| Cerebral Cortex | 3.3 | 4.6 |
| Spinal cord | 0.2 | 0.1 |
| CNS ca. (glio/astro) U87-MG | 51.8 | 64.6 |
| CNS ca. (glio/astro) U-118-MG | 46.3 | 55.5 |
| CNS ca. (astro) SW1783 | 13.4 | 14.1 |
| CNS ca.* (neuro; met) SK-N-AS | 2.1 | 2.0 |
| CNS ca. (astro) SF-539 | 3.1 | 2.5 |
| CNS ca. (astro) SNB-75 | 12.0 | 10.2 |
| CNS ca. (glio) SNB-19 | 0.4 | 1.1 |
| CNS ca. (glio) U25 1 | 2.9 | 4.0 |
| CNS ca. (glio) SF-295 | 100.0 | 100.0 |
| Heart | 4.1 | 5.6 |
| Skeletal Muscle (new lot*) | 5.6 | 7.7 |
| Bone marrow | 0.0 | 0.0 |
| Thymus | 0.2 | 0.1 |
| Spleen | 0.4 | 0.6 |
| Lymph node | 0.1 | 0.0 |
| Colorectal | 0.8 | 1.7 |
| Stomach | 0.2 | 0.6 |
| Small intestine | 3.4 | 3.4 |
| Colon ca. SW480 | 10.2 | 18.3 |
| Colon ca.* (SW480 met)SW620 | 0.0 | 0.0 |
| Colon ca. HT29 | 0.0 | 0.0 |
| Colon ca. HCT-116 | 0.0 | 0.0 |
| Colon ca. CaCo-2 | 1.1 | 1.2 |
| 83219CC Well to Mod Diff(ODO3866) | 1.2 | 1.3 |
| Colon ca. HCC-2998 | 0.8 | 0.9 |
| Gastric ca.* (liver met) NCI-N87 | 0.0 | 0.0 |
| Bladder | 4.6 | 8.1 |
| Trachea | 0.9 | 0.0 |
| Kidney | 5.8 | 5.6 |
| Kidney (fetal) | 5.1 | 4.6 |
| Renal ca. 786-0 | 1.2 | 1.5 |
| Renal ca. A498 | 0.5 | 0.9 |
| Renal ca. RXF 393 | 0.8 | 1.4 |
| Renal ca. ACLIN | 0.9 | 1.2 |
| Renal ca. UO-31 | 6.6 | 10.4 |
| Renal ca. TK-10 | 0.2 | 0.2 |
| Liver | 0.7 | 0.9 |
| Liver (fetal) | 0.3 | 0.4 |
| Liver ca | 0.0 | 0.0 |
| Lung (hepatoblast) HepG2 | 0.1 | 0.5 |
| Lung (fetal) | 0.2 | 0.3 |
| Lung ca. (small cell) LX-1 | 0.0 | 0.0 |
| Lung ca. (small cell) NCI-H69 | 0.2 | 0.3 |
| Lung ca. (s.cell var.) SHP-77 | 0.0 | 0.0 |
| Lung ca. (large cell) NCI-H460 | 20.0 | 27.7 |
| Lung ca. (non-sm. cell) A549 | 0.0 | 0.0 |
| Lung ca. (non-s.cell) NCI-H23 | 0.2 | 0.3 |
| Lung ca (non-s.cell) HOP-62 | 70.7 | 58.2 |
| Lung ca. (non-s.d) NCI-H522 | 1.7 | 2.4 |
| Lung ca. (squam.) SW 900 | 12.8 | 22.5 |
| Lung ca. (squam.) NCI-H596 | 0.6 | 1.0 |
| Mammary gland | 1.1 | 1.3 |
| Breast ca.* (pl.effusion) MCF-7 | 0.2 | 0.2 |
| Breast ca.* (pl.ef) MDA-MB-231 | 0.0 | 0.0 |
| Breast ca.* (pl.effusion) T47D | 0.0 | 0.0 |
| Breast ca. BT-549 | 1.3 | 1.3 |
| Breast ca. MDA-N | 0.0 | 0.0 |
| Ovary | 10.7 | 19.3 |
| Ovarian ca. OVCAR-3 | 18.0 | 16.3 |
| Ovarian ca. OVCAR-4 | 14.5 | 16.5 |
| Ovarian ca. OVCAR-5 | 1.4 | 1.5 |
| Ovarian ca. OVCAR-8 | 2.5 | 2.6 |
| Ovarian ca. IGROV-1 | 15.6 | 9.4 |
| Ovarian ca.* (ascites) SK-OV-3 | 1.3 | 2.4 |
| Uterus | 3.5 | 2.5 |
| Placenta | 8.7 | 1.2 |
| Prostate | 2.0 | 2.9 |
| Prostate ca.* (bone met)PC-3 | 17.2 | 17.4 |
| Testis | 0.2 | 0.4 |
| Melanoma Hs688(A).T | 17.2 | 22.2 |
| Melanoma* (met) Hs688(B).T | 16.2 | 18.7 |
| Melanoma UACC-62 | 2.2 | 2.7 |
| Melanoma M14 | 0.8 | 1.0 |
| Melanoma LOX TMVI | 4.7 | 5.6 |
| Melanoma* (met) 5K-MEL-S | 0.5 | 0.8 |
| Adipose | 2.3 | 4.2 |

TABLE HG
Panel 2D

| | Relative Expression (%) | |
|---|---|---|
| | 2Dtm2366f_ | 2Dtm2937f_ |
| Tissue Name | ag1450 | ag1450 |
| Normal Colon GENPAK 0610003 | 13.7 | 10.8 |
| 83219 CC Well to Mod Diff (ODO3866) | 20.7 | 15.8 |
| 83220 CC NAT (ODO3866) | 9.8 | 5.1 |
| 83221 CC Gr.2 rectosigmoid (ODO3868) | 14.2 | 8.4 |
| 83222 CC NAT (ODO3868) | 3.6 | 1.6 |
| 83235 CC Mod Diff (ODO3920) | 12.2 | 10.7 |
| 83236 CC NAT (ODO3920) | 1.9 | 2.2 |
| 83237 CC Gr.2 ascend colon (ODO3921) | 29.9 | 33.7 |
| 83238 CC NAT (ODO3921) | 9.1 | 6.7 |
| 83241 CC from Partial Hepatectomy (ODO4309) | 5.3 | 4.9 |
| 83242 Liver NAT (ODO4309) | 4.5 | 4.7 |
| 87472 Colon mets to lung (OD04451-01) | 9.3 | 4.4 |
| 87473 Lung NAT (OD04451-02) | 12.3 | 5.9 |
| Normal Prostate Clontech A+6546-1 | 15.4 | 5.7 |
| 84140 Prostate Cancer (OD04410) | 21.0 | 8.7 |
| 84141 Prostate NAT (OD04410) | 42.6 | 33.4 |
| 87073 Prostate Cancer (OD04720-01) | 23.5 | 22.2 |
| 87074 Prostate NAT (OD04720-02) | 32.1 | 25.2 |
| Normal Lung GENPAK 061010 | 15.7 | 12.0 |
| 83239 Lung Met to Muscle (ODO4286) | 1.1 | 0.8 |
| 83240 Muscle NAT (ODO4286) | 1.6 | 0.3 |
| 84136 Lung Malignant Cancer (OD03126) | 40.9 | 20.7 |
| 84137 Lung NAT (OD03126) | 20.3 | 15.9 |
| 84871 Lung Cancer (OD04404) | 100.0 | 100.0 |
| 84872 Lung NAT (OD04404) | 27.2 | 25.9 |
| 84875 Lung Cancer (OD04565) | 31.4 | 29.3 |
| 84876 Lung NAT (OD04565) | 6.8 | 5.1 |
| 85950 Lung Cancer (OD04237-01) | 7.1 | 4.3 |
| 85970 Lung NAT (OD04237-02) | 11.3 | 7.0 |
| 83255 Ocular Mel Met to Liver (ODO4310) | 0.0 | 0.0 |
| 83256 Liver NAT (ODO43 10) | 4.1 | 1.5 |
| 84139 Melanoma Mets to Lung (OD04321) | 7.6 | 4.8 |
| 84138 Lung NAT (OD04321) | 33.9 | 20.4 |
| Normal Kidney GENPAK 061008 | 19.9 | 9.2 |
| 83786 Kidney Ca, Nuclear grade 2 (OD04338) | 32.5 | 25.3 |
| 83787 Kidney NAT (OD04338) | 9.8 | 7.9 |
| 83788 Kidney Ca Nuclear grade 1/2 (OD04339) | 29.5 | 23.7 |
| 83789 Kidney NAT (OD04339) | 3.6 | 2.3 |
| 83790 Kidney Ca, Clear cell type (OD04340) | 4.0 | 3.7 |
| 83791 Kidney NAT (OD04340) | 14.8 | 9.3 |
| 83792 Kidney Ca, Nuclear grade 3 (OD04348) | 4.5 | 3.0 |
| 83793 Kidney NAT (OD04348) | 8.0 | 4.4 |
| 87474 Kidney Cancer (OD04622-01) | 4.5 | 4.1 |
| 87475 Kidney NAT (OD04622-03) | 11.8 | 4.2 |
| 85973 Kidney Cancer (OD04450-01) | 26.4 | 12.9 |
| 85974 Kidney NAT (OD04450-03) | 13.7 | 5.0 |
| Kidney Cancer Clontech 8120607 | 1.7 | 1.0 |
| Kidney NAT Clontech 8120608 | 3.8 | 1.5 |
| Kidney Cancer Clontech 8120613 | 0.4 | 0.4 |
| Kidney NAT Clontech 8120614 | 8.0 | 5.2 |
| Kidney Cancer Clontech 9010320 | 10.7 | 7.0 |
| Kidney NAT Clontech 9010321 | 11.6 | 5.4 |
| Normal Uterus GENPAK 061018 | 11.2 | 5.9 |
| Uterus Cancer GENPAK 064011 | 59.9 | 37.4 |
| Normal Thyroid Clontech A+6570-1 | 16.3 | 5.7 |
| Thyroid Cancer GENPAK 064010 | 33.0 | 17.0 |
| Thyroid Cancer INVITROGEN A302152 | 14.4 | 9.2 |
| Thyroid NAT INVITROGEN A302153 | 11.9 | 7.9 |
| Normal Breast GENPAK 061019 | 20.3 | 10.8 |
| 84877 Breast Cancer (OD04566) | 10.7 | 5.8 |
| 85975 Breast Cancer (OD04590-01) | 10.4 | 7.1 |
| 85976 Breast Cancer Mets (OD04590-03) | 7.6 | 3.0 |
| 87070 Breast Cancer Metastasis (OD04655-05) | 7.4 | 5.4 |
| GENPAK Breast Cancer 064006 | 13.9 | 9.4 |
| Breast Cancer Res. Gen. 1024 | 40.9 | 25.9 |
| Breast Cancer Clontech 9100266 | 9.8 | 5.4 |
| Breast NAT Clontech 9100265 | 13.8 | 10.7 |
| Breast Cancer INVITROGEN A209073 | 45.7 | 33.0 |
| Breast NAT INVITROGEN A2090734 | 11.0 | 5.6 |
| Normal Liver GENPAK 061009 | 4.2 | 2.6 |
| Liver Cancer GENPAK 064003 | 0.6 | 0.3 |
| Liver Cancer Research Genetics RNA 1025 | 2.9 | 2.5 |
| Liver Cancer Research Genetics RNA 1026 | 7.3 | 6.3 |
| Paired Liver Cancer Tissue Research Genetics RNA 6004-T | 7.0 | 2.2 |
| Paired Liver Tissue Research Genetics RINA 6004-N | 0.7 | 0.7 |
| Paired Liver Cancer Tissue Research Genetics RNA 6005-T | 11.8 | 5.7 |
| Paired Liver Tissue Research Genetics RNA 6005-N | 0.6 | 0.7 |
| Normal Bladder GENPAK 061001 | 12.1 | 11.7 |
| Bladder Cancer Research Genetics RNA 1023 | 4.9 | 2.9 |
| Bladder Cancer INVITROGEN A302173 | 59.5 | 27.4 |
| 87071 Bladder Cancer (OD04718-01) | 12.8 | 11.7 |
| 87072 Bladder Normal Adjacent (OD04718-03) | 2.8 | 0.7 |
| Normal Ovary Res. Gen. | 29.7 | 19.8 |
| Ovarian Cancer GENPAK 064008 | 25.2 | 30.8 |
| 87492 Ovary Cancer (OD04768-07) | 2.8 | 2.5 |
| 87493 Ovary NAT (OD04768-08) | 4.0 | 2.6 |
| Normal Stomach GENPAK 061017 | 6.7 | 5.4 |
| Gastric Cancer Clontech 9060358 | 4.9 | 1.6 |
| NAT Stomach Clontech 9060359 | 7.1 | 5.0 |
| Gastric Cancer Clontech 9060395 | 30.1 | 25.5 |
| NAT Stomach Clontech 9060394 | 1.5 | 5.6 |
| Gastric Cancer Clontech 9060397 | 18.3 | 21.9 |
| NAT Stomach Clontech 9060396 | 3.7 | 1.8 |
| Gastric Cancer GENPAK 064005 | 16.3 | 18.0 |

TABLE HD
Panel 4.1D

| | Relative | | Relative |
|---|---|---|---|
| | Expression (%) | | Expression (%) |
| | 4.1dxtm6555f | | 4.1dxtm6555f |
| Tissue Name | _ag1450_a1 | Tissue Name | _ag1450_a1 |
| 93768_Secondary Th1_anti- | 0.3 | 93100_HUVEC | 0.0 |
| CD28/anti-CD3 | | (Endothelial)_IL-lb | |
| 93769_Secondary Th2_anti- | 0.3 | 93779_HUVEC | 0.2 |
| CD28/anti-CD3 | | (Endothelial)_IFN gamma | |
| 93770_Secondary Tr1_anti- | 0.3 | 93102_HUVEC | 0.6 |
| CD28/anti-CD3 | | (Endothelial)_TNF alpha + IFN | |
| | | gamma | |
| 93573_Secondary Th1_resting | 0.2 | 93101_HUVEC | 0.5 |
| day 4-6 in IL-2 | | (Endothelial)_TNF alpha + IL4 | |
| 93572_Secondary_Th2_resting | 0.0 | 93781_HUVEC | 0.4 |
| day 4-6 in IL-2 | | (Endothelial)_IL-11 | |
| 93571_Secondary Tr1_resting | 0.0 | 93583_Lung Microvascular | 0.2 |
| day 4-6 in 11-2 | | Endothelial Cells_none | |
| 93568_primary Th1_anti- | 0.0 | 93584_Lung Microvascular | 0.4 |
| CD28/anti-CD3 | | Endothelial Cells_TNFa (4 | |
| | | ng/ml) and IL1b (1 ng/ml) | |
| 93569-primary Th2_anti- | 0.0 | 92662_Microvascular Dermal | 0.0 |
| CD28/anti-CD3 | | endothelium_none | |
| 93570_primary Tr1_anti- | 0.0 | 92663_Microsvasular Dermal | 0.0 |
| CD28/anti-CD3 | | endothelium_TNFa (4 ng/ml) | |
| | | and IL1b (1 ng/ml) | |
| 93565_primary Th1_resting dy | 0.0 | 93773_Bronchial | 3.6 |
| 4-6 in IL-2 | | epithelium_TNFa (4 ng/ml) and | |
| | | IL1b (1 ng/ml)** | |
| 93566_primary Th2_resting dy | 0.0 | 93347_Small Airway | 5.0 |
| 4-6 in IL-2 | | Epithelium_none | |
| 93567_primary Tr1_resting dy | 0.0 | 93348_Small Airway | 0.4 |
| 4-6 in IL-2 | | Epithelium_TNFa (4 ng/ml) | |
| | | and IL1b (1 ng/ml) | |
| 93351_CD45RA CD4 | 44.2 | 92668_Coronery Artery | 2.4 |
| lymphocyte_anti-CD28/anti- | | SMC_resting | |
| CD3 | | | |
| 93352_CD45RO CD4 | 0.0 | 92669_Coronery Artery | 3.2 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.0 | 93107_astrocytes_resting | 2.8 |
| CD28/anti-CD3 | | | |
| 93353_chronic CD8 | 0.1 | 93108_astrocytes_TNFa (4 | 10.8 |
| Lymphocytes 2ry_resting dy | | ng/ml) and IL1b(1 ng/ml) | |
| 4-6 in IL-2 | | | |
| 93574_chronic CD8 | 0.0 | 92666_KU-812 | 0.3 |
| Lymphocytes 2ry_activated | | (Basophil)_resting | |
| CD3/CD28 | | | |
| 93354_CD4_none | 0.0 | 92667_KU-812 | 0.5 |
| | | (Basophil)_PMA/ionoycin | |
| 93252_Secondary | 0.0 | 93579_CCD1106 | 3.0 |
| Th1/Th2/Tr1_anti-CD95 CH11 | | (Keratinocytes)_none | |
| 93103_LAK cells_resting | 0.2 | 93580_CCD1106 | 1.3 |
| | | (Keratinocytes)_TNFa and | |
| | | IFNg** | |
| 93788_LAK cells_IL-2 | 0.0 | 93791_Liver Cirrhosis | 0.4 |
| 93787_LAK cells_IL-2+IL-12 | 0.0 | 93577_NCI-H292 | 0.0 |
| 93789_LAK cells_IL-2+IFN | 0.0 | 93358_NCI-H292_IL-4 | 0.1 |
| gamma | | | |
| 93790_LAK cells_IL-2+IL-18 | 0.0 | 93360_NCI-H292_IL-9 | 0.3 |
| 93104_LAK | 0.0 | 93359_NCI-H292_IL-13 | 2.3 |
| cells_PMA/ionomycin and IL- | | | |
| 18 | | | |
| 93578_NK Cells IL-2_resting | 0.0 | 93357_NCI-H292_IFN gamma | 0.2 |
| 93109_Mixed Lymphocyte | 0.2 | 93777_HPAEC_- | 0.0 |
| Reaction_Two Way MLR | | | |
| 93110_Mixed Lymphocyte | 1.2 | 93778_HPAEC_IL-1 beta/TNA | 0.5 |
| Reaction_Two Way MLR | | alpha | |
| 93111_Mixed Lymphocyte | 0.0 | 93254_Normal Human Lung | 37.9 |
| Reaction_Two Way MLR | | Fibroblast_none | |
| 93112_Mononuclear Cells | 0.0 | 93253_Normal Human Lung | 74.9 |
| (PBMCs)_resting | | Fibroblast_TNFa (4 ng/ml) and | |
| | 0.0 | IL-lb (1 ng/ml) | |
| 93113_Mononuclear Cells | 2.9 | 93257_Normal Human Lung | 39.0 |
| (PBMCs)_PWM | | Fibroblast_IL-4 | |
| 93114_Mononuclear Cells | 1.5 | 93256_Normal Human Lung | 71.6 |
| (PBMCs)_PHA-L | | Fibroblast_IL-9 | |
| 93249_Ramos (B cell)_none | 0.0 | 93255_Normal Human Lung | 24.9 |
| | | Fibroblast_IL-13 | |
| 93250_Ramos (B | 0.0 | 93258_Normal Human Lung | 41.1 |
| cell)_ionomycin | | Fibroblast_IFN gamma | |
| 93349_B lymphocytes_PWM | 0.1 | 93106_Dermal Fibroblasts | 83.8 |
| | | CCD1070_resting | |
| 93350_B lymphoytes_CD40L | 0.4 | 93361_Dermal Fibroblasts | 76.4 |
| and IL-4 | | CCD 1070_TNF alpha 4 ng/ml | |
| 92665_EOL-1 | 0.0 | 93105_Dermal Fibroblasts | 100.0 |
| (Eosinophil)_dbcAMP | | CCD1070_IL-1 beta 1 ng/ml | |
| differentiated | | | |
| 93248_EOL-1 | 0.0 | 93772_dermal fibroblast_IFN | 8.9 |
| (Eosinophil)_dbcAMP/PMAion | | gamma | |
| omycin | | | |
| 93356_Dendritic Cells_none | 2.5 | 93771_dermal fibroblast_IL-4 | 10.6 |
| 93355_Dendritic Cells_LPS | 20.5 | 93892_Dermal fibroblasts_{—} | 6.1 |
| 100 ng/ml | | none | |
| 93775_Dendritic Cells_anti- | 10.7 | 99202_Neutrophils_TNFa+LPS | 1.1 |
| CD40 | | | |
| 93774_Monocytes_resting | 0.0 | 99203_Neutrophils_none | 1.5 |
| 93776_Monocytes_LPS 50 | 20.4 | 735010_Colon_normal | 0.9 |
| ng/ml | | | |
| 93581_Macrophages_resting | 0.2 | 735019_Lung_none | 3.6 |
| 93582_Macrophages_LPS 100 | 5.8 | 64028-1_Thymus_none | 1.7 |
| ng/ml | | | |
| 93098_HUVEC | 0.0 | 64030-1_Kidney_none | 2.6 |
| (Endothelial) none | | | |
| 93099_HUVEC | 0.0 | | |
| (Endothelial)_starved | | | |

Panel 1.2 Summary: Ag1450 The SC128855163_A gene encodes a putative Wnt5a-like protein. [From OMIM 164975] The Wnt genes belong to a family of protooncogenes with at least 13 known members that are expressed in species ranging from Drosophila to man. The name Wnt denotes the relationship of this family to the Drosophila segment polarity gene ‘wingless’ and to its vertebrate ortholog, Int1, a mouse protooncogene (see 164820). Transcription of Wnt family genes appears to be developmentally regulated in a precise temporal and spatial manner. The Wnt family is considered to be 1 of the 3 major families of signaling molecules in the mouse, the others being the fibroblast growth factor-related family (see 164980) and the transforming growth factor-beta-related family (TGFB; 190180). All of the known vertebrate Wnt genes encode 38- to 43-kD cysteine-rich putative glycoproteins, which have features typical of secreted growth factors: a hydrophobic signal sequence, a conserved asparagine-linked oligosaccharide consensus sequence, and 22 conserved cysteine residues whose relative spacing is maintained.

Results from two experiments performed using the same probe/primer set to examine expression of the SC128855163_A gene were very consistent. The SC128855163_A gene is expressed at varying levels in the majority of samples on this panel. This gene shows its highest expression in a sample derived from a glioblastoma cell line SF-295 (CT=22). Interestingly, there is a strong association with over-expression in cell lines derived from CNS malignancies when compared to the low to moderate expression in the samples derived from normal CNS tissue (see below for further discussion of expression in CNS tissue). In addition, there is consistently high expression of the SC128855163_A gene in melanoma cell lines, ovarian cancer cell lines and lung cancercell lines. Thus, therapeutic modulation of the activity of the SC128855163_A gene product through the use of inhibitory antibodies or small molecule drugs might be of use for the treatment of brain cancer, melanoma, ovarian cancer and/or lung cancer.

Variable levels of SC128855163_A gene expression are detected throughout the brain with highest expression in cerebral cortex (CT=29) and lowest expression in amygdala and cerebellum (CTs=30-31). Wnt-5A signalling is believed to play a critical role in cadherin-mediated cell organization. Cadherins can act as axon guidance and cell adhesion proteins, specifically during development and in the response to injury. Therefore, manipulation of levels or activation of the SC128855163_A protein may be of use in inducing a compensatory synaptogenic response to neuronal death in Alzheimer's disease, Parkinson's disease, Huntington's disease, spinocerebellar ataxia, progressive supranuclear palsy, ALS, head trauma, stroke, or any other disease/condition associated with neuronal loss.

Among the metabolically relevant tissues, the SC128855163_A gene is expressed at moderate levels in pancreas (CT=30-32), adrenal gland (CT=29), thyroid (CT=31), pituitary (CT=28-32) and liver (CT=30). In addition, this gene is expressed at high levels in skeletal muscle (CT=27). These observations suggest that the SC128855163_A Wnt-5A-like protein may be secreted from skeletal muscle as a paracrine or endocrine signalling molecule acting on other insulin-responsive tissues (i.e., adipose and pancreatic beta cells). Thus, this gene product may be a drug target for metabolic diseases involving skeletal muscle, including Type 2 diabetes.

Panel 2D Summary: Ag1450 The expression of the SC128855163_A gene was assessed in two independent runs on Panel 2D using the same probe/primer set and the results are in excellent agreement. This gene is expressed most highly in a sample of lung cancer tissue. Overall, the SC128855163_A gene appears to be over-expressed in cancer tissues when compared to the adjacent normal tissue in the following samples: colon, lung, kidney, breast, and gastric cancer. Thus, therapeutic modulation of the activity of the SC128855163_A gene product, using antibodies or small molecule drugs, may be of use in the treatment of colon, lung, kidney, breast or gastric cancers. These results are consistent with the observation that the Wnt-5A gene appears to be up-regulated in a number of human malignancies; this suggests that the homologous SC128855163_A gene may have similar activities (Ref. 1).

Panel 4.1D Summary: Ag1450 Among the samples on Panel 4.1D, the SC128855163_A gene is expressed in fibroblasts and in LPS-activated monocytes, macrophages and dendritic cells. This transcript encodes a putative Wnt5a-like molecule. WNTs are secreted signalling molecules that regulate cell fate and behavior and are involved in embryonic development and hematopoiesis. During inflammation, the Wnt5a-like protein encoded for by the SC128855163_A gene could potentiate the inflammatory response by acting as an autocrine factor and stimulating monocyte differentiation into dendritic cells as well as by allowing dendritic cells to mature into potent antigen presenting cells. Alternatively, the SC128855163_A gene product may influence the differentiation of other cell types in the microenvironment including synovial tissues (Ref. 2). Therefore, antibodies which block the function of this protein could be important reducing or blocking inflammation associated with rheumatoid arthritis, asthma, allergy, psoriasis, IBD and Crohn's disease.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26 and 28;
(b) a variant of a mature form of an amino acid sequence selected from the group consisting of SEQ ID NOS:2, 4, 6, 8, 10, 12, 14, 16, 18, 20, 22, 24, 26 and 28, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS:2, 4, 6, 8, 10, 12, 14, 16, 18, 20, 22, 24, 26 and 28; and
(d) a variant of an amino acid sequence selected from the group consisting of SEQ ID NOS:2, 4, 6, 8, 10, 12, 14, 16, 18, 20, 22, 24, 26 and 28, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting SEQ ID NOS:2, 4, 6, 8, 10, 12, 14, 16, 18, 20, 22, 24, 26 and 28.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:1, 3, 5, 7, 9, 11, 13, 15, 17, 19, 21, 23, 25, and 27.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26 and 28;
(b) a variant of a mature form of an amino acid sequence selected from the group consisting of SEQ ID NOS:2, 4, 6, 8, 10, 12, 14, 16, 18, 20, 22, 24, 26 or 28, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS :2, 4, 6, 8, 10, 12, 14, 16, 18, 20, 22, 24, 26 and 28;
(d) a variant of an amino acid sequence selected from the group consisting SEQ ID NOS:2, 4, 6, 8, 10, 12, 14, 16, 18, 20, 22, 24, 26 and 28, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, 22, 24, 26 and 28, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 21, 23, 25, and 27.

9. The nucleic acid molecule of claim 5, wherein said nucleic acid molecule comprises a nucleotide sequence selected from the group consisting of:
(a) a nucleotide sequence selected from the group consisting of SEQ ID NOS:1, 3, 5, 7, 9, 11, 13, 15, 17, 19, 21, 23, 25, and 27;
(b) a nucleotide sequence differing by one or more nucleotides from a nucleotide sequence selected from the group consisting of SEQ ID NOS:1, 3, 5, 7, 9, 11, 13, 15, 17, 19, 21, 23, 25, and 27, provided that no more than 20% of the nucleotides differ from said nucleotide sequence;
(c) a nucleic acid fragment of (a); and
(d) a nucleic acid fragment of (b).

10. Th e nucleic acid molecule of claim 5, wherein said nucleic acid molecule hybridizes under stringent conditions to a nucleotide sequence chosen from the group consisting SEQ ID NOS:1, 3, 5, 7, 9, 11, 13, 15, 17, 19, 21, 23, 25, and 27, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide, whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a NOVX-associated disorder, said method comprising administering to a subject in which such treatment or prevention is desired the antibody of claim 15 in an amount sufficient to treat or prevent said NOVX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to cancers.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20, 22, 24, 26 and 28, or a biologically active fragment thereof.

49. A method of treating a pathological state in a mammal, the method comprising administering to the mammal the antibody of claim 15 in an amount sufficient to alleviate the pathological state.


SEQUENCE LISTING (61 sequences)

SEQ ID NO: 1 - DNA, 2752 bp, Homo sapiens
ccgcggggccccgcgcccggcccgcccgcctgcccgcccgcggccatggccgtccggcccggcctgtggccagcgctcctgggcatagtcctcgccgcttggctccgcggctcgggtgcccagcagagtgccaccgtggccaacccagtgcctggtgccaacccggacctgcttccccacttcctggtggagcccgaggatgtgtacatcgtcaagaacaagccagtgctgcttgtgtgcaaggccgtgcccgccacgcagatcttcttcaagtgcaacggggagtgggtgcgccaggtggaccacgtgatcgagcgcagcacagacgggagcagtgggctgcccaccatggaggtccgcattaatgtctcaaggcagcaggtcgagaaggtgttcgggctggaggaatactggtgccagtgcgtggcatggagctcctcgggcaccaccaagagtcagaaggcctacatccgcatagccagattgcgcaagaacttcgagcaggagccgctggccaaggaggtgtccctggagcagggcatcgtgctgccctgccgtccaccggagggcatccctccagccgaggtggagtggctccggaacgaggacctggtggacccgtccctggaccccaatgtatacatcacgcgggagcacagcctggtggtgcgacaggcccgccttgctgacacggccaactacacctgcgtggccaagaacatcgtggcacgtcgccgcagcgcctccgctgctgtcatcgtctacgtgaacggtgggtggtcgacgtggaccgagtggtccgtctgcagcgccagctgtgggcgcggctggcagaaacggagccggagctgcaccaacccggcgcctctcaacgggggcgctttctgtgaggggcagaatgtccagaaaacagcctgcgccaccctgtgcccagtagacggcagctggagcccgtggagcaagtggtcggcctgtgggctggactgcacccactggcggagccgtgagtgctctgacccagcaccccgcaacggaggggaggagtgccagggcactgacctggacacccgcaactgtaccagtgacctctgtgtacacagtgcttctggccctgaggacgtggccctctatgtgggcctcatcgccgtggccgtctgcctggtcctgctgctgcttgtcctcatcctcgtttattgccggaagaaggaggggctggactcagatgtggctgactcgtccattctcacctcaggcttccagcccgtcagcatcaagcccagcaaagcagacaacccccatctgctcaccatccagccggacctcagcaccaccaccaccacctaccagggcagtctctgtccccggcaggatgggcccagccccaagttccagctcaccaatgggcacctgctcagccccctgggtggcggccgccacacactgcaccacagctctcccacctctgaggccgaggagttcgtctcccgcctctccacccagaactacttccgctccctgccccgaggcaccagcaacatgacctatgggaccttcaacttcctcgggggccggctgatgatccctaatacaggtatcagcctcctcatccccccagatgccataccccgagggaagatctatgagatctacctcacgctgcacaagccggaagacgtgaggttgcccctagctggctgtcagaccctgctgagtcccatcgttagctgtggaccccctggcgtcctgctcacccggccagtcatcctggctatggaccactgtggggagcccagccctgacagctggagcctgcgcctcaaaaagcagtcgtgcgagggcagctgggaggatgtgctgcacctgggcgaggaggcgccctcccacctctactactgccagctggaggccagtgcctgctacgtcttcaccgagcagctgggccgctttgccctggtgggagaggccctcagcgtggctgccgccaagcgcctcaagctgcttctgtttgcgccggtggcctgcacctccctcgagtacaacatccgggtctactgcctgcatgacacccacgatgcactcaaggaggtggtgcagctggagaagcagctggggggacagctgatccaggagccacgggtcctgcacttcaaggacagttaccacaacctgcgcctatccatccacgatgtgcccagctccctgtggaagagtaagctccttgtcagctaccaggagatccccttttatcacatctggaatggcacgcagcggtacttgcactgcaccttcaccctggagcgtgtcagccccagcactagtgacctggcctgcaagctgtgggtgtggcaggtggagggcgacgggcagagcttcagcatcaacttcaacatcaccaaggacacaaggtttgctgagctgctggctctggagagtgaagcgggggtcccagccctggtgggccccagtgccttcaagatccccttcctcattcggcagaagataatttccagcctggacccaccctgtaggcggggtgccgactggcggactctggcccagaaactccacctggacagccatctcagcttctttgcctccaagcccagccccacagccatgatcctcaacctgtgggaggcgcggcacttccccaacggcaacctcagccagctggctgcagcagtggctggactgggccagccagacgctggcctcttcacagtgtcggaggctgagtgctgaggccggccag

SEQ ID NO: 2 - protein, 898 aa, Homo sapiens
MAVRPGLWPALLGIVLAAWLRGSGAQQSATVANPVPGANPDLLPHFLVEPEDVYIVKNKPVLLVCKAVPATQIFFKCNGEWVRQVDHVIERSTDGSSGLPTMEVRINVSRQQVEKVFGLEEYWCQCVAWSSSGTTKSQKAYIRIARLRKNFEQEPLAKEVSLEQGIVLPCRPPEGIPPAEVEWLRNEDLVDPSLDPNVYITREHSLVVRQARLADTANYTCVAKNIVARRRSASAAVIVYVNGGWSTWTEWSVCSASCGRGWQKRSRSCTNPAPLNGGAFCEGQNVQKTACATLCPVDGSWSPWSKWSACGLDCTHWRSRECSDPAPRNGGEECQGTDLDTRNCTSDLCVHSASGPEDVALYVGLIAVAVCLVLLLLVLILVYCRKKEGLDSDVADSSILTSGFQPVSIKPSKADNPHLLTIQPDLSTTTTTYQGSLCPRQDGPSPKFQLTNGHLLSPLGGGRHTLHHSSPTSEAEEFVSRLSTQNYFRSLPRGTSNMTYGTFNFLGGRLMIPNTGISLLIPPDAIPRGKIYEIYLTLHKPEDVRLPLAGCQTLLSPIVSCGPPGVLLTRPVILAMDHCGEPSPDSWSLRLKKQSCEGSWEDVLHLGEEAPSHLYYCQLEASACYVFTEQLGRFALVGEALSVAAAKRLKLLLFAPVACTSLEYNIRVYCLHDTHDALKEVVQLEKQLGGQLIQEPRVLHFKDSYHNLRLSIHDVPSSLWKSKLLVSYQEIPFYHIWNGTQRYLHCTFTLERVSPSTSDLACKLWVWQVEGDGQSFSINFNITKDTRFAELLALESEAGVPALVGPSAFKIPFLIRQKIISSLDPPCRRGADWRTLAQKLHLDSHLSFFASKPSPTAMILNLWEARHFPNGNLSQLAAAVAGLGQPDAGLFTVSEAEC

SEQ ID NO: 3 - DNA, 604 bp, Homo sapiens
aggaactcaacatgactagtcaatgcttgctggattgggccttggtgctacttctcaccactactgcattctctctggactgtcactttcaaaggtgcaagggcaactgggagattttagaacatttaaaaaacctaggagaaaaatttcctctgcaatgtctaaaggacaggagcaacttcagattcttccaggtttctaaaagtaacctgttttcaaaggaaaatgccctcattgccaaaaaagaaatgttacagcagatattcaacactttcagccttaatgtctcccaatctttttggaatgaaagcagcttggagagattcctaagtagactttatcagcaaatagagaagacagaggtgtgtttggagcaggaaaccaggaaagagggccgttcactcttgcaaagggggaataccatatttagactaaaaaattatttccaagggattcacaactacttacaccaccaaaattatagcaactgtgcctgggaggtcatccatgttgaaatccgaaggggtctactatttattgaacagtgcacaagaagactccaataccaagaaacaggttatttacataaataactaatttggag

SEQ ID NO: 4 - protein, 193 aa, Homo sapiens
MTSQCLLDWALVLLLTTTAFSLDCHFQRCKGNWEILEHLKNLGEKFPLQCLKDRSNFRFFQVSKSNLFSKENALIAKKEMLQQIFNTFSLNVSQSFWNESSLERFLSRLYQQIEKTEVCLEQETRKEGRSLLQRGNTIFRLKNYFQGIHNYLHHQNYSNCAWEVIHVEIRRGLLFIEQCTRRLQYQETGYLHK

SEQ ID NO: 5 - DNA, 3465 bp, Homo sapiens
  misc_feature (1)..(3465): Wherein N is G or A or T or C
gatgtatggtctgccctgggcttgtctgttccctcctgagcctgagccccttaccttcctgaccccatgaagcacacactggctctgctggctcccctgctgggcctgggcctggggctggccctgagtcagctggctgcaggggccacagactgcaagttccttggcccggcagagcacctgacattcaccccagcagccagggcccggtggctggcccctcgagttcgtgcgccaggactcctggactccctctatggcaccgtgcgccgcttcctctcggtggtgcagctcaatcctttcccttcagagttggtaaaggccctactgaatgagctggcctccgtgaaggtgaatgaggtggtgcggtacgaggcgggctacgtggtatgcgctgtgatcgcgggcctctacctgctgctggtgcccactgccgggctttgcttctgctgctgccgctgccaccggcgctgcgggggacgagtgaagacagagcacaaggcgctggcctgtgagcgcgcggccctcatggtcttcctgctgctgaccaccctcttgctgctgattggtgtggtctgtgcctttgtcaccaaccagcgcacgcatgaacagatgggccccagcatcgaggccatgcctgagaccctgctcagcctctggggcctggtctctgatgtcccccaagagctgcaggccgtggcacagcaattctccctgccccaggagcaagtctcagaggagctggatggtgttggtgtgagcattgggagcgcgatccacactcagctcaggagctccgtgtaccccttgctggcggccgtgggcagtttgggccaggtcctgcaggtctccgtgcaccacctgcaaaccttgaatgctacagtggtagagctgcaggccgggcagcaggacctggagccagccatccgggaacaccgggaccgcctccttgagctgctgcaggaggccaggtgccagggagattgtgcaggggccctgagctgggcccgcaccctggagctgggtgctgacttcagccaggtgccctctgtggaccatgtcctgcaccagctaaaaggtgtccccgaggccaacttctccagcatggtccaggaggagaacagcaccttcaacgcccttccagccctggctgccatgcagacatccagcgtggtgcaagagctgaagaaggcagtggcccagcagccggaaggggtgaggacactggctgaagggttcccgggcttggaggcagcttcccgctgggcccaggcactgcaggaggtggaggagagcagccgcccctacctgcaggaggtgcagagatacgagacctacaggtggatcgtgggctgcgtgctgtgctccgtggtcctattcgtggtgctctgcaacctgctgggcctcaatctgggcatctggggcctgtctgccagggacgaccccagccacccagaagccaagggcgaggctggagcccgcttcctcatggcaggtgtgggcctcagcttcctctttgctgcacccctcatcctcctggtgttcgccaccttcctggtgggtggcaacgtgcagacgctggtgtgccagagctgggagaacggcgagctctttgagtttgcagacaccccagggaacctgcccccgtccatgaacctgtcgcaacttcttggcctgaggaagaacatcagcatccaccaagcctatcagcagtgcaaggaaggggcagcgctctggacagtcctgcagctcaacgactcctacgacctggaggagcacctggatatcaaccagtataccaacaagctacggcaggagttgcagagcctgaaagtagacacacagagcctggacctgctgagctcagccgcccgccgggacctggaggccctgcagagcagtgggcttcagcgcatccactaccccgacttcctcgttcagatccagaggcccgtggtgaagaccagcatggagcagctggcccaggagctgcaaggactggcccaggcccaagacaattctgtgctggggcagcggctgcaggaggaggcccaaggactcagaaaccttcaccaggagaaggtcgtcccccagcagagccttgtggcaaagctcaacctcagcgtcagggccctggagtcctctgccccgaatctccaggtggctgctgttggtggggacctggagacctcagatgtcctagccaatgtcacctacctgaaaggagagctgcctgcctgggcagccaggatcctgaggaatgtgagtgagtgtttcctggcccgggagatgggctacttctcccagtacgtggcctgggtgagagaggaggtgactcagcgcattgccacctgccagcccctctccggagccctggacaacagccgtgtgatcctgtgtgacatgatggctgacccctggaatgccttctggttctgcctggcatggtgcaccttcttcctgatccccagcatcatctttgccgtcaagacctccaaatacttccgtcctatccggaaacgcctcagctccaccagctctgaggagactcagctcttccacatcccccgggttacctccctgaagctgtagggccttgtgggtgtgntctnttgccctgntgcnaattttccangccccgatttaaccctgccnngtggaaacgcgcagggagttggggtctcgggagcctanctccacaatatccctgggtcccatgcatgaccaccggcagttgctgcggcccaccccgctcctgggcctgtgcctcccttgcctcctccagatggccgcctgcctcatcgaacccccaatctgatctgcacattccaccaggccacccttctgaggcagctgcgagtccagctggacttgagtggcagagagcagctggggcggctgtgccctgccaggaggatgctgccccaagcctgccggctggcaggtctgagaaccatccggatcagtcctgtccaatagagacatgatgcaagccacagatgtcacttaaaatgagccagtaggcacagtaaagaaagaaaaaaaggttaaataatttcaacgatatgttttattaaccccattgtaaatgattagcactcaacccttagattgaaatagggtattaagagtgagaggccgaggctcagccgccaggctttgatgagatgctgcctggtcaagtggatcctgtccagcacagccccacagggctcaggcagaggtggctcaggacgggtggggctggtgtgcatcctttgccgaagctttctgcacacccgtgacagcagcagctatgctgagtggggtggacggggagaaaggtgaaggggctttaagaatgagtgtcccacgggcctggtgtacgaagacaccttcaaactcatttacgcgcagttcttctctcagggagatggcaccacctatgcacacttcctcttcaacgcctgtgatgcgtacgggaac

SEQ ID NO: 6 - protein, 841 aa, Homo sapiens
MKHTLALLAPLLGLGLGLALSQLAAGATDCKFLGPAEHLTFTPAARARWLAPRVRAPGLLDSLYGTVRRFLSVVQLNPFPSELVKALLNELASVKVNEVVRYEAGYVVCAVIAGLYLLLVPTAGLCFCCCRCHRRCGGRVKTEHKALACERAALMVFLLLTTLLLLIGVVCAFVTNQRTHEQMGPSIEAMPETLLSLWGLVSDVPQELQAVAQQFSLPQEQVSEELDGVGVSIGSAIHTQLRSSVYPLLAAVGSLGQVLQVSVHHLQTLNATVVELQAGQQDLEPAIREHRDRLLELLQEARCQGDCAGALSWARTLELGADFSQVPSVDHVLHQLKGVPEANFSSMVQEENSTFNALPALAAMQTSSVVQELKKAVAQQPEGVRTLAEGFPGLEAASRWAQALQEVEESSRPYLQEVQRYETYRWIVGCVLCSVVLFVVLCNLLGLNLGIWGLSARDDPSHPEAKGEAGARFLMAGVGLSFLFAAPLILLVFATFLVGGNVQTLVCQSWENGELFEFADTPGNLPPSMNLSQLLGLRKNISIHQAYQQCKEGAALWTVLQLNDSYDLEEHLDINQYTNKLRQELQSLKVDTQSLDLLSSAARRDLEALQSSGLQRIHYPDFLVQIQRPVVKTSMEQLAQELQGLAQAQDNSVLGQRLQEEAQGLRNLHQEKVVPQQSLVAKLNLSVRALESSAPNLQVAAVGGDLETSDVLANVTYLKGELPAWAARILRNVSECFLAREMGYFSQYVAWVREEVTQRIATCQPLSGALDNSRVILCDMMADPWNAFWFCLAWCTFFLIPSIIFAVKTSKYFRPIRKRLSSTSSEETQLFHIPRVTSLKL

SEQ ID NO: 7 - DNA, 3261 bp, Homo sapiens
atgaagcacacactggctctgctggctcccctgctgggcctgggcctggggctggccctgagtcagctggctgcaggggccacagactgcaagttccttggcccggcagagcacctgacattcaccccagcagccagggcccggtggctggcccctcgagttcgtgcgccaggactcctggactccctctatggcaccgtgcgccgcttcctctcggtggtgcagctcaatcctttcccttcagagttggtaaaggccctactgaatgagctggcctccgtgaaggtgaatgaggtggtgcggtacgaggcgggctacgtggtatgcgctgtgatcgcgggcctctacctgctgctggtgcccactgccgggctttgcttctgctgctgccgctgccaccggcgctgcgggggacgagtgaagacagagcacaaggcgctggcctgtgagcgcgcggccctcatggtcttcctgctgctgaccaccctcttgctgctgattggtgtggtctgtgcctttgtcaccaaccagcgcacgcatgaacagatgggccccagcatcgaggccatgcctgagaccctgctcagcctctggggcctggtctctgatgtcccccaagagctgcaggccgtggcacagcaattctccctgccccaggagcaagtctcagaggagctggatggtgttggtgtgagcattgggagcgcgatccacactcagctcaggagctccgtgtaccccttgctggcggccgtgggcagtttgggccaggtcctgcaggtctccgtgcaccacctgcaaaccttgaatgctacagtggtagagctgcaggccgggcagcaggacctggagccagccatccgggaacaccgggaccgcctccttgagctgctgcaggaggccaggtgccagggagattgtgcaggggccctgagctgggcccgcaccctggagctgggtgctgacttcagccaggtgccctctgtggaccatgtcctgcaccagctaaaaggtgtccccgaggccaacttctccagcatggtccaggaggagaacagcaccttcaacgcccttccagccctggctgccatgcagacatccagcgtggtgcaagagctgaagaaggcagtggcccagcagccggaaggggtgaggacactggctgaagggttcccgggcttggaggcagcttcccgctgggcccaggcactgcaggaggtggaggagagcagccgcccctacctgcaggaggtgcagagatacgagacctacaggtggatcgtgggctgcgtgctgtgctccgtggtcctattcgtggtgctctgcaacctgctgggcctcaatctgggcatctggggcctgtctgccagggacgaccccagccacccagaagccaagggcgaggctggagcccgcttcctcatggcaggtgtgggcctcagcttcctctttgctgcacccctcatcctcctggtgttcgccaccttcctggtgggtggcaacgtgcagacgctggtgtgccagagctgggagaacggcgagctctttgagtttgcagacaccccagggaacctgcccccgtccatgaacctgtcgcaacttcttggcctgaggaagaacatcagcatccaccaagcctatcagcagtgcaaggaaggggcagcgctctggacagtcctgcagctcaacgactcctacgacctggaggagcacctggatatcaaccagtataccaacaagctacggcaggagttgcagagcctgaaagtagacacacagagcctggacctgctgagctcagccgcccgccgggacctggaggccctgcagagcagtgggcttcagcgcatccactaccccgacttcctcgttcagatccagaggcccgtggtgaagaccagcatggagcagctggcccaggagctgcaaggactggcccaggcccaagacaattctgtgctggggcagcggctgcaggaggaggcccaaggactcagaaaccttcaccaggagaaggtcgtcccccagcagagccttgtggcaaagctcaacctcagcgtcagggccctggagtcctctgccccgaatctccaggtggctgctgttggtggggacctggagacctcagatgtcctagccaatgtcacctacctgaaaggagagctgcctgcctgggcagccaggatcctgaggaatgtgagtgagtgtttcctggcccgggagatgggctacttctcccagtacgtggcctgggtgagagaggaggtgactcagcgcattgccacctgccagcccctctccggagccctggacaacagccgtgtgatcctgtgtgacatgatggctgacccctggaatgccttctggttctgcctggcatggtgcaccttcttcctgatccccagcatcatctttgccgtcaagacctccaaatacttccgtcctatccggaaacgcctcagggcttccgaggagaaaccctcagggctctgggtttgtccctgtgtcagggctgagggtctggggggaaggtcccctcttcaccatatctccactgctaccttgctggccccagagaccaccctgcccaaccaaaccactcagggccttgggccctctgcagatctcatccaggatttattggtgtccagtgggagaatactgggaattgccaaggccgtctctgggaagtctgcagatgcccgtgtgcccacaacagatggccgcctgcctcatcgaacccccaatctgatctgcacattccaccaggccacccttctgaggcagctgcgagtccagctggacttgagtggcagagagcagctggggcggctgtgccctgccaggaggatgctgccccaagcctgccggctggcaggactggctctctgtgcttccctgcaggctgtgactggcccgggctccctgctgccaattcttccagttcaaatcaacctgggggtcccatcttacaccatagccagggaagtgacaaaggcgtcggacggcagcctcctgggggacctcgggcacacaccacttagcaagaaggagggtatcaagtggcagaggccgaggctcagccgccaggctttgatgagatgctgcctggtcaagtggatcctgtccagcacagccccacagggctcaggcagaggtggctcaggacgggtggggctggtgtgcatcctttgccgaagctttctgcacacccgtgacagcagcagctatgctgagtggggtggacggggagaaaggtga

SEQ ID NO: 8 - protein, 841 aa, Homo sapiens; identical to SEQ ID NO: 6

SEQ ID NO: 9 - DNA, 2007 bp, Homo sapiens
agcaatggggcctggagcttcaggggacggggtcaggactgagacagctccacacatagcactggactccagagttggtctgcacgcctacgacatcagcgtggtggtcatctactttgtcttcgtcattgctgtggggatctggtcgtccatccgtgcaagtcgagggaccattggcggctatttcctggccgggaggtccatgagctggtggccagtgattggagcatctctgatgtccagcaatgtgggcagtggcttgttcatcggcctggctgggacaggggctgccggaggccttgccgtaggtggcttcgagtggaacgcaacctggctgctcctggcccttggctgggtcttcgtccctgtgtacatcgcagcaggtgtggtcacaatgccgcagtatctgaagaagcgatttgggggccagaggatccagatgtacatgtctgtcctgtctctcatcctctacatcttcaccaagatctcggtagacatcttctctggagccctcttcatccagatggcattgggctggaacctgtacctctccacagggatcctgctggtggtgactgccgtctacaccattgcaggtggtggcctcatggccgtgatctacacagatgctctgcagacggtgatcatggtagggggagccctggtcctcatgtttctgggtaaggaagagaccggctggtacccaggcctggagcagcggtacaggcaggccatccctaatgtcacagtccccaacaccacctgtcacctcccacggcccgatgctttccacatgcttcgggaccctgtgagcggggacatcccttggccaggtctcattttcgggctcacagtgctggccacctggtgttggtgcacagaccaggtaatcgtgcagcggtctctctcggccaagagtctgtctcatgccaagggaggctccgtgctggggggctacctgaagatcctccccatgttcttcatcgtcatgcctggcatgatcagccgggccctgttcccagagattgcgtgtatgtgtgtgcctgtgtgtacacatgcatgtgcagcaaggaagaggaaggaaggagtcctgcaggggttggtggtggcagttcgtctctccccaggtctgcgggggctgatgattgccgtgatcatggccgctctcatgagctcactcacctccatcttcaacagcagcagcaccctgttcaccattgatgtgtggcagcgcttccgcaggaagtcaacagagcaggagctgatggtggtgggcagggtgtttgtggtgttcctggttgtcatcagcatcctctggatccccatcatccaaagctccaacagtgggcagctcttcgactacatccaggctgtcaccagttacctggccccacccatcaccgctctcttcctgctggccatcttctgcaagagggtcacagagcagggagctttctggggcctcgtgtttggcctgggagtggggcttctgcgtatgatcctggagttctcatacccagcgccagcctgtggggaggtggaccggaggccagcagtgctgaaggacttccactacctgtactttgcaatcctcctctgcgggctcactgccatcgtcattgtcattgtcagcctctgtacaactcccatccctgagctccacacatacatttattgtggcactattcacaatagcaaagactttgaaccaatccaaatatccaacaatgagcaggccctgagcccagcagagaaggctgcgctagaacagaagctgacaagcattgaggaggagtcttctggctttgtccctccagcctggagctggttctgtgggctctctggaacaccggagcaggccctgagcccagcagagaaggctgcgctagaacagaagctgacaagcattgaggaggagccactctggagacatgtctgcaacatcaatgctgtccttttgctggccatcaacatcttcctctggggctattttgcgtgattccacag

SEQ ID NO: 10 - protein, 664 aa, Homo sapiens
MGPGASGDGVRTETAPHIALDSRVGLHAYDISVVVIYFVFVIAVGIWSSIRASRGTIGGYFLAGRSMSWWPVIGASLMSSNVGSGLFIGLAGTGAAGGLAVGGFEWNATWLLLALGWVFVPVYIAAGVVTMPQYLKKRFGGQRIQMYMSVLSLILYIFTKISVDIFSGALFIQMALGWNLYLSTGILLVVTAVYTIAGGGLMAVIYTDALQTVIMVGGALVLMFLGKEETGWYPGLEQRYRQAIPNVTVPNTTCHLPRPDAFHMLRDPVSGDIPWPGLIFGLTVLATWCWCTDQVIVQRSLSAKSLSHAKGGSVLGGYLKILPMFFIVMPGMISRALFPEIACMCVPVCTHACAARKRKEGVLQGLVVAVRLSPGLRGLMIAVIMAALMSSLTSIFNSSSTLFTIDVWQRFRRKSTEQELMVVGRVFVVFLVVISILWIPIIQSSNSGQLFDYIQAVTSYLAPPITALFLLAIFCKRVTEQGAFWGLVFGLGVGLLRMILEFSYPAPACGEVDRRPAVLKDFHYLYFAILLCGLTAIVIVIVSLCTTPIPELHTYIYCGTIHNSKDFEPIQISNNEQALSPAEKAALEQKLTSIEEESSGFVPPAWSWFCGLSGTPEQALSPAEKAALEQKLTSIEEEPLWRHVCNINAVLLLAINIFLWGYFA

SEQ ID NO: 11 - DNA, 2153 bp, Homo sapiens
atggctctgcagatgttcgtgacttacagtccttggaattgtttgctactgctagtggctcttgagtgttctgaagcatcttctgatttgaatgaatctgcaaattccactgctcagtatgcatctaacgcttggtttgctgctgccagctcagagccagaggaagggatatctgtttttgaactggattatgactatgtgcaaattccttatgaggtcactctctggatacttctagcatcccttgcaaaaataggtttccacctctaccacaggctgccaggcctcatgccagaaagctgcctcctcatcctggtgggggcgctggtgggcggcatcatcttcggcaccgaccacaaatcgcctccggtcatggactccagcatctacttcctgtatctcctgccacccatcgttctggagggcggctacttcatgcccacccggcccttctttgagaacatcggctccatcctgtggtgggcagtattgggggccctgatcaacgccttgggcattggcctctccctctacctcatctgccaggtgaaggcctttggcctgggcgacgtcaacctgctgcagaacctgctgttcggcagcctgatctccgccgtggacccagtggccgtgctagccgtgtttgaggaagcgcgcgtgaacgagcagctctacatgatgatctttggggaggccctgctcaatgatggcattactgtggtgttatacaatatgttaattgcctttacaaagatgcataaatttgaagacatagaaactgtcgacattttggctggatgtgcccgattcatcgttgtggggcttggaggggtattgtttggcatcgtttttggatttatttctgcatttatcacacgtttcactcagaatatctctgcaattgagccactcatcgtcttcatgttcagctatttgtcttacttagctgctgaaaccctctatctctccggcatcctggcgatcacagcctgcgcagtaacaatgaaaaagtacgtggaagaaaacgtgtcccagacatcatacacgaccatcaagtacttcatgaagatgctgagcagcgtcagcgagaccttgatcttcatcttcatgggtgtgtccactgtgggcaagaatcacgagtggaactgggccttcatctgcttcaccctggccttctgccaaatctggagagccatcagtgtatttgctctcttctatatcagtaaccagtttcggactttccccttctccatcaaggaccagtgcatcattttctacagtggtgttcgaggagctggaagtttttcacttgcatttttgcttcctctgtctctttttcctaggaagaaaatgtttgtcactgctactctagtagttatatactttactgtatttattcagggaatcacagttggccctctggtcaggtacctggatgttaaaaaaaccaataaaaaagaatccatcaatgaagagcttcatattcgtctgatggatcacttaaaggctggaatcgaagatgtgtgtgggcactggagtcactaccaagtgagagacaagtttaagaagtttgatcatagatacttacggaaaatcctcatcagaaagaacctacccaaatcaagcattgtttctttgtacaagaagctggaaatgaagcaagccatcgagatggtggagactgggatactgagctctacagctttctccataccccatcaggcccagaggatacaaggaatcaaaagactttcccctgaagatgtggagtccataagggacattctgacatccaacatgtaccaagttcggcaaaggaccctgtcctacaacaaatacaacctcaaaccccaaacaagtgagaagcaggctaaagagattctgatccgccgccagaacaccttaagggagagcatgaggaaaggtcacagcctgccctggggaaagccggctggcaccaagaatatccgctacctctcctacccctacgggaatcctcagtctgcaggaagagacacaagggctgctgggttctcaggtaagctgcccacctggctgctcctttggttgaggttcggtcgaggtggacagctgaccatggacacggcagggaccatcacaggtcccatagtcctttgctccaaaaaaaatag

SEQ ID NO: 12 - protein, 717 aa, Homo sapiens
MALQMFVTYSPWNCLLLLVALECSEASSDLNESANSTAQYASNAWFAAASSEPEEGISVFELDYDYVQIPYEVTLWILLASLAKIGFHLYHRLPGLMPESCLLILVGALVGGIIFGTDHKSPPVMDSSIYFLYLLPPIVLEGGYFMPTRPFFENIGSILWWAVLGALINALGIGLSLYLICQVKAFGLGDVNLLQNLLFGSLISAVDPVAVLAVFEEARVNEQLYMMIFGEALLNDGITVVLYNMLIAFTKMHKFEDIETVDILAGCARFIVVGLGGVLFGIVFGFISAFITRFTQNISAIEPLIVFMFSYLSYLAAETLYLSGILAITACAVTMKKYVEENVSQTSYTTIKYFMKMLSSVSETLIFIFMGVSTVGKNHEWNWAFICFTLAFCQIWRAISVFALFYISNQFRTFPFSIKDQCIIFYSGVRGAGSFSLAFLLPLSLFPRKKMFVTATLVVIYFTVFIQGITVGPLVRYLDVKKTNKKESINEELHIRLMDHLKAGIEDVCGHWSHYQVRDKFKKFDHRYLRKILIRKNLPKSSIVSLYKKLEMKQAIEMVETGILSSTAFSIPHQAQRIQGIKRLSPEDVESIRDILTSNMYQVRQRTLSYNKYNLKPQTSEKQAKEILIRRQNTLRESMRKGHSLPWGKPAGTKNIRYLSYPYGNPQSAGRDTRAAGFSGKLPTWLLLWLRFGRGGQLTMDTAGTITGPIVLCSKKN

SEQ ID NO: 13 - DNA, 251 bp, Homo sapiens
gacaggattccacagctttgcactcctggctctgctttctctgcaaccatgtctgacaaacccagcatggctgagattgagacactcaataagcagagattgaagaaggcagaaacacaagagataaatccaccgccttcaagagaaacaaacgaaagaagcaaacaggtgaattataatgagctgtgagctgcgaataggtactgcacattccatgggcattgccttcttattttacttcttttagctgt

SEQ ID NO: 14 - protein, 46 aa, Homo sapiens [sequence omitted]

SEQ ID NO: 15 - DNA, 2144 bp, Homo sapiens
atgcttcacacggccatatcatgctggcagccattcctgggtctggctgtggtgttaatcttcatgggatccaccattggctgccccgctcgctgtgagtgctctgcccagaacaaatctgttagctgtcacagaaggcgattgatcgccatcccagagggcattcccatcgaaaccaaaatcttggacctcagtaaaaacaggctaaaaagcgtcaaccctgaagaattcatatcatatcctctgctggaagagatagacttgagtgacaacatcattgccaatgtggaaccaggagcattcaacaatctctttaacctgcgttccctccgcctaaaaggcaatcgtctaaagctggtccctttgggagtattcacggggctgtccaatctcactaagcttgacattagtgagaataagattgtcattttactagactacatgttccaagatctacataacctgaagtctctagaagtgggggacaatgatttggtttatatatcacacagggcattcagtgggcttcttagcttggagcagctcaccctggagaaatgcaacttaacagcagtaccaacagaagccctctcccacctccgcagcctcatcagcctgcatctgaagcatctcaatatcaacaatatgcctgtgtatgcctttaaaagattgttccacctgaaacacctagagattgactattggcctttactggatatgatgcctgccaatagcctctacggtctcaacctcacatccctttcagtcaccaacaccaatctgtctactgtacccttccttgcctttaaacacctggtatacctgactcaccttaacctctcctacaatcccatcagcactattgaagcaggcatgttctctgacctgatccgccttcaggagcttcatatagtgggggcccagcttcgcaccattgagcctcactccttccaagggctccgcttcctacgcgtgctcaatgtgtctcagaacctgctggaaactttggaagagaatgtcttctcctcccctagggctctggaggtcttgagcattaacaacaaccctctggcctgtgactgccgccttctctggatcttgcagcgacagcccaccctgcagtttggtggccagcaacctatgtgtgctggcccagacaccatccgtgagaggtctttcaaggatttccatagcactgccctttctttttactttacctgcaaaaaacccaaaatccgtgaaaagaagttgcagcatctgctagtagatgaagggcagacagtccagctagaatgcagtgcagatggagacccgcagcctgtgatttcctgggtgacaccccgaaggcgtttcatcaccaccaagtccaatggaagagccaccgtgttgggtgatggcaccttggaaatccgctttgcccaggatcaagacagcgggatgtatgtttgcatcgctagcaatgctgctgggaatgataccttcacagcctccttaactgtgaaaggattcgcttcagatcgttttctttatgcgaacaggacccctatgtacatgaccgactccaatgacaccatttccaatggcagcaatgccaatactttttccctggaccttaaaacaatactggtgtctacagctatgggctgcttcacattcctgggagtggttttattttgttttcttctcctttttgtgtggagccgagggaaaggcaagcacaaaaacagcattgaccttgagtatgtgcccaaaaaaaaccatggtgctgttgtggaaggggaggtagctggacccaggaggttcaacatgaaaatgatttgaaggcccacccctcacattactgtctctttgtcaatgtgggtaatcagtaagacagtatggcacagtaaattactagattaagaggcagccatgtgcagctgcccctgtatcaaaagcagggtctatggaagcaggaggacttccaatggagactctccatcgaaaggcaggcaggcaggcatgtgtcagagcccttcacacagtgggatactaagtgtttgcgttgcaaatattggcgttctggggatctcagtaatgaacctgaatatttggctcacactcacggacaattattcagcattttctaccactgcaaaaaaaaa

SEQ ID NO: 16 - protein, 606 aa, Homo sapiens
MLHTAISCWQPFLGLAVVLIFMGSTIGCPARCECSAQNKSVSCHRRRLIAIPEGIPIETKILDLSKNRLKSVNPEEFISYPLLEEIDLSDNIIANVEPGAFNNLFNLRSLRLKGNRLKLVPLGVFTGLSNLTKLDISENKIVILLDYMFQDLHNLKSLEVGDNDLVYISHRAFSGLLSLEQLTLEKCNLTAVPTEALSHLRSLISLHLKHLNINNMPVYAFKRLFHLKHLEIDYWPLLDMMPANSLYGLNLTSLSVTNTNLSTVPFLAFKHLVYLTHLNLSYNPISTIEAGMFSDLIRLQELHIVGAQLRTIEPHSFQGLRFLRVLNVSQNLLETLEENVFSSPRALEVLSINNNPLACDCRLLWILQRQPTLQFGGQQPMCAGPDTIRERSFKDFHSTALSFYFTCKKPKIREKKLQHLLVDEGQTVQLECSADGDPQPVISWVTPRRRFITTKSNGRATVLGDGTLEIRFAQDQDSGMYVCIASNAAGNDTFTASLTVKGFASDRFLYANRTPMYMTDSNDTISNGSNANTFSLDLKTILVSTAMGCFTFLGVVLFCFLLLFVWSRGKGKHKNSIDLEYVPKKNHGAVVEGEVAGPRRFNMKMI

SEQ ID NO: 17 - DNA, 2187 bp, Homo sapiens
aatcatgaggaacctataacccttttggccacatgcaaaaaagcaagacccgtgaccaaggtgtagactaagaagtggagtcatgcttcacacggccatatcatgctggcagccattcctgggtctggctgtggtgttaatcttcatgggacccaccattggctgccccgctcgctgtgagtgctctgcccagaacaaatctgttagctgtcacagaaggcgattgatcgccatcccagagggcattcccatcgaaaccaaaatcttgaacctcagtaaaaacaggctaaaaagcgtcaaccctgaagaattcatatcatatcctctgctggaagagatagacttgagtgacaacatcattgccaatgtggaaccaggagcattcaacaatctctttaacctgcgttccctccgcctaaaaggcaatcgtctaaagctggtccctttgggagtattcacggggctgtccaatctcactaagcttgacattagtgagaataagattgtcattttactagactacatgttccaagatctacataacctgaagtctctagaagtgggggacaatgatttggtttatatatcacacagggcattcagtgggcttcttagcttggagcagctcaccctggagaaatgcaacttaacagcagtaccaacagaagccctctcccacctccgcagcctcatcagcctgcatctgaagcatctcaatatcaacaatatgcctgtgtatacctttaaaagattgttccacctgaaacacctagagattgactattggcctttactggatatgatgcctgccaatagcctctacggtctcaacctcacacccctttcagtcaccaacaccaatctgtctactgtacccttccttgcctttaaacacctggtatacctgactcaccttaacctctcctacaatcccatcagcactattgaagcaggcatgttctctgacctgatccgccttcaggagcttcatatagtgggggcccagcttcgcaccattgagcctcactccttccaagggctccgcttcctacgcgtgctcaatgtgtctcagaacctgctggaaactttggaagagaatgtcttctcctcccctagggctctggaggtcttgagcattaacaacaaccctctggcctgtgactgccgccttctctggatcttgcagcgacagcccaccctgcagtttggtggccagcaacctatgtgtgctggcccagacaccatccgtgagaggtctttcaaggatttccatagcactgccctttctttttactttacctgcaaaaaacccaaaatccgtgaaaagaagttgcagcatctgctagtagatgaagggcagacagtccagctagaatgcagtgcagatggagacccgcagcctgtgatttcctgggtgacaccccgaaggcgtttcatcaccaccaagtccaatggaagagccaccgtgttgggtgatggcaccttggaaatccgctttgcccaggatcaagacagcgggatgtatgtttgcatcgctagcaatgctgctgggaatgataccttcacagcctccttaactgtgaaaggattcgcttcagatcgttttctttatgcgaacaggacccctatgtacatgaccgactccaatgacaccatttccaatggcaccaatgccaatactttttccctggaccttaaaacaatactggtgtctacagctatgggctgcttcacattcctgggagtggttttattttgttttcttctcctttttgtgtggagccgagggaaaggcaagcacaaaaacagcattgaccttgagtatgtgcccagaaaaaacagtggtgctgttgtggaaggggaggtagctggacccaggaggttcaacatgaaaatgatttgaaggcccacccctcacattactgtctctttgtcaatgtgggtaatcagtaagacagtatggcacagtaaattactagattaagaggcagccatgtgcagctgcccctgtatcaaaagcagggtctatggaagcaggaggacttccaatggagactctccatcgaaaggcaggcaggcaggcatgtgtcagagcccttcacacagtgggatactaagtgtttgcgttgcaaatattggcgttctggggatctcagtaatgaacctgaatatttggctcacactcac

SEQ ID NO: 18 - protein, 606 aa, Homo sapiens
MLHTAISCWQPFLGLAVVLIFMGPTIGCPARCECSAQNKSVSCHRRRLIAIPEGIPIETKILNLSKNRLKSVNPEEFISYPLLEEIDLSDNIIANVEPGAFNNLFNLRSLRLKGNRLKLVPLGVFTGLSNLTKLDISENKIVILLDYMFQDLHNLKSLEVGDNDLVYISHRAFSGLLSLEQLTLEKCNLTAVPTEALSHLRSLISLHLKHLNINNMPVYTFKRLFHLKHLEIDYWPLLDMMPANSLYGLNLTPLSVTNTNLSTVPFLAFKHLVYLTHLNLSYNPISTIEAGMFSDLIRLQELHIVGAQLRTIEPHSFQGLRFLRVLNVSQNLLETLEENVFSSPRALEVLSINNNPLACDCRLLWILQRQPTLQFGGQQPMCAGPDTIRERSFKDFHSTALSFYFTCKKPKIREKKLQHLLVDEGQTVQLECSADGDPQPVISWVTPRRRFITTKSNGRATVLGDGTLEIRFAQDQDSGMYVCIASNAAGNDTFTASLTVKGFASDRFLYANRTPMYMTDSNDTISNGTNANTFSLDLKTILVSTAMGCFTFLGVVLFCFLLLFVWSRGKGKHKNSIDLEYVPRKNSGAVVEGEVAGPRRFNMKMI

SEQ ID NO: 19 - DNA, 1215 bp, Homo sapiens
gctcctttcttccctctccagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgaaggtggaactgcagcactgtggataacacctctgtttttggcagggtgatgcagataggtagccgcgagacggccttcacatacgcggtgagcgcagcaggggtggtgaacgccatgagccgggcgtgccgcgagggcgagctgtccacctgcggctgcagccgcgccgcgcgccccaaggacctgccgcgggactggctctggggcggctccggcgccaccaacaaaaaaggctaccgctccgccaaggagatcgtgcacgcccgcgaacgaggacgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtggctacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgctcccaggacccgcttatttatagaaagtac

SEQ ID NO: 20 - protein, 380 aa, Homo sapiens
LQKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGSGATNKKGYRSAKEIVHARERGRIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 21 - DNA, 4113 bp, Homo sapiens
attaattctggctccacttgttgctcggcccaggttggggagaggacggagggtggccgcagcgggttcctgagtgaattacccaggagggactgagcacagcaccaactagagaggggtcagggggtgcgggactcgagcgagcaggaaggaggcagcgcctggcaccagggctttgactcaacagaattgagacacgtttgtaatcgctggcgtgccccgcgcacaggatcccagcgaaaatcagatttcctggtgaggttgcgtgggtggattaatttggaaaaagaaactgcctatatcttgccatcaaaaaactcacggaggagaagcgcagtcaatcaacagtaaacttaagagacccccgatgctcccctggtttaacttgtatgcttgaaaattatctgagagggaataaacatcttttccttcttccctctccagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgacggtggaactgcagcacagcggataacacctctgtctttgggagagtcatgcagataggcagccgcgagacggccttcacccacgcggtgagcgccgcgggcgtggtcaacgccatcagccgggcctgccgcgagggcgagctctccacctgcggctgcagccggacggcgcggcccaaggacctgccccgggactggctgtggggcggctgtggggacaacgtggagtacggctaccgcttcgccaaggagtttgtggacgcccgggagcgagagaagaactttgccaaaggatcagaggagcagggccgggtgctcatgaacctgcaaaacaacgaggccggtcgcagggctgtgtataagatggcagacgtagcctgcaaatgccacggcgtctcggggtcctgcagcctcaagacctgctggctgcagctggccgagttccgcaaggtcggggaccggctgaaggagaagtacgacagcgcggccgccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtgggtacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgctcccaggacccgcttatttatagaaagtacagtgattctggtttttggtttttagaaatattttttatttttccccaagaattgcaaccggaaccattttttttcctgttaccatctaagaactctgtggtttattattaatattataattattatttggcaataatgggggtgggaaccacgaaaaatatttattttgtggatctttgaaaaggtaatacaagacttcttttggatagtatagaatgaagggggaaataacacataccctaacttagctgtgtggacatggtacacatccagaaggtaaagaaatacattttctttttctcaaatatgccatcatatgggatgggtaggttccagttgaaagagggtggtagaaatctattcacaattcagcttctatgaccaaaatgagttgtaaattctctggtgcaagataaaaggtcttgggaaaacaaaacaaaacaaaacaaacctcccttccccagcagggctgctagcttgctttctgcattttcaaaatgataatttacaatggaaggacaagaatgtcatattctcaaggaaaaaaggtatatcacatgtctcattctcctcaaatattccatttgcagacagaccgtcatattctaatagctcatgaaatttgggcagcagggaggaaagtccccagaaattaaaaaatttaaaactcttatgtcaagatgttgatttgaagctgttataagaattgggattccagatttgtaaaaagacccccaatgattctggacactagattttttgtttggggaggttggcttgaacataaatgaaatatcctgtattttcttagggatacttggttagtaaattataatagtagaaataatacatgaatcccattcacaggtttctcagcccaagcaacaaggtaattgcgtgccattcagcactgcaccagagcagacaacctatttgaggaaaaacagtgaaatccaccttcctcttcacactgagccctctctgattcctccgtgttgtgatgtgatgctggccacgtttccaaacggcagctccactgggtcccctttggttgtaggacaggaaatgaaacattaggagctctgcttggaaaacagttcactacttagggatttttgtttcctaaaacttttattttgaggagcagtagttttctatgttttaatgacagaacttggctaatggaattcacagaggtgttgcagcgtatcactgttatgatcctgtgtttagattatccactcatgcttctcctattgtactgcaggtgtaccttaaaactgttcccagtgtacttgaacagttgcatttataaggggggaaatgtggtttaatggtgcctgatatctcaaagtcttttgtacataacatatatatatatatacatatatataaatataaatataaatatatctcattgcagccagtgatttagatttacagcttactctggggttatctctctgtctagagcattgttgtccttcactgcagtccagttgggattattccaaaagttttttgagtcttgagcttgggctgtggccccgctgtgatcataccctgagcacgacgaagcaacctcgtttctgaggaagaagcttgagttctgactcactgaaatgcgtgttgggttgaagatatctttttttcttttctgcctcacccctttgtctccaacctccatttctgttcactttgtggagagggcattacttgttcgttatagacatggacgttaagagatattcaaaactcagaagcatcagcaatgtttctcttttcttagttcattctgcagaatggaaacccatgcctattagaaatgacagtacttattaattgagtccctaaggaatattcagcccactacatagatagctttttttttttttttttttttttaataaggacacctctttccaaacaggccatcaaatatgttcttatctcagacttacgttgttttaaaagtttggaaagatacacatcttttcatacccccccttaggaggttgggctttcatatcacctcagccaactgtggctcttaatttattgcataatgatatccacatcagccaactgtggctctttaatttattgcataatgatattcacatcccctcagttgcagtgaattgtgagcaaaagatcttgaaagcaaaaagcactaattagtttaaaatgtcacttttttggtttttattatacaaaaaccatgaagtactttttttatttgctaaatcagattgttcctttttagtgactcatgtttatgaagagagttgagtttaacaatcctagcttttaaaagaaactatttaatgtaaaatattctacatgtcattcagatattatgtatatcttctagcctttattctgtacttttaatgtacatatttctgtcttgcgtgatttgtatatttcactggtttaaaaaacaaacatcgaaaggcttattccaaatggaag

SEQ ID NO: 22 - protein, 380 aa, Homo sapiens
LQKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTADNTSVFGRVMQIGSRETAFTHAVSAAGVVNAISRACREGELSTCGCSRTARPKDLPRDWLWGGCGDNVEYGYRFAKEFVDAREREKNFAKGSEEQGRVLMNLQNNEAGRRAVYKMADVACKCHGVSGSCSLKTCWLQLAEFRKVGDRLKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 23 - DNA, 1214 bp, Homo sapiens
ctcctttcttccctctccagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgaaggtggaactgcagcactgtggataacacctctgtttttggcagggtgatgcagataggcagccgcgagacggccttcacatacgcggtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtggctacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgcccccaggacccgcttatttatagaaagtac

SEQ ID NO: 24 - protein, 365 aa, Homo sapiens
MAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 25 - DNA, 4213 bp, Homo sapiens
atggctccactccgcgcgctgctgtcctacctgctgcctttgcactgtgcgctctgcgccgccgcgggcagccggaccccagagctgcacctctctggaaagctcagtgactatggtgtgacagtgccctgcagcacagactttcggggacgcttcctctcccacgtggtgtctggcccagcagcagcctctgcagggagcatggtagtggacacgccacccacactaccacgacactccagtcacctccgggtggctcgcagccctctgcacccaggagggaccctgtggcctggcagggtggggcgccactccctctacttcaatgtcactgttttcgggaaggaactgcacttgcgcctgcggcccaatcggaggttggtagtgccaggatcctcagtggagtggcaggaggattttcgggagctgttccggcagcccttacggcaggagtgtgtgtacactggaggtgtcactggaatgcctggggcagctgttgccatcagcaactgtgacggattgtgtgcaggccctgcgggcctcatccgcacagacagcaccgacttcttcattgagcctctggagcggggccagcaggagaaggaggccagcgggaggacacatgtggtgtaccgccgggaggccgtccagcaggactttggcctgggagaccttcccaacctgctgggcctggtgggggaccagctgggcgacacagagcggaagcggcggcatgccaagccaggcagctacagcatcgaggtgctgctggtggtggacgactcggtggttcgcttccatggcaaggagcatgtgcagaactatgtcctcaccctcatgaatatcgtgagtgtagatgagatttaccacgatgagtccctgggggttcatataaatattgccctcgtccgcttgatcatggttggctaccgacagtccctgagcctgatcgagcgcgggaacccctcacgcagcctggagcaggtgtgtcgctgggcacactcccagcagcgccaggaccccagccacgctgagcaccatgaccacgttgtgttcctcacccggcaggactttgggccctcagggtatgcacccgtcactggcatgtgtcaccccctgaggagctgtgccctcaaccatgaggatggcttctcctcagccttcgtgatagctcatgagaccggccacgtgctcggcatggagcatgacggtcaggggaatggctgtgcagatgagaccagcctgggcagcgtcatggcgcccctggtgcaggctgccttccaccgcttccattggtcccgctgcagcaagctggagctcagccgctacctcccctcctacgactgcctcctcgatgacccctttgatcctgcctggccccagcccccagagctgcctgggatcaactactcaatggatgagcagtgccgctttgactttggcagtggctaccagacctgcttggcattcaggacctttgagccctgcaagcagctgtggtgcagccatcctgacaacccgtacttctgcaagaccaagaaggggcccccgctggatgggactgagtgtgcacccggcaagtggtgcttcaaaggtcactgcatctggaagtcgccggagcagacatatggccaggatggaggctggagctcctggaccaagtttgggtcatgttcgcggtcatgtgggggcggggtgcgatcccgcagccggagctgcaacaacccctccccagcctatggaggccgcctgtgcttagggcccatgttcgagtaccaggtctgcaacagcgaggagtgccctgggacctacgaggacttccgggcccagcagtgtgccaagcgcaactcctactatgtgcaccagaatgccaagcacagctgggtgccctacgagcctgacgatgacgcccagaagtgtgagctgatctgccagtcggcggacacgggggacgtggtgttcatgaaccaggtggttcacgatgggacacgctgcagctaccgggacccatacagcgtctgtgcgcgtggcgagtgtgtgcctgtcggctgtgacaaggaggtggggtccatgaaggcggatgacaagtgtggagtctgcgggggtgacaactcccactgcaggactgtgaaggggacgctgggcaaggcctccaagcaggcaggagctctcaagctggtgcagatcccagcaggtgccaggcacatccagattgaggcactggagaagtccccccaccgcattgtggtgaagaaccaggtcaccggcagcttcatcctcaaccccaagggcaaggaagccacaagccggaccttcaccgccatgggcctggagtgggaggatgcggtggaggatgccaaggaaagcctcaagaccagcgggcccctgcctgaagccattgccatcctggctctccccccaactgagggtggcccccgcagcagcctggcctacaagtacgtcatccatgaggacctgctgccccttatcgggagcaacaatgtgctcctggaggagatggacacctatgagtgggcgctcaagagctgggccccctgcagcaaggcctgtggaggagggatccagttcaccaaatacggctgccggcgcagacgagaccaccacatggtgcagcgacacctgtgtgaccacaagaagaggcccaagcccatccgccggcgctgcaaccagcacccgtgctctcagcctgtgtgggtgacggaggagtggggtgcctgcagccggagctgtgggaagctgggggtgcagacacgggggatacagtgcctgctgcccctctccaatggaacccacaaggtcatgccggccaaagcctgcgccggggaccggcctgaggcccgacggccctgtctccgagtgccctgcccagcccagtggaggctgggagcctggtcccagtgctctgccacctgtggagagggcatccagcagcggcaggtggtgtgcaggaccaacgccaacagcctcgggcattgcgagggggataggccagacactgtccaggtctgcagcctgcccgcctgtaacaagatatcatcaacggagccctgcacgggagacaggtctgtcttctgccagatggaagtgctcgatcgctactgctccattcccggctaccaccggctctgctgtgtgtcctgcatcaagaaggcctcgggccccaaccctggcccagaccctggcccaacctcactgccccccttctccactcctggaagccccttaccaggaccccaggaccctgcagatgctgcagagcctcctggaaagccaacgggatcagaggaccatcagcatggccgagccacacagctcccaggagctctggatacaagctccccagggacccagcatccctttgcccctgagacaccaatccctggagcatcctggagcatctcccctaccacccccggggggctgccttggggctggactcagacacctacgccagtccctgaggacaaagggcaacctggagaagacctgagacatcccggcaccagcctccctgctgcctccccggtgacatgagctgtgccctgccatcccactggcacgtttacactctgtgtactgccccgtgactcccagctcagaggacacacatagcagggcaggcgcaagcacagacttcattttaaatcattcgccttcttctcgtttggggctgtgatgctctttaccccacaaagcggggtgggaggaagacaaagatcagggaaagccctaatcggagatacctcagcaagctgcccccggcgggactgaccctctcagggcccctgttggtctcccctgccaagaccagggtcaactattgctccctcctcacagaccctgggcctgggcaggtctgaatcccggctggtctgtagctagaagctgtcagggctgcctgccttcccggaactgtgaggacccctgtggaggccctgcatatttggcccctctccccagaaaggcaaagcagggccagggtaggtgggggactgttcacagccaggccgagaggaggggggcctgggaatgtggcatgaggcttcccagctgcagggctggagggggtggaacacaaggtgatcgcaggcccaactcctggaagccaagagctccatgcagttccaccagctgaggccaggcagcagaggccagtttgtctttgctggccagaagatggtgctcatggcca

SEQ ID NO: 26 - protein, 1210 aa, Homo sapiens
  VARIANT (1185): Wherein Xaa is any amino acid as defined in the specification
MAPLRALLSYLLPLHCALCAAAGSRTPELHLSGKLSDYGVTVPCSTDFRGRFLSHVVSGPAAASAGSMVVDTPPTLPRHSSHLRVARSPLHPGGTLWPGRVGRHSLYFNVTVFGKELHLRLRPNRRLVVPGSSVEWQEDFRELFRQPLRQECVYTGGVTGMPGAAVAISNCDGLCAGPAGLIRTDSTDFFIEPLERGQQEKEASGRTHVVYRREAVQQDFGLGDLPNLLGLVGDQLGDTERKRRHAKPGSYSIEVLLVVDDSVVRFHGKEHVQNYVLTLMNIVSVDEIYHDESLGVHINIALVRLIMVGYRQSLSLIERGNPSRSLEQVCRWAHSQQRQDPSHAEHHDHVVFLTRQDFGPSGYAPVTGMCHPLRSCALNHEDGFSSAFVIAHETGHVLGMEHDGQGNGCADETSLGSVMAPLVQAAFHRFHWSRCSKLELSRYLPSYDCLLDDPFDPAWPQPPELPGINYSMDEQCRFDFGSGYQTCLAFRTFEPCKQLWCSHPDNPYFCKTKKGPPLDGTECAPGKWCFKGHCIWKSPEQTYGQDGGWSSWTKFGSCSRSCGGGVRSRSRSCNNPSPAYGGRLCLGPMFEYQVCNSEECPGTYEDFRAQQCAKRNSYYVHQNAKHSWVPYEPDDDAQKCELICQSADTGDVVFMNQVVHDGTRCSYRDPYSVCARGECVPVGCDKEVGSMKADDKCGVCGGDNSHCRTVKGTLGKASKQAGALKLVQIPAGARHIQIEALEKSPHRIVVKNQVTGSFILNPKGKEATSRTFTAMGLEWEDAVEDAKESLKTSGPLPEAIAILALPPTEGGPRSSLAYKYVIHEDLLPLIGSNNVLLEEMDTYEWALKSWAPCSKACGGGIQFTKYGCRRRRDHHMVQRHLCDHKKRPKPIRRRCNQHPCSQPVWVTEEWGACSRSCGKLGVQTRGIQCLLPLSNGTHKVMPAKACAGDRPEARRPCLRVPCPAQWRLGAWSQCSATCGEGIQQRQVVCRTNANSLGHCEGDRPDTVQVCSLPACNKISSTEPCTGDRSVFCQMEVLDRYCSIPGYHRLCCVSCIKKASGPNPGPDPGPTSLPPFSTPGSPLPGPQDPADAAEPPGKPTGSEDHQHGRATQLPGALDTSSPGTQHPFAPETPIPGASWSISPTTPGGLPWGWTQTPTPVPEDKGQPGEDLRHPGTSLPAASPVTXAVPCHPTGTFTLCVLPRDSQLRGHT

SEQ ID NO: 27 - DNA, 1390 bp, Homo sapiens
cttgagtggccaaggcaagatgggtcaaagtcaaagtggtggtcatggtcttggagctggaaagaaggatgatagggacaagaaaaagaaatatgaacctcctataccagctagagtgaggaagaagaagaaaacaaagggaccagatgctgccagcaaactgccactgatgacacctcacactctgtgccagttaaaattattgaaattagagataattaaatactgtcttctcatgaaggaagaattcattagaaatcaggaacaaatgaaactattagaaggaaagcaagaggaggaaagatcaaaagtggatgatctgagggggacccccatgtcagtagtaaccttggaagagattattgatgacaatcatgccatcatgtctacatctgtgggctcagagcatctgtgggctcagagcattcttgtagacaaggatctgctggaacctggctgctcggtcctgctcaaccacaaggttcgtgctgtgatatgggtgctgatggatgacacggataccctagtcacaatgatgaaggtggaaaagaccccccaggagacctgtgttgatactggggggttggacagccaaattcaggaaattaaggaatttgtggagcttcctctcacacattctgaatattatgaagagatgggtataaagccccctaagggagtcattcactatggtccacctggcacaggtaaaaccttgttagccaaagcagtagcaaaccacatcttagccactttcttgcaagtgatcagctctgaatttattcagaaatacctacatgatgggcccaaactcatatgggaattgtttctagttgctgaagaacatgcaccttccatcatgtttattgatgaaattgatgctattaggacaaaaagatgtgactcaaattctgatagtgagagagaaattcagcaaataatgctggaaatgttgaaccagttggatggatttgattcaaggggagatgtgaaagttatcatatccacaagccgaatagaaactttggatctagcacttatcagaccaggctacactgacaggaagctcaagttccccctgcctgatgaaaagactaagaagcacatctttcagatgcacacaagcaggattacgctggccaatgatacaatcctggacaactccatcatggctaaagatgacctctcttgtacagacctcaaggcaatctgcacagaagctagtctgatggccttaaaagaacatggaatgaaagtaacaaatgaaaacttcaaaaaatctcaagaaaatgttctttataaagaacaggaagacacccccaaggggctctgtctcggaagcaagagaaagaaggggaaggggccagactcattttaacaaccagatatt

SEQ ID NO: 28 - protein, 452 aa, Homo sapiens
MGQSQSGGHGLGAGKKDDRDKKKKYEPPIPARVRKKKKTKGPDAASKLPLMTPHTLCQLKLLKLEIIKYCLLMKEEFIRNQEQMKLLEGKQEEERSKVDDLRGTPMSVVTLEEIIDDNHAIMSTSVGSEHLWAQSILVDKDLLEPGCSVLLNHKVRAVIWVLMDDTDTLVTMMKVEKTPQETCVDTGGLDSQIQEIKEFVELPLTHSEYYEEMGIKPPKGVIHYGPPGTGKTLLAKAVANHILATFLQVISSEFIQKYLHDGPKLIWELFLVAEEHAPSIMFIDEIDAIRTKRCDSNSDSEREIQQIMLEMLNQLDGFDSRGDVKVIISTSRIETLDLALIRPGYTDRKLKFPLPDEKTKKHIFQMHTSRITLANDTILDNSIMAKDDLSCTDLKAICTEASLMALKEHGMKVTNENFKKSQENVLYKEQEDTPKGLCLGSKRKKGKGPDSF

SEQ ID NO: 29 - DNA, 22 bp, Artificial Sequence
  Description of Artificial Sequence Oligonucleotide primers
ctgcacttcaaggacagttacc

SEQ ID NO: 30 - DNA, 25 bp, Artificial Sequence
  Description of Artificial Sequence Oligonucleotide primers
ctatccatccacgatgtgcccagct

SEQ ID NO: 31 - DNA, 22 bp, Artificial Sequence
  Description of Artificial Sequence Oligonucleotide primers
tgacaaggagcttactcttcca

SEQ ID NO: 32 - DNA, 19 bp, Artificial Sequence
  Description of Artificial Sequence Oligonucleotide primers
ccgttcactcttgcaaagg

SEQ ID NO: 33 - DNA, 28 bp, Artificial Sequence
  Description of Artificial Sequence Oligonucleotide primers
tccaagggattcacaactacttacacca

SEQ ID NO: 34 - DNA, 22 bp, Artificial Sequence
  Description of Artificial Sequence Oligonucleotide primers
ggcacagttgctataattttgg

SEQ ID NO: 35 - DNA, 20 bp, Artificial Sequence
  Description of Artificial Sequence Oligonucleotide primers
ctcctggactccctctatgg

SEQ ID NO: 36 - DNA, 26 bp, Artificial Sequence
  Description of Artificial Sequence Oligonucleotide primers
ctctcggtggtgcagctcaatccttt

SEQ ID NO: 37 - DNA, 20 bp, Artificial Sequence
  Description of Artificial Sequence Oligonucleotide primers
gggcctttaccaactctgaa

SEQ ID NO: 38 - DNA, 22 bp, Artificial Sequence
  Description of Artificial Sequence Oligonucleotide primers
gacctcagatgtcctagccaat

SEQ ID NO: 39 - DNA, 26 bp, Artificial Sequence
  Description of Artificial Sequence Oligonucleotide primers
cacctacctgaaaggagagctgcctg

SEQ ID NO: 40 - DNA, 22 bp, Artificial Sequence
  Description of Artificial Sequence Oligonucleotide primers
ccaggaaacactcactcacatt

SEQ ID NO: 41 - DNA, 22 bp, Artificial Sequence
  Description of Artificial Sequence Oligonucleotide primers
ccagaggatccagatgtacatg

SEQ ID NO: 42 - DNA, 30 bp, Artificial Sequence
  Description of Artificial Sequence Oligonucleotide primers
tcctgtctctcatcctctacatcttcacca

SEQ ID NO: 43 - DNA, 22 bp, Artificial Sequence
  Description of Artificial Sequence Oligonucleotide primers
gggctccagagaagatgtctac

SEQ ID NO: 44 - DNA, 22 bp, Artificial Sequence
  Description of Artificial Sequence Oligonucleotide primers
ccagaggatccagatgtacatg

SEQ ID NO: 45 - DNA, 27 bp, Artificial Sequence
  Description of Artificial Sequence Oligonucleotide primers
tcctctacatcttcaccaagatctcgg

SEQ ID NO: 46 - DNA, 22 bp, Artificial Sequence
  Description of Artificial Sequence Oligonucleotide primers
agggctccagagaagatgtcta

SEQ ID NO: 47 - DNA, 22 bp, Artificial Sequence
  Description of Artificial Sequence Oligonucleotide primers
ctggtcaggtacctggatgtta

SEQ ID NO: 48 - DNA, 26 bp, Artificial Sequence
  Description of Artificial Sequence Oligonucleotide primers
tccatcaatgaagagcttcatattcg

SEQ ID NO: 49 - DNA, 22 bp, Artificial Sequence
  Description of Artificial Sequence Oligonucleotide primers
cagcctttaagtgatccatcag

SEQ ID NO: 50 - DNA, 21 bp, Artificial Sequence
  Description of Artificial Sequence Oligonucleotide primers
ttgaagaaggcagaaacacaa

SEQ ID NO: 51 - DNA, 26 bp, Artificial Sequence
  Description of Artificial Sequence Oligonucleotide primers
ccgccttcaagagaaacaaacgaaag

SEQ ID NO: 52 - DNA, 20 bp, Artificial Sequence
  Description of Artificial Sequence Oligonucleotide primers
cgcagctcacagctcattat

SEQ ID NO: 53 - DNA, 22 bp, Artificial Sequence
  Description of Artificial Sequence Oligonucleotide primers
caatatgcctgtgtatgccttt

SEQ ID NO: 54 - DNA, 26 bp, Artificial Sequence
  Description of Artificial Sequence Oligonucleotide primers
aaaagattgttccacctgaaacacct

SEQ ID NO: 55 - DNA, 22 bp, Artificial Sequence
  Description of Artificial Sequence Oligonucleotide primers
tccagtaaaggccaatagtcaa

SEQ ID NO: 56 - DNA, 22 bp, Artificial Sequence
  Description of Artificial Sequence Oligonucleotide primers
acagcagtaccaacagaagccc

SEQ ID NO: 57 - DNA, 22 bp, Artificial Sequence
  Description of Artificial Sequence Oligonucleotide primers
tcccacctccgcagcctcatca

SEQ ID NO: 58 - DNA, 24 bp, Artificial Sequence
  Description of Artificial Sequence Oligonucleotide primers
atattgacatgcttcagatgcagg

SEQ ID NO: 59 - DNA, 22 bp, Artificial Sequence
  Description of Artificial Sequence Oligonucleotide primers
ccaagttcttcctagtggcttt

SEQ ID NO: 60 - DNA, 26 bp, Artificial Sequence
  Description of Artificial Sequence Oligonucleotide primers
tttctccttcgcccaggttgtaattg

SEQ ID NO: 61 - DNA, 22 bp, Artificial Sequence
  Description of Artificial Sequence Oligonucleotide primers
atacctagcgaccaccaagaat